(19) United States
(12) Patent Application Publication
(10) Pub. No.: US 2025/0264464 A1
(43) Pub. Date: Aug. 21, 2025

(54) COMPOSITIONS AND METHODS FOR ANALYTE DETECTION USING BIOLUMINESCENCE

(71) Applicant: Promega Corporation, Madison, WI (US)

(72) Inventors: Thomas Kirkland, Madison, WI (US); Melanie Dart, Madison, WI (US); Zhiyang Zeng, Madison, WI (US); Thomas Smith, Madison, WI (US); Keith Wood, Madison, WI (US); Thomas Machleidt, Madison, WI (US); Virginia Kincaid, Madison, WI (US); Hui Wang, Madison, WI (US); Wenhui Zhou, Madison, WI (US); Emily Jost, Madison, WI (US); Lance Encell, Madison, WI (US); Mary Hall, Madison, WI (US); Robin Hurst, Madison, WI (US)

(21) Appl. No.: 19/072,777

(22) Filed: Mar. 6, 2025

Related U.S. Application Data
(63) Continuation of application No. 16/845,802, filed on Apr. 10, 2020, now abandoned.
(60) Provisional application No. 62/832,052, filed on Apr. 10, 2019.

(51) Int. Cl. G01N 33/543; C12Q 1/66

(52) U.S. Cl. CPC G01N 33/54388; G01N 33/54306; G01N 33/54366; C12Q 1/66; C12Q 2563/103

(57) ABSTRACT

Provided herein are systems and methods for the detection of an analyte or analytes in a sample. In particular, the present disclosure provides compositions, assays, and methods for detecting and/or quantifying a target analyte using a bioluminescent complex comprising substrates, peptides, and/or polypeptides capable of generating a bioluminescent signal that correlates to the presence, absence, or amount of the target analyte.

### CROSS REFERENCE TO RELATED APPLICATIONS

The present application is a continuation of U.S. patent application Ser. No. 16/845,802, filed Apr. 10, 2020, which claims priority to and the benefit of U.S. Provisional Patent Application No. 62/832,052, filed Apr. 10, 2019, which is incorporated herein by reference in its entirety and for all purposes.

### SEQUENCE LISTING

The text of the computer readable sequence listing filed herewith, titled “PRMG-35851_303_SequenceListing.xml”, created Mar. 6, 2025, having a file size of 993,616 bytes, is hereby incorporated by reference in its entirety.

### FIELD

Provided herein are systems and methods for the detection of an analyte or analytes in a sample. In particular, the present disclosure provides compositions, assays, and methods for detecting and/or quantifying a target analyte using a bioluminescent complex comprising substrates, peptides, and/or polypeptides capable of generating a bioluminescent signal that correlates to the presence, absence, or amount of the target analyte.

### BACKGROUND

Biological processes rely on covalent and non-covalent interactions between molecules, macromolecules, and molecular complexes. In order to understand such processes, and to develop techniques and compounds to manipulate them for research and clinical and other practical applications, it is necessary to have tools available to detect and monitor these interactions and/or components involved in such interactions. The study of these interactions, particularly under physiological conditions (e.g., at normal expression levels for monitoring protein interactions), requires high sensitivity.

Creation of better assays for use in the field and in clinical settings is an ongoing area of urgent need. Speed, sensitivity, selectivity, robustness, simplicity, quantitative versus qualitative capabilities, and cost are all critical factors affecting the relevance of a diagnostic bioassays, and thus their utility to and adoption by the relevant community. Rapid diagnostic tests are not only relevant to clinical settings, but also can be applied to environmental, industrial, and direct to consumer contexts.

### SUMMARY

Provided herein are compositions and formulations comprising a luminogenic substrate and a target analyte binding agent comprising a target analyte binding element and one of a polypeptide component of a bioluminescent complex, or a peptide component of a bioluminescent complex.

In accordance with these embodiments, the polypeptide component of the target analyte binding agent comprises at least 60% sequence identity with SEQ ID NO: 5; at least 60% sequence identity with SEQ ID NO: 9; or at least 60% sequence identity with SEQ ID NO: 12.

In some embodiments, the peptide component of the target analyte binding agent comprises at least 60% sequence identity with SEQ ID NO: 10; at least 60% sequence identity with SEQ ID NO: 11; at least 60% sequence identity with SEQ ID NO: 13; or at least 60% sequence identity with SEQ ID NO: 14.

In some embodiments, the composition comprises a complementary peptide or polypeptide component of the bioluminescent complex, wherein the target analyte binding agent and the complementary peptide or polypeptide component of the bioluminescent complex form a bioluminescent analyte detection complex in the presence of a target analyte.

In some embodiments, the composition that comprises the luminogenic substrate and the target analyte binding agent are combined in a dried formulation, and the complementary peptide or polypeptide component of the bioluminescent complex comprises a liquid formulation, wherein the liquid formulation is added to the dried formulation and forms the bioluminescent analyte detection complex in the presence of the target analyte upon rehydration.

In some embodiments, the composition comprising the luminogenic substrate, the target analyte binding agent, and the complementary peptide or polypeptide component of the bioluminescent complex are combined in a dried formulation, wherein the dried formulation forms the bioluminescent analyte detection complex in the presence of the target analyte upon rehydration.

In some embodiments, the complementary peptide or polypeptide component comprises a second target analyte binding element that forms the bioluminescent analyte detection complex in the presence of the target analyte.

In some embodiments, the polypeptide component of the target analyte binding agent comprises at least 60% sequence identity with SEQ ID NO: 6, and wherein the complementary peptide or polypeptide component of the bioluminescent complex comprises at least 60% sequence identity with SEQ ID NO: 10.

In some embodiments, the polypeptide component of the target analyte binding agent comprises at least 60% sequence identity with SEQ ID NO: 6, and wherein the complementary peptide or polypeptide component of the bioluminescent complex comprises at least 60% sequence identity with SEQ ID NO: 14.

Embodiments of the present disclosure also include a composition comprising a dried formulation comprising (a) a first target analyte binding agent comprising a first target analyte binding element and a polypeptide component having at least 60% sequence identity with SEQ ID NO: 9, and (b) a second target analyte binding agent comprising a second target analyte binding element and a complementary peptide component having at least 60% sequence identity with SEQ ID NO: 10.

In some embodiments, the dried formulation further comprises a luminogenic substrate.

In some embodiments, the composition further comprises a liquid formulation comprising the target analyte.

Embodiments of the present disclosure also include a composition comprising a dried formulation comprising (a) a first target analyte binding agent comprising a first target analyte binding element and a polypeptide component having at least 60% sequence identity with SEQ ID NO: 12, and (b) a second target analyte binding agent comprising a second target analyte binding element and a complementary peptide component having at least 60% sequence identity with SEQ ID NO: 14.

In some embodiments, the dried formulation further comprises a luminogenic substrate.

In some embodiments, the composition further comprises a liquid formulation comprising the target analyte.

Embodiments of the present disclosure also include a composition comprising a dried formulation comprising (a) a first target analyte binding agent comprising a first target analyte binding element and a peptide component having at least 60% sequence identity with SEQ ID NO: 13, (b) a second target analyte binding agent comprising a second target analyte binding element and a complementary peptide component having at least 60% sequence identity with SEQ ID NO: 15, and (c) a complementary polypeptide component having at least 60% sequence identity with SEQ ID NO: 12.

In some embodiments, the dried formulation further comprises a luminogenic substrate.

In some embodiments, the composition further comprises a liquid formulation comprising the target analyte.

Embodiments of the present disclosure also include a composition comprising (a) a dried formulation comprising a first target analyte binding agent comprising a target analyte binding element and a polypeptide component having at least 60% sequence identity with SEQ ID NO: 9, and (b) a liquid formulation comprising a second target analyte binding agent comprising a target analyte binding element and a complementary peptide component having at least 60% sequence identity with SEQ ID NO: 10 or SEQ ID NO: 11.

Embodiments of the present disclosure also include a composition comprising (a) a dried formulation comprising a first target analyte binding agent comprising a target analyte binding element and a peptide component having at least 60% sequence identity with SEQ ID NO: 10 or SEQ ID NO: 11, and (b) a liquid formulation comprising a second target analyte binding agent comprising a target analyte binding element and a complementary polypeptide component having at least 60% sequence identity with SEQ ID NO: 9.

Embodiments of the present disclosure also include a composition comprising (a) a dried formulation comprising a first target analyte binding agent comprising a target analyte binding element and a polypeptide component having at least 60% sequence identity with SEQ ID NO: 12, and (b) a liquid formulation comprising a second target analyte binding agent comprising a target analyte binding element and a complementary peptide component having at least 60% sequence identity with SEQ ID NO: 14.

In some embodiments, the dried formulation further comprises a luminogenic substrate.

In some embodiments, the liquid formulation further comprises a luminogenic substrate.

In some embodiments, the liquid formulation further comprises a sample comprising a target analyte, and wherein a bioluminescent analyte detection complex forms upon combining the dried formulation and the liquid formulation in the presence of the target analyte.

In some embodiments, the composition further comprises a second complementary peptide or polypeptide component of the bioluminescent complex, wherein the target analyte binding agent, the first complementary peptide or polypeptide component of the bioluminescent complex, and the second complementary peptide or polypeptide component of the bioluminescent complex form a bioluminescent analyte detection complex in the presence of a target analyte.

In some embodiments, the composition comprising the target analyte binding agent comprises a dried formulation, and wherein the first complementary peptide or polypeptide component and the second complementary peptide or polypeptide of the bioluminescent complex comprise a liquid formulation; wherein the liquid formulation is added to the dried formulation and forms the bioluminescent analyte detection complex in the presence of the target analyte upon rehydration.

In some embodiments, the composition comprising the target analyte binding agent, and either the first or the second complementary peptide or polypeptide component are combined in a dried formulation, and wherein the first or the second complementary peptide or polypeptide component that is not present in the dried formulation comprises a liquid formulation; wherein the liquid formulation is added to the dried formulation and forms the bioluminescent analyte detection complex in the presence of the target analyte upon rehydration.

In some embodiments, the target analyte binding agent, the first complementary peptide or polypeptide component, and the second complementary peptide or polypeptide component are combined in a dried formulation that forms the bioluminescent analyte detection complex in the presence of the target analyte upon rehydration.

In some embodiments, the dried formulation further comprises a luminogenic substrate.

In some embodiments, the liquid formulation further comprises a luminogenic substrate.

In some embodiments, the liquid formulation further comprises a sample comprising a target analyte, and wherein a bioluminescent analyte detection complex forms upon combining the dried formulation and the liquid formulation in the presence of the target analyte.

In some embodiments, either the first or the second complementary peptide or polypeptide component comprises a second target analyte binding element that forms the bioluminescent analyte detection complex in the presence of the target analyte upon rehydration.

In some embodiments, the polypeptide component of the target analyte binding agent comprises at least 60% sequence identity with SEQ ID NO: 6, and wherein either the first or the second complementary peptide or polypeptide component of the bioluminescent complex comprises at least 60% sequence identity with either SEQ ID NO: 13 or SEQ ID NO: 15.

Embodiments of the present disclosure also include a composition comprising (a) a dried formulation comprising a first target analyte binding agent comprising a target analyte binding element and a polypeptide component having at least 60% sequence identity with SEQ ID NO: 6, and (b) a liquid formulation comprising a second target analyte binding agent comprising a target analyte binding element and a complementary peptide component having at least 60% sequence identity with SEQ ID NO: 13 or SEQ ID NO: 15, and a second complementary peptide component having at least 60% sequence identity with SEQ ID NO: 13 or SEQ ID NO: 15.

Embodiments of the present disclosure also include (a) a dried formulation comprising a first target analyte binding agent comprising a target analyte binding element and a polypeptide component having at least 60% sequence identity with SEQ ID NO: 6, and a second target analyte binding agent comprising a target analyte binding element and a complementary peptide component having at least 60% sequence identity with SEQ ID NO: 13 or SEQ ID NO: 15, and (b) a liquid formulation comprising a second complementary peptide component having at least 60% sequence identity with SEQ ID NO: 13 or SEQ ID NO: 15.

Embodiments of the present disclosure also include (a) a dried formulation comprising a first target analyte binding agent comprising a target analyte binding element and a polypeptide component having at least 60% sequence identity with SEQ ID NO: 6, and complementary peptide component having at least 60% sequence identity with SEQ ID NO: 13 or SEQ ID NO: 15, and (b) a liquid formulation comprising a second target analyte binding agent comprising a target analyte binding element and a complementary peptide component having at least 60% sequence identity with SEQ ID NO: 13 or SEQ ID NO: 15.

Embodiments of the present disclosure also include (a) a dried formulation comprising a first target analyte binding agent comprising a target analyte binding element and a peptide component having at least 60% sequence identity with SEQ ID NO: 13, and a second target analyte binding agent comprising a target analyte binding element and a complementary peptide component having at least 60% sequence identity with SEQ ID NO: 15, and (b) a liquid formulation comprising a complementary polypeptide component having at least 60% sequence identity with SEQ ID NO: 6.

Embodiments of the present disclosure also include (a) a dried formulation comprising a complementary polypeptide component having at least 60% sequence identity with SEQ ID NO: 6, and (b) a liquid formulation comprising a first target analyte binding agent comprising a target analyte binding element and a peptide component having at least 60% sequence identity with SEQ ID NO: 13, and a second target analyte binding agent comprising a target analyte binding element and a complementary peptide component having at least 60% sequence identity with SEQ ID NO: 15.

Embodiments of the present disclosure also include a composition comprising a dried formulation comprising a first target analyte binding agent comprising a target analyte binding element and a peptide component having at least 60% sequence identity with SEQ ID NO: 13, a second target analyte binding agent comprising a target analyte binding element and a complementary peptide component having at least 60% sequence identity with SEQ ID NO: 15, and a complementary polypeptide component having at least 60% sequence identity with SEQ ID NO: 6.

In some embodiments, the dried formulation further comprises a luminogenic substrate.

In some embodiments, the liquid formulation further comprises a luminogenic substrate.

In some embodiments, the liquid formulation further comprises a sample comprising a target analyte, and wherein a bioluminescent analyte detection complex forms upon combining the dried formulation and the liquid formulation in the presence of the target analyte.

In some embodiments, a bioluminescent signal produced in the presence of the luminogenic substrate is substantially increased when the target analyte binding agent contacts one or more of the complementary peptide or polypeptide components of the bioluminescent complex, as compared to a bioluminescent signal produced by the target analyte binding agent and the luminogenic substrate alone.

In some embodiments, the target analyte is a target antibody.

In some embodiments, the target analyte binding agent comprises an element that binds non-specifically to antibodies.

In some embodiments, the target analyte binding agent comprises an element that binds specifically to an antibody.

In some embodiments, the target antibody is an antibody against a pathogen, toxin, or therapeutic biologic.

In some embodiments, a target analyte binding element is selected from the group consisting of an antibody, a polyclonal antibody, a monoclonal antibody, a recombinant antibody, an antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, an oligonucleotide probe, a peptide nucleic acid, a DARPin, an aptamer, an affimer, a protein domain, and a purified protein.

In some embodiments, the luminogenic substrate is selected from coelenterazine, coelenterazine-h, coelenterazine-h-h, furimazine, JRW-0238, JRW-1404, JRW-1482, JRW, 1667, JRW-1743, JRW-1744, and other coelenterazine analogs or derivatives.

In some embodiments, the composition further comprises a polymer.

In some embodiments, the polymer is a naturally-occurring biopolymer. In some embodiments, the naturally-occurring biopolymer is selected from pullulan, trehalose, maltose, cellulose, dextran, and a combination of any thereof. In some embodiments, the naturally-occurring biopolymer is pullulan.

In some embodiments, the polymer is a cyclic saccharide polymer or a derivative thereof. In some embodiments, the polymer is hydroxypropyl P-cyclodextrin.

In some embodiments, the polymer is a synthetic polymer. In some embodiments, the synthetic polymer is selected from polystyrene, poly(meth)acrylate, and a combination of any thereof. In some embodiments, the synthetic polymer is a block copolymer comprising at least one poly(propylene oxide) block and at least one poly(ethylene oxide) block. In some embodiments, the synthetic polymer is poloxamer 188.

In some embodiments, the composition further comprises a substance to reduce autoluminescence.

In some embodiments, the substance to reduce autoluminescence is ATT (6-Aza-2-thiothymine), a derivative or analog of ATT, a thionucleoside, thiourea, and the like.

In some embodiments, the composition further comprises a buffer, a surfactant, a reducing agent, a salt, a radical scavenger, a chelating agent, a protein, or any combination thereof. In some embodiments, the is surfactant selected from polysorbate 20, polysorbate 40, and polysorbate 80.

In some embodiments, the composition is used in conjunction with an analyte detection platform to detect an analyte in a sample.

In some embodiments, sample is selected from blood, serum, plasma, urine, stool, cerebral spinal fluid, interstitial fluid, saliva, a tissue sample, a water sample, a soil sample, a plant sample, a food sample, a beverage sample, an oil, and an industrial fluid sample.

Embodiments of the present disclosure also include a method of detecting an analyte in a sample comprising combining any of the compositions described above with a sample comprising a target analyte.

In some embodiments, detecting the target analyte in the sample comprises detecting a bioluminescent signal generated from an analyte detection complex.

In some embodiments, the method further comprises quantifying a bioluminescent signal generated from the analyte detection complex.

In some embodiments, the bioluminescent signal generated from the analyte detection complex is proportional to the concentration of the analyte.

In some embodiments, one or more of the components of the composition exhibits enhanced stability within the composition compared to the component in solution alone.

Embodiments of the present disclosure also include systems and methods for the detection of an analyte or analytes in a sample. In particular, the present disclosure provides compositions, assays, and methods for detecting and/or quantifying a target analyte using a bioluminescent complex comprising substrates, peptides, and/or polypeptides capable of generating a bioluminescent signal that correlates to the presence, absence, or amount of the target analyte.

Embodiments of the present disclosure include a lateral flow detection system. In accordance with these embodiments, the system includes an analytical membrane that includes a detection region and a control region. In some embodiments, the detection region includes a first target analyte binding agent immobilized to the detection region, a conjugate pad comprising a second target analyte binding agent, and a sample pad. In some embodiments, the first target analyte binding agent and the second target analyte binding agent form a bioluminescent analyte detection complex in the at least one detection region when a target analyte is detected in a sample.

In some embodiments, the first target analyte binding agent includes a target analyte binding element and is non-luminescent. In some embodiments, the second target analyte binding agent includes a target analyte binding element and a bioluminescent polypeptide. In some embodiments, the bioluminescent polypeptide has at least 60% sequence identity with SEQ ID NO: 5.

In some embodiments, the first target analyte binding agent includes a target analyte binding element and a polypeptide component of a bioluminescent complex, and the second target analyte binding agent includes a target analyte binding element and a peptide component of a bioluminescent complex. In some embodiments, a bioluminescent signal produced in the presence of a luminogenic substrate is substantially increased when the first target analyte binding agent contacts the second target analyte binding agent, as compared to a bioluminescent signal produced by the first target analyte binding agent and the luminogenic substrate alone.

In some embodiments, the first target analyte binding agent includes a target analyte binding element and a peptide component of a bioluminescent complex, and the second target analyte binding agent includes a target analyte binding element and a polypeptide component of a bioluminescent complex. In some embodiments, a bioluminescent signal produced in the presence of a luminogenic substrate is substantially increased when the first target analyte binding agent contacts the second target analyte binding agent, as compared to a bioluminescent signal produced by the first target analyte binding agent and the luminogenic substrate alone.

In some embodiments, the polypeptide component of a bioluminescent complex has at least 60% sequence identity with SEQ ID NO: 6. In some embodiments, the polypeptide component of a bioluminescent complex has at least 60% sequence identity with SEQ ID NO: 10. In some embodiments, the polypeptide component of a bioluminescent complex has at least 60% sequence identity with SEQ ID NO: 12. In some embodiments, the polypeptide component of a bioluminescent complex has at least 60% sequence identity with SEQ ID NO: 14.

In some embodiments, the first target analyte binding agent includes a target analyte binding element and a first peptide component of a tripartite bioluminescent complex, and the second target analyte binding agent includes a target analyte binding element and a second peptide component of the tripartite bioluminescent complex. In some embodiments, a bioluminescent signal produced in the presence of a luminogenic substrate is substantially increased when the first target analyte binding agent contacts the second target analyte binding agent and a polypeptide component of the tripartite bioluminescent complex as compared to a bioluminescent signal produced by (i) the first target analyte binding agent, the second target analyte binding agent, and/or the polypeptide component and (ii) the luminogenic substrate alone.

In some embodiments, the first peptide component of a tripartite bioluminescent complex has at least 60% sequence identity with SEQ ID NO: 11. In some embodiments, the second first peptide component of a tripartite bioluminescent complex has at least 60% sequence identity with SEQ ID NO: 13. In some embodiments, the polypeptide component of a tripartite bioluminescent complex has at least 60% sequence identity with SEQ ID NO: 12.

In some embodiments, the target analyte is a target antibody. In some embodiments, the first target analyte binding element includes an agent that binds non-specifically to antibodies. In some embodiments, the second target analyte binding element comprises an agent that binds specifically to the target antibody. In some embodiments, the target antibody is an antibody against a pathogen, toxin, or therapeutic biologic.

In some embodiments, a target analyte binding element is selected from the group consisting of an antibody, a polyclonal antibody, a monoclonal antibody, a recombinant antibody, an antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, an oligonucleotide probe, a peptide nucleic acid, a DARPin, an aptamer, an affimer, a protein domain, and a purified protein.

In some embodiments, the system further includes a luminogenic substrate. In some embodiments, the luminogenic substrate is selected from coelenterazine, coelenterazine-h, coelenterazine-h-h, furimazine, JRW-0238, JRW-1404, JRW-1482, JRW-1667, JRW-1743, JRW-1744, and other coelenterazine analogs or derivatives. In some embodiments, the luminogenic substrate is applied to the system as part of a composition that includes the luminogenic substrate and a polymer selected from pullulan, trehalose, maltose, cellulose, dextran, polystyrene, poly(meth)acrylate, and a combination of any thereof. In some embodiments, the luminogenic substrate is applied to the system as part of a composition that includes the luminogenic substrate and a substance to reduce autoluminescence such as ATT (6-Aza-2-thiothymine), a derivative or analog of ATT, a thionucleoside, thiourea, and the like.

In some embodiments, the composition is applied to at least one of the sample pad, the conjugation pad, the detection region, and the control region.

In some embodiments, the analytical membrane includes a plurality of detection regions with each detection region comprising a distinct target analyte binding agent having distinct target analyte binding elements.

In some embodiments, the system further includes a device for detecting or quantifying bioluminescent signals from the analyte detection complex.

Embodiments of the present disclosure also include a conjugate pad comprising at least one target analyte binding agent. In accordance with these embodiments, the at least one target analyte binding agent includes a target analyte binding element and one of: a bioluminescent polypeptide comprising at least 60% sequence identity with SEQ ID NO: 5; a polypeptide comprising at least 60% sequence identity with SEQ ID NO: 9; a peptide comprising at least 60% sequence identity with SEQ ID NO: 10; a peptide comprising at least 60% sequence identity with SEQ ID NO: 11; a peptide comprising at least 60% sequence identity with SEQ ID NO: 13; a polypeptide comprising at least 60% sequence identity with SEQ ID NO: 12; a peptide comprising at least 60% sequence identity with SEQ ID NO: 14; or a fluorophore capable of being activated by energy transfer from an Oplophorus luciferase.

In some embodiments, the target analyte binding agent includes a target analyte binding element and one of: a bioluminescent polypeptide of SEQ ID NO: 5; a polypeptide of SEQ ID NO: 9; a peptide of SEQ ID NO: 10; a peptide of SEQ ID NO: 11; a peptide of SEQ ID NO: 13; a polypeptide of SEQ ID NO: 12; a peptide of SEQ ID NO: 14; or a fluorophore capable of being activated by energy transfer from an Oplophorus luciferase.

In some embodiments, the conjugate pad further includes a luminogenic substrate. In some embodiments, the luminogenic substrate is selected from coelenterazine, coelenterazine-h, coelenterazine-h-h, furimazine, JRW-0238, JRW-1404, JRW-1482, JRW-1667, JRW-1743, JRW-1744, and other coelenterazine analogs or derivatives. In some embodiments, the luminogenic substrate contained on or within the conjugate pad as part of a composition that includes the luminogenic substrate and a polymer selected from pullulan, trehalose, maltose, cellulose, dextran, polystyrene, poly(meth)acrylate, and a combination of any thereof. In some embodiments, the luminogenic substrate is applied to the system as part of a composition that includes the luminogenic substrate and a substance to reduce autoluminescence such as ATT (6-Aza-2-thiothymine), a derivative or analog of ATT, a thionucleoside, thiourea, and the like.

Embodiments of the present disclosure also include an analytical membrane that includes a detection region and a control region. In accordance with these embodiments, the detection region includes at least one target analyte binding agent immobilized to the detection region.

In some embodiments, the at least one target analyte binding agent includes a target analyte binding element and one of: a bioluminescent polypeptide comprising at least 60% sequence identity with SEQ ID NO: 5; a polypeptide comprising at least 60% sequence identity with SEQ ID NO: 9; a peptide comprising at least 60% sequence identity with SEQ ID NO: 10; a peptide comprising at least 60% sequence identity with SEQ ID NO: 11; a peptide comprising at least 60% sequence identity with SEQ ID NO: 13; a polypeptide comprising at least 60% sequence identity with SEQ ID NO: 12; a peptide comprising at least 60% sequence identity with SEQ ID NO: 14; or a fluorophore capable of being activated by energy transfer from an Oplophorus luciferase.

In some embodiments, the target analyte binding agent includes a target analyte binding element and one of: a bioluminescent polypeptide of SEQ ID NO: 5; a polypeptide of SEQ ID NO: 9; a peptide of SEQ ID NO: 10; a peptide of SEQ ID NO: 11; a peptide of SEQ ID NO: 13; a polypeptide of SEQ ID NO: 12; a peptide of SEQ ID NO: 14; or a fluorophore capable of being activated by energy transfer from an Oplophorus luciferase.

In some embodiments, the analytical membrane further includes a plurality of detection regions with each detection region comprising a distinct target analyte binding agent having distinct target analyte binding elements. In some embodiments, the analytical membrane further includes a luminogenic substrate. In some embodiments, the luminogenic substrate is selected from coelenterazine, coelenterazine-h, coelenterazine-h-h, furimazine, JRW-0238, JRW-1404, JRW-1482, JRW-1667, JRW-1743, JRW-1744, and other coelenterazine analogs or derivatives.

In some embodiments, the luminogenic substrate is reversibly conjugated to the conjugate pad as part of a composition including the luminogenic substrate and a polymer selected from pullulan, trehalose, maltose, cellulose, dextran, polystyrene, poly(meth)acrylate, and a combination of any thereof. In some embodiments, the luminogenic substrate is part of a composition that includes the luminogenic substrate and a substance that reduces autoluminescence such as ATT (6-Aza-2-thiothymine), a derivative or analog of ATT, a thionucleoside, thiourea, and the like.

Embodiments of the present disclosure also include a solid phase detection platform comprising a detection region. In accordance with these embodiments, the detection region includes at least one target analyte binding agent conjugated to the detection region. In some embodiments, the at least one target analyte binding agent includes a target analyte binding element and one of: a bioluminescent polypeptide comprising at least 60% sequence identity with SEQ ID NO: 5; a polypeptide comprising at least 60% sequence identity with SEQ ID NO: 9; a peptide comprising at least 60% sequence identity with SEQ ID NO: 10; a peptide comprising at least 60% sequence identity with SEQ ID NO: 11; a peptide comprising at least 60% sequence identity with SEQ ID NO: 13; a polypeptide comprising at least 60% sequence identity with SEQ ID NO: 12; a peptide comprising at least 60% sequence identity with SEQ ID NO: 14; or a fluorophore capable of being activated by energy transfer from an Oplophorus luciferase.

In some embodiments, the target analyte binding agent includes a target analyte binding element and one of: a bioluminescent polypeptide of SEQ ID NO: 5; a polypeptide of SEQ ID NO: 9; a peptide of SEQ ID NO: 10; a peptide of SEQ ID NO: 11; a peptide of SEQ ID NO: 13; a polypeptide of SEQ ID NO: 12; a peptide of SEQ ID NO: 14; or a fluorophore capable of being activated by energy transfer from an Oplophorus luciferase.

In some embodiments, the detection platform includes: a first target analyte binding agent comprising a target analyte binding element and a polypeptide comprising at least 60% sequence identity with SEQ ID NO: 6 conjugated to the detection region; and a second target analyte binding agent comprising a target analyte binding element and a peptide comprising at least 60% sequence identity with SEQ ID NO: 10 applied to the detection region.

In some embodiments, the detection platform includes: a first target analyte binding agent comprising a target analyte binding element and a polypeptide comprising at least 60% sequence identity with SEQ ID NO: 10 conjugated to the detection region; and a second target analyte binding agent comprising a target analyte binding element and a peptide comprising at least 60% sequence identity with SEQ ID NO: 6 applied to the detection region.

In some embodiments, the detection platform includes: a first target analyte binding agent comprising a target analyte binding element and a peptide comprising at least 60% sequence identity with SEQ ID NO: 11 conjugated to the detection region; a second target analyte binding agent comprising a target analyte binding element and a peptide comprising at least 60% sequence identity with SEQ ID NO: 13 applied to the detection region; and a polypeptide comprising at least 60% sequence identity with SEQ ID NO: 12 applied to the detection region.

In some embodiments, the detection platform includes: a first target analyte binding agent comprising a target analyte binding element and a polypeptide comprising at least 60% sequence identity with SEQ ID NO: 6 conjugated to the detection region; and a second target analyte binding agent comprising a target analyte binding element and a polypeptide comprising at least 60% sequence identity with ID NO: 14 applied to the detection region.

In some embodiments, the detection platform includes: a first target analyte binding agent comprising a target analyte binding element and a polypeptide comprising at least 60% sequence identity with SEQ ID NO: 14 conjugated to the detection region; and a second target analyte binding agent comprising a target analyte binding element and a polypeptide comprising at least 60% sequence identity with SEQ ID NO: 6 applied to the detection region.

In some embodiments, the detection platform includes: a first target analyte binding agent comprising a target analyte binding element and a bioluminescent polypeptide at least 60% sequence identity with SEQ ID NO: 5 conjugated to the detection region; and a second target analyte binding agent comprising a target analyte binding element and a fluorophore capable of being activated by energy transfer from the bioluminescent polypeptide applied to the detection region.

In some embodiments, the detection platform includes: a first target analyte binding agent comprising a target analyte binding element and a bioluminescent polypeptide at least 60% sequence identity with SEQ ID NO: 5 applied to the detection region; and a second target analyte binding agent comprising a target analyte binding element and a fluorophore capable of being activated by energy transfer from the bioluminescent polypeptide conjugated to the detection region.

In some embodiments, the detection platform further includes a plurality of detection regions with each detection region comprising a distinct target analyte binding agent having distinct target analyte binding elements. In some embodiments, the detection platform further includes a control region. In some embodiments, the detection platform further includes a luminogenic substrate. In some embodiments, the luminogenic substrate is selected from coelenterazine, coelenterazine-h, coelenterazine-h-h, furimazine, JRW-0238, JRW-1404, JRW-1482, JRW-1667, JRW-1743, JRW-1744, and other coelenterazine analogs or derivatives. In some embodiments, the luminogenic substrate is reversibly conjugated to the conjugate pad as part of a composition comprising the luminogenic substrate and a polymer selected from pullulan, trehalose, maltose, cellulose, dextran, polystyrene, poly(meth)acrylate, and a combination of any thereof. In some embodiments, the luminogenic substrate is part of a composition comprising the luminogenic substrate and a substance that reduces autoluminescence such as ATT (6-Aza-2-thiothymine), a derivative or analog of ATT, a thionucleoside, thiourea, and the like.

Embodiments of the present disclosure also include a solution phase detection platform that includes at least one detection receptacle and a lyophilized tablet (lyocake). In accordance with these embodiments, the lyocake comprises a target analyte binding agent comprising a target analyte binding element and one of: a bioluminescent polypeptide comprising at least 60% sequence identity with SEQ ID NO: 5; a polypeptide comprising at least 60% sequence identity with SEQ ID NO: 9; a peptide comprising at least 60% sequence identity with SEQ ID NO: 10; a peptide comprising at least 60% sequence identity with SEQ ID NO: 11; a peptide comprising at least 60% sequence identity with SEQ ID NO: 13; a polypeptide comprising at least 60% sequence identity with SEQ ID NO: 12; a peptide comprising at least 60% sequence identity with SEQ ID NO: 14; or a fluorophore capable of being activated by energy transfer from an Oplophorus luciferase.

In some embodiments, the target analyte binding agent comprises a target analyte binding element and one of: a bioluminescent polypeptide of SEQ ID NO: 5; a polypeptide of SEQ ID NO: 9; a peptide of SEQ ID NO: 10; a peptide of SEQ ID NO: 11; a peptide of SEQ ID NO: 13; a polypeptide of SEQ ID NO: 12; a peptide of SEQ ID NO: 14; or a fluorophore capable of being activated by energy transfer from an Oplophorus luciferase.

In some embodiments, the lyocake comprises: a first target analyte binding agent comprising a target analyte binding element and a polypeptide comprising at least 60% sequence identity with SEQ ID NO: 6; and a second target analyte binding agent comprising a target analyte binding element and a peptide comprising at least 60% sequence identity with SEQ ID NO: 10.

In some embodiments, the lyocake comprises: a first target analyte binding agent comprising a target analyte binding element and a peptide comprising at least 60% sequence identity with SEQ ID NO: 11; a second target analyte binding agent comprising a target analyte binding element and a peptide comprising at least 60% sequence identity with SEQ ID NO: 13; and a polypeptide comprising at least 60% sequence identity with SEQ ID NO: 12.

In some embodiments, the lyocake comprises: a first target analyte binding agent comprising a target analyte binding element and a polypeptide comprising at least 60% sequence identity with SEQ ID NO: 6; and a second target analyte binding agent comprising a target analyte binding element and a polypeptide comprising at least 60% sequence identity with ID NO: 14.

In some embodiments, the lyocake comprises: a first target analyte binding agent comprising a target analyte binding element and a bioluminescent polypeptide at least 60% sequence identity with SEQ ID NO: 5; and a second target analyte binding agent comprising a target analyte binding element and a fluorophore capable of being activated by energy transfer from the bioluminescent polypeptide.

In some embodiments, the detection platform comprises a 96-well microtiter plate comprising a plurality of detection receptacles, and at least two distinct target analyte binding agents comprising distinct target analyte binding elements.

In some embodiments, the lyocake comprises a luminogenic substrate. In some embodiments, the luminogenic substrate is selected from coelenterazine, coelenterazine-h, coelenterazine-h-h, furimazine, JRW-0238, JRW-1404, JRW-1482, JRW-1667, JRW-1743, JRW-1744, and other coelenterazine analogs or derivatives.

In some embodiments, the lyocake comprises a luminogenic substrate and a polymer selected from pullulan, trehalose, maltose, cellulose, dextran, polystyrene, poly(meth)acrylate, and a combination of any thereof.

In some embodiments, the lyocake comprises a luminogenic substrate and a substance to reduce autoluminescence such as ATT (6-Aza-2-thiothymine), a derivative or analog of ATT, a thionucleoside, thiourea, and the like.

In some embodiments, the detection platform further comprises at least one sample. In some embodiments, the sample is selected from blood, serum, plasma, urine, stool, cerebral spinal fluid, interstitial fluid, saliva, a tissue sample, a water sample, a soil sample, a plant sample, a food sample, a beverage sample, an oil, and an industrial fluid sample.

Embodiments of the present disclosure also include a method of detecting an analyte in a sample using the lateral flow assay systems described above. In accordance with these embodiments, the method includes applying a sample to the sample pad, facilitating flow of the sample from the sample pad to the conjugate pad, and then from the conjugate pad to the detection region and the control region on the analytical membrane. In some embodiments, the first target analyte binding agent, the second target analyte binding agent, and the target analyte form the analyte detection complex in the at least one detection region when the target analyte is detected in the sample.

In some embodiments, the sample is a sample from a subject selected from blood, serum, plasma, urine, stool, cerebral spinal fluid, interstitial fluid, tissue, and saliva. In some embodiments, the sample is selected from a water sample, a soil sample, a plant sample, a food sample, a beverage sample, an oil, and an industrial fluid sample. In some embodiments, detecting the target analyte in the sample comprises detecting a bioluminescent signal generated from the analyte detection complex.

In some embodiments, the method further comprises quantifying a bioluminescent signal generated from the analyte detection complex. In some embodiments, the method further comprises diagnosing a subject from which the sample was obtained as having or not having a disease based on the detection of the analyte.

Embodiments of the present disclosure also include a method of detecting an analyte in a sample using the solid phase detection platform described above. In accordance with these embodiments, the method includes exposing a sample to the detection region and control region. In some embodiments, the at least one target analyte binding agent and the at least one target analyte form an analyte detection complex in the at least one detection region when the target analyte is detected in the sample.

In some embodiments, the sample is a sample from a subject selected from blood, serum, plasma, urine, stool, cerebral spinal fluid, interstitial fluid, tissue, and saliva. In some embodiments, the sample is selected from a water sample, a soil sample, a plant sample, a food sample, a beverage sample, an oil, and an industrial fluid sample. In some embodiments, detecting the target analyte in the sample comprises detecting a bioluminescent signal generated from the analyte detection complex.

In some embodiments, the method further comprises quantifying a bioluminescent signal generated from the analyte detection complex. In some embodiments, the method further comprises diagnosing a subject from which the sample was obtained as having or not having a disease based on the detection of the analyte.

Embodiments of the present disclosure also include a method of producing a substrate for use in a bioluminescent assay. In accordance with these embodiments, the method includes applying a solution onto a substrate. In some embodiments, the solution contains at least one target analyte binding agent comprising a target analyte binding element and one of a polypeptide component of a bioluminescent complex or a peptide component of a bioluminescent complex. In some embodiments, the method includes drying the substrate containing the solution.

In some embodiments, the solution further includes a complementary peptide or polypeptide component of the bioluminescent complex. In some embodiments, the target analyte binding agent and the complementary peptide or polypeptide component of the bioluminescent complex form a bioluminescent analyte detection complex in the presence of a target analyte.

In some embodiments, the solution comprises a protein buffer and at least one excipient. In some embodiments, the solution comprises a luminogenic substrate.

In some embodiments, the substrate comprising the dried solution is W-903 paper, FTA paper, FTA Elute paper, FTA DMPK paper, Ahlstrom A-226 paper, M-TFN paper, FTA paper, FP705 paper, Bode DNA collection paper, nitrocellulose paper, nylon paper, cellulose paper, Dacron paper, cotton paper, and polyester papers, or combinations thereof. In some embodiments, the substrate is a mesh comprising plastic, nylon, metal, or combinations thereof.

In some embodiments, drying the substrate containing the solution comprises drying at a temperature from about 30° C. to 40° C. for a period of time between about 30 mins and 2 hours. In some embodiments, drying the substrate containing the solution comprises lyophilizing and/or freezing the substrate.

In some embodiments, the method further comprises drying the at least one target analyte binding agent and/or the complementary peptide or polypeptide component of the bioluminescent complex onto a first substrate, and drying the luminogenic substrate onto a second substrate.

In accordance with these embodiments, a bioluminescent signal is generated upon exposure of the substrate containing the solution to the target analyte, and in some embodiments, the bioluminescent signal is proportional to the concentration of the target analyte.

In some embodiments, the at least one target analyte binding agent and/or the complementary peptide or polypeptide component of the bioluminescent complex exhibit(s) enhanced stability when dried on the substrate.

Embodiments of the present disclosure include a composition comprising a luminogenic substrate, a target analyte binding agent comprising a target analyte binding element and a polypeptide component of a bioluminescent complex, and a complementary polypeptide component of the bioluminescent complex. In accordance with these embodiments, the target analyte binding agent and the complementary polypeptide component of the bioluminescent complex are capable of forming a bioluminescent analyte detection complex in the presence of a target analyte.

In some embodiments, the composition further comprises a second target analyte binding agent comprising a second target analyte binding element and a second polypeptide component of a bioluminescent complex.

In some embodiments, the first and second target analyte binding agents bind separate portions of the same target analyte.

In some embodiments, the first and second polypeptide components of the bioluminescent complex bind the complementary polypeptide component of the bioluminescent complex to form a bioluminescent analyte detection complex in the presence of the target analyte.

In some embodiments, the first and the second polypeptide components are linked to a modified dehalogenase capable of forming a covalent bond with a haloalkane substrate.

In some embodiments, the first and the second target analyte binding elements comprise a haloalkane substrate.

In some embodiments, the first or second polypeptide components of the first and second target analyte binding agents comprise: at least 60% sequence identity with SEQ ID NO: 10; at least 60% sequence identity with SEQ ID NO: 11; at least 60% sequence identity with SEQ ID NO: 13; or at least 60% sequence identity with SEQ ID NO: 15.

In some embodiments, the complementary polypeptide component comprises: at least 60% sequence identity with SEQ ID NO: 6; at least 60% sequence identity with SEQ ID NO: 9; or at least 60% sequence identity with SEQ ID NO: 12.

In some embodiments, the target analyte binding element is selected from the group consisting of an antibody, a polyclonal antibody, a monoclonal antibody, a recombinant antibody, an antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, an oligonucleotide probe, a peptide nucleic acid, a DARPin, an aptamer, an affimer, a protein domain, and a purified protein.

In some embodiments, the target analyte is an antibody, and wherein the target analyte binding element of the first target analyte binding agent comprises antigen recognized by the antibody, and wherein the target analyte binding element of the second target analyte binding agent comprises an Fc binding region.

In some embodiments, the first and/or second target analyte binding agents further comprise a fluorophore coupled to the first and/or second polypeptide components of the bioluminescent complex.

In some embodiments, one or more components of the composition is in the form of a lyophilized tablet (lyocake) capable of forming a bioluminescent complex when reconstituted in a solution to detect and/or quantify the target analyte.

In some embodiments, the composition comprises a solution-phase detection platform capable of detecting and/or quantifying the target analyte.

In some embodiments, the polypeptide components and the luminogenic substrate are in the form of a lyophilized tablet (lyocake) capable of forming a bioluminescent complex when reconstituted in a solution to detect and/or quantify the target analyte.

Embodiments of the present disclosure also includes a method of detecting an analyte in a sample comprising combining any of the compositions described above with a sample comprising a target analyte.

In some embodiments, detecting the target analyte in the sample comprises detecting a bioluminescent signal generated from an analyte detection complex.

In some embodiments, the method further comprises quantifying a bioluminescent signal generated from the analyte detection complex.

In some embodiments, the bioluminescent signal generated from the analyte detection complex is proportional to the concentration of the analyte.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a representative schematic diagram of a lateral flow assay for detecting and/or quantifying a target analyte(s) in a sample based on bioluminescent complex formation, according to one embodiment of the present disclosure.

FIG. 2 shows a representative schematic diagram of a solid phase detection platform for detecting and/or quantifying target analytes in a sample based on bioluminescent complex formation, according to one embodiment of the present disclosure.

FIG. 3 shows representative images demonstrating that components of the bioluminescent complexes produce detectable bioluminescence after being applied to a solid support substrate (e.g., membrane), dried, and stored at room temperature.

FIG. 4 shows representative images demonstrating that components of the bioluminescent complexes produce detectable bioluminescence after being applied to membrane and paper-based solid support substrates.

FIG. 5 shows a representative assay schematic (left) and a representative graph (right) demonstrating the ability of components of the bioluminescent complexes to be used as reporters on target analyte binding agents for target analyte detection.

FIG. 6 shows a representative depiction of an assay platform using components of the bioluminescent complexes as reporters on target analyte binding agents for target analyte detection.

FIGS. 7A-7E show representative stability tests of an assay platform using components of the bioluminescent complexes as reporters on target analyte binding agents for target analyte detection, according to one embodiment of the present disclosure (FIG. 7A at 4° C.;

FIG. 7B at 25° C.; FIG. 7C at 37° C.; FIG. 7D at 37° C. with NanoLuc added; and FIG. 7E at 4° C. and 37° C. with HiBiT added).

FIGS. 8A-8B show representative tests of storage conditions of an assay platform using components of the bioluminescent complexes as reporters on target analyte binding agents for target analyte detection, according to one embodiment of the present disclosure (FIG. 8A at 4° C. and 25° C.; FIG. 8B at 4° C. and 25° C. with a sucrose-based protein buffer).

FIGS. 9A-9C show representative images from a solid phase assay platform (FIG. 9A) in which a bioluminescence signal was produced in complex sampling environments (whole blood in FIG. 9B and serum in FIG. 9C) indicating target analyte detection.

FIG. 10A-10B shows that RLU signal derived from Whatman 903 paper spots after rehydration with an assay buffer can be measured either quantitatively (FIG. 10A) or qualitatively (FIG. 10B).

FIGS. 11A-11B show representative graphs demonstrating the ability of a high affinity dipeptide, Pep263, to form bioluminescent complexes (Pep263 is a peptide comprising the 39 and 010 stands of the NanoTrip complex; see, e.g., U.S. patent application Ser. No. 16/439,565 (PCT/US2019/036844), which is herein incorporated by reference in its entirety).

FIG. 12 shows representative results of a solid phase assay demonstrating qualitative assessment of bioluminescence from paper punches placed into a standard microtiter plate using a standard camera from an iPhone (e.g., iPhone 6S) or from an imager (e.g., LAS4000).

FIGS. 13A-13B include quantitative analysis of the same solid phase assay depicted in FIG. 12, but luminescence was detected using a luminometer on day 3 of storage at 25° C. (raw RLU values are provided in FIG. 13A; RLU values over background are provided in FIG. 13B).

FIGS. 14A-14C include a quantitative time course of the same solid phase assay as depicted in FIGS. 12-13, demonstrating stability of all the proteins in the experimental conditions at all temps tested over the time frame. Maximum RLU values are provided at 4° C. (FIG. 14A), 25° C. (FIG. 14B), and 37° C. (FIG. 14C).

FIGS. 15A-15D include representative RLU signal kinetic results collected on day 0 of an accelerated stability study performed under two buffer conditions at 25° C. and 60° C. (raw RLU values are provided in FIGS. 15A and 15B; RLU values over background are provided in FIGS. 15C and 15D).

FIGS. 16A-16B include time-course results for an accelerated stability study of the proteins placed using the conjugation buffer conditions defined in FIG. 15. Maximum RLU values are provided at 25° C. (FIG. 16A) and 60° C. (FIG. 16B).

FIG. 17 shows a comparison of the impact of buffer conditions on luminescence from NanoLuc dried onto a nitrocellulose membrane.

FIG. 18 shows the effects of membrane blocking and sucrose pre-treatment on lateral flow assays performed in a running buffer of 20×SSC, 1% BSA, pH 7.0, and 10 μM N205 (Live Cell Substrate; LCS).

FIG. 19 shows the effects of membrane blocking and sucrose pre-treatment on lateral flow assays performed in a running buffer of 0.01 μM PBS, 1% BSA, pH 7.0, and 10 μM Permeable Cell Substrate (PCS).

FIG. 20 shows the effects of membrane blocking and sucrose pre-treatment on lateral flow assays performed in a running buffer of 5× LCS dilution buffer+5× LCS—diluted to 1× in PBS.

FIG. 21 shows effects of membrane properties on bioluminescent reagent absorption and capillary action in a lateral flow assay.

FIGS. 22A-22B show bioluminescent signal from NanoBiT/HiBiT complementation on nitrocellulose (left) and Whatman grade 541 (right) papers (FIG. 22A), and a compilation image from a corresponding movie taken across total exposure time (FIG. 22B).

FIG. 23 shows bioluminescent signal from NanoBiT/HiBiT complementation on Whatman 903 paper, with a spike of additional substrate and liquid at 20 minutes.

FIG. 24 shows bioluminescent signal from NanoBiT/HiBiT complementation on Whatman 903 paper.

FIGS. 25A-25C show bioluminescent signal resulting from reconstitution with a dipeptide of LgTrip and substrate in Whatman 903 paper, which was prepared with BSA (FIG. 25B) or without BSA (FIG. 25A); FIG. 25C shows maximum RLU signals obtained for each concentration tested in FIG. 25B.

FIGS. 26A-26B show bioluminescent signal resulting from reconstitution with a dipeptide of LgTrip and substrate from a lyocake (FIG. 26A), along with a titration of the dipeptide; FIG. 26B shows maximum RLU signals obtained for each concentration tested in FIG. 26A.

FIG. 27 shows bioluminescent signal in three different solid phase materials (Whatman 903, Ahlstrom 237, and Ahlstrom 6613H) resulting from reconstitution with a dipeptide added to dried LgTrip and substrate, or NanoLuc added to dried LgTrip and substrate.

FIG. 28 shows bioluminescent signal generated from Whatman 903 spots containing Lg/Trip/substrate and stored under ambient conditions over 25 days; spots were exposed to 1 nM dipeptide in PBS.

FIGS. 29A-29C show bioluminescent signal (RLU) for NanoLuc (FIG. 29A), LgBiT (FIG. 29B), and LgTrip (FIG. 29C) that were dried in Whatman 903 papers with various protein buffer formulations and reconstituted with furimazine.

FIGS. 30A-30C show bioluminescent signal (Bₘₐₓ) for NanoLuc (FIG. 30A), LgBiT (FIG. 30B), and LgTrip (FIG. 30C) that were dried in Whatman 903 papers with various protein buffer formulations and reconstituted with furimazine, as shown in FIG. 29.

FIGS. 31A-31B show bioluminescent background levels for LgBiT (FIG. 31A) and LgTrip (FIG. 31B) that were dried in Whatman 903 papers with various protein buffer formulations and reconstituted with furimazine, as shown in FIG. 29.

FIGS. 32A-32F show bioluminescent signal (RLU signal kinetics) after reconstitution with furimazine in FIGS. 32A-32C; Bₘₐₓ in FIGS. 32D-32F) for NanoLuc (FIGS. 32A and 32D), LgBiT (FIGS. 32B and 32E), and LgTrip (FIGS. 32C and 32F) that were dried in Whatman 903 papers with various protein buffer formulations and reconstituted with furimazine after 6 days of storage at 60° C.

FIG. 33 includes representative embodiments of all-in-one lyophilized cakes (“lyocakes”) or tablets containing all necessary reagents to perform an analyte detection test supporting several types of assay formats including cuvettes, test tubes, large volumes in bottles, snap test type assays, etc.

FIG. 34 shows bioluminescent signal from substrate movement across a lateral flow strip containing NanoLuc from a compilation image corresponding to a movie taken across total exposure time.

FIG. 35 shows bioluminescent signal from NanoLuc movement across a lateral flow strip from a compilation image corresponding to a movie taken across total exposure time.

FIG. 36 shows various tracers generated by tethering fumonisin B1 to a peptide tag (e.g., comprising SEQ ID NO: 10) via a biotin/streptavidin linkage, via a HaloTag linkage, or directly (e.g., via sulfo-SE labeling described in, for example, U.S. patent application Ser. No. 16/698,143 (PCT/US2019/063652), herein incorporated by reference), which can be used in competitive binding assays in accordance with the materials and methods described herein.

FIG. 37 shows an exemplary competitive binding assay in which varying concentrations of unlabeled fumonisin B1 disrupts the bioluminescent complex and results in decreased luminescence and the ability to detect/quantify the amount of fumonisin B1 in a sample.

FIGS. 38A-38B show bioluminescent signal resulting from a lyophilized cake containing LgBiT and substrate when reconstituted with a dipeptide in PBS (FIG. 38A); FIG. 38B shows maximum RLU signals obtained for each concentration tested in FIG. 38A.

FIG. 39 shows the bioluminescent signal resulting from reconstitution of LgBiT or LgTrip 3546 that was lyophilized directly into a standard 96-well plate with or without substrate; reconstitution was performed with dipeptide in PBS with or without substrate.

FIGS. 40A-40C show the bioluminescent signal resulting from the complementation of LgBiT-protein G, SmBiT-TNFα, and substrate in Whatman 903 paper spots (FIGS. 40A-40B) and in a lyocake format (FIG. 40C) after reconstitution with varying concentrations of the target analyte Remicade in PBS.

FIGS. 41A-41C show the bioluminescent signal resulting from the complementation of LgTrip, SmTrip9-protein G, HiBiT-TNFα, and substrate in Whatman 903 paper spots (FIG. 41A) and in a lyocake format (FIG. 41B-41C) after reconstitution with varying concentrations of the target analyte Remicade in PBS.

FIGS. 42A-42E show the bioluminescent signal resulting from the complementation of bioluminescent complexes dried down in a form that does not include a substrate (FIGS. 42B-42C: mesh-based lyocakes; FIGS. 42D-42E: mesh-based film); the substrate is added separately to generate the bioluminescent signal in the presence of the analyte.

FIG. 43 shows lyophilized cake formations and colorimetric pHs of four different furimazine substrate formulations.

FIG. 44 shows the kinetic activity performance of various furimazine (Fz) substrate formulations in the presence of purified NanoLuc (Nluc) enzyme.

FIG. 45 shows the activity performance of a furimazine substrate formulation that had been stored at 60° C. for the indicated time in days.

FIGS. 46A-46B show thermal stability over time in days of various furimazine substrate formulations maintained at ambient temperature (FIG. 46A) or 60° C. (FIG. 46B) as analyzed by HPLC for absolute furimazine concentration remaining after reconstitution in PBS, pH 7.0 containing 0.01% BSA.

FIG. 47 shows the amount of furimazine remaining for various furimazine substrate formulations after 12 days of reconstitution in water as analyzed by HPLC indicating liquid stability.

FIG. 48 shows a schematic representation of the homogenous tripartite immunoassay for the analyte interleukin-6 (IL-6).

FIG. 49 shows an example of an SDS-PAGE gel of antibody labeling with tripartite-HaloTag fusion proteins. Variants of SmTrip9 or SmTriplO were fused to HaloTag and expressed, purified, and used to label mouse anti-human IL-6 antibodies.

FIGS. 50A-50B show the signal kinetics of a solution-based homogeneous tripartite IL-6 immunoassay with and without IL-6 (raw RLUs in FIG. 50A, and fold response in FIG. 50B).

FIGS. 51A-51B show the dose response curve of recombinant human IL-6 for the solution-based homogeneous IL-6 tripartite immunoassay (log graph in FIG. 51A; linear graph in FIG. 51B).

FIGS. 52A-52C show the lyophilized cake product (FIG. 52A; #1 and #2) and IL-6 immunoassay performance and shelf-stability of various formulated, single reagent lyophilized cakes without furimazine (Fz; FIG. 52B) and with furimazine (Fz; FIG. 52C) after reconstitution following storage at ambient temperature for the indicating time in days.

FIGS. 53A-53B show cake appearance (FIG. 53A) and performance (FIG. 53B) and shelf-stability of a formulated, lyophilized single-reagent IL-6 tripartite immunoassays stored for 90 days at ambient storage.

FIG. 54 shows the signal kinetics of a single reagent, lyophilized tripartite IL-6 immunoassay post-reconstitution.

FIG. 55 shows the compatibility of a lyophilized single reagent IL-6 immunoassay with complex human matrices.

FIGS. 56A-56B show a lyophilized single-reagent, IL-6 tripartite immunoassay in a pre-filled 96-well microtiter plate (FIG. 56A) and a rhIL-6 dose response curve using the lyophilized, single reagent, IL-6 tripartite immunoassay assay plate following reconstitution (FIG. 56B).

FIGS. 57A-57B show the assay performance of the solution-based IL-6 tripartite immunoassay in single formulation excipients (FIG. 57A) and in various formulated solutions (FIG. 57B).

FIG. 58 shows a schematic representation of the homogenous tripartite immunoassay for the model analyte cardiac troponin I.

FIGS. 59A-59B show dose response curves for the solution-based, homogeneous cardiac troponin I tripartite immunoassay using recombinant human cardiac tropoinin I in raw RLUs (FIG. 59A) and signal over background (FIG. 59B).

FIG. 60 shows the assay performance in raw RLUs of the single-reagent, formulated lyophilized troponin cardiac I tripartite immunoassay after reconstitution with 0.01% BSA in PBS or 10% normal pooled human serum diluted in general serum diluent.

FIGS. 61A-61B show raw RLU results of the solution-based, homogeneous IL-6 tripartite immunoassay background signals in the presence of human sera when using assay buffers 0.01% BSA in PBS (FIG. 61A) and in general serum diluent (FIG. 61B).

FIGS. 62A-62B show the raw Bmax RLU results of the solution-based, homogeneous IL-6 tripartite immunoassay in the presence of 50 ng/ml of rhIL-6 in the presence of human sera when using assay buffers 0.01% BSA in PBS (FIG. 62A) and in general serum diluent (FIG. 62B).

FIGS. 63A-63D show the signal to background results of the solution-based, homogeneous IL-6 tripartite immunoassay in the presence or absence of 50 ng/ml rhIL-6 with increasing amounts of normal pooled human serum (FIGS. 63A and 63C) or normal pooled human plasma (FIGS. 63B and 63D) when run in either 0.01% BSA in PBS or General Serum Diluent as assay buffer and NanoGlo (Promega Cat #N113) (FIGS. 63C and 63D) or Live Cell (Promega Cat #N205) substrates (FIGS. 63A and 63B).

FIG. 64 shows the signal-to-background results of the solution-based, homogeneous IL-6 tripartite immunoassay in the presence or absence of 50 ng/ml rhIL-6 with increasing amounts of normal, pooled human sera and pooled human sera that has been depleted of endogenous IgG when using general serum diluent as assay buffer.

FIGS. 65A-65C show the results of background RLU (FIG. 65A), Bmax RLU (FIG. 65B), and resulting signal over background (FIG. 65C) for the solution-based, homogeneous IL-6 tripartite immunoassay in the presence or absence of 50 ng/ml rhIL-6 with increasing amounts of human blood chemistry panel components provided in the VeriChem matrix plus chemistry reference kit.

FIGS. 66A-66C show the results of background RLU (FIG. 66A), Bmax RLU (FIG. 66B), and resulting signal over background (FIG. 66C) for the solution-based, homogeneous IL-6 tripartite immunoassay in the presence or absence of 50 ng/ml rhIL-6 with increasing amounts of pooled normal human urine and NanoGlo (Promega Cat #N113) or Live Cell (Promega Cat #N205) substrates.

FIGS. 67A-67C show the raw RLU activity assay response of reconstituted lyophilized formulated furimazine tested with purified NanoLuc enzyme (Nluc) (FIG. 67A), formulated LgTrip polypeptide (SEQ ID NO: 12) tested with purified di-peptide (SEQ ID NO: 14) (FIG. 67B), and formulated furimazine and LgTrip polypeptide (SEQ ID NO: 12) tested with purified di-peptide (SEQ ID NO: 14) combined analyzing the thermal stability of the lyophilized vials (FIG. 67C).

FIG. 68 shows a schematic representation of a homogenous tripartite immunoassay for three anti-TNFα biologics: Remicade, Enbrel, and Humira.

FIGS. 69A-69C show the assay performance in raw RLUs of the solution-based, homogenous tripartite (LgTrip 3546+SmTrip9 pep521+SmTrip10) immunoassays quantitating the anti-TNFα biologics Remicade, Humira, and Enbrel.

FIGS. 70A-70B show the kinetic assay performance displayed as raw RLUs of reconstituted formulated, lyophilized single-reagent immunoassays for detection of Remicade using NanoTrip (tripartite-NanoLuc; FIG. 70A) and NanoBiT (FIG. 70B).

FIG. 71 shows the thermal stability at ambient temperatures of the single-reagent, lyophilized NanoBiT (“Bits”) and NanoTrip (“Trips;” tripartite NanoLuc) immunoassay systems for the detection of Remicade. Lyocakes were reconstituted at the time points indicated in the absence or presence of 100 nM Remicade, and the resulting raw RLU were analyzed.

FIGS. 72A-72D show representative results using the NanoBiT system to detect Remicade in which the formulated components are separated into two separate cakes prior to use in the assay: (FIG. 72A) an image of two separate, lyophilized components with one containing LgBiT-TNFα fusion protein and furimazine (yellow), and the other containing the SmBiT-protein G fusion protein (white); (FIG. 72B) an image after manually combining the two lyophilized components in FIG. 72A; (FIG. 72C) an image of the reconstituted lyophilized components; and (D) kinetic bioluminescence RLU signals resulting in the presence of increasing amounts of Remicade.

FIG. 73 shows the resulting kinetic bioluminescence RLU signal resulting in the presence of increasing amounts of Remicade using the dual-lyophilized NanoTrip immunoassay system, whereby the TNFα+furimazine and protein G fusion proteins were formulated, lyophilized separately, and then combined prior to reconstitution.

FIG. 74 shows a schematic representation of the homogenous, NanoTrip (tripartite NanoLuc), cell-based immunoassay system for detection of anti-EGFR biologics (e.g., panitumumab).

FIG. 75 shows a panitumumab dose response curve using the homogenous, cell-based NanoTrip immunoassay system for anti-EGFR biologics.

FIG. 76 shows a panitumumab dose response curve using the homogeneous, cell-based NanoTrip immunoassay system for anti-EGFR biologics testing different variants of SmTrip9 (SEQ ID NO: 13) fused to protein G.

FIGS. 77A-77B show a Remicade dose response curve using the homogeneous, solution-based NanoTrip immunoassay system for anti-TNFα biologics testing different variants of SmTrip9 (SEQ ID NO: 13) fused to protein G (FIG. 77A), and a Remicade dose response curve using the lyophilized NanoTrip immunoassay system for anti-TNFα biologics (FIG. 77B).

FIG. 78 shows a schematic representation of the tripartite IL-6 immunoassay system using antibodies directly labeled with reactive peptides (e.g., SEQ ID NO: 18).

FIGS. 79A-79C show denaturing SDS-PAGE gel analysis of directly-labeled antibody conjugates.

FIG. 80 shows the raw RLU output from IL-6 titration in the presence of anti-IL-6 antibody pairs directly labeled with reactive peptides HW-0984 (SEQ ID NO: 20), HW-1010 (SEQ ID NO: 24), and HW-0977 (SEQ ID NO: 18).

FIG. 81 shows the raw RLU output from IL-6 titration in the presence of anti-IL-6 antibody pairs directly labeled with reactive peptides HW-0984 (SEQ ID NO: 20) and HW-1053 (SEQ ID NO: 19).

FIG. 82 shows the raw RLU output from IL-6 titration in the presence of anti-IL-6 antibody pairs labeled with reactive peptides HW-1042 (SEQ ID NO: 20), HW-1050 (SEQ ID NO: 27), HW-1052 (SEQ ID NO: 25), HW-1043 (SEQ ID NO: 24) and HW-1055 (SEQ ID NO: 25).

FIG. 83 shows the raw RLU output from IL-6 titration in the presence of individual anti-IL-6 antibodies directly labeled with reactive peptides HW-0977 (SEQ ID NO: 18), HW-0984 (SEQ ID NO: 20), HW-1010 (SEQ ID NO: 24), HW-1042 (SEQ ID NO: 20), HW-1050 (SEQ ID NO: 27), HW-1052 (SEQ ID NO: 25), HW-1053 (SEQ ID NO: 19), HW-1043 (SEQ ID NO: 24), and HW-1055 (SEQ ID NO: 25).

FIG. 84 shows the raw RLU output from IL-6 titration in the presence of LgTrip 5146 (SEQ ID NO: 451) and anti-IL-6 antibody pairs labeled with reactive peptides HW-1050 (SEQ ID NO: 27), HW-1043 (SEQ ID NO: 24), and HW-0977 (SEQ ID NO: 18).

FIG. 85 shows a schematic representation of the tripartite IL-6 immunoassay model using antibodies directly labeled with reactive peptides containing fluorophores, enabling BRET between the luciferase and labeled antibodies.

FIG. 86 shows IL-6 induced BRET between the complemented tripartite luciferase and fluorophores on the labeled anti-IL-6 antibodies.

FIGS. 87A-87C show the luminescence derived from luminogenic substrates N113 Fz (FIG. 87A), JRW-1404 (FIG. 87B), and JRW-1482 (FIG. 87C) in complex matrices.

### DETAILED DESCRIPTION

Embodiments of the present disclosure provide systems and methods for the detection of an analyte or analytes in a sample. In particular, the present disclosure provides compositions, assays, and methods for detecting and/or quantifying a target analyte using a bioluminescent complex comprising substrates, peptides, and/or polypeptides capable of generating a bioluminescent signal that correlates to the presence, absence, or amount of the target analyte.

Most rapid diagnostic bioassays are based on immunological principles. Some embodiments of the present disclosure combine immunoassay-based concepts with the advantages of bioluminescence, which include a large linear range and extremely low background, among other advantages. However, despite these advantages, point-of-care bioluminescence-based immunoassays are not yet commercially available. Some reasons for this may be that many currently available luciferases have low signal, which inherently limits their usefulness in immunoassays. Additionally, when a bioluminescent signal output is configured to be conditional (e.g., through complementation or bioluminescence resonance energy transfer (BRET)), the signal can be reduced even further. Many currently available luciferases also have a low tolerance or sensitivity to certain assay conditions, such as high temperatures, non-optimal buffer compositions, and complex sample matrices, thus requiring specialized chemistries to be compatible with point-of-care devices.

Embodiments of the present disclosure also address the need for “all-in-one” assay formats for analyte detection, which until the present application, have not been developed or described in the prior art. For example, Tenda, K. et al. (Angew. Chem. Int. Ed. 57, 15369-15373 (2018)) discloses paper devices where the substrate and bioluminescent components are dried onto separate sections of the paper, rather than being included together in a single-format system. Additionally, Yu, Q. et al. (Science 361, 1122-1126 (2018)) discloses that, although the bioluminescent components can be dried together, the substrate is separately mixed with the analyte-of-interest and subsequently added to the paper rather than drying the substrate and the bioluminescent components in a single format system. As described further herein, embodiments of the present disclosure provide methods, compositions, and systems that include all the necessary components of a bioluminescent detection complex (excluding the analyte-of-interest) in a single-format (e.g., “all-in-one”) system. This contrasts with currently available systems, which include at least one of the necessary bioluminescent components in a separate format/solution. Thus, embodiments of the present disclosure provide surprising and unexpected advantages over currently available bioluminescent analyte detection systems.

To address the need for bioluminescent-based point-of-care immunoassay platforms that are not necessarily limited to the use of typical immunoassay reagents, embodiments of the present disclosure include the use of the NanoLuc® bioluminescent platform, including compositions and methods for the assembly of a bioluminescent complex from two or more peptide and/or polypeptide components. In some embodiments, the peptide and/or polypeptide components are not fragments of a preexisting protein (e.g., are not complementary subsequences of a known polypeptide sequence), but confer bioluminescent activity via structural complementation (See, e.g., WO/2014/151736 (Intl. App. No. PCT/US2014/026354) and U.S. patent application Ser. No. 16/439,565 (PCT/US2019/036844), herein incorporated by reference in their entireties), as described further herein. In some embodiments, peptide and/or polypeptide components are non-luminescent in the absence of complementation and/or complementation enhances bioluminescence of a peptide or polypeptide component. In some embodiments, target analyte binding agents are labeled with the various components of the bioluminescent complexes described herein without comprising the ability of the binding agents to bind their target analytes. Components of the bioluminescent complexes of the present disclosure are configured to be compatible with currently available point-of-care devices and systems such as lateral flow devices, paper-based spot tests, dip stick tests, lab-on-a-chip, microfluidic devices, pre-filled 96-well microtiter plates, and the like.

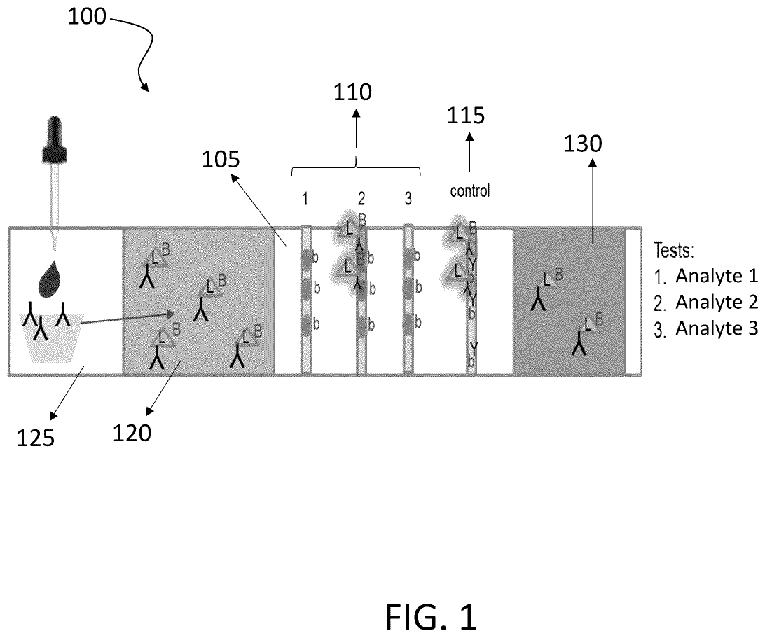

For example, embodiments of the present disclosure incorporate NanoLuc®-based technologies (e.g., NanoBiT, NanoTrip, Nano-Glo (e.g., NANOGLO Live Cell Substrate or NANOGLO LCS (Promega Cat. Nos. N205 and N113)), NanoBRET, etc.) into target analyte detection assays that can be embedded in a solid phase assay or device, including plastics, matrices, and membranes of various composition, and/or used in other assay formats such as lyophilized cakes or tablets for solution phase assays, all of which function reliably even in complex sampling environments (e.g., blood components, food matrix, soil samples, stool, urine, water, and other human and animal biological samples). In some embodiments, NanoLuc®-based reporter systems are incorporated into lateral flow assay (LFA) technology, paper spot tests, and similar devices. LFAs are a commonly used point-of-care technology used to measure a variety of target analytes including, but not limited to, antibodies, bacterial and viral antigens, metabolites, proteins, and the like. As demonstrated in FIG. 1, LFAs can be combined with NanoLuc®-based reporter technology to provide a multiplexed viral infection detection assay to detect anti-viral antibodies at the point of care. The only currently available, approved emergency use immunoassay to detect Zika exposure is a traditional plate based, multi-step sandwich ELISA to detect the presence of anti-Zika IgM in blood samples. In contrast to this system, the multiplexed capability of a NanoLuc®-based bioluminescent reporter platform allows for the rapid detection of multiple antibodies in a sample, whether the antibodies recognize multiple different epitopes of the same virus, or whether they recognize multiple different epitopes on more than one virus. The ability to detect and identify viral infections quickly and sensitively with bioluminescence will aid treatment decisions. In addition to antibodies and antigens, the small size of the component peptides of the bioluminescent complexes described herein allow for the detection of many other target analytes using alternative binding agents and materials, such as, but not limited to, DARPins, aptamers, oligonucleotide probes, peptide nucleic acids (PNAs), and locked nucleic assays (LNAs).

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. Many embodiments herein are described using open “comprising” language. Such embodiments encompass multiple closed “consisting of” and/or “consisting essentially of” embodiments, which may alternatively be claimed or described using such language. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Bioluminescence” refers to production and emission of light by a chemical reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light.

“Complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance to form a complex (e.g., from interaction elements), for example, to place the elements in the proper conformation for complementarity, to co-localize complementary elements, to lower interaction energy for complementation, etc.

“Complex” refers to an assemblage or aggregate of molecules (e.g., peptides, polypeptides, etc.) in direct and/or indirect contact with one another. In one aspect, “contact,” or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules (e.g., a peptide and polypeptide) is formed under assay conditions such that the complex is thermodynamically favored (e.g., compared to a non-aggregated, or non-complexed, state of its component molecules). As used herein the term “complex,” unless described as otherwise, refers to the assemblage of two or more molecules (e.g., peptides, polypeptides or a combination thereof).

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, such as a protein domain linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Fragment” refers to a peptide or polypeptide that results from dissection or “fragmentation” of a larger whole entity (e.g., protein, polypeptide, enzyme, etc.), or a peptide or polypeptide prepared to have the same sequence as such. Therefore, a fragment is a subsequence of the whole entity (e.g., protein, polypeptide, enzyme, etc.) from which it is made and/or designed. A peptide or polypeptide that is not a subsequence of a preexisting whole protein is not a fragment (e.g., not a fragment of a preexisting protein). A peptide or polypeptide that is “not a fragment of a preexisting bioluminescent protein” is an amino acid chain that is not a subsequence of a protein (e.g., natural or synthetic) that: (1) was in physical existence prior to design and/or synthesis of the peptide or polypeptide, and (2) exhibits substantial bioluminescent activity.

As used herein, the term “antibody fragment” refers to a portion of a full-length antibody, including at least a portion of the antigen binding region or a variable region. Antibody fragments include, but are not limited to, Fab, Fab′, F(ab′)₂, Fv, scFv, Fd, variable light chain, variable heavy chain, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis. For example, a “Fab” fragment comprises one light chain and the C_{H1} and variable region of one heavy chain. The heavy chain of a Fab molecule cannot form a disulfide bond with another heavy chain molecule. A “Fab′” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule. An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen. Other antibody fragments will be understood by skilled artisans.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

“Non-luminescent” refers to an entity (e.g., peptide, polypeptide, complex, protein, etc.) that exhibits the characteristic of not emitting a detectable amount of light in the visible spectrum (e.g., in the presence of a substrate). For example, an entity may be referred to as non-luminescent if it does not exhibit detectable luminescence in a given assay. As used herein, the term “non-luminescent” is synonymous with the term “substantially non-luminescent. For example, a non-luminescent polypeptide is substantially non-luminescent, exhibiting, for example, a 10-fold or more (e.g., 100-fold, 200-fold, 500-fold, 1×10³-fold, 1×10⁴-fold, 1×10⁵-fold, 1×10⁶-fold, 1×10⁷-fold, etc.) reduction in luminescence compared to a complex of the polypeptide with its non-luminescent complement peptide. In some embodiments, an entity is “non-luminescent” if any light emission is sufficiently minimal so as not to create interfering background for a particular assay.

“Non-luminescent peptide” and “non-luminescent polypeptide” refer to peptides and polypeptides that exhibit substantially no luminescence (e.g., in the presence of a substrate), or an amount that is beneath the noise, or a 10-fold or more (e.g., 100-fold, 200-fold, 500-fold, 1×10³-fold, 1×10⁴-fold, 1×10⁵-fold, 1×10⁶-fold, 1×10⁷-fold, etc.) when compared to a significant signal (e.g., luminescent complex) under standard conditions (e.g., physiological conditions, assay conditions, etc.) and with typical instrumentation (e.g., luminometer, etc.). In some embodiments, such non-luminescent peptides and polypeptides assemble, according to the criteria described herein, to form a bioluminescent complex. As used herein, a “non-luminescent element” is a non-luminescent peptide or non-luminescent polypeptide. The term “bioluminescent complex” refers to the assembled complex of two or more non-luminescent peptides and/or non-luminescent polypeptides. The bioluminescent complex catalyzes or enables the conversion of a substrate for the bioluminescent complex into an unstable form; the substrate subsequently emits light. When uncomplexed, two non-luminescent elements that form a bioluminescent complex may be referred to as a “non-luminescent pair.” If a bioluminescent complex is formed by three or more non-luminescent peptides and/or non-luminescent polypeptides, the uncomplexed constituents of the bioluminescent complex may be referred to as a “non-luminescent group.”

“Peptide” and “polypeptide” as used herein, and unless otherwise specified, refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

“Preexisting protein” refers to an amino acid sequence that was in physical existence prior to a certain event or date. A “peptide that is not a fragment of a preexisting protein” is a short amino acid chain that is not a fragment or sub-sequence of a protein (e.g., synthetic or naturally-occurring) that was in physical existence prior to the design and/or synthesis of the peptide.

“Sample,” “test sample,” “specimen,” “sample from a subject,” and “patient sample” as used herein may be used interchangeable and may be a sample of blood, such as whole blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

“Subject” and “patient” as used herein interchangeably refers to any vertebrate, including, but not limited to, a mammal and a human. In some embodiments, the subject may be a human or a non-human. The subject or patient may be undergoing forms of treatment. “Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

“Subsequence” refers to peptide or polypeptide that has 100% sequence identify with another, larger peptide or polypeptide. The subsequence is a perfect sequence match for a portion of the larger amino acid chain.

“Substantially” as used herein means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-luminescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., luminescent intensity of a bioluminescent protein or bioluminescent complex).

“Variant” is used herein to describe a peptide or polypeptide that differs in amino acid sequence by the insertion, deletion, or conservative substitution of amino acids, but retain at least one biological activity. “SNP” refers to a variant that is a single nucleotide polymorphism. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid (e.g., replacing an amino acid with a different amino acid of similar properties, such as hydrophilicity, degree, and distribution of charged regions) is recognized in the art as typically involving a minor change. These minor changes can be identified, in part, by considering the hydropathic index of amino acids, as understood in the art. The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Target analyte” or “analyte” as used herein refers to a substance in a sample that can be detected, quantified, measured, tested, and/or monitored, often as part of a method of evaluating a process or condition (e.g., diagnostic or prognostic assay). Target analytes can include, but are not limited to, a protein, a peptide, a polypeptide, an enzyme, a cofactor, a nucleotide, a polynucleotide, DNA, RNA, a small molecule compound, an antibody, and any variation, combination, and derivative thereof.

“Target analyte binding agent” as used herein refers to an agent capable of binding to a target analyte. In some embodiments, target analyte binding agents include agents that can bind multiple substances, such as a target analyte and a solid phase support. In some embodiments, target analyte binding agents include agents that bind both a target analyte (e.g., via a target analyte binding element) and a distinct peptide/polypeptide to form a target analyte detection complex (e.g., to generate a bioluminescent signal). In some embodiments, target analyte binding agents can include target analyte binding elements capable of binding a group or class of analytes (e.g., protein L binding to antibodies); and in other embodiments, target analyte binding agents can include target analyte binding elements capable of binding a specific analyte (e.g., an antigen binding a monoclonal antibody). A target analyte binding agent may be an antibody, antibody fragment, a receptor domain that binds a target ligand, proteins or protein domains that bind to immunoglobulins (e.g., protein A, protein G, protein A/G, protein L, protein M), a binding domain of a proteins that bind to immunoglobulins (e.g., protein A, protein G, protein A/G, protein L, protein M), oligonucleotide probe, peptide nucleic acid, DARPin, aptamer, affimer, a purified protein, or a protein domain (either the analyte itself or a protein that binds to the analyte), and analyte binding domain(s) of proteins etc. Table A provides a lists of exemplary binding moieties that could be used singly or in various combinations in methods, systems, and assays (e.g., immunoassays) herein.

TABLE 1
Exemplary target analyte binding agents.

| Binding Moiety A | Binding Moiety B |
|---|---|
| Protein A | Protein A |
| Ig Binding domain of protein A | Ig binding domain of protein A |
| Protein G | Protein G |
| Ig Binding domain of protein G | Ig binding domain of protein G |
| Protein L | Protein L |
| Ig Binding domain of protein L | Ig binding domain of protein L |
| Protein M | Protein M |
| Ig Binding domain of protein M | Ig binding domain of protein M |
| polyclonal antibody against analyte X | polyclonal antibody: same antibody or second polyclonal |
| | antibody recognizing same target analyte X |
| monoclonal antibody | monoclonal antibody recognizing different epitope on |
| | same target analyte X |
| recombinant antibody | recombinant antibody recognizing different epitope on |
| | same target analyte X |
| scFv | scFv recognizing different epitope on same target analyte |
| | X |
| variable light chain (V_{L}) of antibody (monoclonal, | variable heavy chain (V_{H}) of same antibody (monoclonal, |
| recombinant, polyclonal) recognizing target analyte X | recombinant, polyclonal) recognizing target analyte X |
| protein (e.g. receptor) binding domain 1 that binds | protein (e.g. receptor) binding domain 2 that binds to |
| to analyte X | analyte X |
| (Fab) fragment | (Fab) fragment from different antibody recognizing |
| | different epitope to same target analyte X |
| Fab′ fragment | Fab′ from different antibody recognizing different epitope |
| | to same target analyte X |
| Fv fragment | Fv from different antibody recognizing different epitope |
| | to same target analyte X |
| F(ab′)2 fragment | F(ab′)2 from different antibody recognizing different |
| | epitope to same target analyte X |
| oligonucleotide probe | oligonucleotide probe to same DNA or RNA target but |
| | recognizing non-overlapping sequence |
| DARPin | DARPin recognizing non-overlapping domain of same |
| | target |
| peptide nucleic acid | peptide nucleic acid recognizing same DNA or RNA |
| | target but non-overlapping sequence |
| aptamer | aptamer binding to same target analyte X but recognizing |
| | non-overlapping sequence or epitope |
| affimer | aptamer binding to same target analyte X but recognizing |
| | different epitope |

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Bioluminescence

The present disclosure includes materials and methods related to bioluminescent polypeptides, bioluminescent complexes and components thereof, and bioluminescence resonance energy transfer (BRET).

In some embodiments, provided herein are solid phase and/or lateral flow assays, devices, and systems incorporating bioluminescent polypeptides and/or bioluminescent complexes (of non-luminescent peptide(s) and/or non-luminescent polypeptide components) based on (e.g., structurally, functionally, etc.) the luciferase of Oplophorus gracilirostris, the NanoLuc® luciferase (Promega Corporation; U.S. Pat. Nos. 8,557,970; 8,669,103; herein incorporated by reference in their entireties), the NanoBiT (U.S. Pat. No. 9,797,889; herein incorporated by reference in its entirety), or NanoTrip (U.S. patent application Ser. No. 16/439,565; and U.S. Prov. Appln. Ser. No. 62/941,255; both of which are herein incorporated by reference in their entireties). As described below, in some embodiments, the compositions, assays, devices, methods, and systems herein incorporate commercially available NanoLuc®-based technologies (e.g., NanoLuc® luciferase, NanoBRET, NanoBiT, NanoTrip, NanoGlo, etc.), but in other embodiments, various combinations, variations, or derivations from the commercially available NanoLuc®-based technologies are employed.

a. NanoLuc

PCT Appln. No. PCT/US2010/033449, U.S. Pat. No. 8,557,970, PCT Appln. No. PCT/2011/059018, and U.S. Pat. No. 8,669,103 (each of which is herein incorporated by reference in their entirety and for all purposes) describe compositions and methods comprising bioluminescent polypeptides. Such polypeptides find use in embodiments herein and can be used in conjunction with the compositions, assays, devices, systems, and methods described herein.

In some embodiments, compositions, assays, devices, systems, and methods provided herein comprise a bioluminescent polypeptide of SEQ ID NO: 5, or having at least 60% (e.g., 60%, 65%, 70%, 75%, 80%, 85%, 90%, 95%, 96%, 97%, 98%, 99%, 100%, or ranges therebetween) sequence identity with SEQ ID NO: 5.

In some embodiments, any of the aforementioned bioluminescent polypeptides are linked (e.g., fused, chemically linked, etc.) to a binding element or other component of the assays and systems described herein.

In some embodiments, any of the aforementioned bioluminescent polypeptides, or fusions or conjugates thereof (e.g., with a binding element, etc.), are immobilized to a portion of a device described herein (e.g., a detection or control region of a lateral flow assay, a solid phase detection element, etc.).

b. NanoBiT

PCT Appln. No. PCT/US14/26354 and U.S. Pat. No. 9,797,889 (each of which is herein incorporated by reference in their entirety and for all purposes) describe compositions and methods for the assembly of bioluminescent complexes; such complexes, and the peptide and polypeptide components thereof, find use in embodiments herein and can be used in conjunction with the assays and methods described herein.

In some embodiments, provided herein are non-luminescent (NL) polypeptides having at least 60% (e.g., 60%, 65%, 70%, 75%, 80%, 85%, 90%, 95%, 96%, 97%, 98%, 99%, 100%, or ranges therebetween) sequence identity with SEQ ID NO: 9, but less than 100% (e.g., <99%, <98%, <97%, <96%, <95%, <94%, <93%, <92%, <91%, <90%) sequence identity with SEQ ID NO: 1, SEQ ID NO: 2, and SEQ ID NO: 6.

In some embodiments, provided herein are non-luminescent (NL) peptides having at least 60% (e.g., 60%, 65%, 70%, 75%, 80%, 85%, 90%, 95%, 96%, 97%, 98%, 99%, 100%, or ranges therebetween) sequence identity with SEQ ID NO: 10, but less than 100% (e.g., <99%, <98%, <97%, <96%, <95%, <94%, <93%, <92%, <91%, <90%) sequence identity with SEQ ID NO: 1, SEQ ID NO: 4, and SEQ ID NO: 8.

In some embodiments, provided herein are NL peptides having at least 60% (e.g., 60%, 65%, 70%, 75%, 80%, 85%, 90%, 95%, 96%, 97%, 98%, 99%, 100%, or ranges therebetween) sequence identity with SEQ ID NO: 11, but less than 100% (e.g., <99%, <98%, <97%, <96%, <95%, <94%, <93%, <92%, <91%, <90%) sequence identity with SEQ ID NO: 1, SEQ ID NO: 4, and SEQ ID NO: 8.

In some embodiments, any of the aforementioned NL peptides or NL polypeptides are linked (e.g., fused, chemically linked, etc.) to a binding element or other component of the composition, assays, devices, methods, and systems described herein.

In some embodiments, any of the aforementioned NL peptides or NL polypeptides, or fusions or conjugates thereof (e.g., with a binding element, etc.), are immobilized to a portion of a device described herein (e.g., a detection or control region of a lateral flow assay, a solid phase detection element, etc.).

In some embodiments, provided herein is a lateral flow detection system comprising: an analytical membrane comprising a detection region and a control region, wherein the detection region comprises a first target analyte binding agent immobilized to the detection region; a conjugate pad comprising a second target analyte binding agent; and a sample pad; wherein the first target analyte binding agent comprises a first target analyte binding element and a first NanoBiT-based NL peptide or NL polypeptide component (as described above), and wherein the second target analyte binding agent comprises a second target analyte binding element and a complementary NanoBiT-based NL peptide or NL polypeptide component (as described above). In some embodiments, the first target analyte binding agent and the second target analyte binding agent form an analyte detection complex in the at least one detection region when a target analyte is detected in a sample. In some embodiments, a bioluminescent signal produced in the presence of a luminogenic substrate is substantially increased when the first target analyte binding agent contacts the second target analyte binding agent, as compared to a bioluminescent signal produced by the second target analyte binding agent or the first target analyte binding agent and the luminogenic substrate alone.

In some embodiments, provided herein is solid-phase detection system comprising: an solid phase substrate comprising a first target analyte binding agent and a second target analyte binding agent; wherein the first target analyte binding agent comprises a first target analyte binding element and a first NanoBiT-based NL peptide or NL polypeptide component (as described above), and wherein the second target analyte binding agent comprises a second target analyte binding element and a complementary NanoBiT-based NL peptide or NL polypeptide component (as described above). In some embodiments, the first target analyte binding agent and the second target analyte binding agent form an analyte detection complex in the solid-phase substrate when a target analyte is detected in a sample. In some embodiments, a bioluminescent signal produced in the presence of a luminogenic substrate is substantially increased when the first target analyte binding agent contacts the second target analyte binding agent, as compared to a bioluminescent signal produced by the second target analyte binding agent or the first target analyte binding agent and the luminogenic substrate alone.

c. NanoTrip

U.S. patent application Ser. No. 16/439,565 (PCT/US2019/036844) and U.S. Prov. Appln. Ser. No. 62/941,255 (both of which are herein incorporated by reference in their entireties and for all purposes) describes compositions, systems, and methods for the assembly of bioluminescent complexes. Such complexes, and the peptides and polypeptide components thereof, find use in embodiments herein and can be used in conjunction with the assays and methods described herein.

In some embodiments, provided herein are non-luminescent (NL) polypeptides having at least 60% (e.g., 60%, 65%, 70%, 75%, 80%, 85%, 90%, 95%, 96%, 97%, 98%, 99%, 100%, or ranges therebetween) sequence identity with SEQ ID NO: 12, but less than 100% (e.g., <99%, <98%, <97%, <96%, <95%, <94%, <93%, <92%, <91%, <90%) sequence identity with SEQ ID NO: 1, SEQ ID NO: 2, SEQ ID NO: 6, and SEQ ID NO: 9.

In some embodiments, provided herein are non-luminescent (NL) peptides having at least 60% (e.g., 60%, 65%, 70%, 75%, 80%, 85%, 90%, 95%, 96%, 97%, 98%, 99%, 100%, or ranges therebetween) sequence identity with SEQ ID NO: 11, but less than 100% (e.g., <99%, <98%, <97%, <96%, <95%, <94%, <93%, <92%, <91%, <90%) sequence identity with SEQ ID NO: 1, SEQ ID NO: 4, and SEQ ID NO: 8.

In some embodiments, provided herein are NL peptides having at least 60% (e.g., 60%, 65%, 70%, 75%, 80%, 85%, 90%, 95%, 96%, 97%, 98%, 99%, 100%, or ranges therebetween) sequence identity with SEQ ID NO: 13, but less than 100% (e.g., <99%, <98%, <97%, <96%, <95%, <94%, <93%, <92%, <91%, <90%) sequence identity with SEQ ID NO: 1, SEQ ID NO: 3, and SEQ ID NO: 7.

In some embodiments, provided herein are NL peptides having at least 60% (e.g., 60%, 65%, 70%, 75%, 80%, 85%, 90%, 95%, 96%, 97%, 98%, 99%, 100%, or ranges therebetween) sequence identity with SEQ ID NO: 14, but less than 100% (e.g., <99%, <98%, <97%, <96%, <95%, <94%, <93%, <92%, <91%, <90%) sequence identity with SEQ ID NO: 1, SEQ ID NO: 3, SEQ ID NO: 4, SEQ ID NO: 7, and SEQ ID NO: 8.

In some embodiments, any of the aforementioned NanoTrip-based NL peptide or NL polypeptides are linked (e.g., fused, chemically linked, etc.) to a binding element or other component of the compositions, methods, devices, assays, and systems described herein.

In some embodiments, any of the aforementioned NanoTrip-based NL peptide or NL polypeptides, or fusions or conjugates thereof (e.g., with a binding element, etc.), are immobilized to a portion of a device described herein (e.g., a detection or control region of a lateral flow assay, a solid phase detection element, etc.).

In some embodiments, provided herein is a lateral flow detection system comprising: an analytical membrane comprising a detection region and a control region, wherein the detection region comprises a first target analyte binding agent immobilized to the detection region; a conjugate pad comprising a second target analyte binding agent; and a sample pad; wherein the first target analyte binding agent comprises a first target analyte binding element and a first NanoTrip-based NL peptide (as described above), and wherein the second target analyte binding agent comprises a second target analyte binding element and a complementary NanoTrip-based NL peptide (as described above). In some embodiments, the first target analyte binding agent and the second target analyte binding agent form an analyte detection complex in the at least one detection region in the presence of a NanoTrip-based NL polypeptide component (as described above) when a target analyte is detected in a sample. In some embodiments, a bioluminescent signal produced in the presence of a luminogenic substrate is substantially increased when the first target analyte binding agent contacts the second target analyte binding agent in the presence of a NanoTrip-based NL polypeptide component, as compared to a bioluminescent signal produced by the second target analyte binding agent or the first target analyte binding agent and the luminogenic substrate alone.

In some embodiments, provided herein is a solid-phase detection system comprising: a solid phase (e.g., paper substrate, etc.) comprising a first target analyte binding agent and a second target analyte binding agent, wherein the first target analyte binding agent comprises a first target analyte binding element and a first NanoTrip-based NL peptide (as described above), and wherein the second target analyte binding agent comprises a second target analyte binding element and a complementary, second NL NanoTrip-based peptide (as described above). In some embodiments, the first target analyte binding agent and the second target analyte binding agent form an analyte detection complex in the presence of a NanoTrip-based NL polypeptide (as described above) when a target analyte is detected in a sample. In some embodiments, a bioluminescent signal produced in the presence of a luminogenic substrate is substantially increased when the first target analyte binding agent contacts the second target analyte binding agent and a NanoTrip-based NL polypeptide, as compared to a bioluminescent signal produced by the second target analyte binding agent or the first target analyte binding agent and the luminogenic substrate alone.

d. NanoBRET

As disclosed in PCT Appln. No. PCT/US13/74765 and U.S. patent application Ser. No. 15/263,416 (herein incorporated by reference in their entireties and for all purposes) describe bioluminescence resonance energy transfer (BRET) compositions, systems, and methods (e.g., incorporating NanoLuc®-based technologies); such compositions, systems and methods, and the bioluminescent polypeptide and fluorophore-conjugated components thereof, find use in embodiments herein and can be used in conjunction with the compositions, systems, devices, assays, and methods described herein.

In some embodiments, any of the NanoLuc®-based, NanoBiT-based, and/or NanoTrip-based (described in sections a-c, above) peptides, polypeptide, complexes, fusions, and conjugates may find use in BRET-based applications with the compositions, assays, methods, devices, and systems described herein. For example, in certain embodiments, a first target analyte binding agent comprises a first target analyte binding element and a NanoLuc®-based, NanoBiT-based, and/or NanoTrip-based polypeptide, peptide, or complex, and a second target analyte binding agent comprises a second target analyte binding element and a fluorophore (e.g., fluorescent protein, small molecule fluorophore, etc.), wherein the emission spectrum of the NanoLuc®-based, NanoBiT-based, and/or NanoTrip-based polypeptide, peptide, or complex overlaps the excitation spectrum of the fluorophore. In some embodiments, the NanoLuc®-based, NanoBiT-based, and/or NanoTrip-based polypeptide, peptide, or complex can be prepared in lyophilized form, which can include, or not include, the luminogenic substrate (e.g., furimazine).

In some embodiments, a target analyte binding agent comprises a target analyte binding element and a fluorophore capable of being activated by energy transfer from a bioluminescent polypeptide.

As used herein, the term “energy acceptor” refers to any small molecule (e.g., chromophore), macromolecule (e.g., autofluorescent protein, phycobiliproteins, nanoparticle, surface, etc.), or molecular complex that produces a readily detectable signal in response to energy absorption (e.g., resonance energy transfer). In certain embodiments, an energy acceptor is a fluorophore or other detectable chromophore. Suitable fluorophores include, but are not limited to: xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLuoR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc. In some embodiments, a fluorophore is a rhodamine analog (e.g., carboxy rhodamine analog), such as those described in U.S. patent application Ser. No. 13/682,589, herein incorporated by reference in its entirety.

e. HALOTAG

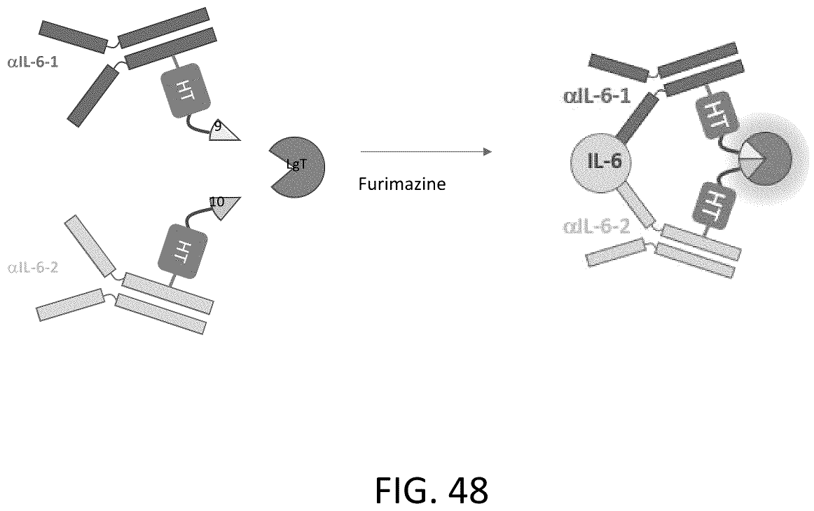

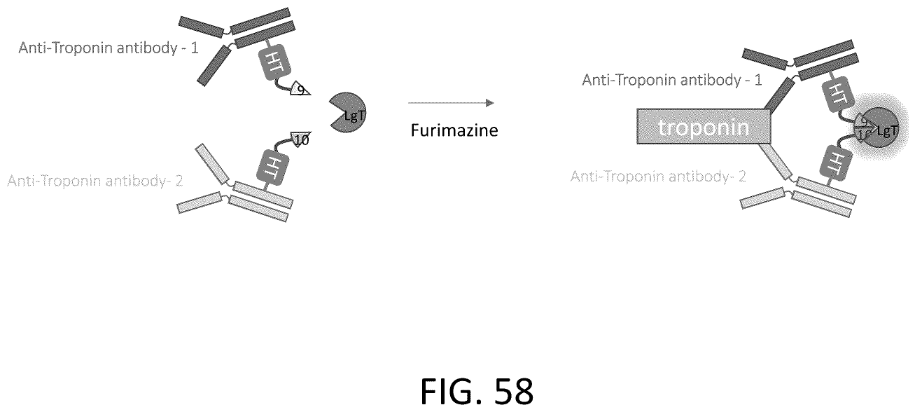

Some embodiments herein comprise a capture protein capable of forming a covalent bond with a capture ligand. The capture protein may be linked to a first element (e.g., a peptide component of a bioluminescent complex) and the capture ligand to a second element (e.g., a target analyte binding element (e.g., an antibody or antigen binding protein)) and the formation of a covalent bond links the first and second elements to each other. In some embodiments, linking the first and second elements creates a target analyte binding agent. In some embodiments, two or more target analyte binding agents so formed can bind to a complementary polypeptide component (e.g., LgTrip) and form a bioluminescent complex in the presence of an analyte (e.g., a target antigen recognized by the target analyte binding element) (See e.g., FIGS. 48 and 58). In some embodiments, the capture ligand is a haloalkane (aka “alkyl halide”). In some embodiments, the capture ligand is a chloroalkane. In some embodiments, the capture ligand is —A—X. In some embodiments, X is Cl. In some embodiments, —A—X is —(CH₂)₆Cl. When the capture ligand is a haloalkane, the capture protein is typically a dehalogenase enzyme modified to form covalent bonds with its substrate (See, e.g., U.S. Pat. Nos. 7,425,436; 7,429,472; 7,867,726; 7,888,086; 7,935,803; U.S. Pat. No. RE42,931; U.S. Pat. Nos. 8,168,405; 8,202,700; 8,257,939; herein incorporated by reference in their entireties).

One such modified dehalogenase is the commercially-available HALOTAG protein (SEQ ID NO: 720). In some embodiments, a capture protein comprises a polypeptide with at least 70% sequence identity (e.g., 75% identity, 80% identity, 85% identity, 90% identity, 95% identity, 98% identity, 99% identity) with SEQ ID NO.: 720. Some embodiment comprise a fusion protein of the capture protein (e.g., HALOTAG) and another peptide/polypeptide element (e.g., a binding moiety, a peptide/polypeptide component of a bioluminescent complex, etc.). In some embodiments, a capture ligand is a haloalkane comprising a halogen (e.g., Cl, Br, F, I, etc.) covalently attached to the end of an alkyl chain (e.g., (CH₂)₄₋₂₄). In some embodiments, the other end of the alkyl chain is attached to a linker or to another element (e.g., a peptide, analyte, etc.). A linker may comprise an alkyl chain or substituted alkyl chain (e.g., C═O, NH, S, O, carbamate, ethylene etc.) such as those disclosed in U.S. patent application Ser. No. 14/207,959, herein incorporated by reference.

### 3. Compositions and Formulations

Embodiments of the present disclosure include compositions and formulations comprising one or more of the peptide and/or polypeptide components of the bioluminescent complexes provided herein. In accordance with these embodiments, compositions and formulations of the present disclosure can include a luminogenic substrate and/or various other components. The compositions and methods provided herein can be used to formulate shelf-stable liquid formulations (e.g., used in a solution phase assay format) and shelf-stable dried formulations (e.g., used in a solid phase assay format) capable of producing a luminescent signal in the presence of an analyte-of-interest, even after storage for prolonged time periods. As described further below, the compositions and formulations of the present disclosure can include one or more components of NanoLuc, NanoBiT, NanoTrip, and NanoBRET as well as the various luminogenic substrates described herein (e.g., furimazine).

In contrast to many currently available fluorescent and colorimetric assays, the compositions and formulations of the present disclosure provide means for conducting bioassays in which one or more of the peptide and/or polypeptide components of the bioluminescent complexes exist in a stable, dried formulation that is capable of being reconstituted in a solution containing, for example, a complementary peptide/polypeptide and/or a luminogenic substrate, such that the bioluminescent complex is formed in the presence of the analyte-of-interest. In some embodiments, the compositions and formulations of the present disclosure provide the means for conducting robust solid phase bioassays in which the bioluminescent signal produced is quantitative and proportional to the concentration of the analyte-of-interest.

In some embodiments, the compositions and formulations of the present disclosure include a luminogenic substrate and a target analyte binding agent that includes a target analyte binding element and a polypeptide component of a bioluminescent complex or a peptide component of a bioluminescent complex. In some embodiments, the polypeptide component of the target analyte binding agent comprises at least 60% sequence identity with SEQ ID NO: 6, at least 60% sequence identity with SEQ ID NO: 9, or at least 60% sequence identity with SEQ ID NO: 12. In some embodiments, the polypeptide component of the target analyte binding agent comprises at least 70% sequence identity with SEQ ID NO: 6, at least 70% sequence identity with SEQ ID NO: 9, or at least 70% sequence identity with SEQ ID NO: 12. In some embodiments, the polypeptide component of the target analyte binding agent comprises at least 80% sequence identity with SEQ ID NO: 6, at least 80% sequence identity with SEQ ID NO: 9, or at least 80% sequence identity with SEQ ID NO: 12. In some embodiments, the polypeptide component of the target analyte binding agent comprises at least 85% sequence identity with SEQ ID NO: 6, at least 85% sequence identity with SEQ ID NO: 9, or at least 85% sequence identity with SEQ ID NO: 12. In some embodiments, the polypeptide component of the target analyte binding agent comprises at least 90% sequence identity with SEQ ID NO: 6, at least 90% sequence identity with SEQ ID NO: 9, or at least 90% sequence identity with SEQ ID NO: 12. In some embodiments, the polypeptide component of the target analyte binding agent comprises at least 95% sequence identity with SEQ ID NO: 6, at least 95% sequence identity with SEQ ID NO: 9, or at least 95% sequence identity with SEQ ID NO: 12.

In other embodiments, the peptide component of the target analyte binding agent comprises at least 60% sequence identity with SEQ ID NO: 10, at least 60% sequence identity with SEQ ID NO: 11, at least 60% sequence identity with SEQ ID NO: 13, or at least 60% sequence identity with SEQ ID NO: 14. In some embodiments, the peptide component of the target analyte binding agent comprises at least 70% sequence identity with SEQ ID NO: 10, at least 70% sequence identity with SEQ ID NO: 11, at least 70% sequence identity with SEQ ID NO: 13, or at least 70% sequence identity with SEQ ID NO: 14. In some embodiments, the peptide component of the target analyte binding agent comprises at least 80% sequence identity with SEQ ID NO: 10, at least 80% sequence identity with SEQ ID NO: 11, at least 80% sequence identity with SEQ ID NO: 13, or at least 80% sequence identity with SEQ ID NO: 14. In some embodiments, the peptide component of the target analyte binding agent comprises at least 85% sequence identity with SEQ ID NO: 10, at least 85% sequence identity with SEQ ID NO: 11, at least 85% sequence identity with SEQ ID NO: 13, or at least 85% sequence identity with SEQ ID NO: 14. In some embodiments, the peptide component of the target analyte binding agent comprises at least 90% sequence identity with SEQ ID NO: 10, at least 90% sequence identity with SEQ ID NO: 11, at least 90% sequence identity with SEQ ID NO: 13, or at least 90% sequence identity with SEQ ID NO: 14. In some embodiments, the peptide component of the target analyte binding agent comprises at least 95% sequence identity with SEQ ID NO: 10, at least 95% sequence identity with SEQ ID NO: 11, at least 95% sequence identity with SEQ ID NO: 13, or at least 95% sequence identity with SEQ ID NO: 14.

In some embodiments, the composition or formulation comprises a complementary peptide or polypeptide component of the bioluminescent complex. In accordance with these embodiments, the target analyte binding agent and the complementary peptide or polypeptide component of the bioluminescent complex can form a bioluminescent analyte detection complex in the presence of a target analyte. In some embodiments, the composition that comprises the luminogenic substrate and the target analyte binding agent can be combined in a dried formulation, and the complementary peptide or polypeptide component of the bioluminescent complex can be formulated as a liquid formulation. In some embodiments, the liquid formulation is added to the dried formulation and forms the bioluminescent analyte detection complex in the presence of the target analyte upon rehydration. In other embodiments, the composition or formulation comprising the luminogenic substrate, the target analyte binding agent, and the complementary peptide or polypeptide component of the bioluminescent complex are combined in a dried formulation, wherein the dried formulation forms the bioluminescent analyte detection complex in the presence of the target analyte upon rehydration.

In some embodiments, the complementary peptide or polypeptide component comprises a second target analyte binding element that forms the bioluminescent analyte detection complex in the presence of the target analyte. In some embodiments, the polypeptide component of the target analyte binding agent comprises at least 60% sequence identity with SEQ ID NO: 6, and wherein the complementary peptide or polypeptide component of the bioluminescent complex comprises at least 60% sequence identity with SEQ ID NO: 10. In some embodiments, the polypeptide component of the target analyte binding agent comprises at least 60% sequence identity with SEQ ID NO: 6, and wherein the complementary peptide or polypeptide component of the bioluminescent complex comprises at least 60% sequence identity with SEQ ID NO: 14.

Embodiments of the present disclosure also include a composition or formulation comprising a dried formulation that includes a first target analyte binding agent comprising a first target analyte binding element and a polypeptide component having at least 60% sequence identity with SEQ ID NO: 9, and a second target analyte binding agent comprising a second target analyte binding element and a complementary peptide component having at least 60% sequence identity with SEQ ID NO: 10. In some embodiments, the dried formulation further comprises a luminogenic substrate. In some embodiments, the composition further comprises a liquid formulation comprising the target analyte.

Embodiments of the present disclosure also include a composition comprising a dried formulation that includes a first target analyte binding agent comprising a first target analyte binding element and a polypeptide component having at least 60% sequence identity with SEQ ID NO: 12, and a second target analyte binding agent comprising a second target analyte binding element and a complementary peptide component having at least 60% sequence identity with SEQ ID NO: 14. In some embodiments, the dried formulation further comprises a luminogenic substrate. In some embodiments, the composition further comprises a liquid formulation comprising the target analyte.

Embodiments of the present disclosure also include a composition comprising a dried formulation that includes a first target analyte binding agent comprising a first target analyte binding element and a peptide component having at least 60% sequence identity with SEQ ID NO: 13, a second target analyte binding agent comprising a second target analyte binding element and a complementary peptide component having at least 60% sequence identity with SEQ ID NO: 15, and a complementary polypeptide component having at least 60% sequence identity with SEQ ID NO: 12. In some embodiments, the dried formulation further comprises a luminogenic substrate. In some embodiments, the composition further comprises a liquid formulation comprising the target analyte.

Embodiments of the present disclosure also include a composition that includes a dried formulation comprising a first target analyte binding agent comprising a target analyte binding element and a polypeptide component having at least 60% sequence identity with SEQ ID NO: 9, and a liquid formulation comprising a second target analyte binding agent comprising a target analyte binding element and a complementary peptide component having at least 60% sequence identity with SEQ ID NO: 10 or SEQ ID NO: 11.

Embodiments of the present disclosure also include a composition that includes a dried formulation comprising a first target analyte binding agent comprising a target analyte binding element and a peptide component having at least 60% sequence identity with SEQ ID NO: 10 or SEQ ID NO: 11, and a liquid formulation that contains a second target analyte binding agent comprising a target analyte binding element and a complementary polypeptide component having at least 60% sequence identity with SEQ ID NO: 9.

Embodiments of the present disclosure also include a composition that includes a dried formulation comprising a first target analyte binding agent comprising a target analyte binding element and a polypeptide component having at least 60% sequence identity with SEQ ID NO: 12, and a liquid formulation comprising a second target analyte binding agent comprising a target analyte binding element and a complementary peptide component having at least 60% sequence identity with SEQ ID NO: 14. In some embodiments, the dried formulation further comprises a luminogenic substrate. In some embodiments, the liquid formulation further comprises a luminogenic substrate. In some embodiments, the liquid formulation further includes a sample comprising a target analyte. In accordance with these embodiments, a bioluminescent analyte detection complex forms upon combining the dried formulation and the liquid formulation in the presence of the target analyte.

In some embodiments, the composition further comprises a second complementary peptide or polypeptide component of the bioluminescent complex. In accordance with these embodiments, the target analyte binding agent, the first complementary peptide or polypeptide component of the bioluminescent complex, and the second complementary peptide or polypeptide component of the bioluminescent complex form a bioluminescent analyte detection complex in the presence of a target analyte.

In some embodiments, the composition comprising the target analyte binding agent are produced as a dried formulation. In some embodiments, the first complementary peptide or polypeptide component and the second complementary peptide or polypeptide of the bioluminescent complex are produced as a liquid formulation. In accordance with these embodiments, the liquid formulation can be added to the dried formulation, which facilitates the formation of the bioluminescent analyte detection complex in the presence of the target analyte upon rehydration.

In some embodiments, the composition comprising the target analyte binding agent, and either the first or the second complementary peptide or polypeptide component are combined in a dried formulation, and the first or the second complementary peptide or polypeptide component that is not present in the dried formulation are produced as a liquid formulation. The liquid formulation can be added to the dried formulation, which facilitates the formation of the bioluminescent analyte detection complex in the presence of the target analyte upon rehydration.

In some embodiments, the target analyte binding agent, the first complementary peptide or polypeptide component, and the second complementary peptide or polypeptide component are combined in a dried formulation that forms the bioluminescent analyte detection complex in the presence of the target analyte upon rehydration. In some embodiments, the dried formulation further comprises a luminogenic substrate. In some embodiments, the liquid formulation further comprises a sample comprising a target analyte, wherein a bioluminescent analyte detection complex forms upon combining the dried formulation and the liquid formulation in the presence of the target analyte.

In some embodiments, either the first or the second complementary peptide or polypeptide component comprises a second target analyte binding element that forms the bioluminescent analyte detection complex in the presence of the target analyte upon rehydration.

In some embodiments, the polypeptide component of the target analyte binding agent comprises at least 60% sequence identity with SEQ ID NO: 12, and wherein either the first or the second complementary peptide or polypeptide component of the bioluminescent complex comprises at least 60% sequence identity with either SEQ ID NO: 13 or SEQ ID NO: 15.

Embodiments of the present disclosure also include a composition that includes a dried formulation comprising a first target analyte binding agent comprising a target analyte binding element and a polypeptide component having at least 60% sequence identity with SEQ ID NO: 12, and a liquid formulation comprising a second target analyte binding agent comprising a target analyte binding element and a complementary peptide component having at least 60% sequence identity with SEQ ID NO: 13 or SEQ ID NO: 15, and further including a second complementary peptide component having at least 60% sequence identity with SEQ ID NO: 13 or SEQ ID NO: 15.

Embodiments of the present disclosure also include a dried formulation comprising a first target analyte binding agent comprising a target analyte binding element and a polypeptide component having at least 60% sequence identity with SEQ ID NO: 12, and a second target analyte binding agent comprising a target analyte binding element and a complementary peptide component having at least 60% sequence identity with SEQ ID NO: 13 or SEQ ID NO: 15, and further including a liquid formulation comprising a second complementary peptide component having at least 60% sequence identity with SEQ ID NO: 13 or SEQ ID NO: 15.

Embodiments of the present disclosure also include a dried formulation comprising a first target analyte binding agent comprising a target analyte binding element and a polypeptide component having at least 60% sequence identity with SEQ ID NO: 12, and complementary peptide component having at least 60% sequence identity with SEQ ID NO: 13 or SEQ ID NO: 15, and a liquid formulation comprising a second target analyte binding agent comprising a target analyte binding element and a complementary peptide component having at least 60% sequence identity with SEQ ID NO: 13 or SEQ ID NO: 15.

Embodiments of the present disclosure also include a dried formulation comprising a first target analyte binding agent comprising a target analyte binding element and a peptide component having at least 60% sequence identity with SEQ ID NO: 13, and a second target analyte binding agent comprising a target analyte binding element and a complementary peptide component having at least 60% sequence identity with SEQ ID NO: 15, and further including a liquid formulation comprising a complementary polypeptide component having at least 60% sequence identity with SEQ ID NO: 12.

Embodiments of the present disclosure also include a dried formulation comprising a complementary polypeptide component having at least 60% sequence identity with SEQ ID NO: 12, and a liquid formulation comprising a first target analyte binding agent comprising a target analyte binding element and a peptide component having at least 60% sequence identity with SEQ ID NO: 13, and a second target analyte binding agent comprising a target analyte binding element and a complementary peptide component having at least 60% sequence identity with SEQ ID NO: 15.

Embodiments of the present disclosure also include a composition comprising a dried formulation comprising a first target analyte binding agent comprising a target analyte binding element and a peptide component having at least 60% sequence identity with SEQ ID NO: 13, a second target analyte binding agent comprising a target analyte binding element and a complementary peptide component having at least 60% sequence identity with SEQ ID NO: 15, and a complementary polypeptide component having at least 60% sequence identity with SEQ ID NO: 12. In some embodiments, the dried formulation further comprises a luminogenic substrate. In some embodiments, the liquid formulation further comprises a luminogenic substrate. In some embodiments, the liquid formulation further comprises a sample comprising a target analyte, and wherein a bioluminescent analyte detection complex forms upon combining the dried formulation and the liquid formulation in the presence of the target analyte.

In some embodiments, a bioluminescent signal produced in the presence of the luminogenic substrate is substantially increased when the target analyte binding agent contacts one or more of the complementary peptide or polypeptide components of the bioluminescent complex, as compared to a bioluminescent signal produced by the target analyte binding agent and the luminogenic substrate alone.

In some embodiments, the target analyte is a target antibody. In some embodiments, the target analyte binding agent comprises an element that binds non-specifically to antibodies. In some embodiments, the target analyte binding agent comprises an element that binds specifically to an antibody. In some embodiments, the target antibody is an antibody against a pathogen, toxin, or therapeutic biologic.

In some embodiments, a target analyte binding element is selected from the group consisting of an antibody, a polyclonal antibody, a monoclonal antibody, a recombinant antibody, an antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, an oligonucleotide probe, a peptide nucleic acid, a DARPin, an aptamer, an affimer, a protein domain, and a purified protein.

In some embodiments, the luminogenic substrate is selected from coelenterazine, coelenterazine-h, coelenterazine-h-h, furimazine, JRW-0238, JRW-1404, JRW-1482, JRW-1667, JRW-1743, JRW-1744, and other coelenterazine analogs or derivatives. In some embodiments, the coelenterazine analogs or derivatives are pro-luminogenic substrates such as those disclosed in U.S. Pat. No. 9,487,520, herein incorporated by reference. In some embodiments, the coelenterazine analogs or derivatives are Enduazine (Promega Corporation) and Vivazine (Promega Corporation).

In some embodiments, the composition further comprises a polymer. In some embodiments, the polymer is a naturally-occurring biopolymer. In some embodiments, the naturally-occurring biopolymer is selected from pullulan, trehalose, maltose, cellulose, dextran, and a combination of any thereof. In some embodiments, the naturally-occurring biopolymer is pullulan. In some embodiments, the polymer is a cyclic saccharide polymer or a derivative thereof. In some embodiments, the polymer is hydroxypropyl β-cyclodextrin.

In some embodiments, the polymer is a synthetic polymer. In some embodiments, the synthetic polymer is selected from polystyrene, poly(meth)acrylate, and a combination of any thereof. In some embodiments, the synthetic polymer is a block copolymer comprising at least one poly(propylene oxide) block and at least one poly(ethylene oxide) block. In some embodiments, the synthetic polymer is poloxamer 188.

In some embodiments, the composition further comprises a buffer, a surfactant, a reducing agent, a salt, a radical scavenger, a chelating agent, a protein, or any combination thereof. In some embodiments, the is surfactant selected from polysorbate 20, polysorbate 40, and polysorbate 80.

In some embodiments, the composition further comprises a substance that reduces autoluminescence. In some embodiments, the substance is ATT (6—Aza-2-thiothymine), a derivative or analog of ATT, a thionucleoside, thiourea, and the like. In some embodiments, the substance is a thionucleoside disclosed in U.S. Pat. No. 9,676,997, herein incorporated by reference. In some embodiments, the substance is thiourea, which use for reducing autoluminescence is disclosed in U.S. Pat. Nos. 7,118,878; 7,078,181; and 7,108,996, herein incorporated by reference.

In some embodiments, the composition is used in conjunction with an analyte detection platform to detect an analyte in a sample. In some embodiments, sample is selected from blood, serum, plasma, urine, stool, cerebral spinal fluid, interstitial fluid, saliva, a tissue sample, a water sample, a soil sample, a plant sample, a food sample, a beverage sample, an oil, and an industrial fluid sample.

Embodiments of the present disclosure also include a method of detecting an analyte in a sample comprising combining any of the compositions described above with a sample comprising a target analyte. In some embodiments, detecting the target analyte in the sample comprises detecting a bioluminescent signal generated from an analyte detection complex. In some embodiments, the method further comprises quantifying a bioluminescent signal generated from the analyte detection complex. In some embodiments, the bioluminescent signal generated from the analyte detection complex is proportional to the concentration of the analyte. In some embodiments, one or more of the components of the composition exhibits enhanced stability within the composition compared to the component in solution alone.

The various embodiments of the compositions and formulations described above demonstrate enhanced stability, as demonstrated in the Examples and FIGS. For example, when produced as a dried formulation such as a lyocake, when dried onto a substrate or matrix (e.g., Whatman 903, Ahlstrom 237, and Ahlstrom 6613H; wells of a 96-well plate, nylon mesh), or when dried in various protein buffer formulations, with or without the luminogenic substrate, the compositions and formulations of the present disclosure exhibit enhanced stability when stored for a prolonged period of time. As provided herein, the compositions and formulations of the present disclosure are able to generate a luminescent signal in the presence of a target analyte after storage for extended periods of time. In some embodiments, the compositions and formulations of the present disclosure exhibit enhanced stability as compared to compositions and formulations that contain the same or similar components of a bioluminescent complex (e.g., complementary peptides/polypeptides, luminogenic substrates), but which are formulated without one or more of the other components of the formulation, and/or are not formulated according to the methods described herein.

In some embodiments, the compositions and formulations of the present disclosure exhibit enhanced stability for at least about 1 day, 2 days, 3 days, 4 days, 5 days, 6 days, 7 days, 2 weeks, 3 weeks, 4 weeks, 1 month, 2 months, 3 months, 4 months, 5 months, 6 months, 7 months, 8 months, 9 months, 10 months, 12 months, and up to 1 year. In some embodiments, the compositions and formulations of the present disclosure exhibit enhanced stability at temperatures ranging from about 0° C. to 65° C., from about 4° C. to 65° C., from about 10° C. to 65° C., from about 15° C. to 65° C., from about 15° C. to 65° C., from about 20° C. to 65° C., from about 25° C. to 65° C., from about 30° C. to 65° C., from about 35° C. to 65° C., from about 37° C. to 65° C., from about 40° C. to 65° C., from about 45° C. to 65° C., from about 50° C. to 65° C., from about 55° C. to 65° C., from about 60° C. to 65° C., from about 4° C. to 55° C., from about 10° C. to 50° C., from about 15° C. to 45° C., and from about 20° C. to 40° C.

### 4. Detection Assays and Systems

Embodiments of the present disclosure include compositions, systems, assays, and methods for detecting one or more analytes in a sample. In accordance with these embodiments, described below are exemplary assays and devices for use with various embodiments herein. The following devices and assays should not be viewed as limiting the full scope of the systems, assays, and methods described herein.

a. Lateral Flow Assays

In certain embodiments, the present disclosure provides compositions and materials for conducting a lateral flow assay (e.g., a lateral flow immunoassay). Lateral flow assays are based on the principles of immunochromatography and can be used to detect, quantify, test, measure, and monitor a wide array of analytes, such as, but not limited to, analytes pertaining to monitoring ovulation, detecting/diagnosing infectious diseases/organisms, analyzing drugs of abuse, detecting/quantifying analytes important to human physiology, security screening, veterinary testing, agriculture applications, environmental testing, product quality evaluation, etc.

As shown in FIG. 1, embodiments of the present disclosure include lateral flow detection systems (100) for detecting and/or quantifying a target analyte based on bioluminescent complex formation. In some embodiments, lateral flow assay systems of the present disclosure include an analytical membrane (105) that is divided into one or more detection regions (110) and one or more control regions (115). The detection region or regions can include a target analyte binding agent immobilized to a portion of the detection region such that it is not displaced when facilitating lateral flow across the analytical membrane. Lateral flow assay systems of the present disclosure can also include a conjugate pad (120) within which a target analyte binding agent is contained. In some embodiments, a target analyte binding agent is contained within the conjugate pad but flows from the conjugate pad and across the analytical membrane towards the detection and control regions when lateral flow occurs. Lateral flow assay systems of the present disclosure can also include a sample pad (125) that is positioned at one distal end of the lateral flow assay system (e.g., opposite an absorbent pad). A sample that contains (or may contain) a target analyte is applied to the sample pad. In some embodiments, a lateral flow assay system also comprises a wicking pad (130) at an end of the device distal to the sample pad. The wicking pad generates capillary flow of the sample from the sample pad through the conjugate pad, analytical membrane, detection region, and control region.

In accordance with these embodiments, upon addition of a sample to the sample pad, the facilitation of lateral flow causes a target analyte within the sample to contact a first target analyte binding agent within the conjugate pad; subsequently, lateral flow causes the target analyte and the first target analyte binding agent to contact a second target analyte binding agent immobilized to a detection region of the analytical membrane. The presence and/or quantity of the target analyte is then determined based on detection of the analyte in the detection region (e.g., in the presence of a luminogenic substrate for the bioluminescent complex) and/or in comparison to the control.

In some embodiments, the above lateral flow systems make use of one or more NanoLuc®-based technologies (e.g., NanoBiT, NanoTrip, NanoBRET, etc.) for detection of the bound target analyte.

In an exemplary embodiment, as shown in FIG. 1, a target analyte is an antibody generated in a subject in response to being exposed to an infectious disease/organism. The first target analyte binding agent includes a both a target analyte binding element that binds the antibody (e.g., a non-specific antibody binding agent (e.g., protein L)) and a first peptide or polypeptide capable of interacting with a distinct peptide or polypeptide to generate a bioluminescent signal (e.g., a NanoBiT non-luminescent peptide or polypeptide or variant thereof (e.g., one of SEQ ID NOs: 9-11 or 12/14)). The second target analyte binding agent can include a target analyte binding element that binds the antibody, such as an epitope of an antigen recognized by the antibody, and a second peptide or polypeptide capable of interacting with a the first peptide or polypeptide to generate a bioluminescent signal (e.g., a NanoBiT non-luminescent peptide or polypeptide or variant thereof (e.g., one of SEQ ID NOs: 9-11 or 12/14)). Once the bioluminescent complex firms at the detection region, the bioluminescent signal can be detected and/or quantified (e.g., in the presence of a luminogenic substrate for the bioluminescent complex), thus indicating the presence/quantity of the antibody in the sample.

As shown in FIG. 1, lateral flow assays of the present disclosure can be configured to test for multiple different analytes such as antibodies generated to distinct diseases/microorganisms, in a single sample from a subject (e.g., multiplexing). In accordance with these embodiments, the analytical membrane can include a plurality of detection regions with each detection region comprising a distinct target analyte binding agent having distinct target analyte binding elements (e.g., distinct disease antigens).

In an alternative lateral flow embodiment to the one depicted in FIG. 1, a target analyte is an antibody generated in a subject in response to being exposed to an infectious disease/organism. The first target analyte binding agent that includes a both a target analyte binding element that binds the antibody (e.g., an epitope of an antigen recognized by the antibody) and a bioluminescent polypeptide (e.g., NanoLuc or a variant thereof (e.g., SEQ ID NO: 5, SEQ ID NO: 6)). The second target analyte binding agent can include a target analyte binding element that binds the antibody, such as a non-specific antibody binding agent (e.g., protein L). Detection of bioluminescence in the detection region (e.g., in the presence of a luminogenic substrate for the bioluminescent complex) then indicates that both target analyte binding agents bound to the target analyte, and therefore the target analyte was present in the sample.

In another exemplary alternative embodiment, a target analyte is an antibody generated in a subject in response to being exposed to an infectious disease/organism. The first target analyte binding agent includes a both a target analyte binding element that binds the antibody (e.g., a non-specific antibody binding agent (e.g., protein L), a target-specific (e.g., antibody) binding agent) and a first non-luminescent (NL) peptide tag (e.g., SEQ ID NO: 13 or 11 or variants thereof) capable of interacting with a second non-luminescent (NL) peptide (e.g., SEQ ID NO: 11 or 13 or variants thereof) and a non-luminescent (NL) polypeptide (e.g., SEQ ID NO: 12 or variants thereof) to generate a bioluminescent signal. The second target analyte binding agent includes a target analyte binding element that binds the antibody (e.g., a target-specific (e.g., antibody) binding agent, a non-specific antibody binding agent (e.g., protein L)) and a second NL peptide tag (e.g., SEQ ID NO: 11 or 13 or variants thereof). Formation of the bioluminescent complex in the presence of the NL polypeptide component (e.g., SEQ ID NO: 12 or variants thereof) and a luminogenic substrate in the detection region indicates the presence of the target analyte in the sample. The bioluminescent signal is detected and/or quantified to detect/quantity the antibody in the sample.

Additional alternatives to the exemplary embodiments set forth above are contemplated. For example, alternative binding agents, target analytes, detectable elements, order of the various components (e.g., non-specific binding agent/target-specific binding agent, target-specific binding agent/non-specific binding agent, target-specific binding agent/target-specific binding agent, etc.) are described herein and embodiments incorporating various combinations of the components are within the scope herein.

In some embodiments, a target analyte is not an antibody, but is instead a small molecule, peptide, protein, carbohydrate, lipid, etc. In some embodiments, the lateral flow assays and systems described above are configured (e.g., using one or more NanoLuc®-based technologies (e.g., NanoBiT, NanoTrip, NanoBRET, etc.)) for the detection of any such target analytes.

b. Solid Phase Assays

Embodiments of the present disclosure include compositions, assays, systems, devices, and methods for detecting one or more analytes in a sample. In accordance with these embodiments, the present disclosure provides compositions and materials for conducting a solid phase assay (e.g., a solid phase platform for conducting an immunoassay). Solid phase detection platforms are generally the simplest form of an immunoassay and can be used to detect, quantify, test, measure, and monitor a wide array of analytes such as, but not limited to, analytes pertaining to monitoring ovulation, detecting/diagnosing infectious diseases/organisms, analyzing drugs of abuse, detecting/quantifying analytes important to human physiology, veterinary testing, security screening, agriculture applications, environmental testing, and product quality evaluation. In contrast to lateral flow assays, solid phase detection platforms do not involve facilitating the flow of assay reagents across a membrane, but instead typically include a solid support to which components of the assay are attached or contained within (e.g., dipstick test or spot test).

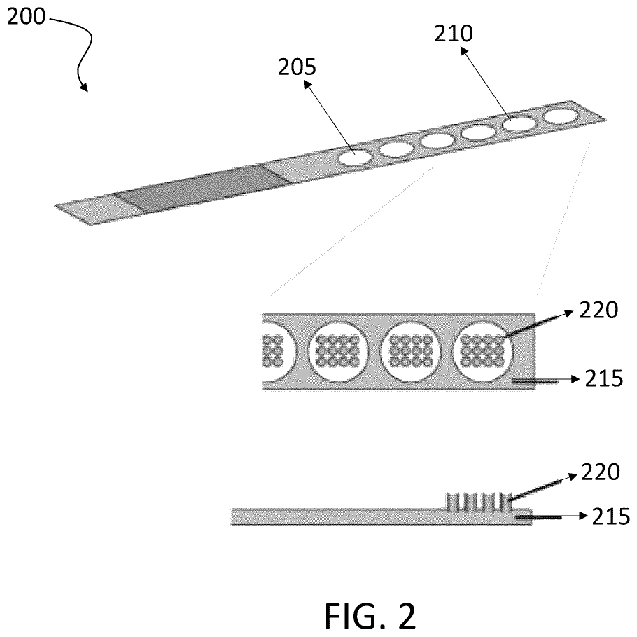

As shown in FIG. 2, embodiments of the present disclosure include solid phase detection platforms (200) for detecting and/or quantifying a target analyte based on bioluminescent complex formation. In some embodiments, solid phase detection platforms of the present disclosure include one or more detection regions (205) and one or more control regions (210) to which a sample is applied. In some embodiments, the detection region or regions includes a target analyte binding agent within and/or conjugated to a portion of the detection region. Solid phase detection platforms of the present disclosure can also include a solid support (215) to which the detection regions and the control regions are attached and demarcated from each other, and which allow for a sample to be applied to the detection and control regions (e.g., dipstick test).

In accordance with these embodiments, a sample or a portion of a sample is applied to the detection and control regions of the solid phase assay platform such that a target analyte contacts a target analyte binding agent (220) conjugated to and/or within the detection region under conditions such that the binding event and/or the immobilization of the target analyte on the solid phase is detectable (e.g., a bioluminescent entity is immobilized, a bioluminescent complex is formed), thereby indicating the presence of the analyte in the sample.

In some embodiments, the solid phase assay platform includes a first target analyte binding agent (e.g., a target-specific binding agent (e.g., target-specific antibody, antigen for the target antibody, etc.)) immobilized on the solid phase. A second target analyte binding agent (e.g., a target-specific binding agent (e.g., target-specific antibody, antigen for the target antibody, etc.), a non-specific binding agent (e.g., protein L)) linked to a bioluminescent polypeptide (e.g., SEQ ID NO: 5 or variants thereof) is added to the solid phase with the sample (e.g., concurrently, sequentially, etc.). If both target analyte binding agent bind to the target analyte, the bioluminescent polypeptide becomes immobilized on the solid phase. Detection/quantification of bioluminescence on the solid phase (e.g., after a wash step) indicates the presence/amount of target analyte in the sample. In some cases, the first target analyte binding agent is conjugated to the detection region, and the second target analyte binding agent (attached to the bioluminescent polypeptide) is applied to the detection region with or without the sample. In some cases, the second target analyte binding agent is conjugated to the detection region, and the first target analyte binding agent (attached to the bioluminescent polypeptide) is applied to the detection region with or without the sample. In accordance with these embodiments, immobilization of bioluminescence at the detection region can be detected and/or quantified when in the presence of a luminogenic substrate (as described further below), thus indicating the presence (or absence) of the antibody in the sample.

In alternative embodiments, a solid phase assay platform utilizes a binary complementation approach, in which a bioluminescent complex is formed upon binding of two non-luminescent (NL) peptide/polypeptide components (e.g., NanoBiT system), to detect a target analyte. Multiple configurations of solid phase assays and systems utilizing a binary complementation approach are within the scope herein. For example, an exemplary system can include (i) a first target analyte binding agent linked to a first NL peptide or NL polypeptide (e.g., SEQ ID NOs: 9 or 10 or variants thereof) capable of interacting with high affinity with a second distinct NL polypeptide or NL peptide (e.g., SEQ ID NOs: 10 or 9 or variants thereof) to generate a bioluminescent signal, and (ii) a second target analyte binding agent linked to the complementary NL polypeptide or NL peptide, wherein the second target analyte binding agent is immobilized to the solid phase. Upon binding of the target analyte binding agents to the target analyte, a bioluminescent complex is formed on the solid phase and the bioluminescent signal is detectable/quantifiable, when in the presence of a luminogenic substrate (as described further below).

In other embodiments, a solid phase assay platform utilizes a tripartite complementation approach, in which a bioluminescent complex is formed upon binding of two non-luminescent (NL) peptide components and a non-luminescent (NL) polypeptide component (e.g., NanoTrip system), to detect a target analyte. In some embodiments, the solid phase assay platform includes: (i) a first target analyte binding agent comprising both a target analyte binding element (e.g., general or specific) and a NL peptide (e.g., SEQ ID NOs: 11 or 13) capable of forming a tripartite bioluminescent complex (e.g., NanoTrip complex), (ii) a second target analyte binding agent comprising both a target analyte binding element (e.g., specific) and a NL peptide (e.g., SEQ ID NOs: 11 or 13) capable of forming a tripartite bioluminescent complex (e.g., NanoTrip complex), (iii) a NL polypeptide component of the tripartite bioluminescent complex (e.g., NanoTrip complex), and (iv) a luminogenic substrate. In some cases, the first target analyte binding agent is conjugated to the detection region, and the second target analyte binding agent is applied to the detection region with or without the sample. In some cases, the second target analyte binding agent is conjugated to the detection region, and the first target analyte binding agent is applied to the detection region with or without the sample. Once the bioluminescent complex forms at the detection region, the bioluminescent signal is detected and/or quantified, thus indicating the presence (or absence) of the antibody in the sample.

In other embodiments, the solid phase assay platform includes (i) a first target analyte binding agent comprising a target analyte binding element and a NanoLuc®-based peptide or polypeptide, (ii) target analyte binding agent comprising a target analyte binding element and a fluorophore, and (iii) optionally the additional peptide/polypeptide components to form a bioluminescent complex (e.g., in embodiments in which the NanoLuc®-based peptide or polypeptide is not a bioluminescent polypeptide, e.g. non-luminescent), wherein upon binding of the first and second target analyte binding agents to a target analyte in a sample, in the presence of any additional components necessary for bioluminescence (e.g., luminogenic substrate, complementary components, etc.), emission from the NanoLuc®-based components (e.g., NanoLuc® protein or bioluminescent complex) excites the fluorophore (e.g., via BRET). In some cases, the first target analyte binding agent is conjugated to the detection region, and the second target analyte binding agent is applied to the detection region with or without the sample. In some cases, the second target analyte binding agent is conjugated to the detection region, and the first target analyte binding agent is applied to the detection region with or without the sample.

As shown in FIG. 2, solid phase platforms of the present disclosure can be configured to test for multiple different analytes, such as antibodies generated to distinct diseases/microorganisms, in a single sample from a subject (e.g., multiplexing). In accordance with these embodiments, the solid phase platforms can include a plurality of detection regions with each detection region comprising a distinct target analyte binding agent having distinct target analyte binding elements (e.g., distinct disease antigens).

In some embodiments, the solid phase platforms of the present disclosure can include a plurality of detection regions such as one or more wells of a microtiter plate, for example. In such embodiments, one or more distinct target analyte binding agents can be conjugated (e.g., coated) to wells of the microtiter plate along one or more of the other detection reagents required to carry out a particular bioluminescent assay (e.g., a second target analyte binding agent, a luminogenic substrate, assay buffer, etc.). In some embodiments, one or more of the other detection reagents (reagents not conjugated to the microtiter plate) required to carry out the assay can be added to the wells of the microtiter plate in the form of a lyophilized cake (lyocake) or tablet and reconstituted as part of the bioluminescent assay.

c. Solution Phase Assays

Embodiments of the present disclosure include compositions, assays, systems, devices, and methods for detecting one or more analytes in a sample. In accordance with these embodiments, the present disclosure provides compositions and materials for conducting a solution phase assay (e.g., a liquid-based format for conducting an immunoassay within a solution). Solution phase detection platforms can be used to detect, quantify, test, measure, and monitor a wide array of analytes such as, but not limited to, analytes pertaining to monitoring ovulation, detecting/diagnosing infectious diseases/organisms, analyzing drugs of abuse, detecting/quantifying analytes important to human physiology, veterinary testing, security screening, agriculture applications, environmental testing, and product quality evaluation. In contrast to lateral flow assays and solid phase detection platforms, solution phase detection platforms typically include a receptacle for the solution/liquid in which reactions involving the detection reagents take place, instead of conjugating one or more of the detection reagents to a solid support or membrane to facilitate detection.

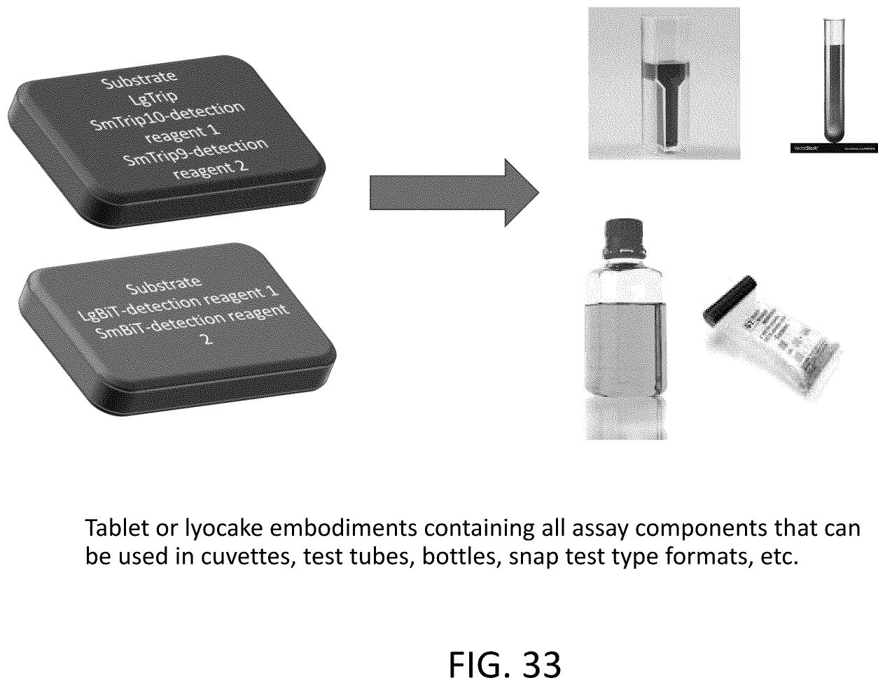

For example, as shown in FIG. 33, embodiments of solution phase platforms of the present disclosure can include one or more components of the bioluminescent complexes in a tablet or lyophilized cake that can be reconstituted in a solution (e.g., buffered solution) to facilitate analyte detection. In some embodiments, the tablet or lyocake can include all the reagents necessary to carry out a reaction to detect an analyte. Such lyocakes or tablets are compatible with many different assay formats, including but not limited to, cuvettes, wells of microtiter plates (e.g., 96-well microtiter plate), test tubes, large volume bottles, SNAP assays, and the like.

In some embodiments, the solution phase assay platform includes a lyocake or tablet comprising one or more of a first target analyte binding agent (e.g., a target-specific binding agent (e.g., target-specific antibody, antigen for the target antibody, etc.)), a second target analyte binding agent (e.g., a target-specific binding agent (e.g., target-specific antibody, antigen for the target antibody, etc.), and a non-specific binding agent (e.g., protein L)) linked to a bioluminescent polypeptide (e.g., SEQ ID NO: 5 and variants thereof). Detection/quantification of bioluminescence in the solution indicates the presence/amount of target analyte in the sample.

In some embodiments, a solution phase assay platform utilizes a binary complementation approach, in which a bioluminescent complex is formed upon binding of two non-luminescent (NL) peptide/polypeptide components (e.g., NanoBiT system), to detect a target analyte. Multiple configurations of solution phase assays and systems utilizing a binary complementation approach are within the scope herein. For example, an exemplary system can include (i) a first target analyte binding agent linked to a first NL peptide or NL polypeptide (e.g., SEQ ID NOs: 9 or 10 or variants thereof) capable of interacting with high affinity with a second distinct NL polypeptide or NL peptide (e.g., SEQ ID NOs: 10 or 9 or variants thereof) to generate a bioluminescent signal, and (ii) a second target analyte binding agent linked to the complementary NL polypeptide or NL peptide. Upon binding of the target analyte binding agents to the target analyte, a bioluminescent complex is formed in the solution and the bioluminescent signal is detectable/quantifiable, when in the presence of a luminogenic substrate (as described further below).

In other embodiments, a solution phase assay platform utilizes a tripartite complementation approach, in which a bioluminescent complex is formed upon binding of two non-luminescent (NL) peptide components and a non-luminescent (NL) polypeptide component (e.g., NanoTrip system), to detect a target analyte. In some embodiments, the solution phase assay platform includes: (i) a first target analyte binding agent comprising both a target analyte binding element (e.g., general or specific) and a NL peptide (e.g., SEQ ID NOs: 11 or 13) capable of forming a tripartite bioluminescent complex (e.g., NanoTrip complex), (ii) a second target analyte binding agent comprising both a target analyte binding element (e.g., specific) and a NL peptide (e.g., SEQ ID NOs: 11 or 13) capable of forming a tripartite bioluminescent complex (e.g., NanoTrip complex), (iii) a NL polypeptide component of the tripartite bioluminescent complex (e.g., NanoTrip complex), and (iv) a luminogenic substrate. Once the bioluminescent complex forms in the solution, the bioluminescent signal is detected and/or quantified, thus indicating the presence (or absence) of the antibody in the sample.

In other embodiments, the solution phase assay platform includes (i) a first target analyte binding agent comprising a target analyte binding element and a NanoLuc®-based peptide or polypeptide, (ii) target analyte binding agent comprising a target analyte binding element and a fluorophore, and (iii) optionally the additional peptide/polypeptide components to form a bioluminescent complex (e.g., in embodiments in which the NanoLuc®-based peptide or polypeptide is not a bioluminescent polypeptide, e.g., non-luminescent), wherein upon binding of the first and second target analyte binding agents to a target analyte in a sample, in the presence of any additional components necessary for bioluminescence (e.g., luminogenic substrate, complementary components, etc.), emission from the NanoLuc®-based components (e.g., NanoLuc® protein or bioluminescent complex) excites the fluorophore (e.g., via BRET).

Solution phase platforms of the present disclosure can be configured to test for multiple different analytes (e.g., multiplexing), such as antibodies generated to distinct diseases/microorganisms in a single sample from a subject. In some embodiments, one or more of the detection reagents required to carry out a bioluminescent reaction to detect/quantify an analyte are present in one or more receptacles of a particular assay platform being used (e.g., individual wells of a 96-well plate), for example, as a lyocake or tablet that is to be reconstituted in a buffered solution. In other embodiments, one or more types of a sample solution are already present in the receptacles, and one or more lyocakes or tables are added to the receptacles and rehydrated to facilitate a bioluminescent reaction. In accordance with these embodiments, the solution phase platforms can include a plurality of receptacles comprising a distinct target analyte binding agent having distinct target analyte binding elements (e.g., distinct disease antigens).

d. Other Assays

Embodiments of the present disclosure include compositions, assays, systems, devices, and methods for detecting one or more analytes in a sample using other assay platforms known in the art. For example, target analytes can be detected and/or measured using the bioluminescent polypeptides and/or complexes described herein in the context of a microfluidic and/or chip-based assay. Because microfluidic systems integrate a wide variety of operations for manipulating fluids, such as chemical or biological samples, these systems are applicable to many different areas, such as biological and medical diagnostics. One type of microfluidic device is a microfluidic chip. Microfluidic chips may include micro-scale features (or micro-features), such as channels, valves, pumps, and/or reservoirs for storing fluids, for routing fluids to and from various locations on the chip, and/or for reacting fluidic reagents.

Microfluidic chips, or labs-on-a-chip (LOC), configured with bioluminescent polypeptides and/or complexes that include peptides and polypeptides capable of generating a bioluminescent signal in the presence of the target analyte offer increased flexibility for automation, integration, miniaturization, and multiplexing. For example, pathogen detection based on microfluidic chips use reaction chambers that are usually on the micro- or nano-scale, which allows devices to be miniaturized and portable; this is particularly advantageous for point-of-care testing. LOC technology allows for the integration of sample preparation, amplification, and signal detection, which reduces the time need to generate results. The high throughput and low consumption of sample and reagents make the technology flexible and relatively cost effective. Nucleic acid-based microfluidic pathogen detection for the detection of bacteria, viruses, and fungi that eliminates the need for PCR or real-time PCR for amplification is a distinct advantage of the bioluminescent complexes of the present disclosure.

### 5. Assay Compositions, Components, and Methods of Manufacturing

Embodiments of the present disclosure also include methods of manufacturing an assay platform for use with bioluminescent peptides and polypeptides for target analyte detection. Although assay platforms may vary depending on various factors, such as the analyte being detected, the complexity of the sampling environment, and the diagnostic parameters, the compositions, materials and methods of the present disclosure can be applied to most currently available assay platforms, such as solid phase assays, lateral flow assays, and microfluidic-based assays.

a. Luminogenic Substrates

In some embodiments, methods of manufacturing assay platforms of the present disclosure include application of a luminogenic substrate. Luminogenic substrates, such as coelenterazine, and analogs and derivatives thereof, can decompose during storage thereby resulting in loss of the substrate before addition to or use in a biological assay. Such decomposition can be the result of instability of the luminogenic substrate in solution over time in a temperature-dependent manner. This decomposition results in waste of the luminogenic substrate and reduced sensitivity and reproducibility of luminescent measurements derived from biological assays that employed the decomposed luminogenic substrate.

Provided herein are compositions that include a luminogenic substrate, such as coelenterazine or an analog or derivative thereof. Exemplary coelenterazine analogs include coelenterazine-h, coelenterazine-h-h, furimazine, JRW-0238, JRW-1404, JRW-1482, JRW-1667, JRW-1743, and JRW-1744.

In some embodiments, the substrate is coelenterazine, which has the following structure:

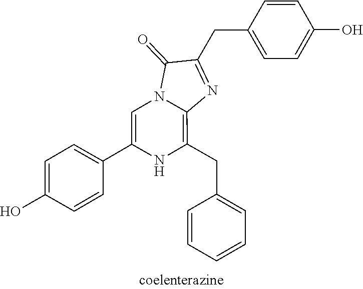 [SMILES: O=c1c(Cc2ccc(O)cc2)nc2c(Cc3ccccc3)[nH]c(-c3ccc(O)cc3)cn1-2]

Exemplary coelenterazine analogs include coelenterazine-h (2-deoxycoelenterazine or 2,8-dibenzyl-6-(4-hydroxyphenyl)imidazo[1,2-a]pyrazin-3(7H)-one), coelenterazine-h-h (dideoxycoelenterazine or 2,8-dibenzyl-6-phenylimidazo[1,2-a]pyrazin-3(7H)-one), furimazine (8-benzyl-2-(furan-2-ylmethyl)-6-phenylimidazo[1,2-a]pyrazin-3(7H)-one), JRW-0238 (8-benzyl-2-(furan-2-ylmethyl)-6-(3-hydroxyphenyl)imidazo[1,2-a]pyrazin-3(7H)-one), JRW-1404 (8-benzyl-6-(2-fluoro-3-hydroxyphenyl)-2-(furan-2-ylmethyl)imidazo[1,2-a]pyrazin-3(7H)-one), JRW-1482 (. 6-(3-amino-2-fluorophenyl)-8-benzyl-2-(furan-2-ylmethyl)imidazo[1,2-a]pyrazin-3(7H)-one), JRW-1667 (6-(3-amino-2-fluorophenyl)-8-(2-fluorobenzyl)-2-(furan-2-ylmethyl)imidazo[1,2-a]pyrazin-3(7H)-one), JRW-1744 (6-(3-amino-2-fluorophenyl)-8-benzyl-2-(furan-2-ylmethyl)imidazo[1,2-a]pyrazin-3(7H)-one), and JRW-1743 (6-(3-amino-2-fluorophenyl)-8-(2-fluorobenzyl)-2-(furan-2-ylmethyl)imidazo[1,2-a]pyrazin-3(7H)-one), which have the following structures:

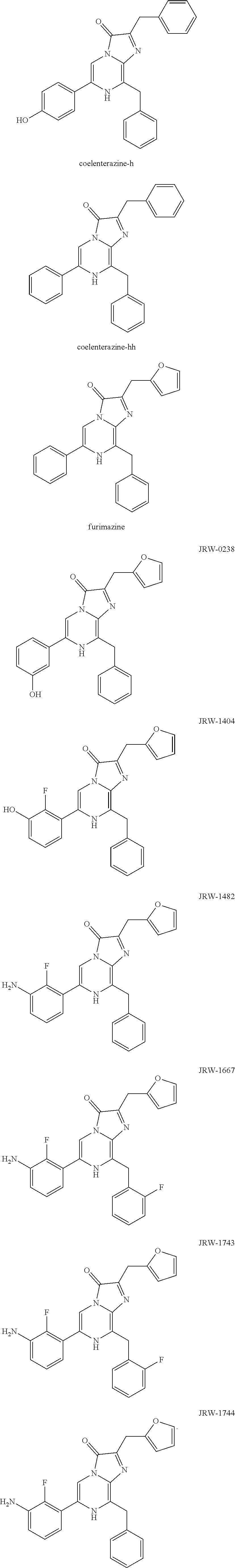

Additional exemplary coelenterazine analogs include coelenterazine-n, coelenterazine-f, coelenterazine-hcp, coelenterazine-cp, coelenterazine-c, coelenterazine-e, coelenterazine-fcp, coelenterazine-i, coelenterazine-icp, coelenterazine-v, 2-methyl coelenterazine, and the like. In some embodiments, the compound may be a coelenterazine analog described in WO 2003/040100; U.S. Pat. Pub. 2008/0248511 (e.g., paragraph [0086]); U.S. Pat. No. 8,669,103; WO 2012/061529; U.S. Pat. Pub. 2017/0233789; U.S. Pat. No. 9,924,073; U.S. Pat. Pub. 2018/0030059; U.S. Pat. No. 10,000,500; U.S. Pat. Pub. 2018/0155350; U.S. patent application Ser. No. 16/399,410 (PCT/US2019/029975); U.S. patent application Ser. No. 16/548,214 (PCT/US2019/047688); U.S. Pat. Pub. 2014/0227759; U.S. Pat. Nos. 9,840,730; 7,268,229; 7,537,912; 8,809,529; 9,139,836; 10,077,244; 9,487,520; 9,924,073; 9,938,564; 9,951,373; 10,280,447; 10,308,975; 10,428,075; the disclosures of which are incorporated by reference herein in their entireties. In some embodiments, coelenterazine analogs include pro-substrates such as, for example, those described in U.S. Pat. Pub. 2008/0248511; U.S. Pat. Pub. 2012/0707849; U.S. Pat. Pub. 2014/0099654; U.S. Pat. Nos. 9,487,520; 9,927,430; 10,316,070; herein incorporated by reference in their entireties. In some embodiments, the compound is furimazine. In some embodiments, the compound is JRW-0238. In some embodiments, the compound is JRW-1743. In some embodiments, the compound is JRW-1744.

Provided herein are compositions that include a luminogenic substrate, such as coelenterazine or an analog or derivative thereof, and a polymer or a paper/fibrous substrate for the manufacture of bioluminescent target analyte detection platforms. Compositions that stabilize and/or enhance the reconstitution efficiency of luminogenic substrates such as coelenterazine or an analog or derivative thereof, are described in U.S. patent application Ser. No. 16/592,310 (PCT/US2019/054501); herein incorporated by reference in its entirety. In some embodiments, the composition stabilizes the compound against decomposition. In some embodiments, the composition stabilizes the compound against decomposition as compared to a composition that does not contain the polymer or paper/fibrous substrate. In some embodiments, the polymer or the paper/fibrous substrate reduces or suppresses the formation of one or more decomposition products from the compound. In some embodiments, the compositions enhance the reconstitution efficiency or reconstitution rate of the substrate.

Additionally, embodiments of the present disclosure include means for stabilizing (e.g., enhancing storage stability) the compositions described further herein. In some embodiments, enhancing the storage stability of the compositions provided herein includes methods and compositions for stabilizing a luminogenic substrate. The luminogenic substrate may be, but is not limited to, coelenterazine, coelenterazine-h, coelenterazine-h-h, furimazine, a derivative thereof, an analog thereof, or any combination thereof. The compositions may include the luminogenic substrate, a thionucleoside, and an organic solvent. The composition may not include or contain a luminogenic enzyme. As provided in U.S. Pat. No. 9,676,997, which is herein incorporated by reference, a thionucleoside may be a compound of formula (I) or a tautomer thereof,

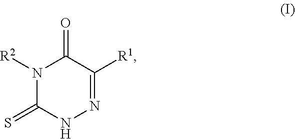

wherein

R¹ is hydrogen, alkyl, substituted alkyl, alkyl-aryl, alkyl-heteroaryl, cycloalkyl, aryl, heteroaryl, carboxylic acid, ester, NR^{a}Rb, imine, hydroxyl, or oxo;

R²is hydrogen, NR^{a}R^{b}, imine, alkyl, or aryl; and

R^{a} and R^{b} are each independently hydrogen, alkyl, or aryl.

In some embodiments, the compound of formula (I) may be ATT (6-methyl-3-thioxo-3,4-dihydro-1,2,4-triazin-5(2H)-one); 3-(4-Amino-5-oxo-3-thioxo-2,3,4,5-tetrahydro-1,2,4-triazin-6-yl)propanoic acid; tetrahydro-2-methyl-3-thioxo-1,2,4-triazine-5,6-dione; 4-((2-furylmethylene)amino)-3-mercapto-6-methyl-1,2,4-triazin-5(4H)-one; 6-benzyl-3-sulfanyl-1,2,4-triazin-5-ol; 4-amino-3-mercapto-6-methyl-1,2,4-triazin-5(4H)-one; 3-(5-oxo-3-thioxo-2,3,4,5-tetrahydro-1,2,4-triazin-6-yl)propanoic acid; (E)-6-methyl-4-((thiophen-2-ylmethylene)amino)-3-thioxo-3,4-dihydro-1,2,4-triazin-5(2H)-one; (E)-6-methyl-4-((3-nitrobenzylidene)amino)-3-thioxo-3,4-dihydro-1,2,4-triazin-5(2H)-one; (E)-4-((4-(diethylamino)benzylidene)amino)-6-methyl-3-thioxo-3,4-dihydro-1,2,4-triazin-5(2H)-one; ATCA ethyl ester; TAK-0021,TAK-0020, TAK-0018, TAK-0009,TAK-0014, TAK-0007, TAK-0008, TAK-0003, and TAK-0004, as provided in U.S. Pat. No. 9,676,997 (incorporated herein by reference); 3-thioxo-6-(trifluoromethyl)-3,4-dihydro-1,2,4-triazin-5(2H)-one; 6-cyclopropyl-3-thioxo-3,4-dihydro-1,2,4-triazin-5(2H)-one; 6-(hydroxymethyl)-3-thioxo-3,4-dihydro-1,2,4-triazin-5(2H)-on; or any combinations thereof.

In some embodiments, a thionucleoside may stabilize the luminogenic substrate against decomposition over time, in the presence of light, in the absence of light, and/or at different temperatures. The thionucleoside may stabilize the luminogenic substrate against decomposition into one or more decomposition products over time, in the presence of light, in the absence of light, and/or at different temperatures. As such, inclusion of the thionucleoside in the compositions described further herein may stabilize the luminogenic substrate against decomposition by suppressing or reducing the formation of the one or more decomposition products as compared to a composition that does not include the thionucleoside. This, in turn, provides the capability of storing or incubating the luminogenic substrate for a period of time at a particular temperature, in the presence of light, and/or in the absence of light without significant decomposition of the luminogenic substrate before use of the luminogenic substrate in an assay. In accordance with these embodiments, the inclusion of a thionucleoside in the compositions described herein can enhance storage stability of the compositions. These embodiments also relate to methods for stabilizing the luminogenic substrate. Such a method may stabilize the luminogenic substrate against decomposition and/or suppress or reduce the formation of the one or more decomposition products. The method may include contacting the luminogenic substrate with an effective amount of the thionucleoside (e.g., 225 mM) in the presence of the organic solvent. This contacting step may include forming the composition described above.

In some embodiments, one or more of the non-luminescent (NL) peptide/polypeptide components that form the bioluminescent complexes described above can be included with or without a luminogenic substrate as part of a composition, such as a lyophilized powder. These compositions can be applied directly, with or without other components, to a portion of a detection platform, or they can be reconstituted as part of a separate solution that is applied to the detection platform.

Coelenterazine and analogs and derivatives thereof may suffer from challenges associated with their reconstitution into buffer systems used in many assays such as the bioluminogenic methods described herein. For example, coelenterazines, or analogs or derivatives thereof, such as furimazine, may dissolve slowly and/or inconsistently in buffer solutions (e.g., due to the heterogeneous microcrystalline nature of the solid material). While dissolution in organic solvent prior to dilution with buffer may provide faster and more consistent results, coelenterazine compounds may suffer from instability in organic solutions on storage, including both thermal instability and photo-instability. In some embodiments, the composition further comprises a polymer. As further described herein, the presence of the polymer may stabilize the compound against decomposition, and the presence of the polymer may improve the solubility of the compound in water or in aqueous solutions.

The polymer may be a naturally-occurring biopolymer or a synthetic polymer. In some embodiments, the polymer is a naturally-occurring biopolymer. Suitable naturally-occurring biopolymers are carbohydrates, including disaccharides (e.g., trehalose and maltose), and polysaccharides (e.g., pullulan, dextran, and cellulose). Mixtures of naturally-occurring biopolymers may also be used. In some embodiments, the polymer is pullulan, which is a polysaccharide that includes maltotriose repeating units. Maltotriose is a trisaccharide that includes three glucose units that are linked via a-1,4 glycosidic bonds. The maltotriose units within the pullulan polymer are linked to each other via a-1,6 glycosidic bonds.

In some embodiments, the polymer is a synthetic polymer. A synthetic polymer may be a homopolymer, copolymer, or block copolymer (e.g., diblock copolymer, triblock copolymer, etc.). Non-limiting examples of suitable polymers include, but are not limited to polyamines, polyethers, polyamides, polyesters, polycarbamates, polyureas, polycarbonates, polystyrenes, polyimides, polysulfones, polyurethanes, polyacetylenes, polyethylenes, polyethyeneimines, polyisocyanates, polyacrylates, polymethacrylates, polyacrylonitriles, and polyarylates. Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), poly(ethylene glycol), poly-L-glutamic acid, poly(hydroxy acids), polyanhydrides, polyorthoesters, poly(ester amides), polyamides, poly(ester ethers), polycarbonates, polyalkylenes (e.g., polyethylene and polypropylene), polyalkylene glycols (e.g., poly(ethylene glycol) (PEG)), polyalkylene terephthalates (e.g., poly(ethylene terephthalate), etc.), polyvinyl alcohols (PVA), polyvinyl ethers, polyvinyl esters (e.g., poly(vinyl acetate), etc.), polyvinyl halides (e.g., poly(vinyl chloride) (PVC), etc.), polyvinylpyrrolidone, polysiloxanes, polystyrene (PS), polyurethanes, derivatized celluloses (e.g., alkyl celluloses, hydroxyalkyl celluloses, cellulose ethers, cellulose esters, nitro celluloses, hydroxypropylcellulose, carboxymethylcellulose, etc.), polymers of acrylic acids (“polyacrylic acids”) (e.g., poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate), polydioxanone and its copolymers (e.g., polyhydroxyalkanoates, polypropylene fumarate), polyoxymethylene, poloxamers, poly(ortho)esters, poly(butyric acid), poly(valeric acid), poly(lactide-co-caprolactone), trimethylene carbonate, and mixtures and copolymers thereof.

In some embodiments, the composition further comprises a paper substrate. As further described herein, the presence of the paper substrate may stabilize the compound against decomposition, and the presence of the paper substrate may improve the solubility of the compound in aqueous solutions. Exemplary paper substrates include, but are not limited to, Whatman brand papers, (e.g., W-903 paper, FTA paper, FTA Elute paper, FTA DMPK paper, etc.), Ahlstrom papers (e.g., A-226 paper, etc.), M-TFN paper, FTA paper, FP705 paper, Bode DNA collection paper, nitrocellulose paper, nylon paper, cellulose paper, Dacron paper, cotton paper, and polyester papers, and combinations thereof.

In addition to the compound and the polymer and/or the paper substrate, the composition may include additional components such as buffers, surfactants, salts, proteins, or any combination thereof. For example, the composition may include a buffer such as a phosphate buffer, a borate buffer, an acetate buffer, or a citrate buffer, or other common buffers such as bicine, tricine, tris(hydroxymethyl)aminomethane (tris), N-[tris(hydroxymethyl)methyl]-3-aminopropanesulfonic acid (TAPS), 3-[N-tris(hydroxymethyl)methylamino]-2-hydroxypropanesulfonic acid (TAPSO), 2-[4-(2-hydroxyethyl)piperazin-1-yl]ethanesulfonic acid (HEPES), N-[tris(hydroxymethyl)methyl]-2-aminoethanesulfonic acid (TES), piperazine-N,N′-bis(2-ethanesulfonic acid) (PIPES), 2-(N-morpholino)ethanesulfonic acid (MES), or the like.

In some embodiments, the composition may include a surfactant. Exemplary surfactants include non-ionic surfactants, anionic surfactants, cationic surfactants, and zwitterionic surfactants. For example, the surfactant may be a non-ionic surfactant such as sorbitan 20.

In some embodiments, the composition may include a salt, such as sodium chloride, potassium chloride, magnesium chloride, or the like.

In some embodiments, the composition may include a protein. For example, the composition can include a carrier protein to prevent surface adsorption of luminogenic enzymes that may be added in downstream assays. In some embodiments, the protein may be bovine serum albumin (BSA).

In some embodiments, the composition may include a substance that reduces autoluminescence. In some embodiments, the substance is ATT (6-Aza-2-thiothymine), a derivative or analog of ATT, a thionucleoside, thiourea, and the like. In some embodiments, the substance is a thionucleoside disclosed in U.S. Pat. No. 9,676,997, herein incorporated by reference. In some embodiments, the substance is thiourea, which use for reducing autoluminescence is disclosed in U.S. Pat. Nos. 7,118,878; 7,078,181; and 7,108,996, herein incorporated by reference.

The composition may be in the form of a lyophilized powder. Such a composition can be prepared by drying a mixture of the components of the composition. For example, the composition can be prepared by dissolving the compound in a solvent (e.g., an organic solvent) to form a first solution, adding the polymer to the first solution to form a second solution, and then drying the second solution to provide the composition. In some embodiments, the drying step may comprise lyophilization. This may provide the composition in the form of a powder. In some embodiments, the drying step may comprise air-drying. This may provide the composition in the form of a malleable disk.

In some embodiments (e.g., those in which the composition includes a polymer rather than a paper substrate), the composition is in the form of a solution. When the composition is a solution, the composition may have a pH of about 5.5 to about 8.0, e.g., about 6.5 to about 7.5. In some embodiments, the composition has a pH of about 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

b. Lateral Flow Components

In some embodiments, the present disclosure provides methods of manufacturing a lateral flow assay platform that includes a conjugate pad, an analytical membrane, a sample pad, and other components necessary for facilitating lateral flow across a membrane (e.g., an absorbent pad). For example, a conjugate pad can include at least one target analyte binding agent reversibly conjugated to the conjugate pad, such that the target analyte binding agent is able to be transferred from the conjugate pad to the analytical membrane when lateral flow is applied, whereupon the target analyte binding agent can bind a target analyte and form a bioluminescent complex. In some embodiments, the target analyte binding agent includes a target analyte binding element to facilitate binding to the target analyte, as well as a bioluminescent polypeptide or component of a bioluminescent complex, such as a bioluminescent polypeptide of SEQ ID NO: 5 (NanoLuc and variants thereof), a non-luminescent (NL) polypeptide of SEQ ID NO: 9 (LgBiT), an NL peptide of SEQ ID NO: 10 (SmBiT), an NL peptide of SEQ ID NO: 11 (HiBiT), an NL polypeptide of SEQ ID NO: 12 (LgTrip-3546), an NL peptide of SEQ ID NO: 13 (SmTrip), an NL peptide of SEQ ID NO: 14 (β9/β10 dipeptide), or variants thereof. In some embodiments, target analyte binding agent comprises a fluorophore capable of being activated by energy transfer (e.g., from a bioluminescent polypeptide or component of a bioluminescent complex).

In some embodiments, the conjugate pad comprises a first target analyte binding agent. In some embodiments, the first target analyte binding agent comprises a first target analyte binding element and a first bioluminescent polypeptide or a first component of a bioluminescent complex (e.g., NL peptide or NL polypeptide). In some embodiments, the target analyte binding agent is stored on or within the conjugate pad such that it remains with the conjugate pad until being displaced by lateral from through the device.

In some embodiments, the conjugate pad comprises a luminogenic substrate, such as coelenterazine, coelenterazine-h, coelenterazine-h-h, furimazine, JRW-0238, JRW-1404, JRW-1482, JRW-1667, JRW-1743, JRW-1744, other coelenterazine analogs or derivatives, a pro-substrate, and/or other substrates (e.g., coelenterazine analog or derivative) described herein. In some embodiments, the luminogenic substrate is reversibly conjugated to the conjugate pad. In some embodiments, the luminogenic substrate is dried on or within the conjugate pad. In some embodiments, the luminogenic substrate is part of a composition comprising the luminogenic substrate and a polymer selected from pullulan, trehalose, maltose, cellulose, dextran, polystyrene, poly(meth)acrylate, and a combination of any thereof (e.g., described in greater detail above and/or in U.S. Prov. Appln. Ser. No. 62/740,622. In some embodiment, the luminogenic substrate is applied as part of a composition or solution, such as a protein buffer. In some embodiment, the protein buffer includes 20 mM Na3PO₄; 5% w/v BSA; 0.25% v/v Tween 20; 10% w/v sucrose. In some embodiments, luminogenic substrate is added to the protein buffer and dried for 1 hour at 37° C. onto a substrate or matrix (e.g., filter paper or membrane). In other embodiments, the luminogenic substrate is applied as a separate reagent as part of an assay method or system.

In some embodiments, the assay platform includes an analytical membrane comprising a detection region and a control region to facilitate the detection of the bioluminescent complex indicating target analyte detection. The detection region can include at least one target analyte binding agent immobilized to the detection region such that it will not be displaced by the application of lateral flow across the membrane. In some embodiments, the analytical membrane includes at least one target analyte binding agent. In some embodiments, the target analyte binding agent comprises a target analyte binding element and a bioluminescent polypeptide or a first component of a bioluminescent complex (e.g., NL peptide or NL polypeptide).

In some embodiments, the analytical membrane includes a plurality of detection regions with each detection region comprising a distinct target analyte binding agent comprising distinct target analyte binding elements (e.g., multiplexing capability).

In some embodiments, the analytical membrane comprises a luminogenic substrate, such as coelenterazine, coelenterazine-h, coelenterazine-h-h, furimazine, JRW-0238, JRW-1404, JRW-1482, JRW-1667, JRW-1743, JRW-1744, other coelenterazine analogs or derivatives, a pro-substrate, or other substrates (e.g., coelenterazine analog or derivative) described herein. In some embodiments, the luminogenic substrate is reversibly conjugated to and/or contained on/within the analytical membrane, for example, as part of a composition comprising the luminogenic substrate and a polymer selected from pullulan, trehalose, maltose, cellulose, dextran, polystyrene, poly(meth)acrylate, and a combination of any thereof. In some embodiment, the luminogenic substrate is applied as part of a composition or solution, such as a protein buffer. In some embodiment, the protein buffer includes 20 mM Na3PO₄; 5% w/v BSA; 0.25% v/v Tween 20; 10% w/v sucrose. In some embodiments, the protein buffer includes 20 mM Na₃PO₄; 5% w/v BSA; 0.25% v/v Tween 20; 5% w/v pullulan. In some embodiments, the protein buffer includes 20 mM Na₃PO₄; 1-5% w/v BSA; 0.25% v/v Tween 20. In some embodiments, the protein buffer includes 20 mM Na₃PO₄; 1-5% w/v Prionex; 0.25% v/v Tween 20. In some embodiments, the protein buffer includes 20 mM Na₃PO₄; 1-5% w/v BSA, 5 mM ATT. In some embodiments, the protein buffer includes 20 mM Na₃PO₄; 1-5% v/v Prionex, 5 mM ATT. In some embodiments, the protein buffer includes 20 mM Na₃PO₄; 1-5% w/v BSA, 5 mM ATT, 5 mM ascorbate. In some embodiments, the protein buffer includes 20 mM Na₃PO₄; 1-5% w/v Prionex, 5 mM ATT, 5 mM ascorbate. In some embodiments, the protein buffer includes 20 mM Na₃PO₄; 1-5% w/v BSA, 5 mM ATT, 5 mM ascorbate. In some embodiments, the protein buffer includes; 1-5% w/v BSA, 5 mM ATT, 5 mM ascorbate. In some embodiments, luminogenic substrate is added to the protein buffer and dried for 1 hour at 37° C. onto a substrate or matrix (e.g., filter paper or membrane). In other embodiments, the luminogenic substrate is applied as a separate reagent as part of an assay method or system.

c. Solid Phase Components

In some embodiments, the present disclosure provides methods of manufacturing a solid phase detection platform (e.g., dipstick assay or spot test) that includes a detection region and a control region. In some embodiments, the detection region comprises at least one target analyte binding agent conjugated to the detection region. In some embodiments, the detection region comprises at least one target analyte binding agent that is not conjugated to the detection region. Such a non-conjugated binding agent may be added to the detection region (e.g., with the sample or as part of a detection reagent) or may reside on or within the detection region, without conjugation. In some embodiments, the non-conjugated binding agent comprises a target analyte binding element and bioluminescent polypeptide or component of a bioluminescent complex, such as a bioluminescent polypeptide of SEQ ID NO: 5 (NanoLuc and variants thereof), a non-luminescent (NL) polypeptide of SEQ ID NO: 9 (LgBiT), an NL peptide of SEQ ID NO: 10 (SmBiT), an NL peptide of SEQ ID NO: 11 (HiBiT), an NL polypeptide of SEQ ID NO: 12 (LgTrip-3546), an NL peptide of SEQ ID NO: 13 (SmTrip), an NL peptide of SEQ ID NO: 14 (β9/β10 dipeptide), or variants thereof.

In some embodiments, the solid phase detection platform includes a plurality of detection regions with each detection region comprising a distinct target analyte binding agent comprising distinct target analyte binding elements (e.g., multiplexing capability). In some embodiments, one or more distinct target analyte binding agents can be conjugated (e.g., coated) to wells of a microtiter plate, along one or more of the other detection reagents required to carry out a particular assay (e.g., a second target analyte binding agent, a luminogenic substrate, assay buffer, etc.). In other embodiments, the detection reagents can be applied as a separate reagent as part of an assay method or system (e.g., as part of a lyocake or tablet and reconstituted as part of the assay).

The detection platform can also include a luminogenic substrate, such as coelenterazine, coelenterazine-h, coelenterazine-h-h, furimazine, JRW-0238, JRW-1404, JRW-1482, JRW-1667, JRW-1743, JRW-1744, other coelenterazine analogs or derivatives, a pro-substrate, or other substrates (e.g., coelenterazine analog or derivative) described herein. In some embodiments, the luminogenic substrate is reversibly conjugated to the detection region. In some embodiments, the luminogenic substrate is stably stored on or within the detection region. In some embodiments, the luminogenic substrate is part of a composition comprising the luminogenic substrate and a polymer selected from pullulan, trehalose, maltose, cellulose, dextran, polystyrene, poly(meth)acrylate, and a combination of any thereof. In some embodiments, the luminogenic substrate is applied as part of a composition or solution, such as a protein buffer, detection reagent, or with the sample. In some embodiments, the protein buffer includes 20 mM Na₃PO₄; 5% w/v BSA; 0.25% v/v Tween 20; 10% w/v sucrose. In some embodiments, luminogenic substrate is added to the protein buffer and dried for 1 hour at 37° C. onto a substrate or matrix (e.g., filter paper, membrane, individual wells of a microtiter plate). In other embodiments, the luminogenic substrate is applied as a separate reagent as part of an assay method or system (e.g., as part of a lyocake or tablet and reconstituted as part of the assay).

Embodiments of the present disclosure also include methods for producing a substrate or matrix for use in a bioluminescent assay. In accordance with these embodiments, the method includes generating a solution or liquid formulation containing at least one target analyte binding agent comprising a target analyte binding element and one of a polypeptide component of a bioluminescent complex or a peptide component of a bioluminescent complex. In some embodiments, the solution includes a protein buffer and at least one excipient, including but not limited to, a surfactant, a reducing agent, a salt, a radical scavenger, a chelating agent, a protein, or any combination thereof. In some embodiment, the solution includes a complementary peptide or polypeptide component of the bioluminescent complex, such that the target analyte binding agent and the complementary peptide or polypeptide component of the bioluminescent complex form a bioluminescent analyte detection complex in the presence of a target analyte. In some embodiments, the solution comprises a luminogenic substrate.

After generating the solution or liquid formulation, the method includes applying the solution to the surface of a substrate or matrix. In some embodiments, the substrate or matrix is W-903 paper, FTA paper, FTA Elute paper, FTA DMPK paper, Ahlstrom A-226 paper, M-TFN paper, FTA paper, FP705 paper, Bode DNA collection paper, nitrocellulose paper, nylon paper, cellulose paper, Dacron paper, cotton paper, and polyester papers, or combinations thereof. In other embodiments, the substrate or matrix is a mesh comprising plastic, nylon, metal, or combinations thereof.

Embodiments of the method also include drying the substrate or matrix after the solution has been applied to the substrate or matrix. In some embodiments, drying the substrate or matrix containing the solution comprises drying the substrate or matrix at a temperature from about 30° C. to 65° C., from about 30° C. to 60° C., from about 30° C. to 55° C., from about 30° C. to 50° C., from about 30° C. to 45° C., or from about 30° C. to 40° C. In some embodiments, the matrix or substrate is dried from about 15 mins to 8 hours, from about 30 mins to 7 hours, from about 45 mins to 6 hours, from about 1 hour to 5 hours, from about 2 hours to 4 hours, from about 30 mins to 2 hours, or from about 30 mins to 1 hour. In some embodiments, drying the substrate containing the solution comprises lyophilizing and/or freezing the substrate.

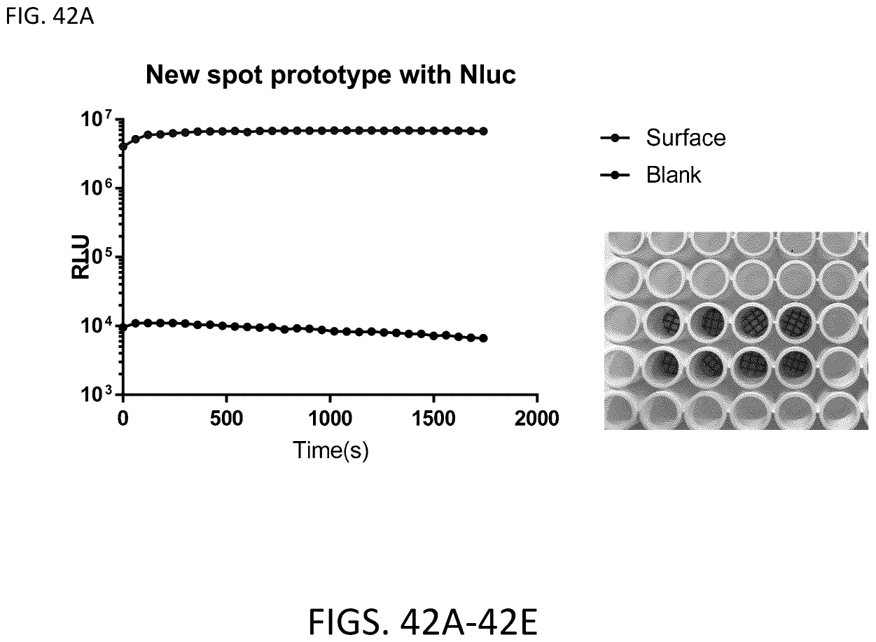

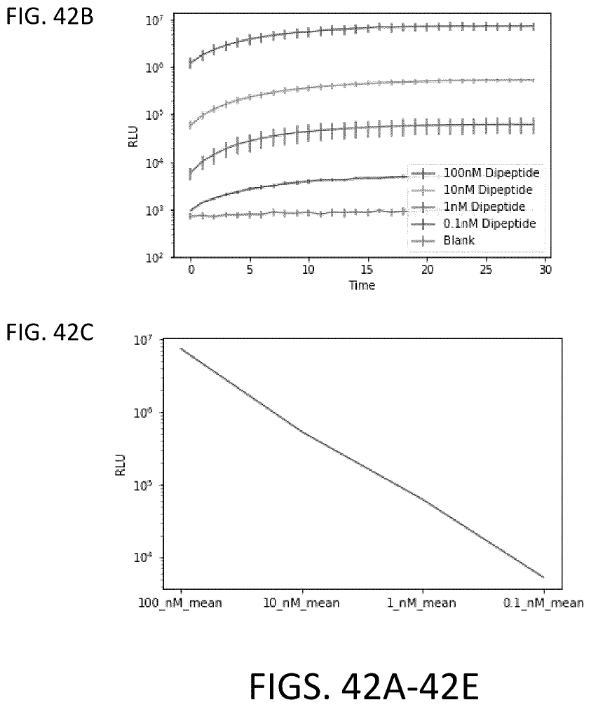

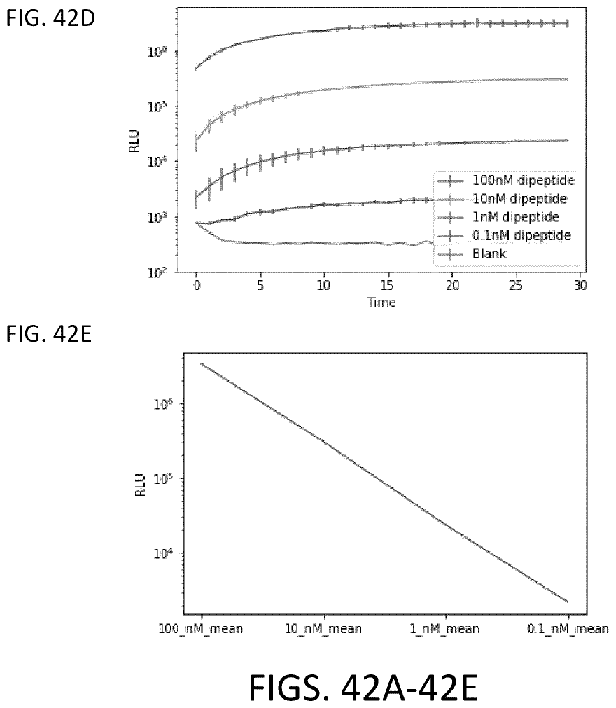

In some embodiments, the method includes drying the at least one target analyte binding agent and/or the complementary peptide or polypeptide component of the bioluminescent complex onto a first substrate, and drying the luminogenic substrate onto a second substrate. In some embodiments, the at least one target analyte binding agent and/or the complementary peptide or polypeptide component of the bioluminescent complex are dried onto a paper based substrate, and the luminogenic substrate is dried onto a mesh (see, e.g., FIGS. 42A-42E).

In accordance with these embodiments, the substrate or matrix can be used in a bioluminescent assay to detect a target analyte. For example, a bioluminescent signal can be generated upon exposure of the substrate or matrix containing the solution to the target analyte. In some embodiments, the bioluminescent signal is proportional to the concentration of the target analyte. In some embodiments, the at least one target analyte binding agent and/or the complementary peptide or polypeptide component of the bioluminescent complex exhibit(s) enhanced stability when dried on the substrate, as described further herein.

d. Solution Phase Components

In some embodiments, the present disclosure provides methods of manufacturing a solution phase detection platform (as described herein) that includes one or more detection regions and control regions (e.g., wells of a 96-well microtiter plate). For example, as shown in FIG. 33, embodiments of solution phase platforms of the present disclosure can include one or more components of the bioluminescent complexes described herein in a tablet or lyophilized cake that can be reconstituted in a solution (e.g., buffered solution) to facilitate analyte detection. In some embodiments, the tablet or lyocake can include all the reagents necessary to carry out a reaction to detect an analyte and are included as part of a solution phase detection platform (e.g., present in one or more wells of a 96-well microtiter plate). Such lyocakes or tablets are compatible with many different assay formats, including but not limited to, cuvettes, wells of microtiter plates (e.g., 96-well microtiter plate), test tubes, large volume bottles, SNAP assays, and the like.

In some embodiments, one or more components of the bioluminescent complexes described herein can be added to a detection region and/or may already be present within a detection region, in the presence or absence of a sample. The detection reagents can then be reconstituted (e.g., rehydrated) as part of carrying out the detection of an analyte in the sample. In some embodiments, the detection reagent comprises a target analyte binding element and bioluminescent polypeptide or component of a bioluminescent complex, such as a bioluminescent polypeptide of SEQ ID NO: 5 (NanoLuc and variants thereof), a non-luminescent (NL) polypeptide of SEQ ID NO: 9 (LgBiT), an NL peptide of SEQ ID NO: 10 (SmBiT), an NL peptide of SEQ ID NO: 11 (HiBiT), an NL polypeptide of SEQ ID NO: 12 (LgTrip-3546), an NL peptide of SEQ ID NO: 13 (SmTrip), an NL peptide of SEQ ID NO: 14 (β9/β10 dipeptide), or variants thereof.

The solution phase detection platform can also include a luminogenic substrate, such as coelenterazine, coelenterazine-h, coelenterazine-h-h, furimazine, JRW-0238, JRW-1404, JRW-1482, JRW-1667, JRW-1743, JRW-1744, other coelenterazine analogs or derivatives, a pro-substrate, or other substrates (e.g., coelenterazine analog or derivative) described herein. In some embodiments, the luminogenic substrate is part of a composition comprising the luminogenic substrate and a polymer selected from pullulan, trehalose, maltose, cellulose, dextran, polystyrene, poly(meth)acrylate, and a combination of any thereof. In some embodiments, the luminogenic substrate is applied as part of a composition or solution, such as a protein buffer, detection reagent, or with the sample. In some embodiments, the luminogenic substrate is applied as a separate reagent as part of an assay method or system, and in other embodiments, it is part of a lyocake or tablet that includes one or more detection reagents.

### 6. Target Analytes

Embodiments of the present disclosure find use in the detection/quantification of target analytes and include target analyte binding agents capable of binding to or interacting with a target analyte via a target analyte binding element. In some embodiments, target analyte binding agents include target analyte binding elements capable of binding a group or class of analytes (e.g., protein L binding generally to antibodies), such binding elements may be referred to herein as “non-specific” or the like; in other embodiments, target analyte binding agents include target analyte binding elements capable of binding a specific analyte (e.g., an antigen binding a monoclonal antibody), such binding elements may be referred to herein as “target specific” or the like.

In some embodiments, target analyte binding agents and corresponding target analyte binding elements are generated to detect one or more analytes associated with a disease state or environmental condition. Target analyte binding elements can be independently selected from the group consisting of an antibody (e.g., polyclonal, monoclonal, and/or recombinant), antibody fragment (e.g., Fab, Fab′, F(ab′)₂, Fv, scFv, Fd, variable light chain, variable heavy chain, diabodies, scFv, etc.), protein A, an Ig binding domain of protein A, protein G, an Ig binding domain of protein G, protein A/G, an Ig binding domain of protein A/G, protein L, a Ig binding domain of protein L, protein M, an Ig binding domain of protein M, an oligonucleotide probe, a peptide nucleic acid, a DARPin, an aptamer, an affimer, a purified protein (e.g., either the analyte itself or a protein that binds to the analyte), and analyte binding domain(s) of proteins.

In some embodiments, target analyte binding elements comprise an antigen or epitope recognized by an antibody (the target analyte) such as an antibody generated by a subject in response to an immunogenic reaction to a pathogen, which can indicate that the subject is infected with the pathogen. In some embodiments, the target analyte is an antibody against Zika virus, Dengue virus, West Nile virus, Yellow Fever virus, and/or Chikungunya virus, and the target analyte binding element is an immunogenic epitope specifically recognized by the antibody. In some embodiments, the target analyte is an antibody against Hep A, B, C, D or E. In some embodiments, the target analyte is an antibody against Mumps, measles, Rubella, RSV, EBV, Herpes, Influenza, Varicella-Zoster, prenatal Zika, or parainfluenza type 1, 2, or 3. In some embodiments, the target analyte is an antibody against Arbovirus, HIV, prenatal Hepatitis, CMV, Hantavirus, polio virus, of parvovirus. In some embodiments, the target analyte is an antibody against Tick borne disease (e.g., Lyme disease). In some embodiments, the target analyte is an antibody against Bordetella pertussis, pneumococcus, chlamydia, streptococcus, M. pneumoniea, S. pneumonie, shigella producing bacteria, E. coli, Enterobacter, syphilis, gonorrhea. In some embodiments, the target analyte is an autoantibody against ANA, Cardiolipin, celiac disease, insulin, GAD65, IA-2, Reticulin, Thyroglobulin, RNP, cytoplasmic neutrophil, thyrptropin receptor, thyroperoxidase, platelet antibody, PLAR2, myocardial, GBM, tissue transglutaminase, or thyroid stimulating. In some embodiments, the target analyte is a toxin or an antibody against a toxin (e.g., diptheria, tetanus). In some embodiments, the target analyte is from a parasite or an antibody against a parasite (e.g., trichinella, trichinosis, Trypanosoma cruzi, Toxoplasma gondii). In some embodiments, the target analyte is a therapeutic biologic or an antibody against the therapeutic biologic (Vedolizumab, Adalimumab, infliximab, certilizumab, entanercept, Opdivo, Keytruda, ipilimumab, Ustekinumab, secukinumab, guselkumab, Tocilizumab, rituximab, panitumumab, trastuzumab, cetuximab, ofatumumab, eptratuzumab, abatacept, tofacitinib).

Other target analytes include known biomarkers associated with a pathogenic organism, such as a virus, bacterium, protozoa, prion, fungus, parasitic nematode, or other microorganism. Disease biomarkers can include markers of the pathogenic organism itself and/or markers of a subject's reaction to an infection by the pathogenic organism. Diseases that can be detected using the assays and methods of the present disclosure include any of the following: Acinetobacter infections (Acinetobacter baumannii), Actinomycosis (Actinomyces sraelii, Actinomyces gerencseriae and Propionibacterium propionicus), African sleeping sickness or African trypanosomiasis (Trypanosoma brucei), AIDS (HIV), Amebiasis (Entamoeba histolytica), Anaplasmosis (Anaplasma species), Angiostrongyliasis (Angiostrongylus), Anisakiasis (Anisakis), Anthrax (Bacillus anthracis), Arcanobacterium haemolyticum infection (Arcanobacterium haemolyticum), Argentine Teagan fever (Junin virus), Ascariasis (Ascaris lumbricoides), Aspergillosis (Aspergillus species), Astrovirus infection (Astroviridae family), Babesiosis (Babesia species), Bacillus cereus infection (Bacillus cereus), Bacterial pneumonia (multiple bacteria), Bacteroides infection (Bacteroides species), Balantidiasis (Balantidium coli), Bartonellosis (Bartonella), Baylisascaris infection (Baylisascaris species), BK virus infection (BK virus), Black Piedra (Piedraia hortae), Blastocystosis (Blastocystis species), Blastomycosis (Blastomyces dermatitidis), Bolivian hemorrhagic fever (Machupo virus), Brazilian hemorrhagic fever (Sabii virus), Brucellosis (Brucella species), Bubonic plague (Yersinia Pestis), Burkholderia infection (usually Burkholderia cepacia and other Burkholderia species), Buruli ulcer (Mycobacterium ulcerans), Calicivirus infection (Caliciviridae family), Campylobacteriosis (Campylobacter species), Candidiasis (usually Candida albicans and other Candida species), Carrion's disease (Bartonella bacilliformis), Cat-scratch disease (Bartonella henselae), Cellulitis (usually Group A Streptococcus and Staphylococcus), Chagas Disease (Trypanosoma cruzi), Chancroid (Haemophilus ducreyi), Chickenpox (Varicella zoster virus or VZV), Chikungunya (Alphavirus), Chlamydia (Chlamydia trachomatis), Cholera (Vibrio cholerae), Chromoblastomycosis (usually Fonsecaea pedrosoi), Chytridiomycosis (Batrachochytrium dendrabatidis), Clonorchiasis (Clonorchis sinensis), Clostridium difficile colitis (Clostridium difficile), Coccidioidomycosis (Coccidioides immitis and Coccidioides posadasii), Colorado tick fever (Colorado tick fever virus or CTFV), Common cold (usually rhinoviruses and coronaviruses), Creutzfeldt-Jakob disease (PRNP), Crimean-Congo hemorrhagic fever (Crimean-Congo hemorrhagic fever virus), Cryptococcosis (Cryptococcus neoformans), Cryptosporidiosis (Cryptosporidium species), Cutaneous larva migrans (usually Ancylostoma braziliense; multiple other parasites), Cyclosporiasis (Cyclospora cayetanensis), Cysticercosis (Taenia solium), Cytomegalovirus infection (Cytomegalovirus), Dengue fever (Dengue viruses: DEN-1, DEN-2, DEN-3 and DEN-4), Desmodesmus infection (Green algae Desmodesmus armatus), Dientamoebiasis (Dientamoeba fragilis), Diphtheria (Corynebacterium diphtheriae), Diphyllobothriasis (Diphyllobothrium), Dracunculiasis (Dracunculus medinensis), Ebola hemorrhagic fever (Ebolavirus or EBOV), Echinococcosis (Echinococcus species), Ehrlichiosis (Ehrlichia species), Enterobiasis (Enterobius vermicularis), Enterococcus infection (Enterococcus species), Enterovirus infection (Enterovirus species), Epidemic typhus (Rickettsia prowazekii), Erythema infectiosum (Parvovirus B19), Exanthem subitum (Human herpesvirus 6 or HHV-6; Human herpesvirus 7 or HHV-7), Fasciolasis (Fasciola hepatica and Fasciola gigantica), Fasciolopsiasis (Fasciolopsis buski), Fatal familial insomnia (PRNP), Filariasis (Filarioidea superfamily), Fusobacterium infection (Fusobacterium species), Gas gangrene (usually Clostridium perfringens; other Clostridium species), Geotrichosis (Geotrichum candidum), Gerstmann-Straussler-Scheinker syndrome (PRNP), Giardiasis (Giardia lamblia), Glanders (Burkholderia mallei), Gnathostomiasis (Gnathostoma spinigerum and Gnathostoma hispidum), Gonorrhea (Neisseria gonorrhoeae), Granuloma inguinale (Klebsiella granulomatis), Group A streptococcal infection (Streptococcus pyogenes), Group B streptococcal infection (Streptococcus agalactiae), Haemophilus influenzae infection (Haemophilus influenzae), Hand, foot and mouth disease (Enteroviruses, mainly Coxsackie A virus and Enterovirus 71 or EV71), Hantavirus Pulmonary Syndrome (Sin Nombre virus), Heartland virus disease (Heartland virus), Helicobacter pylori infection (Helicobacter pylori), Hemolytic-uremic syndrome (Escherichia coli O157:H7, 0111 and 0104:H4), Hemorrhagic fever with renal syndrome (Bunyaviridae family), Hepatitis A (Hepatitis A virus), Hepatitis B (Hepatitis B virus), Hepatitis C (Hepatitis C virus), Hepatitis D (Hepatitis D Virus), Hepatitis E (Hepatitis E virus), Herpes simplex (Herpes simplex virus 1 and 2 (HSV-1 and HSV-2)), Histoplasmosis (Histoplasma capsulatum), Hookworm infection (Ancylostoma duodenale and Necator americanus), Human bocavirus infection (Human bocavirus or HBoV), Human ewingii ehrlichiosis (Ehrlichia ewingii), Human granulocytic anaplasmosis (Anaplasma phagocytophilum), Human metapneumovirus infection (Human metapneumovirus or hMPV), Human monocytic ehrlichiosis (Ehrlichia chaffeensis), Human papillomavirus (HPV) infection (Human papillomavirus or HPV), Human parainfluenza virus infection (Human parainfluenza viruses or HPIV), Hymenolepiasis (Hymenolepis nana and Hymenolepis diminuta), Epstein-Barr virus infectious mononucleosis (Epstein-Barr virus or EBV), Influenza (Orthomyxoviridae family), Isosporiasis (Isospora belli), Kingella kingae infection (Kingella kingae), Kuru (PRNP), Lassa fever (Lassa virus), Legionellosis (Legionella pneumophila), Legionellosis (Legionella pneumophila), Leishmaniasis (Leishmania species), Leprosy (Mycobacterium leprae and Mycobacterium lepromatosis), Leptospirosis (Leptospira species), Listeriosis (Listeria monocytogenes), Lyme disease (Borrelia burgdorferi, Borrelia garinii, and Borrelia afzelii), Lymphatic filariasis (Wuchereria bancrofti and Brugia malayi), Lymphocytic choriomeningitis (Lymphocytic choriomeningitis virus or LCMV), Malaria (Plasmodium species), Marburg hemorrhagic fever (Marburg virus), Measles (Measles virus), Middle East respiratory syndrome (Middle East respiratory syndrome coronavirus), Melioidosis (Burkholderia pseudomallei), Meningococcal disease (Neisseria meningitidis), Metagonimiasis (usually Metagonimus yokagawai), Microsporidiosis (Microsporidia phylum), Molluscum contagiosum (Molluscum contagiosum virus or MCV), Monkeypox (Monkeypox virus), Mumps (Mumps virus), Murine typhus (Rickettsia typhi), Mycoplasma pneumonia (Mycoplasma pneumoniae), Mycetoma (numerous species of bacteria (Actinomycetoma) and fungi (Eumycetoma)), Myiasis (parasitic dipterous fly larvae), Neonatal conjunctivitis (most commonly Chlamydia trachomatis and Neisseria gonorrhoeae), Norovirus (Norovirus), Nocardiosis (usually Nocardia asteroides and other Nocardia species), Onchocerciasis (Onchocerca volvulus), Opisthorchiasis (Opisthorchis viverrini and Opisthorchis felineus), Paracoccidioidomycosis (Paracoccidioides brasiliensis), Paragonimiasis (usually Paragonimus westermani and other Paragonimus species), Pasteurellosis (Pasteurella species), Pediculosis capitis (Pediculus humanus capitis), Pediculosis corporis (Pediculus humanus corporis), Pediculosis pubis (Phthirus pubis), Pertussis (Bordetella pertussis), Plague (Yersinia pestis), Pneumococcal infection (Streptococcus pneumoniae), Pneumocystis pneumonia (Pneumocystis jirovecii), Pneumonia (multiple causes), Poliomyelitis (Poliovirus), Prevotella infection (Prevotella species), Primary amoebic meningoencephalitis (usually Naegleria fowleri), Progressive multifocal leukoencephalopathy (JC virus), Psittacosis (Chlamydophila psittaci), Q fever (Coxiella burnetiid), Rabies (Rabies virus), Relapsing fever (Borrelia hermsii, Borrelia recurrentis, and other Borrelia species), Respiratory syncytial virus infection (Respiratory syncytial virus (RSV)), Rhinosporidiosis (Rhinosporidium seeberi), Rhinovirus infection (Rhinovirus), Rickettsial infection (Rickettsia species), Rickettsialpox (Rickettsia akari), Rift Valley fever (Rift Valley fever virus), Rocky Mountain spotted fever (Rickettsia rickettsia), Rotavirus infection (Rotavirus), Rubella (Rubella virus), Salmonellosis (Salmonella species), Severe Acute Respiratory Syndrome (SARS coronavirus), Scabies (Sarcoptes scabiei), Scarlet fever (Group A Streptococcus species), Schistosomiasis (Schistosoma species), Sepsis (multiple causes), Shigellosis (Shigella species), Shingles (Varicella zoster virus or VZV), Smallpox (Variola major or Variola minor), Sporotrichosis (Sporothrix schenckii), Staphylococcal food poisoning (Staphylococcus species), Staphylococcal infection (Staphylococcus species), Strongyloidiasis (Strongyloides stercoralis), Subacute sclerosing panencephalitis (Measles virus), Syphilis (Treponema pallidum), Taeniasis (Taenia species), Tetanus (Clostridium tetani), Tinea barbae (usually Trichophyton species), Tinea capitis (usually Trichophyton tonsurans), Tinea corporis (usually Trichophyton species), Tinea cruris (usually Epidermophyton floccosum, Trichophyton rubrum, and Trichophyton mentagrophytes), Tinea manum (Trichophyton rubrum), Tinea nigra (usually Hortaea werneckii), Tinea pedis (usually Trichophyton species), Tinea unguium (usually Trichophyton species), Tinea versicolor (Malassezia species), Toxocariasis (Toxocara canis or Toxocara cati), Toxocariasis (Toxocara canis or Toxocara cati), Toxoplasmosis (Toxoplasma gondii), Trachoma (Chlamydia trachomatis), Trichinosis (Trichinella spiralis), Trichomoniasis (Trichomonas vaginalis), Trichuriasis (Trichuris trichiura), Tuberculosis (usually Mycobacterium tuberculosis), Tularemia (Francisella tularensis), Typhoid fever (Salmonella enterica subsp. enterica, serovar typhi), Typhus fever (Rickettsia), Ureaplasma urealyticum infection (Ureaplasma urealyticum), Valley fever (Coccidioides immitis or Coccidioides posadasii), Venezuelan equine encephalitis (Venezuelan equine encephalitis virus), Venezuelan hemorrhagic fever (Guanarito virus), Vibrio vulnificus infection (Vibrio vulnificus), Vibrio parahaemolyticus enteritis (Vibrio parahaemolyticus), Viral pneumonia (multiple viruses), West Nile Fever (West Nile virus), White piedra (Trichosporon beigelii), Yersinia pseudotuberculosis infection (Yersinia pseudotuberculosis), Yersiniosis (Yersinia enterocolitica), Yellow fever (Yellow fever virus), Zygomycosis (Mucorales order (Mucormycosis) and Entomophthorales order (Entomophthoramycosis)), and Zika fever (Zika virus).

### 7. Methods of Detecting, Quantifying, and Diagnosing

Embodiments of the present disclosure include methods of detecting and/or quantifying a target analyte in a sample with an assay platform (e.g., solid phase detection platform or lateral flow assay) that uses bioluminescent polypeptides or bioluminescent complexes (and components thereof; e.g., non-luminescent peptide or polypeptides) for target analyte detection. Embodiments also include methods of diagnosing a disease state or evaluating an environmental condition based on detecting and/or quantifying a target analyte in a sample.

In some embodiments, a method of detecting an analyte in a sample includes using a lateral flow assay system or a solid phase detection platform as described herein. In accordance with these embodiments, the method includes applying a sample to a sample pad; facilitating flow of the sample from the sample pad to a conjugate pad, and then from the conjugate pad to a detection region and a control region on an analytical membrane. The method can include a first target analyte binding agent, a second target analyte binding agent, and a target analyte that form an analyte detection complex in the at least one detection region when the target analyte is detected in the sample. In some embodiments, methods comprise one or more steps of: sample addition, reagent (e.g., detection reagent) addition, washing, waiting, etc.

In some embodiments, the sample is a biological sample from a subject, such as blood, serum, plasma, urine, stool, cerebral spinal fluid, interstitial fluid, and saliva. In other embodiments, the sample is a sample from a natural or industrial environment, such as a water sample, a soil sample, a plant sample, a food sample, a beverage sample, an oil, and an industrial fluid sample. The method includes detecting the target analyte in the sample by detecting a bioluminescent signal generated from the analyte detection complex. In some embodiments, the target analyte in the sample is quantified based on the bioluminescent signal generated from the analyte detection complex. In some embodiments, the method includes diagnosing a subject from which the sample was obtained as having or not having a disease based on the detection of the analyte.

### 8. Competition

Some embodiments herein utilize competition between a labeled analyte and a target analyte in a sample to detect/quantify the target analyte in a sample. Exemplary embodiments comprise the use of (i) an analyte (e.g., identical or similar to the target analyte) labeled with detectable element described herein (e.g., NanoLuc®-based technology (e.g., NanoLuc, NanoBiT, NanoTrip, NanoBRET, or components (e.g., peptides, polypeptides, etc.) of variants thereof)), and (ii) a binding moiety for the target analyte (e.g., fused or linked to a second detectable element described herein (e.g., NanoLuc®-based technology (e.g., NanoLuc, NanoBiT, NanoTrip, NanoBRET, or components (e.g., peptides, polypeptides, etc.) of variants thereof)). In the absence of the target analyte from a sample, the detectable elements produce a detectable signal (e.g., via complementation between the detectable elements, via BRET, etc.) is produced by the system. When the system is exposed to a sample (e.g., biological sample, environmental sample, etc.), the bioluminescent signal is reduced if the target analyte is present in the sample (the labeled analyte will be competed out of the complex).

Various embodiments herein utilize such competition immunoassays for small molecule detection. In some embodiments, the target small molecule is a toxin (e.g., mycotoxin, etc.), metabolite (e.g., amino acid, glucose molecule, fatty acid, nucleotide, cholesterol, steroid, etc.), vitamin (e.g., vitamin A, vitamin B1, vitamin B2, Vitamin B3, vitamin B5, vitamin B7, vitamin B9, vitamin B12, vitamin C, vitamin D, vitamin E, vitamin H or vitamin K, etc.), coenzyme or cofactor (e.g., coenzyme A, coenzyme B, coenzyme M, coenzyme Q, cytidine triphosphate, acetyl coenzyme A, reduced nicotinamide adenine dinucleodtide (NADH), nicotinamide adenine (NAD+), nucleotide adenosine monophosphoate, nucleotide adenosine triphosphate, glutathione, heme, lipoamide, molybdopterin, 3′-phosphoadenosine-5′-phsphosulfate, pyrroloquinoline quinone, tetrahydrobiopterin, etc.), biomarker or antigen (e.g., erythropoietin (EPO), ferritin, folic acid, hemoglobin, alkaline phosphatase, transferrin, apolipoprotein E, CK, CKMB, parathyroid hormone, insulin, cholesteryl ester transfer protein (CETP), cytokines, cytochrome c, apolipoprotein Al, apolipoprotein All, apolipoprotein BI, apolipoprotein B-100, apolipoprotein B48, apolipoprotein CII, apolipoprotein CIII, apolipoprotein E, triglycerides, HD cholesterol, LDL cholesterol, lecithin cholesterol acyltransferase, paraxonase, alanine aminotransferase (ALT), asparate transferase (AST), CEA, HER-2, bladder tumor antigen, thyroglobulin, alpha-fetoprotein, PSA, CA 125, CA 19.9, CA 15.3, leptin, prolactin, osteoponitin, CD 98, fascin, troponin I, CD20, HER2, CD33, EGFR, VEGFA, etc.), drug (cannabinoid (e.g., tetrahydrocannabinol (THC), cannabidiol (CBD) and cannabinol (CBN), etc.), opioid (e.g., heroin, opium, fentanyl, etc.), stimulant (e.g., cocaine, amphetamine, methamphetamine, etc.), club drug (e.g., MDMA, flunitrazepam, gama-hydroxybutyrate, etc.), dissociative drug (e.g., ketamine, phencyclidine, salvia, dextromethorphan, etc.), hallucinogens (e.g., LSD, mescaline, psilocybin, etc.), etc.), explosive (e.g., 2,4,6-trinitrotoluene (TNT) and hexahydro-1,3,5-trinitro-1,3,5-triazine (RDX), pentaerythritol tetranitrate (PETN), etc.), toxic chemical (e.g., tabun (GA), sarin (GB), soman (GD), cyclosarin (GF), 2-(dimethylamino)ethyl N, N-dimethylphosphoramidofluroidate (GV), VE, VG, VM, VP, VR, VS, or VX nerve agent), etc.

In some embodiments, small molecule detection immunoassays, such as the one exemplified in Example 5 and the like, are performed in the solid phase, lateral flow, and other assays and devices described herein.

### 9. EXAMPLES

It will be readily apparent to those skilled in the art that other suitable modifications and adaptations of the methods of the present disclosure described herein are readily applicable and appreciable and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### Solid Phase Materials

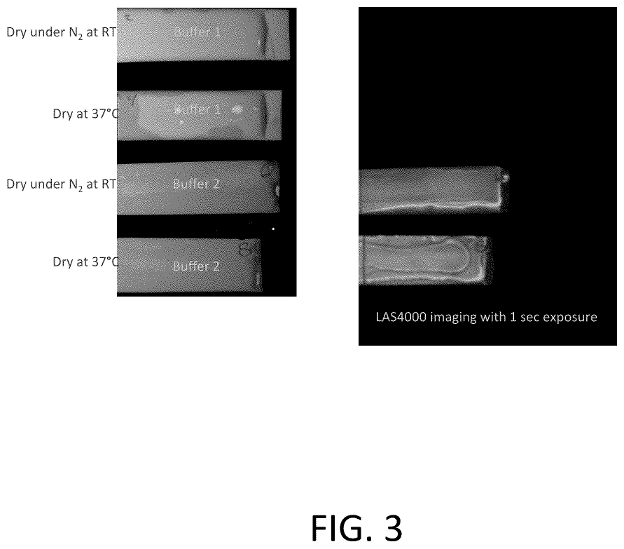

As shows in FIG. 3, components of the bioluminescent complexes of the present disclosure produce detectable bioluminescence after being applied to a solid support substrate (e.g., membrane). Antibodies labeled with NanoLuc® components (e.g., target analyte binding agents) were applied to a membrane that was either blocked (Buffer 1; upper two membranes on left and right panels) or unblocked (Buffer 2; lower two membranes on left and right panels) and then dried at room temperature with nitrogen or at 37° C. without nitrogen. Using an Imagequant LAS4000 imaging platform (1 second exposure), detectable bioluminescence was produced under these conditions. These results demonstrate that components of the bioluminescent complexes of the present disclosure can be successfully used in solid phase and lateral flow assay platforms, which may involve drying reagents and application to solid phase materials, and exposure to various temperatures and processing conditions.

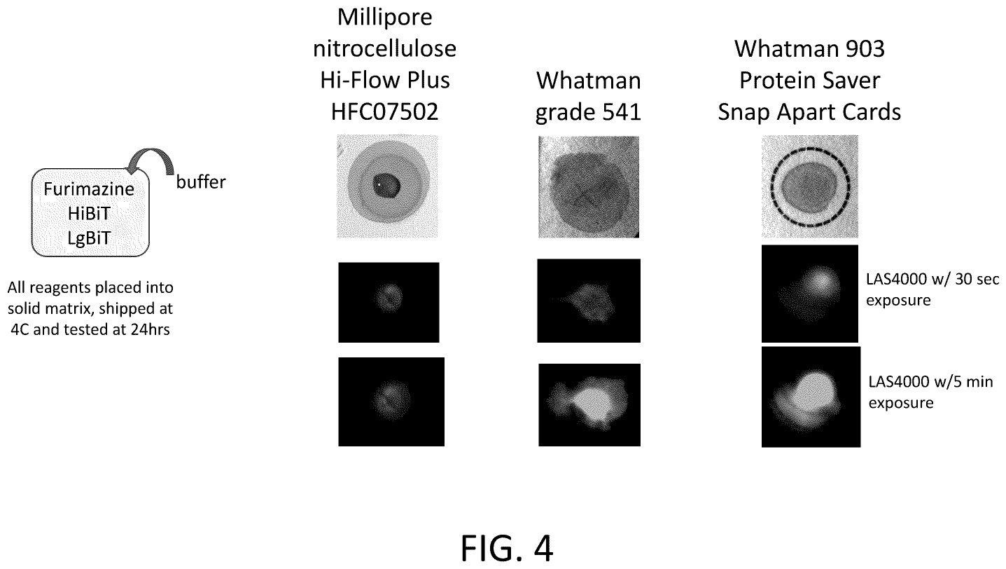

As shown in FIG. 4, components of the bioluminescent complexes produce detectable bioluminescence after being applied to membrane and paper-based solid support matrices. Compositions that included buffer, substrate (e.g., furimazine), and two complementary components of a bioluminescent complex (e.g., HiBiT and LgBiT) were applied to a nitrocellulose membrane (left three panels), or filter paper (Whatman 541 shown in the middle three panels; Whatman 903 shown in right three panels). These components were then dried, shipped at 4° C. and then tested 24 hours later using an LAS4000 imaging platform (30 second and 5 min exposures). Detectable bioluminescence was produced under these conditions, with filter paper matrices allowing for brighter bioluminescent signal than nitrocellulose membranes. Matrices made with glass and synthetic fibers (e.g., Ahlstrom grade 8950) also yielded detectable bioluminescent signal (data not shown) demonstrating that components of the bioluminescent complexes of the present disclosure can be successfully used with various matrix materials.

### Example 2

Detecting Target Analytes with Bioluminescent Complexes

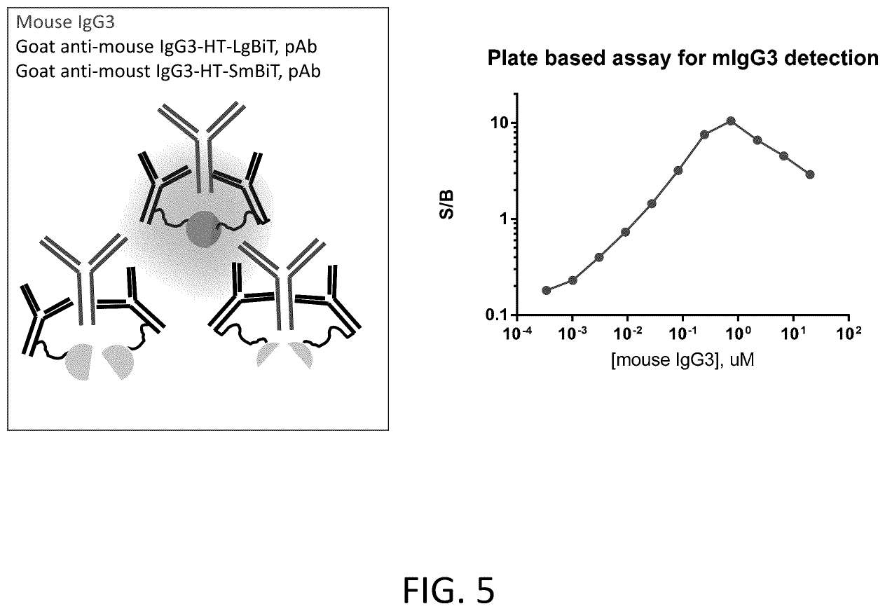

As shown in FIG. 5, components of the bioluminescent complexes (e.g., non-luminescent peptides and polypeptides) of the present disclosure can be used as target analyte binding agents for target analyte recognition. For example, as shown in FIG. 5 (left panel), polyclonal goat anti-mouse IgG3 antibodies (e.g., target analyte binding elements) were conjugated to components of the bioluminescent complexes (e.g., LgBiT and SmBiT). In the presence of the target analyte (e.g., mouse IgG3), a bioluminescent complex was formed, and a bioluminescent signal was produced from the complementary interaction of the components of the bioluminescent complex (FIG. 5, right panel) with increased signal being produced as the concentration of the target analyte increased. These results demonstrate the feasibility of detecting target analytes using the components of the bioluminescent complexes of the present disclosure.

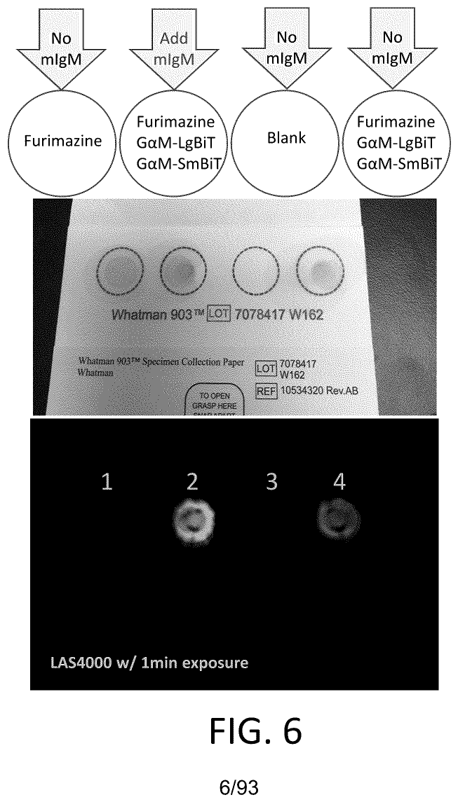

As shown in FIG. 6, embodiments of the present disclosure include a solid phase assay platform using components of the bioluminescent complexes as target analyte binding agents for target analyte recognition. Four test spots were prepared on Whatman 903 filter paper as shown, and target analyte was added thereafter (FIG. 6, top panel). In one embodiment, 20 ng of goat-anti-mouse-conjugated to a component of the bioluminescent complex (e.g., SmBiT), and 20 ng of goat-anti-mouse-conjugated to a complementary component of the bioluminescent complex (e.g., LgBiT) were each prepared in 5 μl of protein buffer (20 mM Na₃PO₄; 5% w/v BSA; 0.25% v/v Tween 20; 10% w/v sucrose) and dried for 1 hour at 37° C. onto the paper in the locations indicated. Additionally, 5 μl of a 5 mM solution of furimazine in ethanol was applied to the spots as indicated under high vacuum for 15 mins (FIG. 6, top panel). The prepared spots were then stored for one week at 4° C. As demonstrated, in the presence of the target analyte (e.g., mouse IgG3; spot #2), a bioluminescent complex was formed, and a bioluminescent signal was produced from the complementary interaction of the components of the bioluminescent complex (FIG. 6, bottom panel). Although background bioluminescent signal was produced with no target analyte present (spot #4), the signal produced in the presence of the target analyte and the luminogenic substrate (e.g., furimazine) is substantially increased as compared to the signal produced with the luminogenic substrate alone (compare spots #2 and #4).

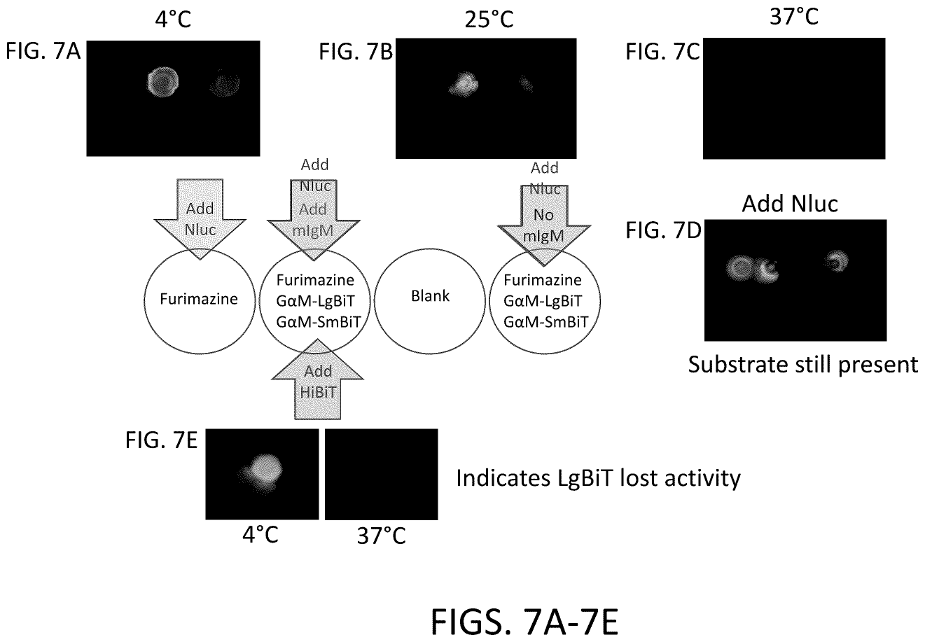

Additional tests of substrate and protein stability were performed and are depicted in FIGS. 7A-7E. These tests were performed as described above with the additional step of adding a fully functional bioluminescent complex (e.g., NanoLuc) after the addition of the target analyte to test luminogenic substrate stability. As demonstrated in FIGS. 7A-7C, components of the bioluminescent complex lose activity when stored at higher temperatures (e.g., 37° C.) for two weeks. The loss of bioluminescent signal does not appear to be due to instability or breakdown of the luminogenic substrate, as the addition of a fully functional bioluminescent complex (e.g., NanoLuc) still produced a signal (FIG. 7D). Additionally, to test whether breakdown of one or more components of the bioluminescent complex was responsible for the reduced bioluminescent signal, a non-antibody conjugated component (e.g., HiBiT) was added that was not subject to storage conditions. As demonstrated in FIG. 7E, addition of the non-antibody conjugated component led to the production of a bioluminescent signal at 4° C. but not 37° C., thus indicating that the degradation of the complementary component of the bioluminescent complex (e.g., LgBiT) was likely leading to the loss of signal.

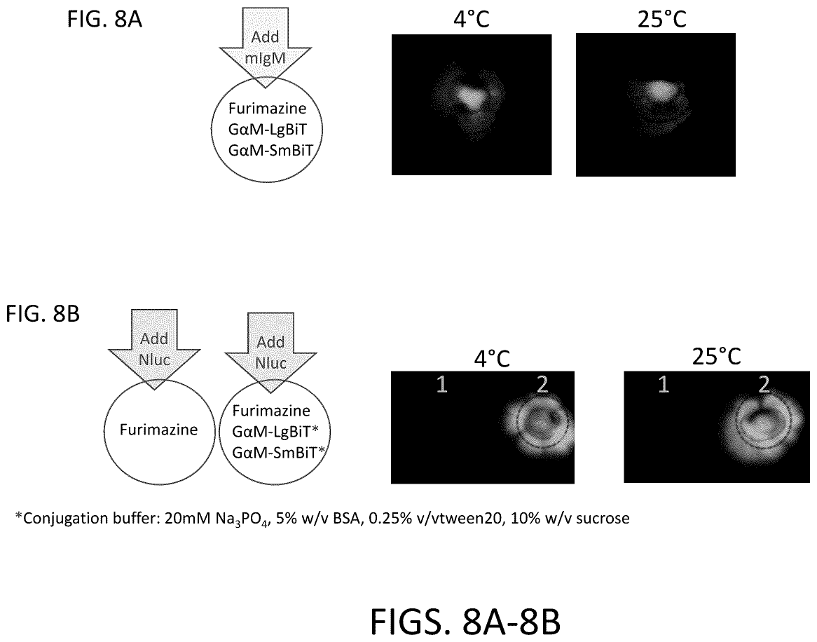

Additional tests of storage conditions were performed and are depicted in FIGS. 8A-8B. These tests were performed as described above, except that the test spots were stored for a total of 3 months. As shown in FIG. 8A, detectable bioluminescent signal was produced in the presence of the target analyte at both 4° C. and 25° C. even after 3 months of storage, albeit with somewhat reduced activity. The addition of a fully functional bioluminescent complex (e.g., NanoLuc) produced a signal (FIG. 8B), but the signal appeared to be dependent upon the use of protein buffer (compare spots #1 and #2) suggesting that the luminogenic substrate is stabilized by the protein buffer.

### Example 3

### Detecting Target Analytes in Complex Sampling Environments

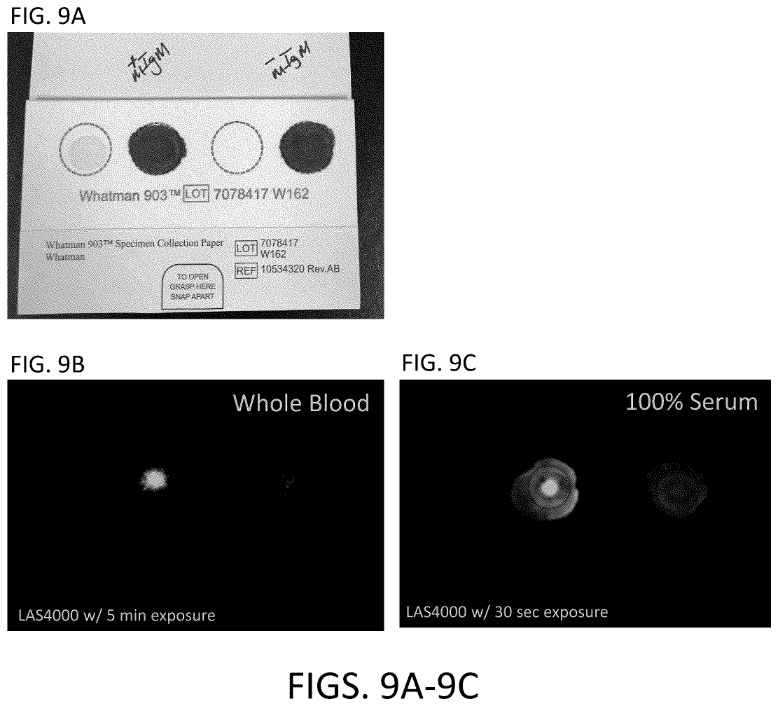

FIGS. 9A-9C include representative images from a solid phase assay platform (e.g., spot test) testing whether bioluminescent complex formation and analyte detection could occur in complex sampling environments. As shown in FIG. 9A, a luminogenic substrate and two complementary components of a bioluminescent complex (HiBiT and LgBiT) were applied to Whatman 903 filter paper, with each component also having a target analyte-binding element (polyclonal anti-mouse IgM), as described above, and stored at 4° C. for 6 weeks. An EDTA-collected whole blood sample (FIG. 9B) and a 100% serum sample (FIG. 9C) were each spiked with 10 pg mouse IgG3 (target analyte) and applied to the spots indicated in FIG. 9A. Corresponding control samples were not spiked with mouse IgG3. These results demonstrate the feasibility of detecting target analytes in complex sampling environments using the components of the bioluminescent complexes of the present disclosure.

### Example 4

### Qualitative and Quantitative Assessment

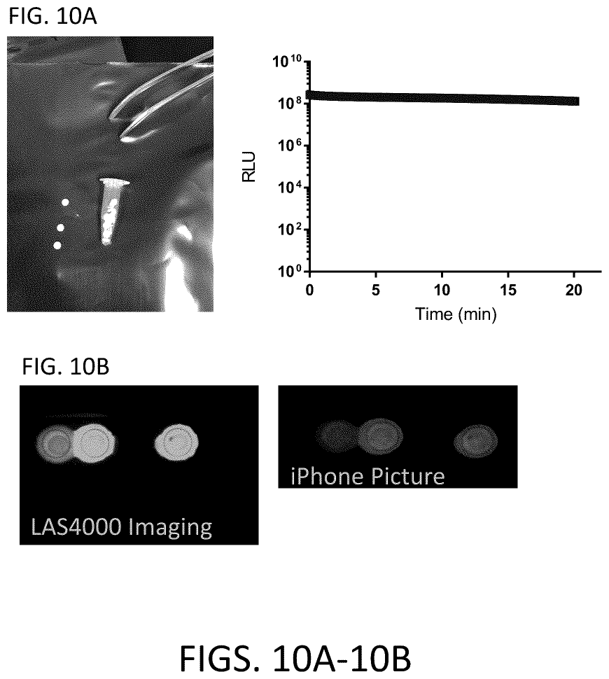

FIGS. 10A-10B include representative results of a solid phase assay demonstrating that bioluminescent signal can be both quantitatively (FIG. 10A) and qualitatively (FIG. 10B) assessed. As shown in FIG. 10A, 10 μM of luminogenic substrate (e.g., furimazine) was applied to filter paper and placed in a microtiter plate. PBS assay buffer containing NanoLuc® enzyme was then added, and bioluminescent signal was quantitatively (FIG. 10A, right panel) and qualitatively assessed (FIG. 10B). Additionally, bioluminescent signal was effectively assessed using a luminometer (FIG. 10B, left panel) as well as a smart phone (FIG. 10B, right panel).

These results demonstrate that the assays and methods of the present disclosure can include comparing levels of bioluminescence corresponding to target analyte detection with various control samples to facilitate rapid quantitative and qualitative assessment. For example, assay formats can include a plurality of control samples with varying concentrations of target analyte that can act as standards against which test samples can be assessed.

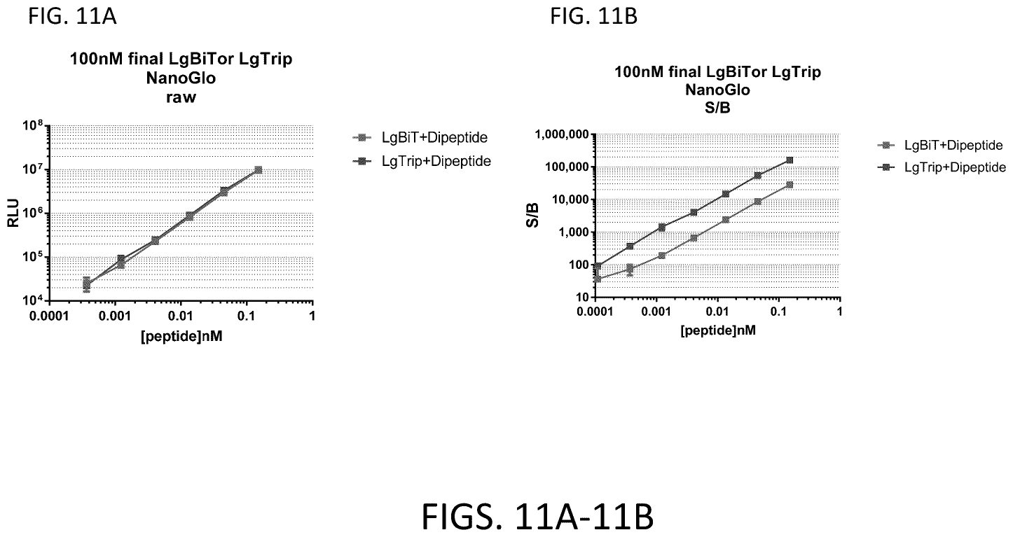

In accordance with these methods, a bioluminescent signal can be assessed both quantitatively and qualitatively using a high affinity dipeptide capable of forming a bioluminescent complex with LgBiT or LgTrip. The results shown in FIGS. 11A-11B include representative graphs (RLUs in FIG. 11A; S/B in FIG. 11B) demonstrating the ability of a high affinity dipeptide, pep263, to form bioluminescent complexes with LgBiT and LgTrip. The high affinity dipeptide pep263 comprises the j9 and 010 stands of the NanoTrip complex. (See, e.g., U.S. patent application Ser. No. 16/439,565 (PCT/US2019/036844), and U.S. Prov. Appln. Ser. No. 62/941,255, both of which are herein incorporated by reference in their entirety.)

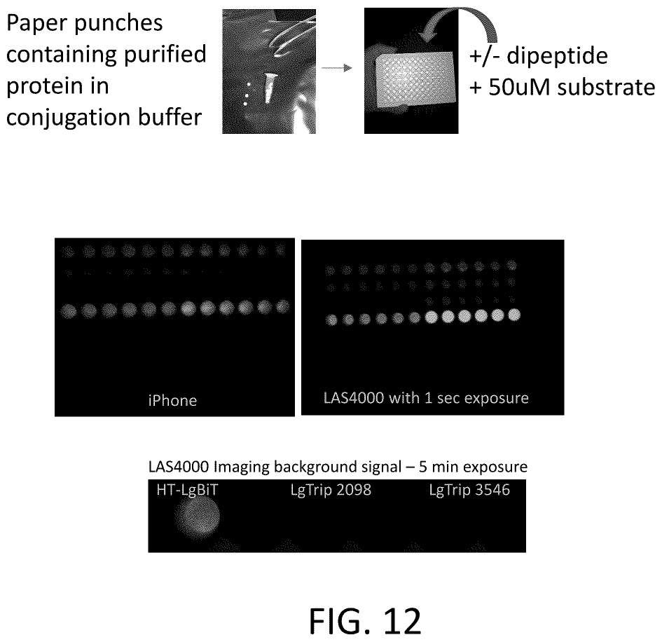

Additionally, FIG. 12 shows representative results of a solid phase assay demonstrating qualitative assessment of bioluminescence from paper punches placed into a standard microtiter plate using a standard camera from an iPhone or from an imager (e.g., LAS4000). This spot test assay assessed the functional stability of different LgBiT components dried onto Whatman 903 paper. Whatman 903 protein saver spot cards (⅛″ punches) were used along with the following protein buffer: 20 mM Na₃PO₄, 5% w/v BSA, 0.25% v/v tween20, 10% w/v sucrose. A 1000× NanoLuc® stock solution was diluted 1:1000 in protein buffer. About 5 μL of this reaction solution was applied to Spot 1. For HT-LgBiT complexes, about 5 μL of 106.8 nM protein per spot was used. About 20 μM stock protein was diluted 1:100 in protein buffer. About 534 μL stock was diluted in 466 μL in protein buffer. About 5 μL of this conjugation solution was added to Spot 2. For LgTrip (2098) complexes, about 5 μL of 106.8 nM protein per spot was used. About 9.6 μM protein stock was obtained by diluting about 11.6 μL of stock in 988 μL of protein buffer to make 1 mL of 106.8 nM solution. About 5 μL of this conjugation solution was added to Spot 3. For LgTrip (3546) complexes, about 5 μL of 106.8 nM protein per spot. About 94 μM protein stock was obtained by diluting about 1.13 μL of LgTrip stock into 998.87 μL protein buffer. About 5 μL of this conjugation solution was added to Spot 4. After all the protein was added, the samples were dried at 30° C. for 1 hour at 4° C., 25° C., and 37° C.

Methods for assessing RLU activity for these experiments included imaging at day 6 for all at 25° C. and 37° C. (following the 4° C. time frame of 1 or 2 days); day 8 at 4° C. for LgTrip 3546; and day 9 for NanoLuc, LgBiT, and LgTrip 2098. Furimazine was tested at 50μM and about 1.2 μM dipeptide was used for NanoBiT and NanoTrip experiments. All spots were placed into a plate with substrate reagents, images were captured with an iPhone and with an LAS4000 imaging system, then inserted into the plate reader. NanoGlo Live Cell Substrate cat #N205B (lot 189096) was used, along with assay buffer 1× PBS, pH 7.0).

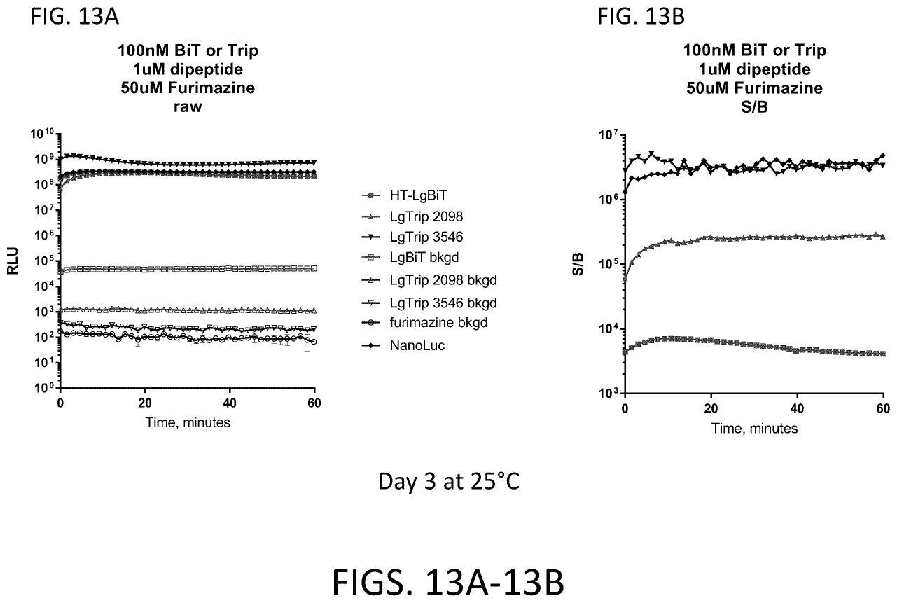

FIGS. 13A-13B show quantitative analysis of the same solid phase assay depicted in FIG. 12, but luminescence was detected using a luminometer on day 3 at 25° C. (RLUs in FIG. 13A; S/B in FIG. 13B). These quantitative data support the qualitative data from FIG. 12. Materials and methods used for FIG. 12 are the same used for FIGS. 13A-13B (e.g., add 1μM dipeptide+50 μM live cell substrate in PBS, pH 7.0 and read on a luminometer). In some cases, the elevated background of LgBiT can decrease the S/B ratio.

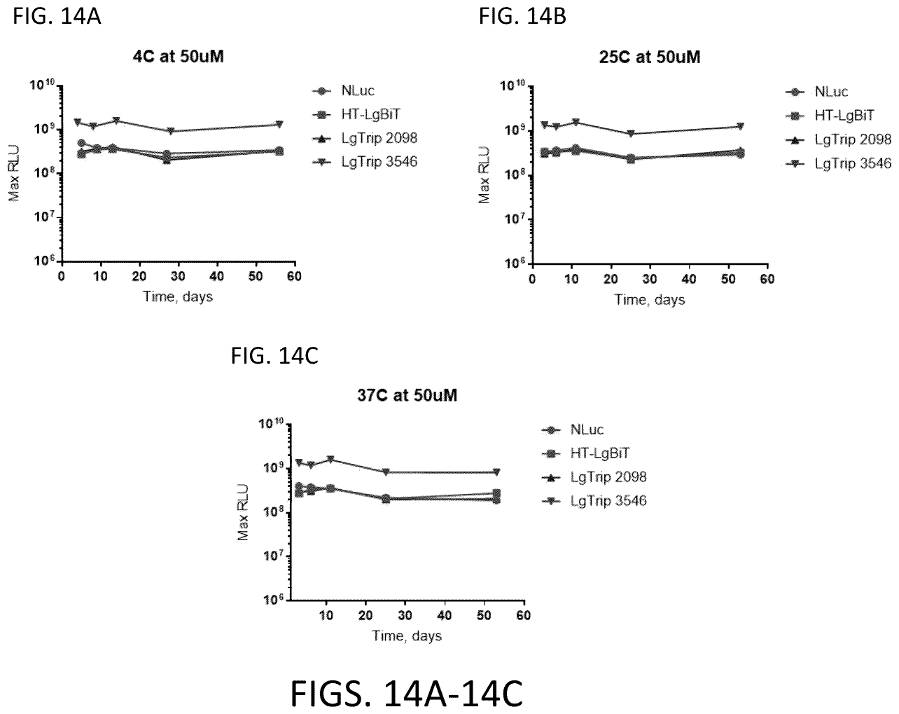

FIGS. 14A-14C show a quantitative time course of the same solid phase assay as depicted in FIGS. 12-13 demonstrating stability of all the proteins in the experimental conditions at all temps tested over the time frame. Bₘₐₓ RLU values at 50μM furimazine over time (0 to 60 days) are shown for 4° C. (FIG. 14A), 25° C. (FIG. 14B), and 37° C. (FIG. 14C). These quantitative data are consistent with FIGS. 12 and 13, demonstrating stability in all the complexes tested and at all temps tested over the time frame. Materials and methods used for FIG. 12 are the same used for FIGS. 14A-14C.

### Example 5

### Buffer Compositions

Experiments were also conducted to test short-term, or accelerated, stability of the complexes in different buffer compositions from 0 to 90 minutes. Methods included using about a 1.068 nM concentration of each protein absorbed and dried on Whatman 903 paper spots (⅛″). Protein samples were prepared and dried on paper spots with either protein buffer or PBS buffer (see each figure for specific buffer composition used). Stock concentrations included NanoLuc at 1000× (0.4 mg/mL), LgBiT-1672-11s-His at 20 μM, and LgTrip (3546) at 94 μM. Protein buffer was comprised of 20 mM Na₃PO₄, 5% w/v BSA, 0.25% v/v tween20, 10% w/v sucrose. Luminescence activity was tested using the dipeptide added with furimazine in 100 μl assay buffer PBS, pH 7.0 (final [dipeptide]=1 nM; final [furimazine]=50μM). Samples were read at time point 0 (fresh out of 4° C.), then placed into 60° C. and 25° C. for continued testing. A 1000× stock solution of NanoLuc was diluted 1:1000 in protein buffer (1 mL), or 10 μL of stock was diluted into 990 μL of protein buffer for a 1.068 nM stock (see each figure for specific buffer composition used). About 5 μL of each concentration was added to a paper spot for testing. For each protein tested (LgBiT and LgTrip), appropriate dilutions were made in each buffer to ensure that about 5 μL of 1.068 nM protein was used per spot. After all protein was added, the samples were dried at 35° C. for 1 hour, and 40 spots per condition and temperature were prepared.

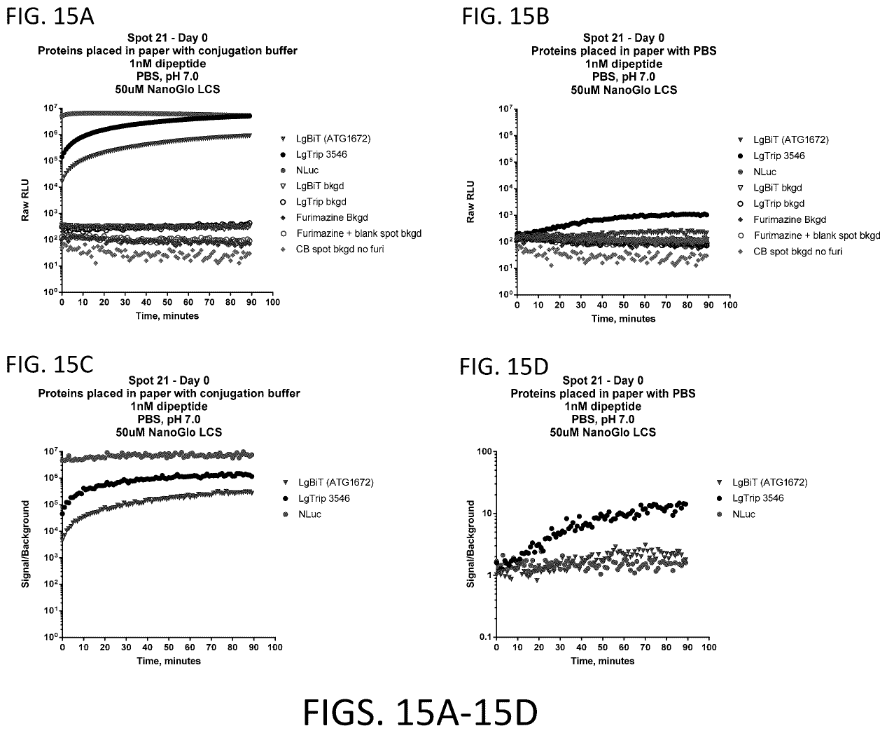

FIGS. 15A-15D show representative results collected on day 0 of an accelerated stability study performed under two buffer conditions at 25° C. and 60° C. (FIGS. 15A and 15C use protein buffer, whereas FIGS. 15B and 15D use PBS). These data demonstrate that the complexes tested did not tolerate PBS as the buffer condition for input into the Whatman 903 paper, as compared to the protein buffer. Buffer conditions appear to affect stability even at early time points. In some cases, LgTrip 3546 exhibited better activity, suggesting somewhat better chemical stability than NanoLuc and LgBiT under these conditions.

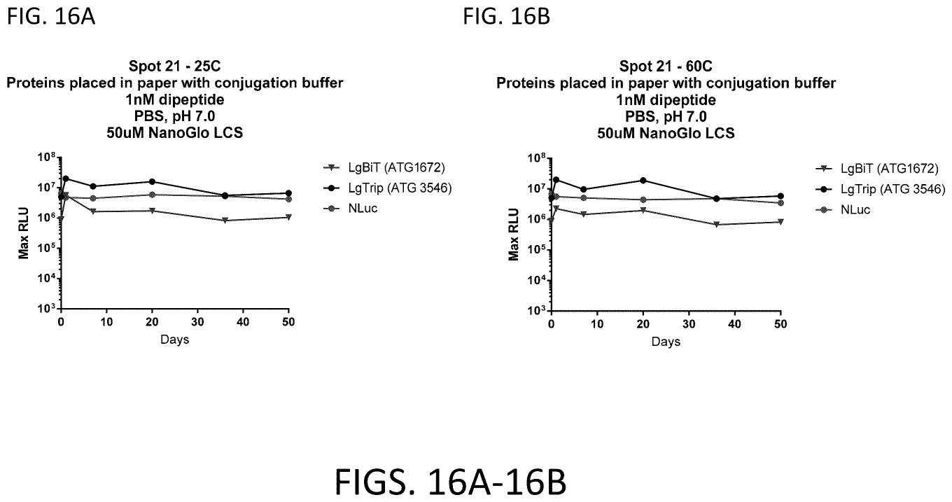

FIGS. 16A-16B show results for the accelerated stability study depicted in FIG. 15, but over a 0 to 50-day time course. FIG. 16A includes results of samples tested in protein buffer at 25° C., and FIG. 16B includes results of samples tested in protein buffer at 60° C. The same materials and methods were used as in FIG. 15. These results demonstrate that the complexes remain stable under these conditions (at 25° C. and 60° C.) up until at least 50 days.

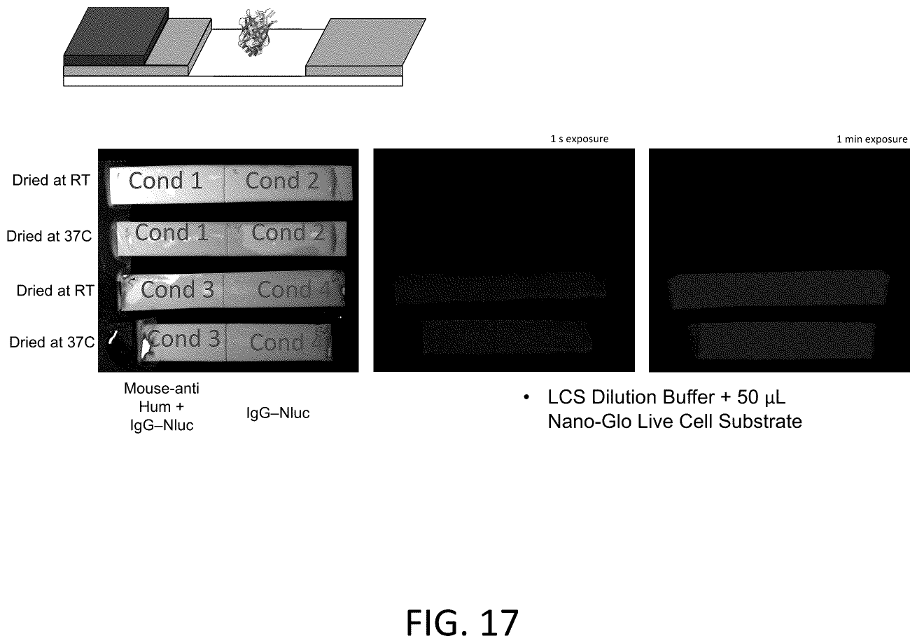

FIG. 17 shows a comparison of the impact of buffer conditions on luminescence from NanoLuc dried onto a nitrocellulose membrane to assess NanoLuc® stability in the context of a lateral flow assay. Four different conditions were tested: Condition 1: Mouse-anti Hum+IgG-Nluc in PBS, pH 7.4; Condition 2: IgG-Nluc in PBS, pH 7.4; Condition 3: Mouse-anti Hum+IgG-Nluc in loading buffer (20 mM Na₃PO₄, 5% w/v BSA, 0.25% v/v tween20, 10% w/v sucrose); Condition 4: IgG Nluc in loading buffer (20 mM Na₃PO₄, 5% w/v BSA, 0.25% v/v tween20, 10% w/v sucrose). Each condition was applied to the membranes and either dried at RT or at 37° C.

For these experiments, the following solutions were prepared: (1) 5 μl mouse/antihuman into 995 1 Addition buffer (0.1 M PBS, pH 7.4); (2) 5 1 anti-mouse-NanoLuc in 995 1 Addition buffer (0.1 M PBS, pH 7.4); (3) 5 1 mouse/antihuman in protein buffer (20 mM Na₃PO₄, 5% w/v BSA, 0.25% v/v tween20, 10% w/v sucrose); and (4) 5 1 anti-mouse-NanoLuc in 995 1 protein buffer (20 mM Na₃PO₄, 5% w/v BSA, 0.25% v/vtween20, 10% w/v sucrose).). About 0.5 ml of solution (1) was loaded into an airbrush and applied to the left side of a nitrocellulose strip (Strip 1 and 2). The strips were allowed to dry either at RT or at 37° C. for 1 hour. About 0.5 ml of solution (2) for was applied to the entire surface of strip 1 and strip 2 and allowed to dry at RT or at 37° C.; forming condition 1 and 2, respectively. About 0.5 ml of solution (3) was loaded into an airbrush and applied to the left side of a nitrocellulose strip (Strip 3 and 4). The strip was allowed to dry either at RT or at 37° C. for 1 hour. About 0.5 ml of solution (4) for was applied to the entire surface of strip 3 and strip 4 and allow to dry at RT or 37° C.; forming condition 3 and 4, respectively. For imaging, a 1× solution of substrate was prepared (4mls PBS+1 ml Nano-Glo LCS Dilution Buffer+50ul Nano-Glo Live Cell Substrate) and overlaid on each strip with 1 ml of substrate solution; imaging began immediately thereafter.

These data demonstrate that buffer formulations are important for activity in lateral flow membranes. In conditions 1-4, where protein was just applied to the membrane in PBS, very little to no light was observed when the membranes were exposed to freshly prepared Nano-Glo Live Cell substrate. In contrast, protein that was prepared with a loading buffer that contained additional components such as Na₃PO₄, BSA, Tween 20, and sucrose showed considerable light output. This suggests that the particular loading buffer used to add the protein to the surface of the membrane is important for stability and function (FIG. 17).

### Example 6

### Lateral Flow Assay Components

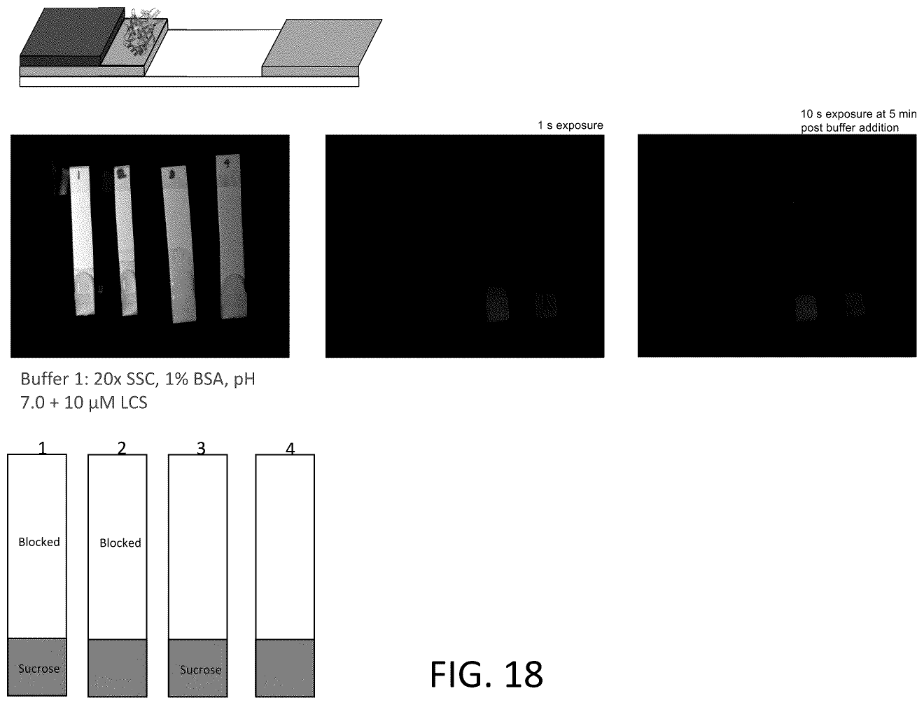

Experiments were conducted to test different membrane blocking agents and assay running buffers to facilitate proper movement of proteins and targets during a lateral flow assay. Four strips were used, and the design of each (with or without sucrose and blocking agent) is shown in the schematic below the far left image of FIG. 18. Briefly, strip 1 included a blocked membrane with sucrose pre-treatment on a conjugation pad; strip 2 included a blocked membrane with no sucrose pre-treatment on a conjugation pad; strip 3 included an unblocked membrane with sucrose pre-treatment on a conjugation pad; and strip 4 included an unblocked membrane with no sucrose pre-treatment on a conjugation pad.

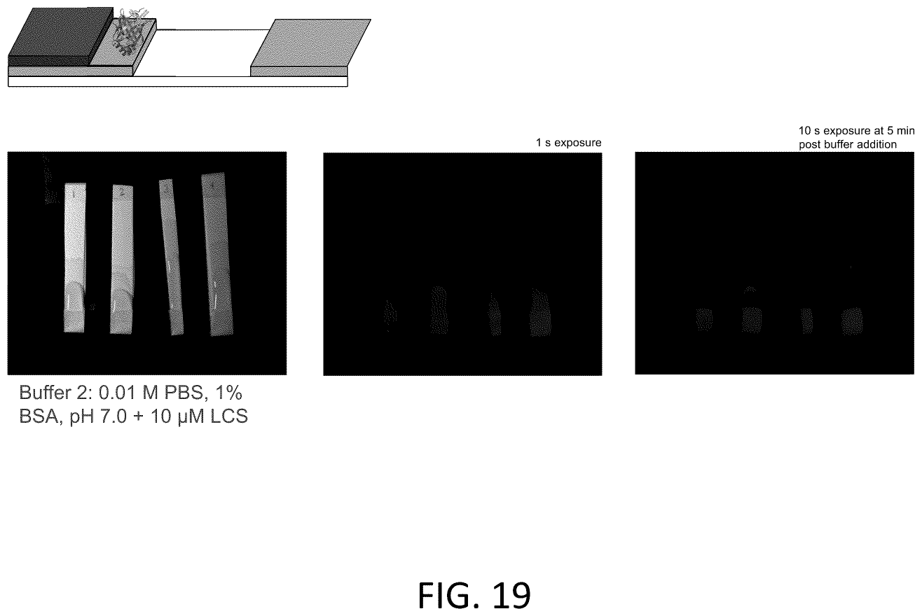

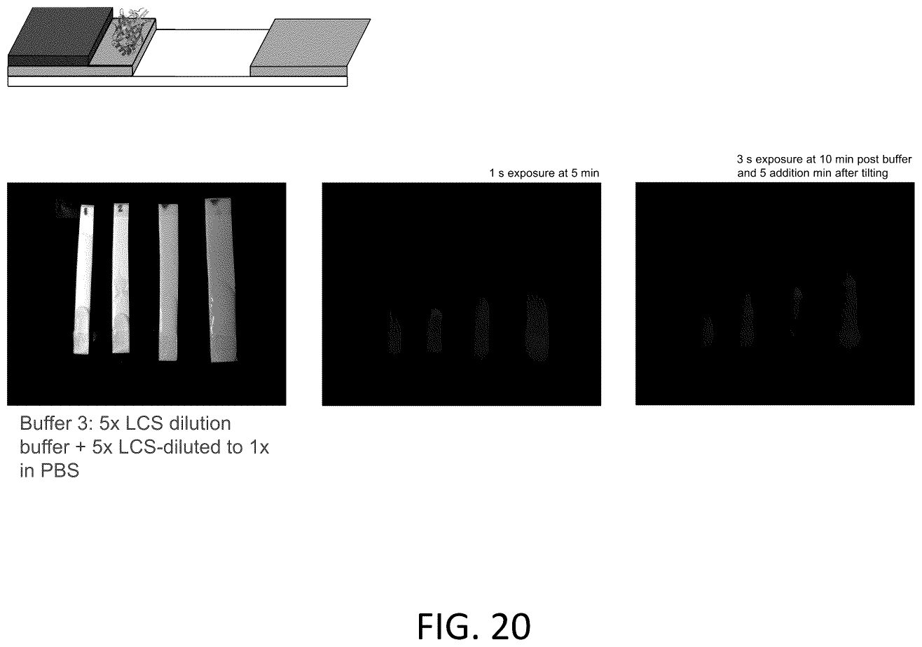

The blocking buffer was comprised of 1% w/v polyvinyl alcohol in 20 mM tris, pH 7.4. Conjugation pre-treatment included 30% sucrose w/v in DI water. The conjugation pad was Ahlstrom grade 8950 (chopped glass with binder, 50 g/m²), and the membrane was nitrocellulose. For blocking, the membrane was soaked in blocking buffer for 30 min at RT, and subsequently remove from buffer, washed with DI water, and dried for 30 min at 35° C. For secondary pre-treatment, sucrose solution was applied to the membrane pad near where conjugation reagent (substrate) will be applied. The membrane was dried for 1 hr at 35° C. To prepare the proteins, about 5 μL anti-mouse-NanoLuc was added to 995 1 protein buffer. About 1 ml of protein solution was placed into an airbrush and a light coating was applied to the conjugation pad. This was allowed to dry for 1 hr at 35° C. Strips were then assembled on backing card. Additionally, for FIGS. 18-20, the following buffers compositions were tested: Buffer 1 was comprised of 20×SSC, 1% BSA, pH 7.0+10 μM LCS (FIG. 18). Buffer 2 was comprised of 0.01 μM PBS, 1% BSA, pH 7.0+10 μM PCS (FIG. 19). And Buffer 3 was comprised of 5× LCS dilution buffer+5× LCS—diluted to 1× in PBS (FIG. 20).

FIG. 18 shows the effects of membrane blocking and sucrose pre-treatment on lateral flow assays performed in a running buffer of 20×SSC, 1% BSA, pH 7.0+10 μM LCS. FIG. 19 shows the effects of membrane blocking and sucrose pre-treatment on lateral flow assays performed in a running buffer of 0.01 μM PBS, 1% BSA, pH 7.0+10 μM Permeable Cell Substrate (PCS). FIG. 20 shows the effects of membrane blocking and sucrose pre-treatment on lateral flow assays performed in a running buffer of 5× LCS dilution buffer+5× LCS—diluted to 1× in PBS. These data demonstrate that membrane treatment and protein buffers do affect assay fluid flow within the conjugation pad and across the lateral flow membrane.

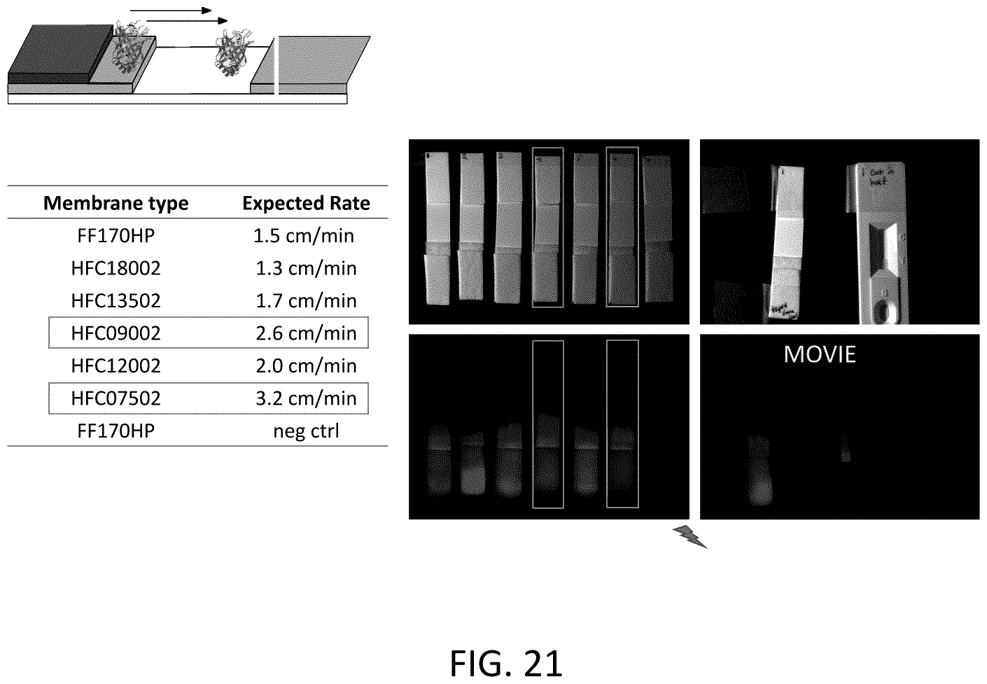

Experiments were also conducted to assess different membranes and membrane properties within the context of a lateral flow assay such as the effects of membrane properties on absorption and capillary action. FIG. 21 shows the effects of membrane properties on bioluminescent reagent absorption and capillary action in a lateral flow assay. Membranes containing different pore sizes were tested for flow efficiency. Each membrane was unblocked and contain a 30% w/v sucrose pretreatment on approximately the bottom ⅓ of the strip. Other materials included a Conjugation pad (Ahlstrom grade 8950, chopped glass with binder, 50 g/m²); a Sample Pad (Cellulose glass fiber CFSP203000 (Millipore)); and an Absorption pad (Cotton linters, grade 238 (Ahlstrom)). The following membrane conditions were tested:
1. nitrocellulose FF170HP (Ahlstrom)
2. nitrocellulose Hi-Flow Plus HFC18002 (Millipore)-180 sec/4 cm
3. nitrocellulose Hi-Flow Plus HFC13502 (Millipore)-135 sec/4 cm
4. nitrocellulose Hi-Flow Plus HFC09002 (Millipore)-90 sec/4 cm
5. nitrocellulose Hi-Flow Plus HFC12002 (Millipore)-120 sec/4 cm
6. nitrocellulose Hi-Flow Plus HFC07502 (Millipore)-75 sec/4 cm
7. nitrocellulose FF170HP (Ahlstrom)—NEGATIVE CONTROL.

Running buffer was comprised of 5× LCS dilution buffer+5× LCS-diluted to 1× in PBS. Membranes were pre-treated by applying 30% sucrose solution to the membrane, covering ˜1.5 cm of the bottom of the strip, the allowed to dry at 35° C. for 1 hour. Proteins were prepared by adding about 5 μL anti-mouse-NanoLuc in 995 μL protein buffer. About 1 mL of protein solution was added to an airbrush, which was used to lightly coat conjugation pad. This was allowed to dry at 35° C. for 1 hour. The negative control for these experiments contained protein buffer without protein, which was applied with an airbrush in the same manner as the test conditions. Strips were assembled on backing card. The conjugation pad, sample pad, and wicking pad were cut to be 2 cm×1 cm. The sample pad and conjugation pad were overlapped by ˜1.8 cm. The total dimensions of the strip were about 6 cm×1 cm.

An imaging program was created to capture 5 sec exposure images every 30 seconds for a total of about 10 minutes. Imaging was repeated if it appeared that there was still NanoLuc flowing across the membrane. Images were stacked into movies using ImageJ, and the final images included in FIG. 21 are the accumulative signal of all images taken over time.

These results suggest that strips 4 and 6 (boxed in FIG. 21) had the most complete NanoLuc traveling out of conjugation pad and into sample reservoir, based on the conditions used in these experiments.

### Example 7

### Bioluminescent Complex Formation

Experiments were conducted to evaluate bioluminescent complex formation in the presence of various reagents on membrane and filter paper. Experiments were designed and conducted according to the schematic below, which shows the four different conditions tested.

For these experiments, 2.5 μL of HaloTag-HiBiT was added to 498 μL protein buffer. About 5 μL of this solution was spotted on both the membrane and filter paper in quadrants 1, 3, and 4 (see above schematic) and allowed to dry at 37° C. for 1 hour. About 2.5 μL of ATG-1672-11S-6His was diluted in 498 μL of protein buffer, and about 5 μL was spotted directly onto nitrocellulose membrane and filter paper in quadrants 2, 3, and 4 (see above schematic). Membranes were allowed to dry at RT for 1 hour. Furimazine was prepared as a 5 mM stock solution in EtOH. About 5 μL of this solution was spotted onto both the membrane, and the filter paper in quadrants 1, 2, and 3 and immediately placed under high vacuum for 15 minutes. About 2.5 μL of stock protein (20 μM) was diluted in 498 μL of NanoGLO buffer, which does not contain substrate. About 5 μL was added to the quadrant indicated above and subsequently read in a luminometer.

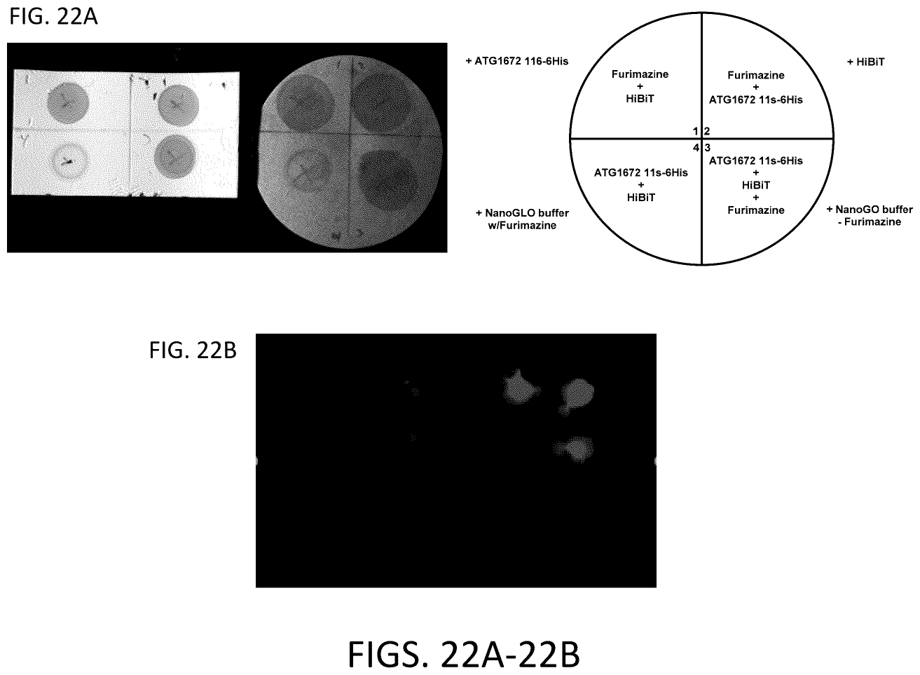

FIGS. 22A-22B show bioluminescent signal from NanoBiT/HiBiT complementation on nitrocellulose (left) and Whatman grade 541 (right) papers (FIG. 22A), and a compilation image from a corresponding movie taken across total exposure time (movies can be made available upon request). Images were captured at increasing exposure times starting with 1 sec and ending with 10 min exposure (Is, 3s, 10s, 30s, 1m, 2, 3, 4, 5, 10m) for a total time (26 min) after the addition of the reagents indicated 26.

These results suggest that filter paper may provide an increased signal as compared to the membrane. Also, the conditions present in quadrant 4 did not produce detectable luminescence, which could indicate that complex formation was impeded by one or more of the other reagents present.

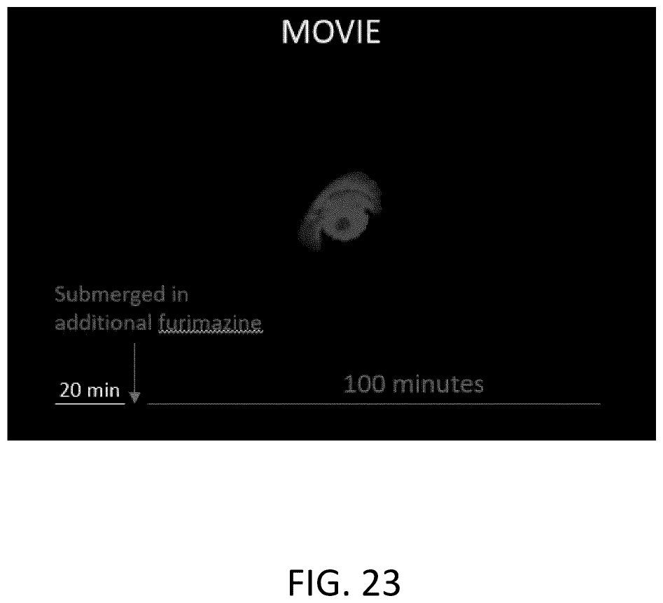

Experiments were conducted to assess the effects of increased substrate concentration on complex formation. FIG. 23 shows bioluminescent signal from NanoBiT/HiBiT complementation on Whatman grade 903 paper, with a spike of additional substrate and liquid at 20 minutes. FIG. 23 is a representative compilation image from a corresponding movie taken across total exposure time (movies can be made available upon request). About 2.5 1 of purified LgBiT or HiBiT was diluted in 498 1 1× LCS Buffer and added directly to the filter paper (consistent with the conditions in quadrant 1) in a 10 μL volume (2:1 LgBiT to HiBiT ratio). The original substrate was NanoBRET NanoGlo (5 μl was added at 5 mM), and the additional submerging substrate was NanoBRET NanoGlo (5 mM stock), diluted 1:5 in 1× NanoGlo buffer, which was diluted to 1× in PBS. About 500 1 was added to cover the filter paper. Images were captured at repeating 30 sec exposures during the entire time duration.

Spiking in additional substrate (furimazine) in an excess of liquid volume showed that signal returns, suggesting that as components start to move within the additional fluid, more complexes may be forming due to their increased mobility. This experiment also indicates that the enzyme retains activity with substrate concentration being the limiting factor that can be remedied by the addition of excess substrate.

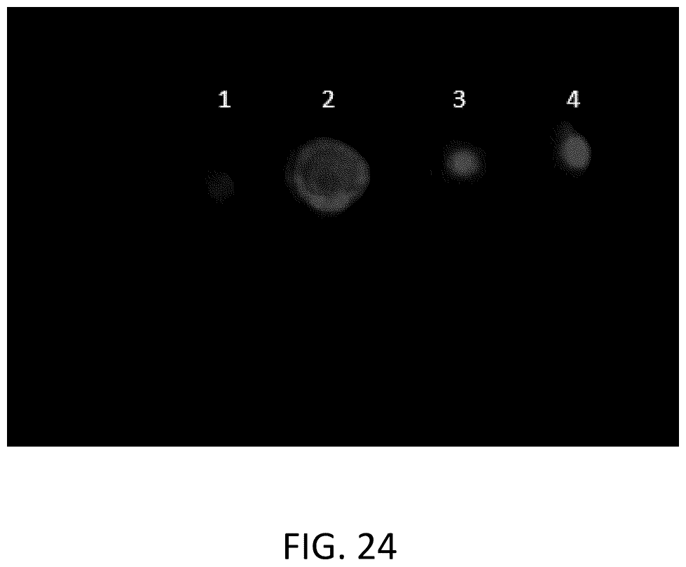

FIG. 24 shows bioluminescent signal from NanoBiT/HiBiT complementation on Whatman 903 paper, instead of Whatman 541 paper, with the experimental conditions consistent with those in the above schematic diagram (quadrants 1-4 in FIG. 22). Buffer was added to rehydrate the membrane near the end of the experiment. FIG. 24 is a representative compilation image from a corresponding movie taken across total exposure time (movies can be made available upon request). The conditions in quadrant 2 appear to provide the strongest luminescent signal.

### Example 8

Spot Tests with LgTrip and Substrate

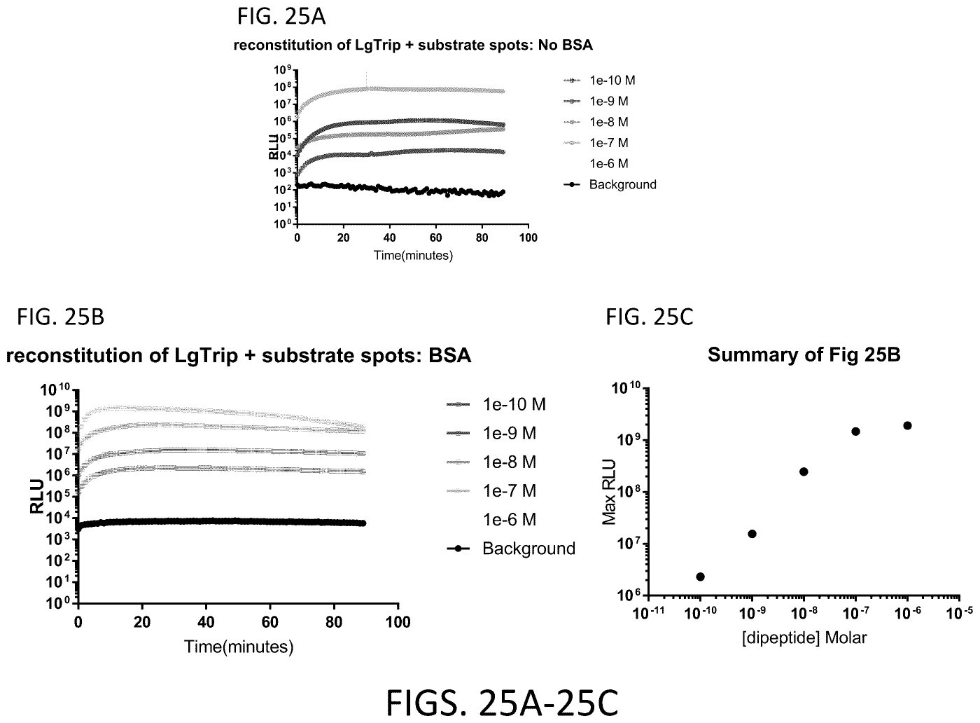

Experiments were conducted to assess the feasibility of an “all-in-one” spot by first testing paper matrix containing LgTrip 3546 and furimazine to which an analyte-of-interest can be added (e.g., dipeptide). FIGS. 25A-25C show bioluminescent signal resulting from reconstitution with dipeptide of LgTrip 3546 and substrate in Whatman 903 paper, in the presence (FIG. 25B) and absence (FIG. 25A) of BSA, along with a serial dilution of the dipeptide with BSA (FIG. 25C). Two sets of spots were made, each spot being comprised of the following components: 1) 5 mM ATT, 5 mM ascorbate, 5 μM LgTrip 3546, and 1 mM furimazine; 2) 5% BSA, 5 mM ATT, 5 mM ascorbate, 5 μM LgTrip 3546, and 1 mM furimazine.

To prepare the spots, a vial containing 200 μL of 5 μM LgTrip 3546, 5 mM ATT, and 5 mM ascorbic acid was prepared. About 5 μL of this solution was added to each spot, and the spots were then allowed to dry at 35° C. for 1 hour. After drying, 1 mM stock of furimazine in ethanol was prepared. About 5 μL of this solution was added to each spot and allowed to dry at 35° C. for an additional 30 minutes. For luminescent measurements, at the time of testing, 1.2 mM dipeptide stock in water was serial diluted down to le⁻¹⁰ μM in PBS, pH 7.0. About 100 μL of each dipeptide stock was added to a 96-well plate containing a spot and kinetic measurements were started immediately.

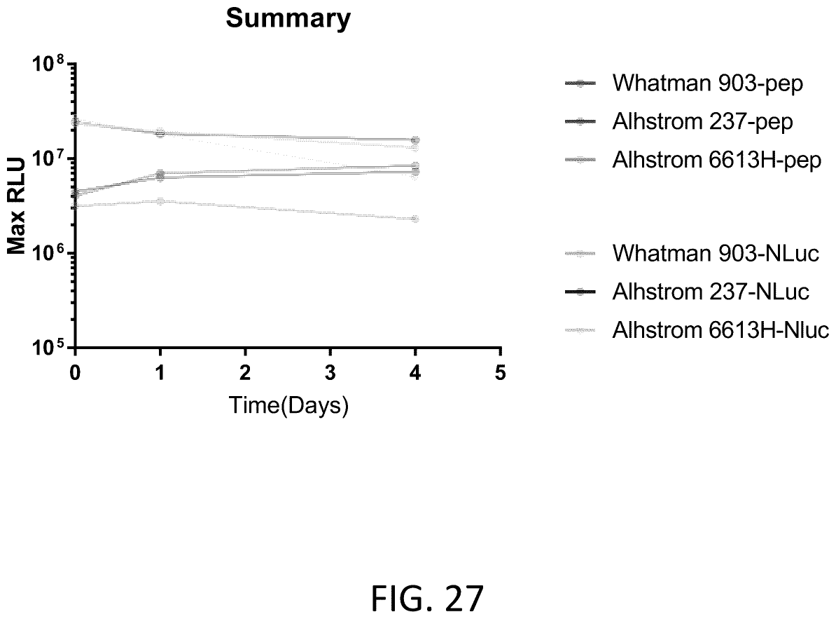

These data demonstrate that a stable, concentration dependent response was observed with the addition of the dipeptide (FIG. 25). This experiment highlights that a paper-format containing LgTrip 3546 and substrate can be made and then reconstituted in buffered aqueous media containing a potential analyte of interest (e.g., dipeptide). Different materials were then tested with substrate and LgTrip 3546 input. Either fresh dipeptide was added at 1 nM to test NanoTrip and substrate activity, or fresh Nluc was added to isolate the substrate. FIG. 27 shows bioluminescent signal in three different solid phase materials (Whatman 903, Ahlstrom 237, and Ahlstrom 6613H) resulting from reconstitution with dipeptide of LgTrip 3546 and substrate, or NanoLuc added to dried LgTrip 3546 and substrate. Alhstrom 6613H seems to be detrimental to signal output over time as it appears that the luminescent signal is decreased in both conditions. Overall, the stability of the assay components can be affected by the composition of the solid matrix materials in which they are imbedded.

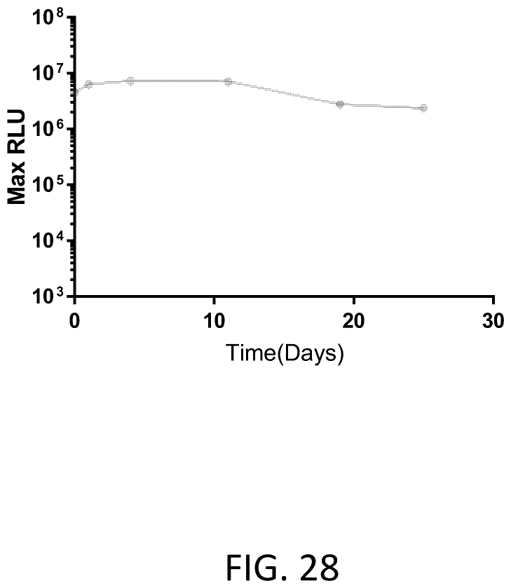

FIG. 28 shows bioluminescent signal from Whatman 903 paper that contains both LgTrip 3546 as well as substrate and stored under ambient conditions for over 25 days. Spots were exposed to 1 nM dipeptide in PBS at the time of testing. Overall, this experiment shows that there is no significant loss of signal from the materials after extended storage times under ambient temperature.

### Example 9

### Lyophilized Cake Containing LgTrip and Substrate

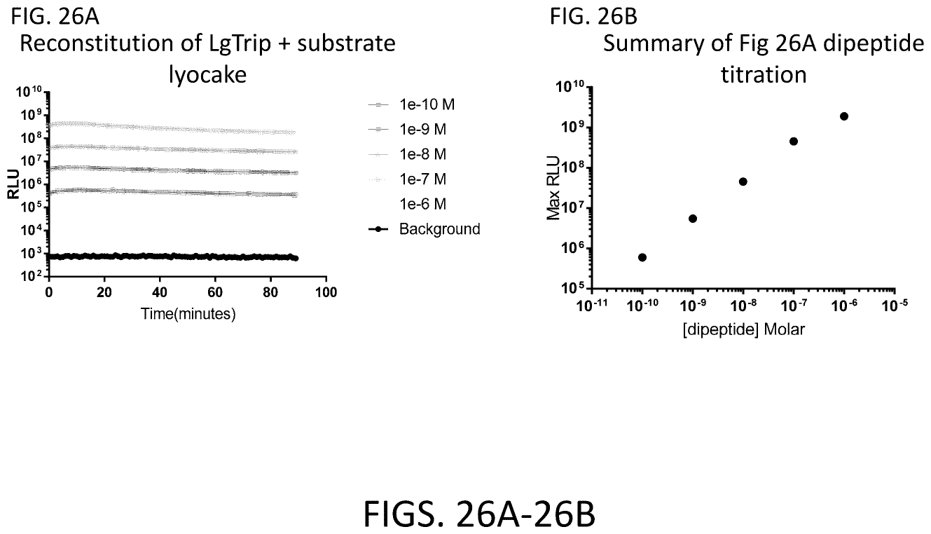

FIGS. 26A-26B show bioluminescent signal resulting from reconstitution with dipeptide of LgTrip 3546 and substrate from a lyocake (FIG. 26A) along with the summary data of the titration of the dipeptide (FIG. 26B). To prepare the lyocakes, 5% w/v pullulan was added to water containing 26.3 mM ATT and 11.3 mM ascorbic acid (solution 1). Solution 1 was then aliquoted out into 35 μL volumes in snap-cap vials. About 10 μL of 95 μM LgTrip 3546 protein was then added to each vial and pipetted to mix (solution 2). A 10 mM stock solution of furimazine in ethanol was prepared, and 5 μL of this solution was added to each vial and mixed (solution 3). Vials containing solution 3 were placed on dry ice to freeze for 1 hour, and then lyophilized overnight. For luminescent measurements, at the time of testing, 1.2 mM dipeptide stock added to water was serial diluted down to le⁻¹⁰ μM in PBS, pH 7.0. About 100 μL of each dipeptide stock was added to a lyophilized vial containing LgTrip 3546 and substrate, pipetted briefly to mix, and then placed into a 96-well plate, and kinetic measurements were started immediately.

These data demonstrate that a stable, concentration dependent bioluminescent response was observed with the addition of the dipeptide (FIG. 26). This experiment highlights that a solid format lyophilized cake or tablet containing LgTrip 3546 and substrate can be made and then reconstituted in aqueous media containing a potential analyte of interest (e.g., dipeptide).

### Example 10

### Protein Buffer Formulations

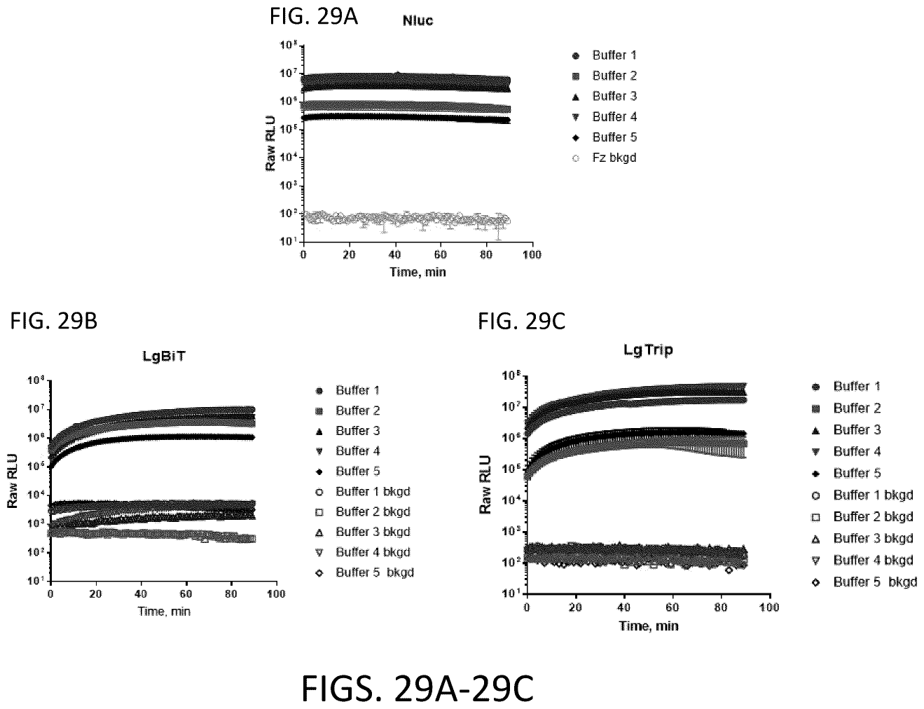

For FIGS. 29-33, experiments were conducted to test the compatibility of protein components with different protein buffer formulations, according to the experimental design shown in the schematic diagram below.

For these experiments, Whatman 903 protein saver spot cards were used with the following protein buffer formulations:
Protein buffer 1: 20 mM Na₃PO₄, 5% w/v BSA, 0.25% v/v tween20, 10% w/v sucrose
Protein buffer 2: 20 mM Na₃PO₄, 0.25% v/v tween20, 10% w/v sucrose
Protein buffer 3: 20 mM Na₃PO₄, 5% w/v BSA, 0.25% v/v tween20
Protein buffer 4: 20 mM Na₃PO₄, 5% w/v BSA, 0.25% v/v tween20, 2.5% pullulan
Protein buffer 5: 20 mM Na₃PO₄, 0.25% v/v tween20, 2.5% pullulan.

For NanoLuc, a 1000× stock solution was diluted 1:1000 in protein buffer (1 mL). For a 1.068 nM stock solution, 3 1 was diluted into 297 μl of protein buffer. About 5 μL of each concentration was spotted on the filter paper. For LgBiT-1672-11s-His, 5 μL of 1.068 nM protein per spot was used. About 10 μL was diluted in 990 μL protein buffer for a 2e⁻⁷ μM stock. About 100 μL of a 100 nM protein solution was then prepared, and about 10 μL stock was diluted into 990 μL protein buffer for 1 nM stock. About 5 μL of each concentration was spotted onto filter paper. For LgTrip 3546, about 5 μL of 1.068 nM protein was used per spot. About 1.1 μL of LgBiT-1672 stock was diluted into 998.94 μL protein buffer. About 3 μL stock was diluted into 297 μL protein buffer. About 5 μL of each concentration was spotted onto filter paper. After all protein was added, the samples were dried at 30° C. for about 1 hour. About 40 spots were made for each condition (see above schematic diagram). Spots were tested on day 0 for a baseline and then placed at 60° C. and tested 6 days later. RLU activity was tested by addition of 1 nM of high affinity dipeptide+50 μM live cell substrate in PBS, pH 7.0.

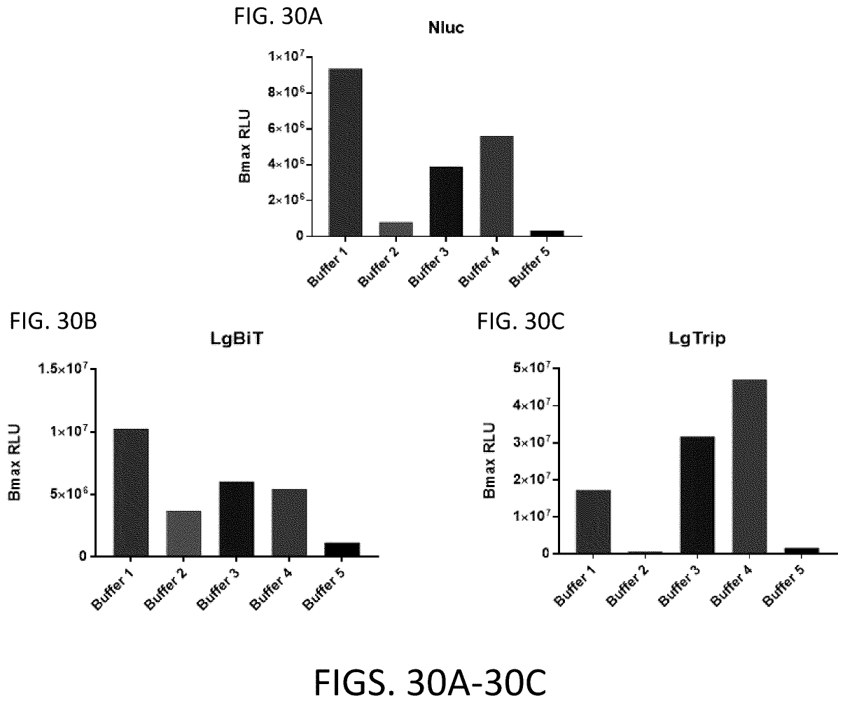

FIGS. 29A-29C show bioluminescent signal, measured by RLUs, in the various protein buffer formulations described above for NanoLuc (FIG. 29A), LgBiT-1672 (FIG. 29B), and LgTrip 3546 (FIG. 29C), and FIGS. 30A-30C show bioluminescent signal, measured by Bₘa, in various protein buffer formulations for NanoLuc (FIG. 30A), LgBiT-1672 (FIG. 30B), and LgTrip 3546 (FIG. 30C). Together, these data suggest that BSA is an important component in the protein buffer formulations tested, with NanoLuc and LgTrip 3546 exhibiting the largest decreases in RLU (buffers 2 and 5).

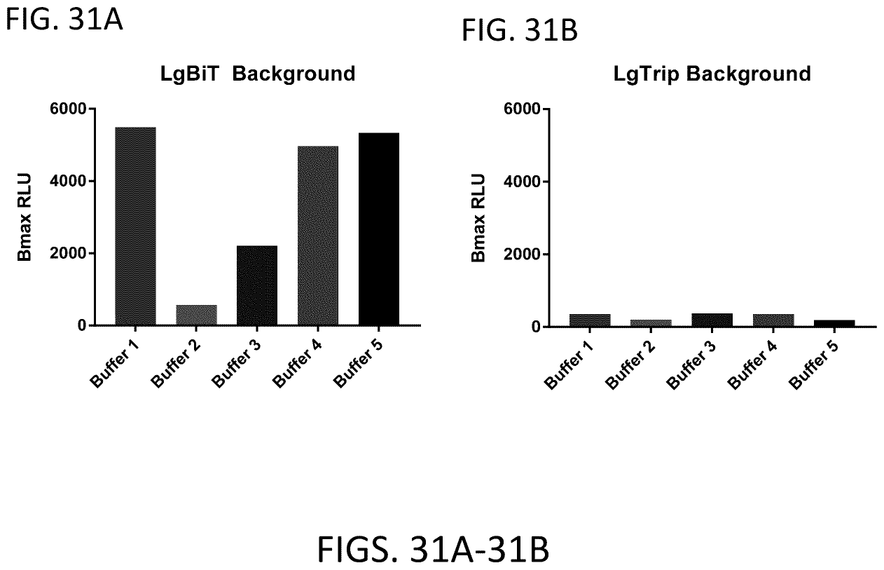

Experiments were also conducted to assess luminescent background levels in the various protein buffer compositions described above. FIGS. 31A-31B show bioluminescent background levels in various protein buffer compositions for LgBiT-1672 (FIG. 31A) and LgTrip 3546 (FIG. 31B). These data suggest that BSA or pullulan are important components of the protein buffer formulations for LgBiT-1672 for minimizing background luminescence, but there appears to be little to no effect on LgTrip 3546 background levels under these conditions.

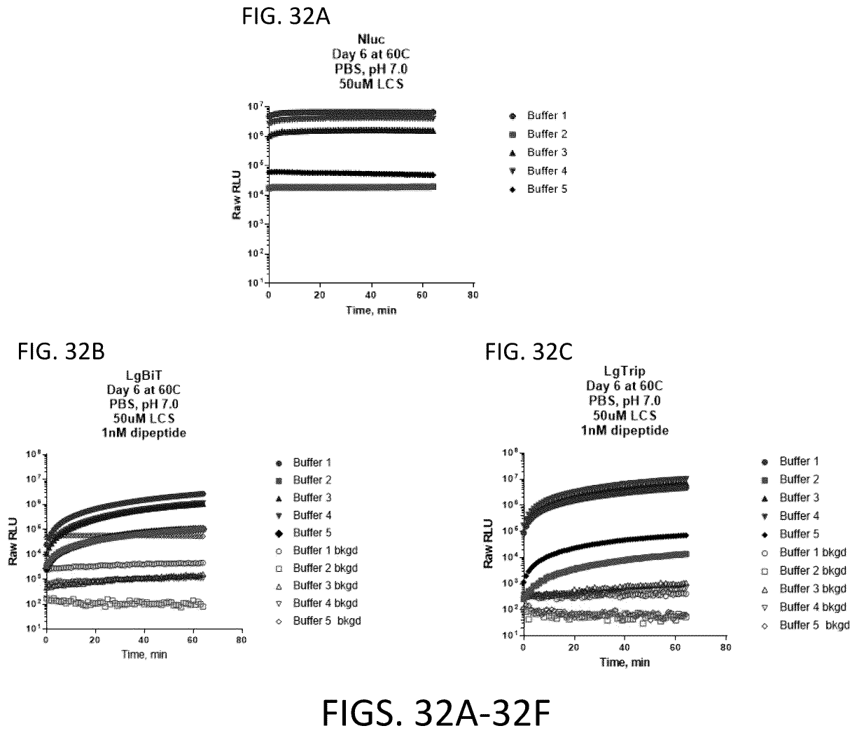

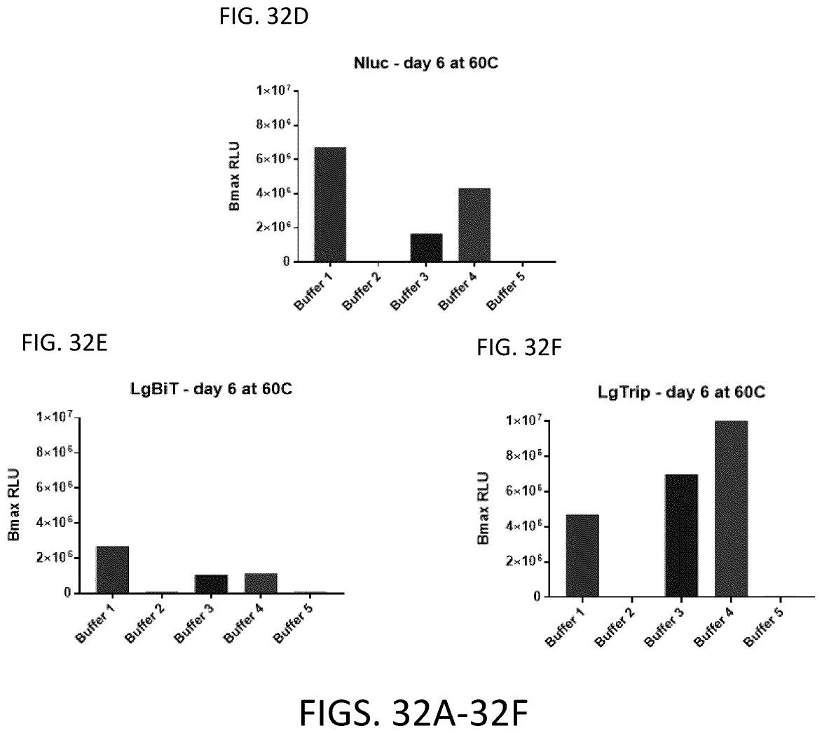

In FIGS. 32A-32F, the kinetics of the above conditions were assessed after addition of dipeptide and substrate in PBS. More specifically, FIGS. 32A-32F show bioluminescent signal (RLUs in FIGS. 32A-32C; Bₘₐₓ in FIGS. 32D-32F) in various protein buffer formulations for NanoLuc® (FIGS. 32A and 32D), LgBiT-1672 (FIGS. 32B and 32E), and LgTrip 3546 (FIGS. 32C and 32F), after 6 days at 60° C. These data indicate that proteins are stable and maintain activity after 6 days at 60° C. under these conditions, and suggest that BSA is an important component for all proteins buffer formulations. Additionally, FIG. 33 includes representative embodiments of all-in-one lyophilized cakes (“lyocakes”) or tablets containing all the necessary reagents to perform an analyte detection test supporting several types of assay formats, including but not limited to, cuvettes, test tubes, large volumes in bottles, snap test type assays, and the like.

### Example 11

### Lateral Flow Assays

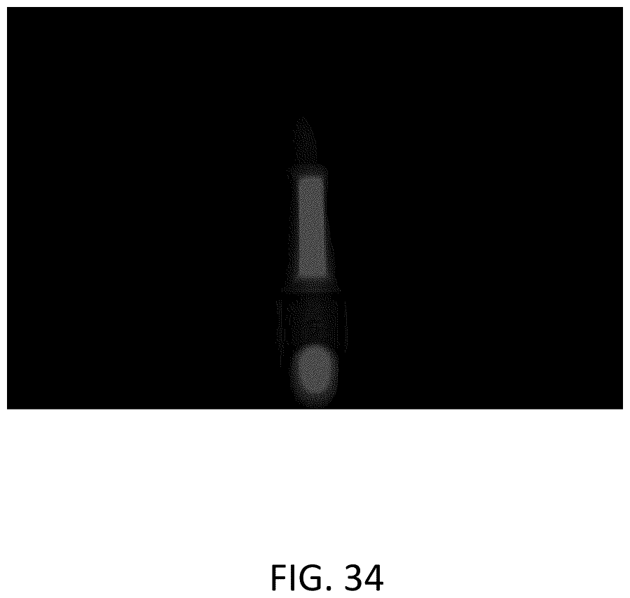

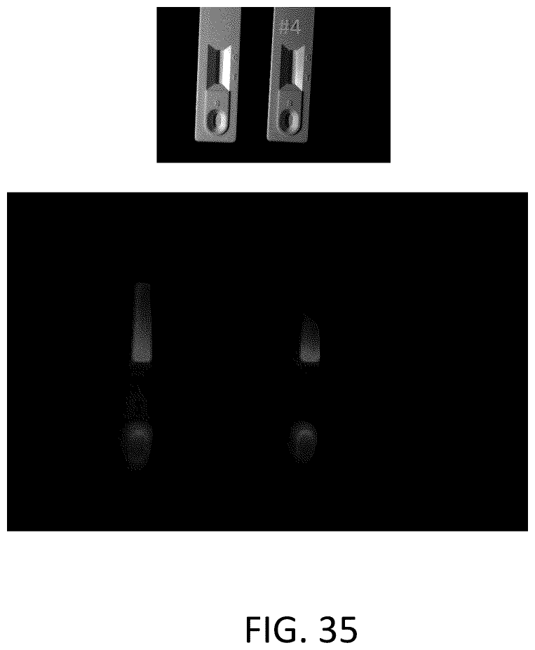

For FIGS. 34 and 35, lateral flow assays were performed using the information obtained in the above experiments, and according to the experimental design shown in the schematic diagram below.

The materials used for these experiments included a Conjugation pad (Ahlstrom grade 8950, chopped glass with binder, 50 g/m²), a Sample Pad (Cellose glass fiber CFSP203000 (Millipore)), an Absorption pad (Cotton linters, grade 238 (Ahlstrom)), a Membrane (nitrocellulose Hi-Flow Plus HFC07502 (Millipore), #6 from strip-test 2), and Running buffer (5× LCS dilution buffer+5× LCS diluted to 1× in PBS). Membranes were prepared by applying 30% sucrose solution to the membrane covering about 1.5 cm of the bottom of the strip. The membrane was allowed to dry at 35° C. for 1 hour. Strips were initially cut to be 4.5 cm×1 cm.
Protein preparations were carried out according to the conditions below:
Condition 1: 5 μL mouse anti-NanoLuc antibody diluted in 995 μL protein buffer, applied evenly across the conjugation pad with an air brush, and dried in oven at 37° C. Dilute 2.5 μL mouse antibody in 0.5 mL of protein buffer and applied directly to membrane.
Condition 2: Dilute 2.5 μL of NanoLuc in 0.5 mL of protein buffer and applied directly to membrane. Allowed to dry at 37° C. for 1 hour.
Condition 3: Treat entire membrane directly with 5 μL of NanoLuc diluted to 1 mL in protein buffer. Applied evenly with airbrush. Allowed to dry at 37° C. for 1 hour.
Condition 4: 2.5 μL mouse anti-NanoLuc antibody in 997 μL protein buffer. Applied evenly across conjugation pad with airbrush. Allowed to dry at 37° C. for 1 hour.
Condition 5: 1 μL mouse anti-NanoLuc antibody in 999 μL protein buffer. Applied evenly across conjugation pad with airbrush. Allowed to dry at 37° C. for 1 hour.

Strips were assembled on backing card with conjugation pad, sample pad, and wicking pad cut to 1 cm×1 cm. Once strips were assembled, they were cut in half lengthwise to a final dimension of 4.5 cm×0.5 cm. For imaging analysis, about 250 1 1× LCS buffer+LCS was diluted in PBS. Images were captured at 5 sec exposures with 5 sec wait time in between images; representative images are compilation images from corresponding movies taken across total exposure time (movies can be made available upon request). Total read time was 2:40 minutes.

FIG. 34 shows bioluminescent signal from substrate movement across a lateral flow strip from a compilation image corresponding to a movie taken across total exposure time. Substrate was added to the sample window of the lateral flow assay cassette and real time imaging shows substrate movement across the strip, and NanoLuc® activity can be seen throughout the test window (strip #3 in schematic above). By 70 seconds, the substrate flowed across the entire sample window.

FIG. 35 shows bioluminescent signal from NanoLuc® movement across a lateral flow strip from a compilation image corresponding to a movie taken across total exposure time (strip #s 4 and 5 in the schematic above). Under these conditions, strip #5 appeared to outperform strip #4 with, as demonstrated by the NanoLuc® flowing out of the conjugation pad and into the liquid flow across the membrane to the strip containing the mouse anti-NanoLuc antibody.

### Example 12

### Fumonisin Detection

Experiments were conducted during development of embodiments herein to demonstrate the use of NanoLuc®-based technologies in a competition-type immunoassay for the detection of a fumonisin B1, an exemplary small molecule toxin. Such assays can be performed in the devices and systems described herein, and with other small molecule targets and target analytes.

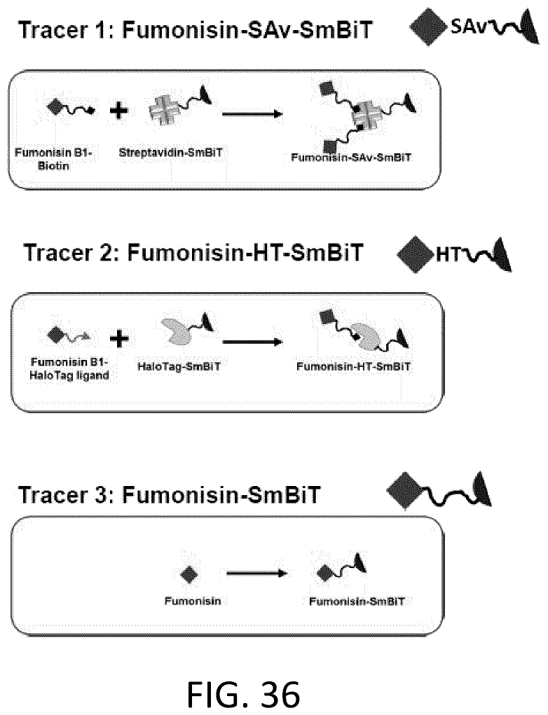

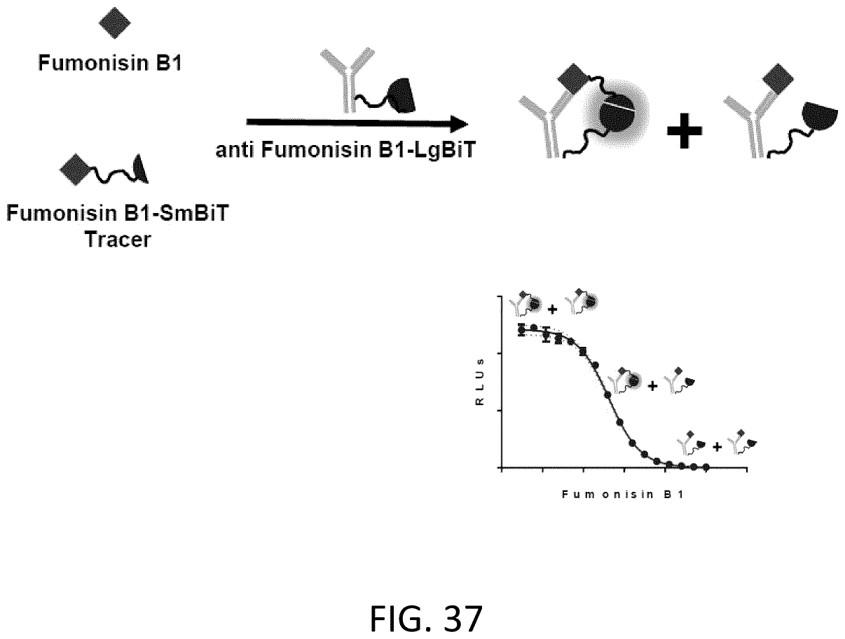

In an exemplary assay, tracers were generated by tethering fumonisin B1 to a NLpeptide tag (e.g., a peptide tag comprising SEQ ID NO: 10) via a biotin/streptavidin linkage, via a HaloTag linkage, or directly (FIG. 36). In some embodiments, the tracers can be combined with an anti-fumonisin B1 antibody linked to a polypeptide complement of the NLpeptide tag (e.g., a complement comprising SEQ ID NO: 9). A bioluminescent complex can form between the peptide tag and the polypeptide component upon binding of the antibody to the fumonisin B1. Exposure to varying concentrations of unlabeled Fumonisin B1 disrupts the bioluminescent complex and results in decreased luminescence, and the ability to detect/quantify the amount of fumonisin B1 in a sample (FIG. 37).

### Example 13

### Lyophilized Cake Containing LgBiT and Substrate

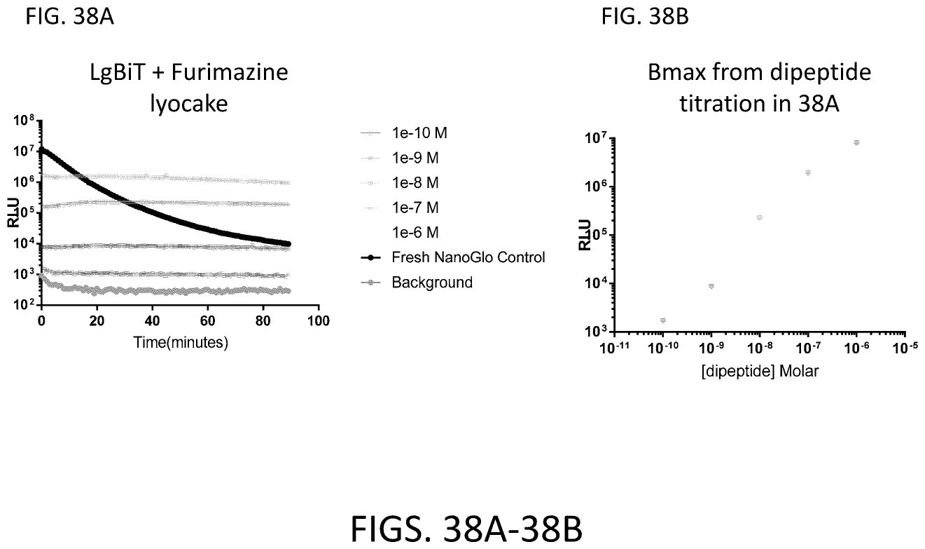

FIGS. 38A-38B show bioluminescent signal resulting from reconstitution with dipeptide of LgBiT and substrate from a lyocake (FIG. 38A) along with a titration of the dipeptide (FIG. 38B). To prepare a lyocake with LgBiT: 5% w/v pullulan in water containing 5 mM ATT and 5 mM ascorbic acid was prepared (solution 1). Solution 1 was then aliquoted out into 45 1 μl volumes in snap-cap vials. About 5 μl of 20 μM LgBiT protein was then added to each vial and pipetted to mix (solution 2). A 10 mM stock solution of furimazine in ethanol was prepared, and 5 μl of this solution was added to each vial and mixed (solution 3). Vials containing solution 3 were placed on dry ice to freeze for 1 hour, and then lyophilized overnight.

For luminescent measurements, at time of testing, 1.2 mM dipeptide stock in water was serial diluted down to le⁻¹⁰ μM in PBS, pH 7.0. 100 μl of each dipeptide stock was added to a lyophilized vial containing LgBiT and substrate, pipetted briefly to mix, and then placed into a 96-well plate and kinetic measurements were started immediately.

These data demonstrate that a stable, concentration dependent bioluminescent response was observed with the addition of the dipeptide. This experiment highlights that a solid format containing LgBiT and substrate can be made and then reconstituted in aqueous media containing a potential analyte of interest (e.g., dipeptide).

### Example 14

### Substrate and LgTrip 3546 or LgBiT Lyophilization

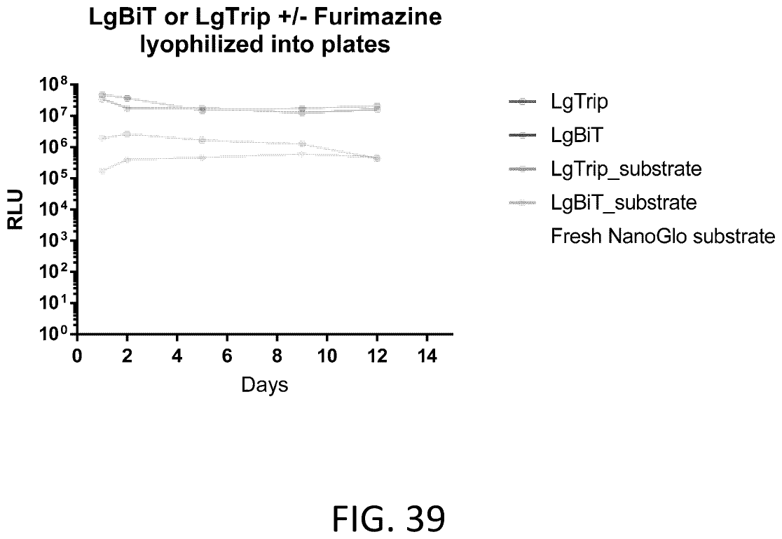

FIG. 39 shows bioluminescent signal resulting from reconstitution with dipeptide of LgBiT, or LgTrip 3546, and substrate from a lyocake prepared directly into a standard 96-well tissue culture treated plate (Costar 3917). To prepare a lyocake in plates: 2.5% w/v pullulan in water containing 5 mM ATT and 5 mM ascorbic acid was prepared (solution 1, pH 6.5). Solution 1 was then aliquoted out into 45 μl volumes into each well of the plate. 2.6 μl of 95 μM LgTrip 3546 protein was then added to each vial and pipetted to mix forming condition 1 (LgTrip 3546 alone). Additionally, 5 μl of 20 μM LgBiT protein was added to each vial and pipetted to mix, forming condition 2 (LgBiT alone). 5 μl of ethanol was then add to each well of condition 1 and 2 as a vehicle control.

Conditions 3 (LgTrip 3546/substrate) and 4 (LgBiT/substrate) were prepared as described above: 2.5% w/v pullulan in water containing 5 mM ATT and 5 mM ascorbic acid was prepared (solution 1, pH 6.5). Solution 1 was then aliquoted out into 45 μl volumes into each well of the plate. About 2.6 μl of 95 μM LgTrip 3546 protein or 5 μl of 20 μM LgBiT protein was added to each vial and pipetted to mix. Approximately 5 μl of 10 mM furimazine in ethanol was then added to each well forming condition 3 and 4 respectively. The plate was then placed in a cooler with dry ice to freeze for 1 hour, followed by lyophilization overnight.

For luminescent measurements, at time of testing, 1.2 mM dipeptide stock in water was serial diluted down to le⁻⁹ μM in PBS, pH 7.0 (FIG. 39). Fresh NanoGlo® substrate was then added to this stock for a final concentration of 10 μM substrate. 100 μl of this solution was added to wells that contained condition 1 (LgTrip 3546) and 2 (LgBiT). Conditions 3 (LgTrip 3546/substrate) and 4 (LgBiT/substrate) only received 100 μl of le⁻⁹ μM dipeptide in PBS. After testing, the plates were wrapped in tin foil and left on the bench at ambient temperature.

This data demonstrates that a lyocake containing either LgBiT or LgTrip 3546 and substrate can be prepared directly within a 96-well plate and reconstituted in the presence of an analyte of interest (dipeptide) leading to stable and robust signal.

### Example 15

### Paper Based All-In-One Analyte Detection Systems

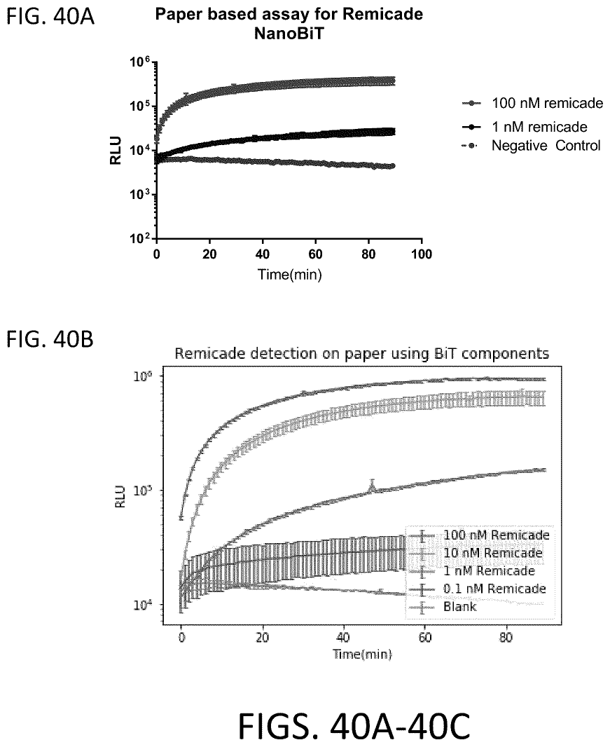

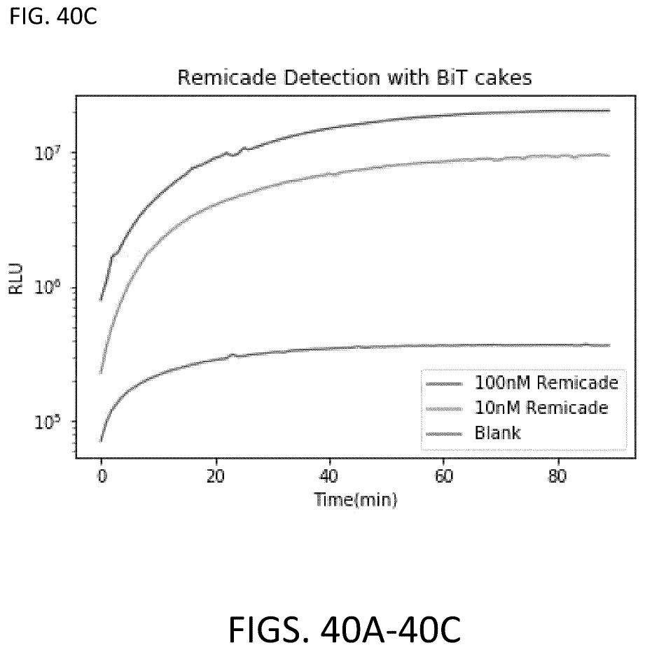

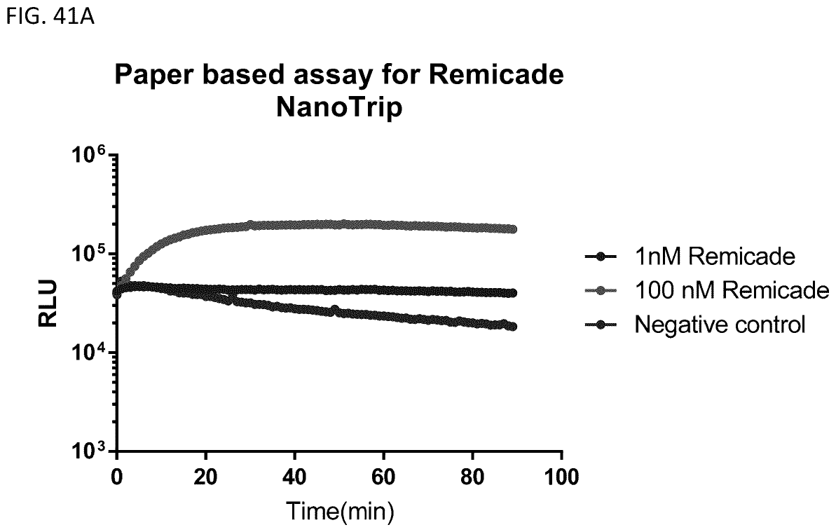

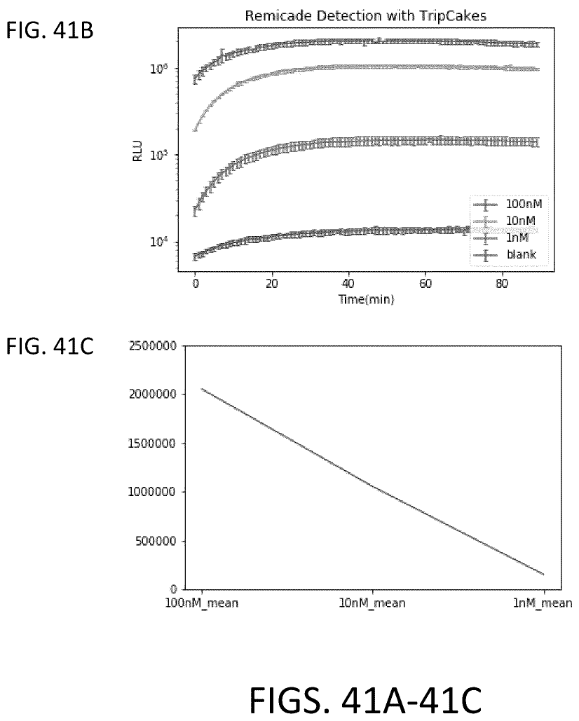

Experiments were conducted to test the efficacy of paper-based detection platforms containing NanoBiT (FIGS. 40A-40B) and NanoTrip (FIG. 41A) complementation systems. Paper spots were created from punching ⅛″ diameter circles from Whatman903 spot paper. The spots were treated with 5 μl of a master mix solution containing: 5% w/v BSA, 5 mM ATT, 5 mM ascorbate, 40 nM LgBiT-protein G fusion, and 20 nM SmBiT-TNFα in water, pH 6.5. The spots were allowed to dry at 35 C for 1 hour. A 200 μM solution of furimazine in ethanol was prepared, and 5 1 μl of this solution was added to each spot. The spots were allowed to dry for an additional 30-60 minutes at 35° C. At the time of testing, spots were plated into individual wells of a 96-well NBS plate (Costar 3917), and reconstituted with Opti-MEM assay buffer that contained either 0 nM (blank), 1 nM, or 100 nM Remicade.

FIGS. 40A-40B include assay results using NanoBiT components. In the condition where the spots were exposed to assay buffer containing 1 nM Remicade, there was an increase in overall light output compared to the blank condition/control, which contained no Remicade. An increase in signal is observed as the concentration of Remicade was increased to 100 nM. As shown in FIG. 40B, Remicade was prepared in opti-MEM assay buffer at 100 nM, 10 nM, 1 nM, and 0.1 nM concentrations. At time of testing, 100 1 μl of each solution containing Remicade was added to a well of a 96-well plate containing a spot, and RLU output was measured.

Similar experiments were performed, as shown in FIG. 41A using NanoTrip components. Spots were created from punching ⅛″ diameter circles from Whatman903 spot paper. Each the spot was treated with 5 μl of a master mix solution containing: 5% w/v BSA, 5 mM ATT, 5 mM ascorbate, 20 μM LgTrip 3546, 100 nM TNFα-15gs-VSHiBiT, SmTrip9 Pep521-15gs-protein G in water, pH 6.5. The spots were allowed to dry at 35° C. for 1 hour. A 200 μM solution of furimazine in ethanol was prepared and 5 μl of this solution was added to each spot. The spots were allowed to dry for an additional 30 minutes at 35° C. At the time of testing, spots were plated into individual wells of a 96-well NBSplate (Costar 3917), and reconstituted with opti-MEM assay buffer that contained either 0 nM (blank), 1 nM, or 100 nM Remicade. The results are shown in FIG. 41A.

These experiments show that it is possible to build and all-in-one, paper-based bioluminescent assay platforms for the detection of an analyte-of-interest using both NanoBiT and NanoTrip complementation systems. In addition, these experiments demonstrate that it is possible to quantify the amount of analyte present in the sample matrix based on a change in overall light output. Increasing the concentration of the analyte-of-interest (i.e. Remicade) led to a proportional increase in the bioluminescent signal (the bioluminescent signal generated from the analyte detection complex is proportional to the concentration of the analyte).

### Example 16

### Lyocake Based All-In-One Analyte Detection Systems

Experiments were also conducted to test the efficacy of lyocake-based detection platforms containing NanoBiT (FIG. 40C) and NanoTrip (FIGS. 41B-41C) complementation systems.

As shown in FIG. 40C, stability conditions were tested when drying down the components of the bioluminescent complexes. About 45 μl of a master mix solution was added to 1.5 mL, plastic snap-cap vials. The master mix included: 5% w/v pullulan, 5 mM ATT, 5 mM ascorbate, 40 nM LgBiT-protein G fusion, and 20 nM SmBiT-TNFα, at pH 6.5. About 5-10 μl of the substrate furimazine in ethanol was added to each vial, mixed, and placed in dry ice for about 1 hour. The frozen samples were then lyophilized overnight to form a lyocake. At the time of testing, solutions of 100 nM and 10 nM Remicade were prepared in Opti-MEM assay buffer. About 100 μl of these solutions were added to the vials containing the NanoBiT Cake, pipetted to mix, and then transferred to a Costar 3600 96-well plate. A blank control was prepared that lacked the analyte Remicade. The results in FIG. 40C demonstrate a proportional increase in signal as the analyte concentration increased, even when all the components of the bioluminescent complex, including the substrate, are frozen and stored in the form of a lyocake, and subsequently exposed to the analyte-of-interest.

In FIGS. 41B-41C, stability conditions were tested when drying down the components of the bioluminescent complexes. About 45 μl of a master mix solution was added to 1.5 mL, plastic snap-cap vials. The master mix included: 5% w/v pullulan, 5 mM ATT, 5 mM ascorbate, 9 μM LgTrip 3546, 225 nM SmTrip9-Protein G, and 45 nM SmBiT-TNFα, at pH 6.5. About 5-10 μl of the substrate furimazine in ethanol was added to each vial, mixed, and placed in dry ice for about 1 hour. The frozen samples were then lyophilized overnight to form a lyocake. At the time of testing, solutions of 100 nM, 10 nM and 1 nM Remicade were prepared in Opti-MEM assay buffer. About 100 μl of these solutions were added to the vials containing the NanoTrip Cake, pipetted to mix, and then transferred to a Costar 3600 96-well plate. A blank control was prepared that lacked the analyte Remicade. The results in FIG. 41B-41C demonstrate a proportional increase in signal as the analyte concentration increased, even when all the components of the bioluminescent complex, including the substrate, are frozen and stored in the form of a lyocake, and subsequently exposed to the analyte-of-interest.

In the condition where the spots were exposed to assay buffer containing 1 nM Remicade, there was an increase in overall light output compared to the blank condition, which contained no Remicade. An increase in signal was observed as the concentration of Remicade increased to 100 nM. These experiments show that it is possible to build and all-in-one lyocake-based, bioluminescent-based assay platforms for the detection of an analyte-of-interest using both NanoBiT and NanoTrip complementation systems. In addition, these experiments demonstrate that it is possible to quantify the amount of analyte present in the sample matrix based on a change in overall light output. Increasing the concentration of the analyte-of-interest (i.e. Remicade) led to a proportional increase in the bioluminescent signal (the bioluminescent signal generated from the analyte detection complex is proportional to the concentration of the analyte).

### Example 17

Mesh-Based Systems to Separate Substrate from Bioluminescent Complexes for Analyte Detection

Experiments were conducted to investigate the conditions required to generate a bioluminescent signal when peptide and polypeptide components of the bioluminescent complexes provided herein were produced in a format that does not include the substrate. For example, in one embodiment, an amount of a solution (e.g., containing an analyte-of-interest) is added to a mesh or matrix that has the luminogenic substrate adhered (“caked”) to it. Addition of the solution acts to reconstitute the substrate on the mesh, and this solution subsequently interacts with the surface of paper containing the dried down peptides and polypeptides of the bioluminescent complexes of the present disclosure, thus generating a bioluminescent signal (FIG. 42A). The mesh format does not hinder the ability to detect the bioluminescent signal; any bioluminescence detected comes from the surface of the paper, and not from any solution phase that is formed during the experiment.

As shown in FIG. 42A, bioluminescence is detectable using this format. Whatman 903 paper spots were made to have about 0.25 inch diameters, similar to the nylon mesh. The master mix, which was used to generate the paper spots containing the bioluminescent peptide/polypeptide components, included: 5% w/v BSA, 5 mM ATT, 5 mM ascorbate, 10 μM NanoLuc, at pH 6.5. About 10-20 1 μl of the master mix was added to the spots and then dried at about 35° C. for about 1 hour. To generate the mesh containing the substrate, a solution of about 0.75% pullulan in water was prepared. About 450 μl of this solution was added to a plastic snap-cap vial. About 50 μl of 10 mM furimazine in EtOH was added to the vial and pipetted to mix. About 25 μl of this solution was added to the top of the mesh-spots. The mesh spots were then frozen on dry-ice, and lyophilized overnight. At time of testing, the mesh containing the lyocake substrate was placed on top of the spots containing the NanoLuc® protein. The complete system was then added to the well of a 96-well costar 3600 plate. About 10 μl of PBS was then added to the top of the mesh to reconstitute the material and the plate was read for RLU light output.

Experiments were also conducted using LgTrip 3546 bioluminescent components with the mesh-based format. The master mix, which was used to generate the paper spots containing the bioluminescent peptide/polypeptide components, included: 5% w/v BSA, 5 mM ATT, 5 mM ascorbate, 100 nM LgTrip 3546, at pH 6.5. About 10-20 μl of the master mix was added to the spots and then dried at about 35° C. for about 1 hour. To generate the mesh containing the substrate, a solution of about 0.75% pullulan in water was prepared. About 450 μl of this solution was added to a plastic snap-cap vial. About 50 μl of 10 mM furimazine in EtOH was added to the vial and pipetted to mix. About 25 μl of this solution was added to the top of the mesh-spots. The mesh spots were then frozen on dry-ice, and lyophilized overnight. At the time of testing, dipeptide ranging from 100 nM to 0.1 nM was prepared in PBS. The spots were placed in wells, and the screen containing the substrate was placed on the surface of the spots. About 10 μl of the solutions containing each concentration of peptide was added to the surface of the screen and RLU's were recorded (FIGS. 42B-42C). The blank control did not contain any dipeptide.

Experiments were also conducted using LgTrip 3546 bioluminescent components with the mesh-based format and by forming a pullulan film. The master mix, which was used to generate the paper spots containing the bioluminescent peptide/polypeptide components, included: 5% w/v BSA, 5 mM ATT, 5 mM ascorbate, 100 nM LgTrip 3546, at pH 6.5. About 10-20 1 μl of the master mix was added to the spots and then dried at about 35° C. for about 1 hour. To generate the mesh containing the substrate, a solution of about 2.0% pullulan in water was prepared. About 450 μl of this solution was added to a plastic snap-cap vial. About 50 μl of 10 mM furimazine in EtOH was added to the vial and pipetted to mix. About 25 μl of this solution was added to the top of the mesh-spots. The spots were then allowed to dry under ambient conditions, in the dark, overnight. This method resulted in the formation of a pullulan film that filled the holes of the mesh. At the time of testing, dipeptide ranging from 100 nM to 0.1 nM was prepared in PBS. The spots were placed in wells, and the screen containing the substrate was placed on the surface of the spots. About 10 μl of the solutions containing each concentration of peptide was added to the surface of the screen and RLU's were recorded (FIGS. 42D-42E). The blank control did not contain any dipeptide.

These experiments show that it is feasible to detect bioluminescent signal in a mesh-based format in which the peptide/polypeptide components are separate from the substrate. In addition, in the context of this format, these experiments demonstrate that increasing the concentration of the analyte-of-interest (i.e. dipeptide) leads to a proportional increase in the bioluminescent signal (the bioluminescent signal generated from the analyte detection complex is proportional to the concentration of the analyte).

### Example 18

### Testing Different Formulated, Lyophilized Substrates for Cake Appearance, Reconstituted Kinetic Activity Performance, and Accelerated Thermal Stability

To evaluate the potential application of lyophilization for preservation of the furimazine substrate, formulations containing furimazine were prepared. The 20× stock formulations were as follows:
Condition 1: 100 μM furimazine in ethanol, 5 mM azothiothymine, 5 mM ascorbic acid, 2.5% pullulan w/v, ddH20 (Millipore);
Condition 3: 100 μM furimazine in ethanol, 5 mM azothiothymine, 5 mM ascorbic acid, 2.5% pullulan w/v, 20 mM HEPES buffer (pH 8.0), 90 mM glycine, 20 mM histidine, 25 mg/ml sucrose, 0.01% polysorbate 80;
Condition 5: 40 μM furimazine in 85% ethanol+15% glycol, 200 mM MES buffer (pH 6.0), 200 mM hydroxyproyl beta cyclodextrin (m.w. 1396 Da), 600 mM sodium ascorbate, 2.5% pullulan w/v; and

Condition 7: 20 μM furimazine in ethanol, 200 mM MES buffer (pH 6.0), 200 mM hydroxyproyl beta cyclodextrin (m.w. 1396 Da), 600 mM sodium ascorbate, 2.5% pullulan w/v.

One mL aliquots of 20× stock solution was dispensed into 10 mL amber glass vials, and a runner stopper was partially inserted into the vial. Vials were loaded into a lyophilizer (Virtis Genesis 12EL lyophilizer) with shelves pre-chilled to 4.7° C. Product then underwent a freezing step with a shelf temperature of −50° C. for 2 hr after which time the condenser step started. During the run, the condenser temperature ran between −5° C. and −87° C. A vacuum pull down ran next at the pressure set-points of 75 and 200 mTorr. Sublimation lasted ˜7.5 hr, and desorption lasted ˜16.1 hr. At the end of the lyophilization process, the vials were back-filled with nitrogen and sealed with fully inserted stoppers at −600 Torr of pressure.

Vials were stored at 25° C. or 60° C. and tested at various timepoints post-lyophilization. For activity-based assays, furimazine cakes were reconstituted with 10 mL of PBS containing 0.01% BSA. The vials were shaken manually and allowed to equilibrate at room temperature for 5 minutes. Fifty l of the reconstituted substrate was added to 50 μl of 1 ng/mL purified NANOLUC enzyme (Promega) that was reconstituted in the same BSA buffer (final [NanoLuc]=0.5 ng/ml). The controls used were the NANOGLO Live Cell Substrate (Promega Cat. N205) or NANOGLO substrate (Promega Cat. N113) according to manufacturer's protocol, but were diluted into PBS containing 0.01% BSA instead of the dilution buffer provided in the kit (Promega). Assays were performed in solid, white, nonbinding surface (NBS) plates (Costar) and analyzed on a GLOMAX Discover Multimode Microplate Reader (Promega) collecting total luminescence using kinetic or endpoint reads, depending on the experiment. For analysis of absolute [furimazine], reconstituted samples were analyzed on HPLC for absorbance spectra at wavelength 245 nm and the absolute amount remaining from day 0 was plotted.

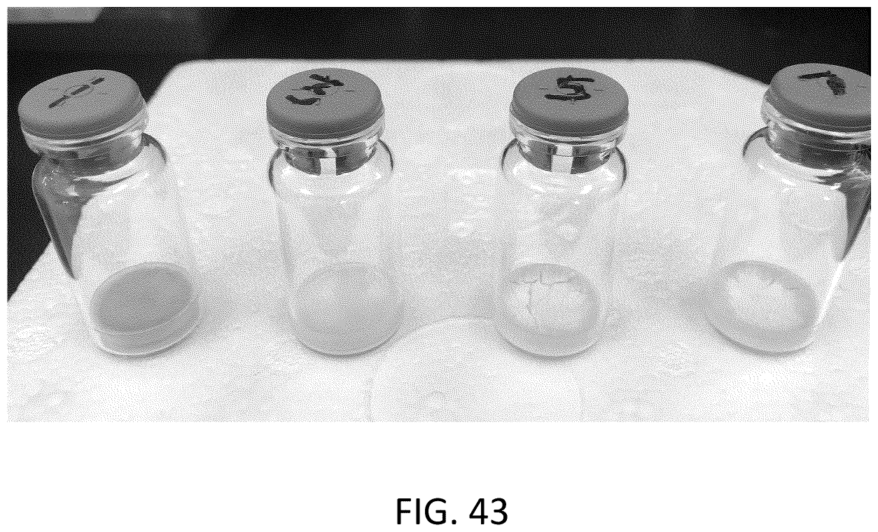

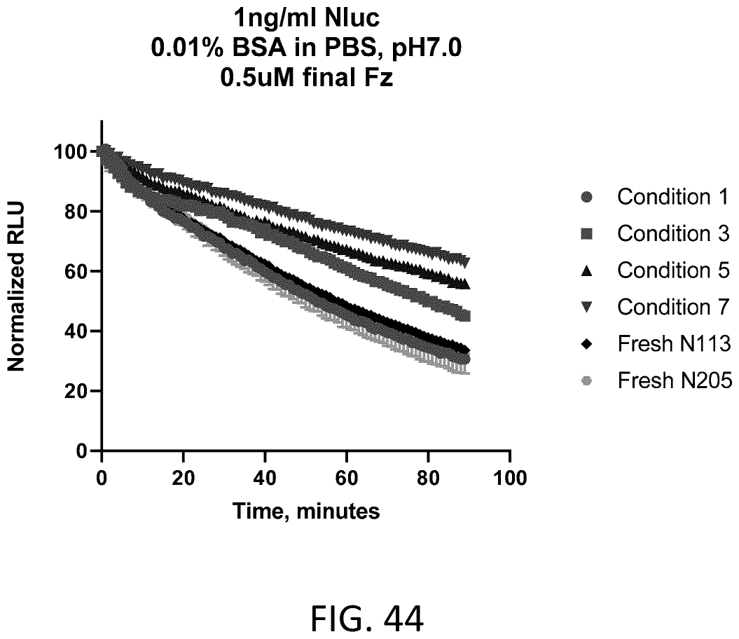

The appearance of the lyophilized cakes resulting from these formulations are displayed in FIG. 43, which shows that all 4 conditions tested produced an intact cake, although conditions 5 and 7 did display some cracking. A pH indicator that was supplied for these vials indicated that the resulting cakes had pH values of about 2-3 for Condition 1, pH values of about 7.5 for Condition 3, and pH values of about 6 for Conditions 6 and 7. Signal kinetics of the reconstituted furimazine, when tested with purified NanoLuc, compared to that of furimazine in standard organic storage buffer (N113 and N205) and maintained at −20° C., indicated there was no observable loss in performance due to the formulated buffer and lyophilization process itself, with an improved half-life for conditions 5 and 7 (FIG. 44).

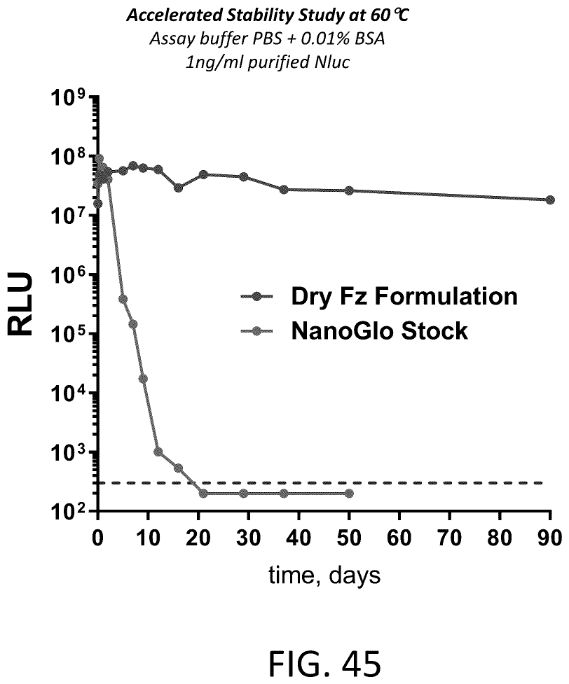

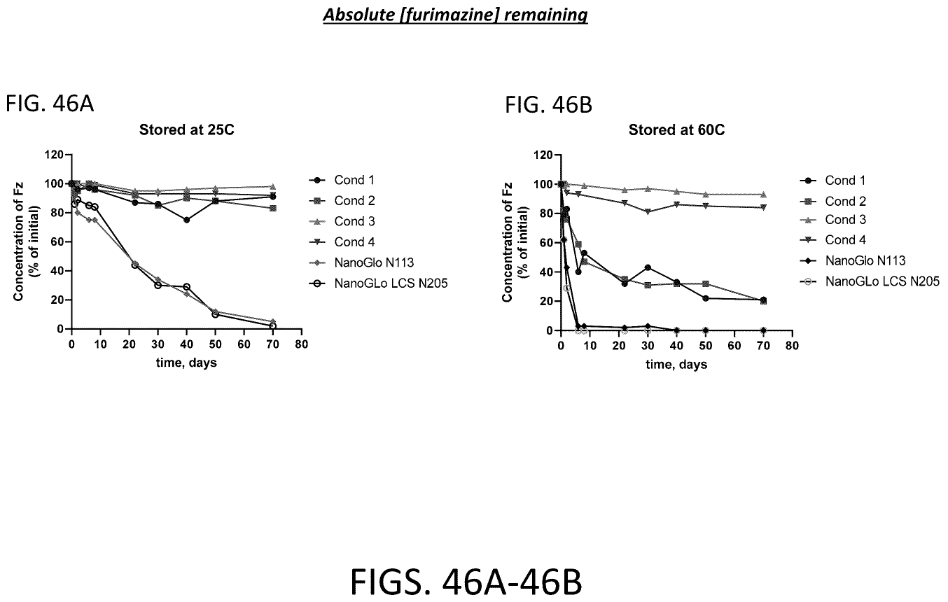

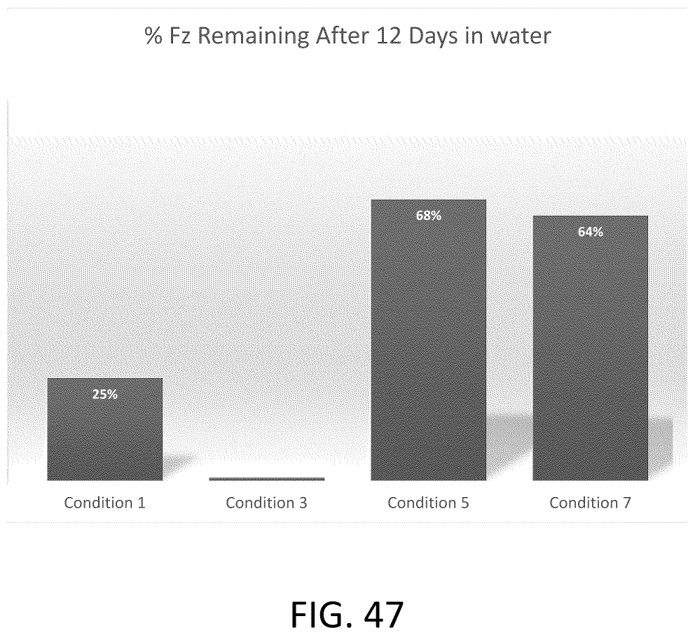

Accelerated thermal stability studies indicated no loss of activity for 3 months for the formulated and lyophilized furimazine for Condition 1, which in stark contrast to the furimazine stored in organic solvent, which lost all activity in about 10 days when stored at this elevated temperature (FIG. 45). HPLC analysis for the absolute [furimazine] remaining after storage at 25° C. and 60° C. supported the activity findings with the formulated and lyophilized substrate containing significantly higher purity of furimazine relative to furimazine in the standard organic storage buffer (FIGS. 46A and 46B). To determine the liquid stability of the formulated, lyophilized furimazine, vials were reconstituted with water and allowed to remain in solution for 12 days prior to analysis by HPLC for total remaining furimazine as compared to day 0. Liquid stability of conditions 5 and 7 were found to be superior (FIG. 47).

### Example 19

### Development of a Solution-Based, Homogeneous Human Interleukin-6 Tripartite Immunoassay Using HaloTag-Peptide Fusions to Chemically Conjugate Monoclonal Antibody Pairs

The basic principle of the homogeneous NanoLuc tripartite (NanoTrip) immunoassay is depicted in FIG. 48. First, a pair of antibodies that target non-overlapping epitopes on IL-6 are chemically conjugated to SmTrip9 (SEQ ID NO: 13) or HiBiT (SEQ ID NO: 11) using the HaloTag® technology. When the labeled antibodies bind an IL-6 analyte, the complementary subunits are brought into proximity thereby reconstituting a bright luciferase in the presence of the LgTrip 3546 protein (SEQ ID NO: 12) and furimazine substrate. This assay is quantitative because the amount of luminescence generated by a standard plate-reading luminometer is directly proportional to the amount of target analyte present.

Genetic fusions containing the SmTrip9 variants (SmTrip9 Pep521; SEQ ID NO: 16) or SmTriplO variants (SmTriplO Pep289 or VSHiBiT; SEQ ID NO: 17 separated by either a 2× or 3× Gly-Ser-Ser-Gly linker to the amino terminus of HaloTag was achieved using the pFN29A HIS6HaloTag T7 Flexi Vector (Promega). Glycerol stocks of E. coli expressing HisTag-HaloTag fusion protein was used to inoculate 50 mL starter cultures, which were grown overnight at 37° C. in LB media containing 25 μg/ml kanamycin. Starter cultures were diluted 1:100 into 500 mL fresh LB media containing 25 μg/mL kanamycin, 0.12% glucose, and 0.2% rhamnose. Cultures were grown for 22-24 h at 25° C. Cells were pelleted by centrifugation (10,000 rpm) for 30 min at 4° C. and re-suspended in 50 mL PBS. 1 mL protease inhibitor cocktail (Promega), 0.5 mL RQ1 DNase (Promega), and 0.5 mL of 10 mg/mL lysozyme (Sigma) were added, and the cell suspension was incubated on ice with mild agitation for 1 h. Cells were lysed by sonication at 15% power at 5 s intervals for 1.5 min (3 min total) and subsequently centrifuged at 10,000 rpm for 30 min at 4° C. Supernatant was collected, and protein purified using HisTag columns (GE) following manufacturer's recommended protocol. Protein was eluted using 500 mM imidazole, dialyzed in PBS, characterized using SDS-PAGE gel and was >95% pure. Proteins were stored in 50% glycerol at −20° C.

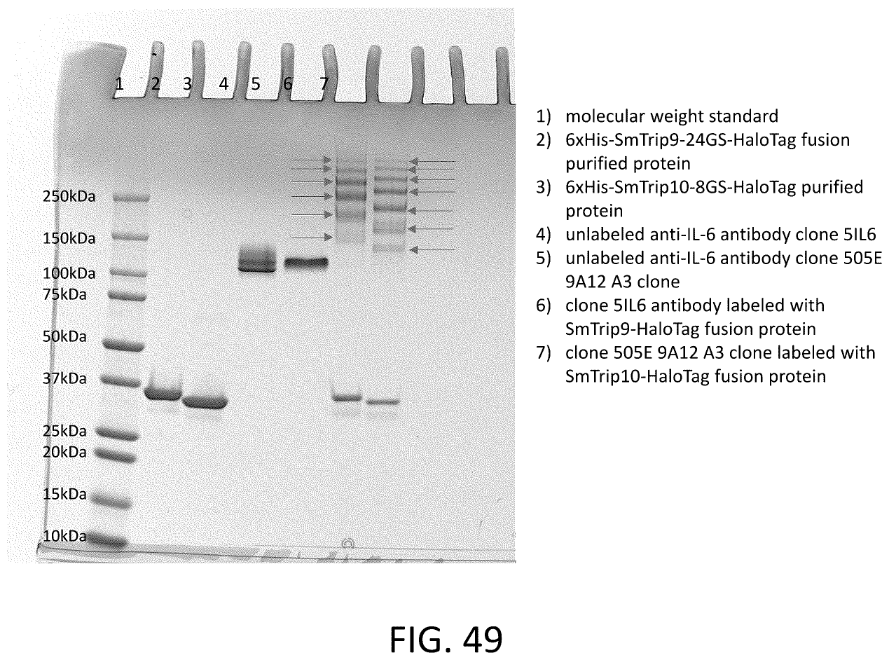

To chemically conjugate the antibodies to the HaloTag-peptide fusion proteins, antibodies were buffered exchanged 2× into 10 mM sodium bicarbonate buffer (pH 8.5) using Zeba spin desalting columns (ThermoFisher). Antibodies were then primed with 200μM amine-reactive HaloTag Succinimidyl Ester (04) Ligand (Promega) for 2 hr shaking at 1000 rpm at 22° C. Unreacted ligand was removed with two passes through Zeba spin columns in PBS buffer. Then, antibodies were covalently labeled with 30 μM of the HaloTag fusion protein overnight at 4° C. while shaking. Excess unreacted HaloTag fusion protein was removed using HaloLink Resin (Promega). Non-denaturing SDS-PAGE gel was used to characterize the conjugated antibodies. Mouse anti-human IL-6 monoclonal antibodies used in the human IL-6 immunoassay were clone 5IL6 (Thermo cat #M620) and clone 505E 9A12 A3 (Thermo cat #AHC0662). SDS-PAGE gels were performed on the labeled antibodies and it was determined that each antibody was labeled with a variable number of peptide-HaloTag fusion proteins, with the primary species containing 3-5 peptide labels (FIG. 49).

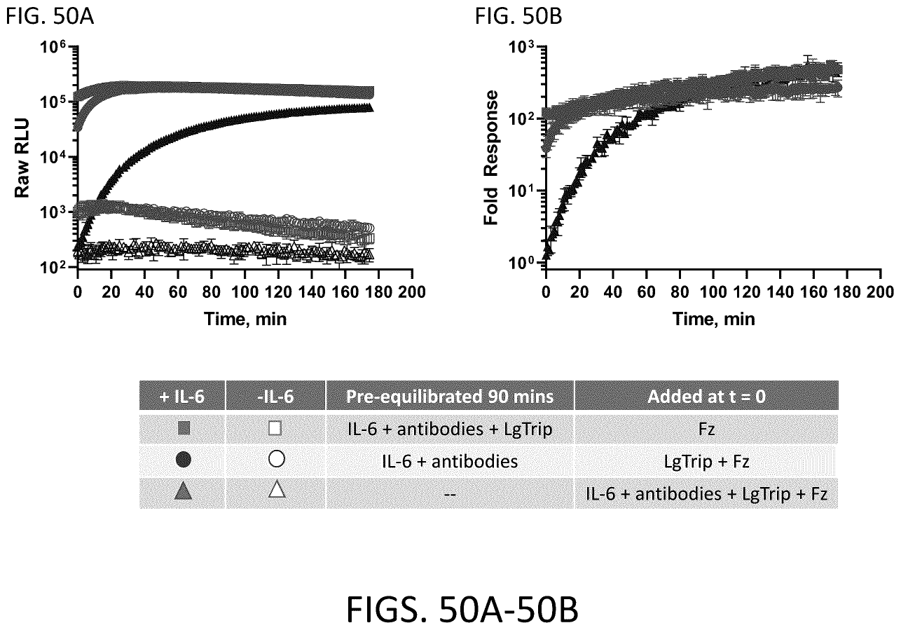

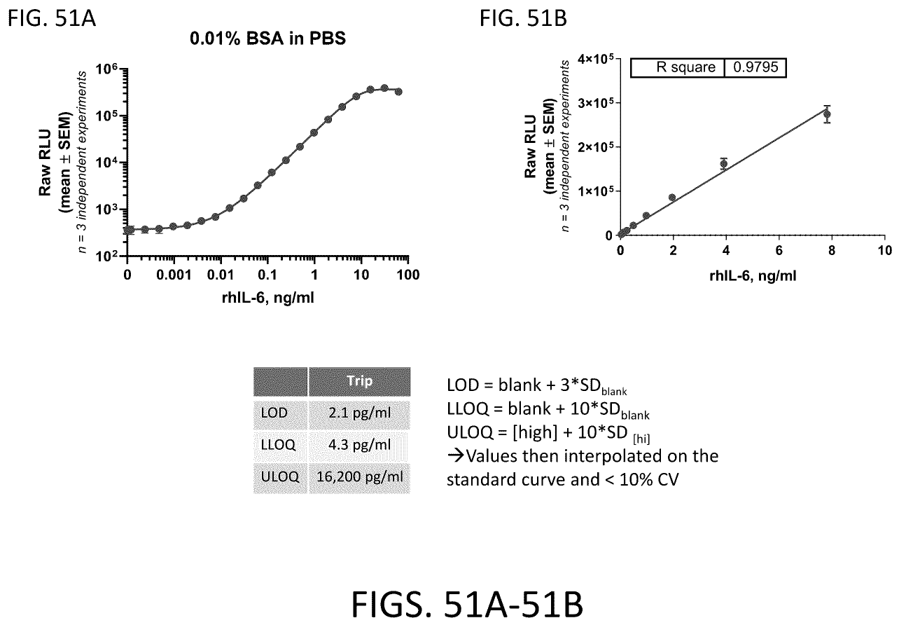

Binding kinetic studies were performed to establish maximum light output and signal duration of the fully complemented system as show in FIG. 50. The signal kinetics were compared between conditions: (1) peptide labeled antibodies and LgTrip 3546 (SEQ ID NO: 12) were pre-equilibrated with rhIL-6 for 90 minutes with addition of furimazine at time 0, (2) peptide labeled antibodies are pre-equilibrated with rhIL-6 for 90 minutes with addition of LgTrip 3546 and furimzine at time 0, and (3) all assay reagents are added to rhIL-6 at time 0. Condition 2 tracks the binding kinetics of LgTrip 3546 (SEQ ID NO: 12) to the peptide labeled antibodies:rhIL-6 complex. Condition 3 tracks the binding kinetics of the antibodies to the analyte and the LgTrip 3546 to the peptides. FIG. 50A displays the raw RLUs and FIG. 50B displays the fold response as calculated by taking the RLU value generated in the presence of 5 ng/ml rhIL-6 divided by the background signal generated in the absence of rhIL-6. The assay buffer used was 0.01% BSA in PBS, pH 7.0, and assay reagent concentrations were 7 ng/ml for each peptide labeled antibody, 1 μM LgTrip 3546 (SEQ ID NO: 12) protein, and furimazine. FIG. 51 displays the dose response curve for the solution-based homogenous IL-6 immunoassay performed in a standard assay buffer consisting of 0.01% BSA in PBS, pH 7.0. This assay was shown to be extremely sensitive with a limit of detection (LOD) of 2.1 pg/ml, which resulted in a broad dynamic range of over 3-4 orders of magnitude, and maintained low variability (CVs <10%) throughout the linear range. For these experiments, 7 ng/ml of each peptide labeled antibody and 1 μM LgTrip 3546 (SEQ ID NO: 12) protein were incubated in the presence of rhIL-6 for 90 minutes. Furimazine was added, and luminescence signal analyzed.

### Example 20

### Lyophilized, Single-Reagent Tripartite Immunoassays in Vials

To evaluate the potential application of lyophilization for preservation of the entire IL-6 tripartite immunoassay in a single vial, formulations containing peptide labeled antibodies (SmTrip9 Pep521 (SEQ ID NO: 16) and SmTriplO Pep289 (SEQ ID NO: 17)), LgTrip 3546 (SEQ ID NO: 12), and furimazine were prepared. The 20× stock formulations are as follows:
Formulation A: 20 mM HEPES buffer (pH 8.0), 90 mM glycine, 20 mM histidine, 25 mg/ml sucrose, 0.01% polysorbate 80, 0.6 ug/ml clone 5IL6 antibody labeled with HaloTag-SmTrip9 Pep521 (SEQ ID NO: 16), 1.2 ug/ml 505E A12 A3 antibody labeled with HaloTag-SmTriplO Pep289 (SEQ ID NO: 17), and 20 μM LgTrip 3546 (SEQ ID NO: 17).
Formulation B: 20 mM HEPES buffer (pH 8.0), 90 mM glycine, 20 mM histidine, 25 mg/ml sucrose, 0.01% polysorbate 80, 0.6 ug/mi clone 5IL6 antibody labeled with HaloTag-SmTrip9 Pep521 (SEQ ID NO: 16), 1.2 ug/ml 505E A12 A3 antibody labeled with HaloTag-SmTriplO Pep289 (SEQ ID NO: 17), 20 μM LgTrip 3546 (SEQ ID NO: 12), and 100 μM furimazine in ethanol.
Formulation C: 5 mM azothiothymine, 5 mM ascorbic acid, 2.5% pullulan w/v, 20 mM HEPES buffer (pH 8.0), 90 mM glycine, 20 mM histidine, 25 mg/ml sucrose, 0.01% polysorbate 80, 0.6 ug/ml clone 5IL6 antibody labeled with HaloTag-SmTrip9 Pep521 (SEQ ID NO: 16) 1.2 ug/ml 505E A12 A3 antibody labeled with HaloTag-SmTriplO Pep289 (SEQ ID NO: 17), 20 μM LgTrip 3546 (SEQ ID NO: 12), and 100 μM furimazine in ethanol.

One mL aliquots of 20× stock solution was dispensed into 10 mL amber glass vials, and a runner stopper was partially inserted into the vial. Vials were loaded into the lyophilizer (Virtis Genesis 12EL lyophilizer) with shelves pre-chilled to 4.7° C. Product then underwent a freezing step with a shelf temperature of −50° C. for 2 hr after which time the condenser step started. During the run, the condenser temperature ran between −5° C. and −87° C. A vacuum pulled down ran next at the pressure set-points of 75 and 200 mTorr. Sublimation lasted ˜7.5 hr, and desorption lasted ˜16.1 hr. At the end of the lyophilization process, the vials were back-filled with nitrogen and sealed with fully inserted stoppers at −600 Torr of pressure.

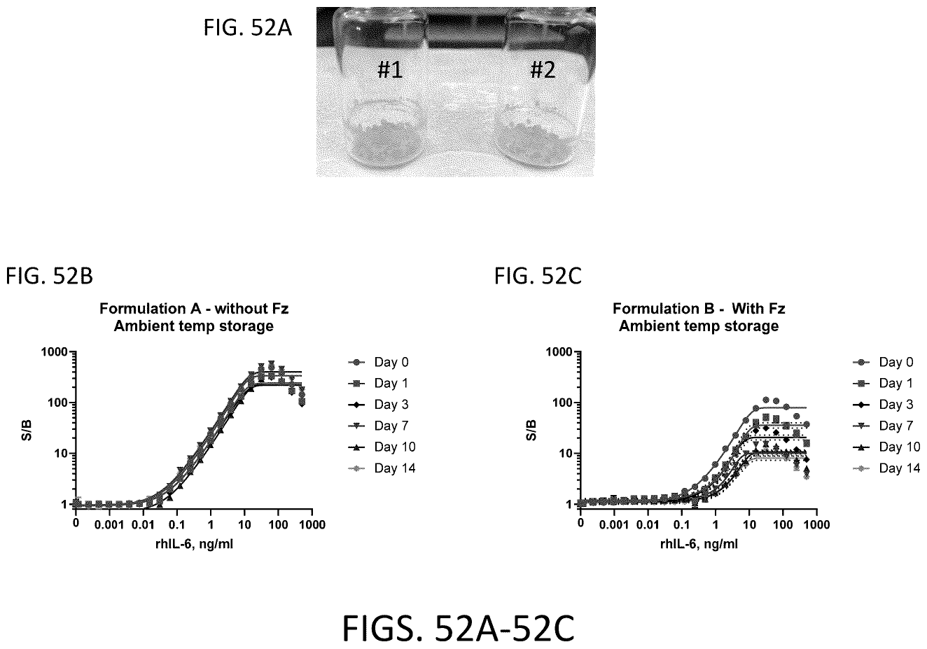

FIG. 52A displays the resulting lyophilized product for single-reagent, IL-6 NanoTrip (tripartite NanoLuc) immunoassays using formulations A and B..

Vials were stored at 25° C. and tested at various timepoints post-lyophilization. For activity-based assays, single-reagent cakes were reconstituted with 10 mL of PBS containing 0.01% BSA. The vials were shaken manually and allowed to equilibrate at room temperature for 5 minutes. 50 μl of the reconstituted substrate was added to 50 μl of recombinant human IL-6 (source) reconstituted in the same BSA buffer. Formulation A requires the addition of furimazine, in which NANOGLO Live Cell Substrate (Promega N205) was used. Assays were performed in solid, white, nonbinding surface (NBS) plates (Costar) and analyzed on a GLOMAX Discover Multimode Microplate Reader (Promega) collecting total luminescence using kinetic or endpoint reads, depending on the experiment. FIG. 52B displays the signal/background assay performance of formulation A over a two-week time course at ambient temps showing that this formulation is shelf-stable and displays an excellent dose response curve over the time tested. However, when furimazine is added (i.e. Formulation B), reduced shelf-stability is observed (FIG. 52C).

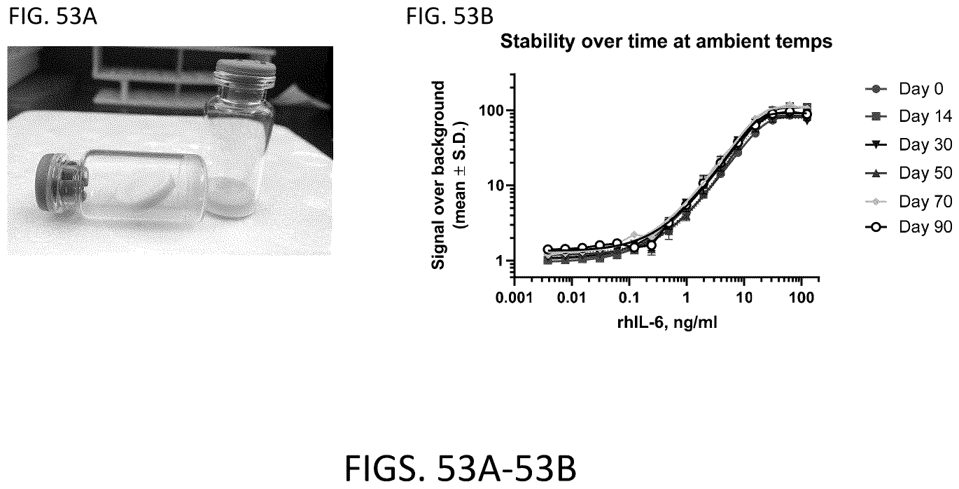

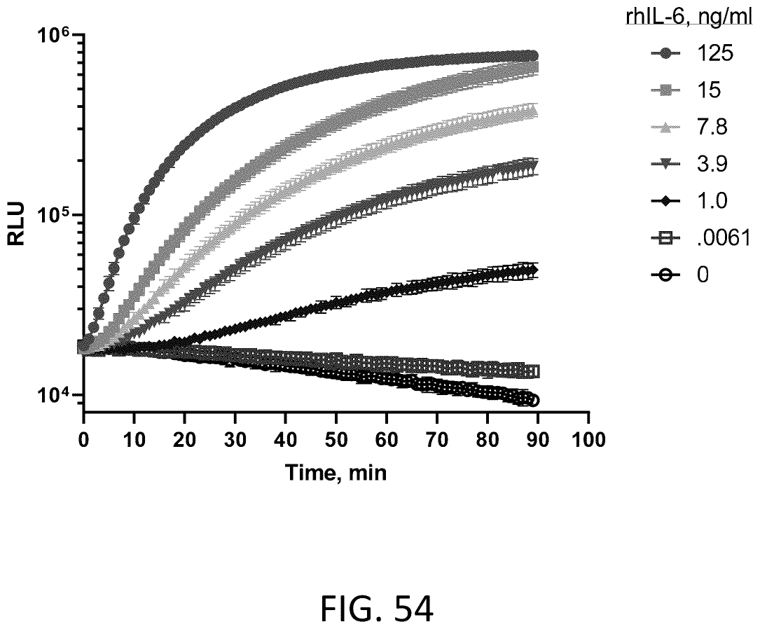

FIG. 53A displays the resulting lyophilized product for a single-reagent, IL-6 NanoTrip (tripartite NanoLuc) immunoassay using formulation C. This formula results in a very desirable cake that is intact and mobile from the glass sides without any fragmenting. FIG. 53B displays the signal/background assay performance of formulation C over a 3 month time course of storage at ambient temperatures showing that this formulation is shelf-stable and displays an excellent dose response curve that is unchanged over the time tested. FIG. 54 shows the kinetic profile of an IL-6 dose response of lyophilized formulation C post reconstitution in PBS containing 0.01% BSA.

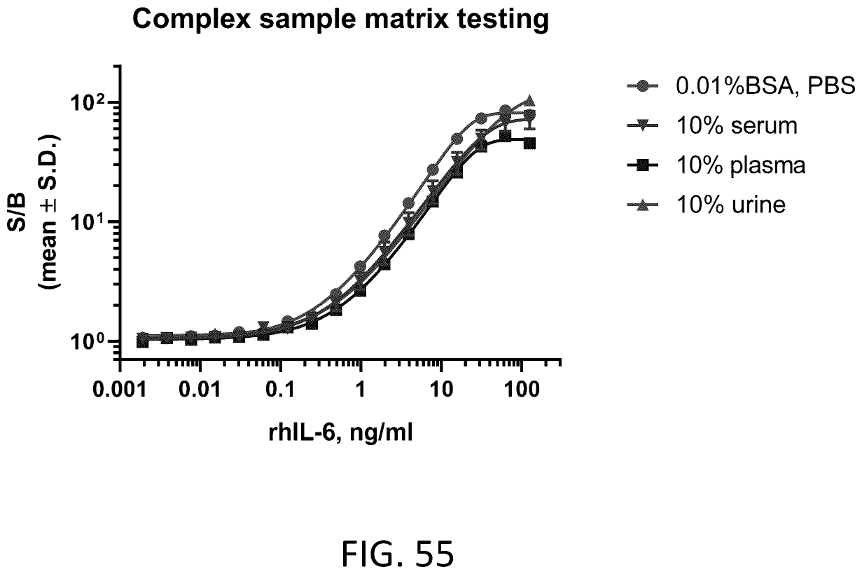

To determine the lyophilized assay compatibility with complex human matrices, lyophilized cakes produced with formulation C were reconstituted in PBS (pH 7.0) containing 0.01% BSA. 50 μl was added to wells of 96-well microtiter plates containing 50 ul of rhIL-6 in 20% normal pooled human serum, citrate collected plasma, or urine. In all experiments, plates were incubated at room temperature for 90 minutes. Final concentration of the assay reagents in all experiments were 60 ng/ml SmTrip10-labeled antibody, 30 ng/ml SmTrip9-labeled antibody, 1 μM LgTrip 3546, and 5 μM furimazine. Luminescence was analyzed. FIG. 55 displays the signal/background results from these experiments indicating complex sample matrix compatibility with the single-reagent IL-6 NanoTrip immunoassay produced with formulation C.

### Example 21

### Lyophilized, Single-Reagent Tripartite Immunoassays in Pre-Filled, 96-Well Microtiter Plates

To evaluate the potential application of lyophilization for preservation of the entire IL-6 NanoTrip (tripartite NanoLuc) immunoassay directly into a 96-well microtiter plates, formulations containing 5 mM azothiothymine, 5 mM ascorbic acid, 2.5% pullulan w/v, 20 mM HEPES buffer (pH 8.0), 90 mM glycine, 20 mM histidine, 25 mg/ml sucrose, 0.01% polysorbate 80, 0.12 ug/ml clone 5IL6 antibody labeled with HaloTag-SmTrip9 Pep521 (SEQ ID NO: 16), 0.24 ug/ml 505E A12 A3 antibody labeled with HaloTag-SmTrip10 Pep289 (SEQ ID NO: 17), 4 pM LgTrip 3546 (SEQ ID NO: 12), and 100 μM furimazine in ethanol (same as formulation C in the previous example, but with a 4× reagent addition instead of a 20× stock reagent as used in the vials) were used.

Approximately 25 μl aliquots of 4× stock solution was dispensed into 96-well microtiter plates. Two types of plates were used: non-binding surface (Costar 3600) and non-treated surface (Costar 3912). Plates were loaded into the lyophilizer (Virtis Genesis 12EL lyophilizer) with shelves pre-chilled to 4.7° C. Product then underwent a freezing step with a shelf temperature of −50° C. for 2 hr after when time the condenser step started. During the run, the condenser temperature ran between −5° C. and −87° C. A vacuum pull down ran next at the pressure set-points of 75 and 200 mTorr. Sublimation lasted ˜7.5 hr, and desorption lasted ˜16.1 hr. At the end of the lyophilization process, the plates were back-filled with nitrogen and sealed with adhesive plate cover.

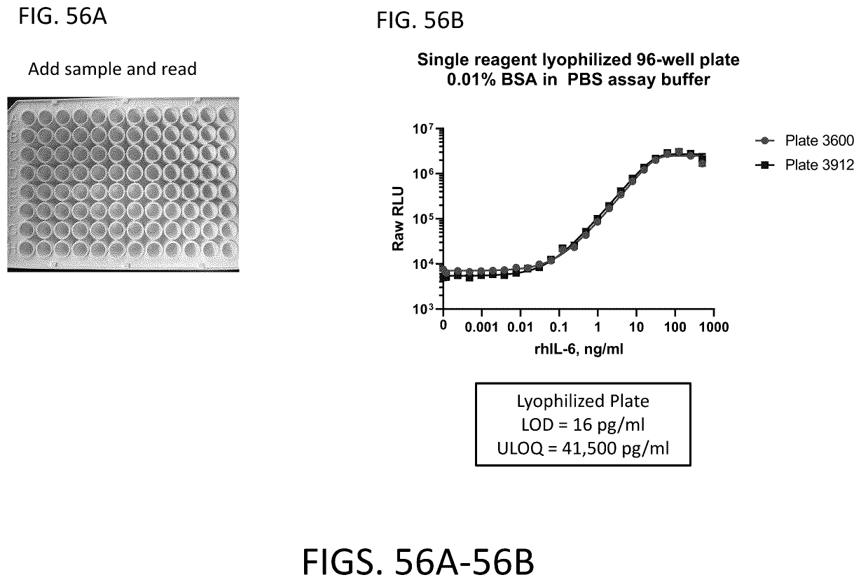

FIG. 56A depicts one of the plates with the lyophilized material in the bottom of the wells. The lyophilized cakes stayed in an intact cake, but were mobile when using the nonbinding surface plates. The lyophilized material stayed “stuck” on the bottom of the wells in the non-treated plates. FIG. 56B shows the resulting bioluminescence when 1× rhIL-6 was added directly to the wells and analyzed for luminescence using a GLOMAX luminometer. The resulting dose response curve showed excellent reconstitution and performance in both plates.

### Example 22

### Testing the Effects of Individual Excipients in Formulations Using the Solution-Based, Homogeneous IL-6 Tripartite Immunoassay

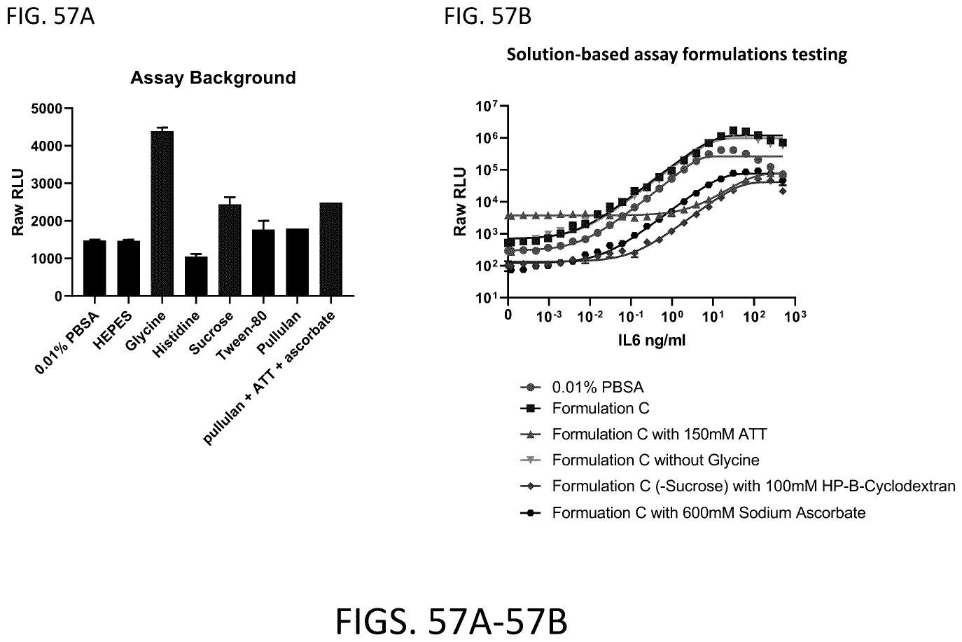

To determine the effects of assay performance of individual excipients used in the lyophilized formulations for the single-reagent NanoTrip (tripartite NanoLuc) immunoassays, the IL-6 model system in the solution-based assay was used with the effects of various excipients analyzed. FIG. 57A displays the assay background signals for the solution-based homogenous IL-6 immunoassay performed in a standard assay buffer consisting of 0.01% BSA in PBS, pH 7.0, and with the addition of various individual excipients as indicated on the X-axis. FIG. 57B displays the IL-6 dose response curve when the assay was performed in different buffers consisting of formulation C from Example 20 and modified versions of formulation C. For these experiments, 30 ng/ml 5IL6 antibody labeled with HaloTag-SmTrip9 Pep521 (SEQ ID NO: 16), 60 ng/ml 505E A12 A3 antibody labeled with HaloTag-SmTriplO Pep289 (SEQ ID NO: 17), and 1 μM LgTrip 3546 (SEQ ID NO: 12) were incubated in the presence of rhIL-6 for 90 minutes. Furimazine (Promega Live Cell Substrate N205) was added according to manufacturer's instruction, but using the formulation indicated as buffer. Luminescent signal was analyzed using a GLOMAX luminometer. These experiments demonstrated that iterative experimentation is required to determine appropriate buffer components for NanoTrip immunoassays.

### Example 23

### Creating a Solution-Based and Lyophilized, Single-Reagent Tripartite Immunoassays in Vials for the Target Analyte Human Cardiac Troponin I

The basic principle of the homogeneous NanoTrip (NanoLuc tripartite) cardiac troponin I immunoassay is depicted in FIG. 58. First, a pair of antibodies that target non-overlapping epitopes on human cardiac troponin I were chemically conjugated to SmTrip9 (or variants thereof) or HiBiT (or variants thereof) using the HaloTag® technology. When the labeled antibodies bind a cardiac troponin I analyte, the complementary subunits are brought into proximity thereby reconstituting a bright luciferase in the presence of the LgTrip 3546 protein and furimazine substrate. This assay is quantitative because the amount of luminescence generated by a standard plate-reading luminometer is directly proportional to the amount of target analyte present.

Genetic fusions containing SmTrip9 Pep521 (SEQ ID NO: 16) or SmTriplO Pep289 (SEQ ID NO: 17) separated by either a 2× or 3× Gly-Ser-Ser-Gly linker to the amino terminus of HaloTag was achieved using the pFN29A HIS6HaloTag T7 Flexi Vector (Promega). Glycerol stocks of E. coli expressing HisTag-HaloTag fusion protein were used to inoculate 50 mL starter cultures, which were grown overnight at 37° C. in LB media containing 25 μg/ml kanamycin. Starter cultures were diluted 1:100 into 500 mL fresh LB media, containing 25 μg/mL kanamycin, 0.12% glucose, and 0.2% rhamnose. Cultures were grown for 22-24 h at 25° C. Cells were pelleted by centrifugation (10,000 rpm) for 30 min at 4° C. and re-suspended in 50 mL PBS. 1 mL protease inhibitor cocktail (Promega), 0.5 mL RQ1 DNase (Promega), and 0.5 mL of 10 mg/mL lysozyme (Sigma) were added, and the cell suspension was incubated on ice with mild agitation for 1 h. Cells were lysed by sonication at 15% power at 5 s intervals for 1.5 min (3 min total) and subsequently centrifuged at 10,000 rpm for 30 min at 4° C. Supernatant was collected, and protein purified using HisTag columns (GE) following the manufacturer's recommended protocol. Protein was eluted using 500 mM imidazole, dialyzed in PBS, characterized using SDS-PAGE gel and was >95% pure. Proteins were stored in 50% glycerol at −20° C.

To chemically conjugate the antibodies to the HaloTag-peptide fusion proteins, antibodies were buffered exchanged 2× into 10 mM sodium bicarbonate buffer (pH 8.5) using Zeba spin desalting columns (ThermoFisher). Antibodies were then primed with 200μM amine reactive HaloTag Succinimidyl Ester (04) Ligand (Promega) for 2 hr shaking at 1000 rpm at 22° C. Unreacted ligand was removed with two passes through Zeba spin columns in PBS buffer. Then, antibodies were covalently labeled with 30 μM of the HaloTag fusion protein overnight at 4° C. while shaking. Excess unreacted HaloTag fusion protein was removed using HaloLink Resin (Promega). Non-denaturing SDS-PAGE gel was used to characterize the conjugated antibodies. Anti-human cardiac troponin I monoclonal antibodies used in the human cardiac troponin I immunoassay were recombinant rabbit clone 1H11 μL19 (Invitrogen) and monoclonal mouse antibody clone 16A11 (Invitrogen).

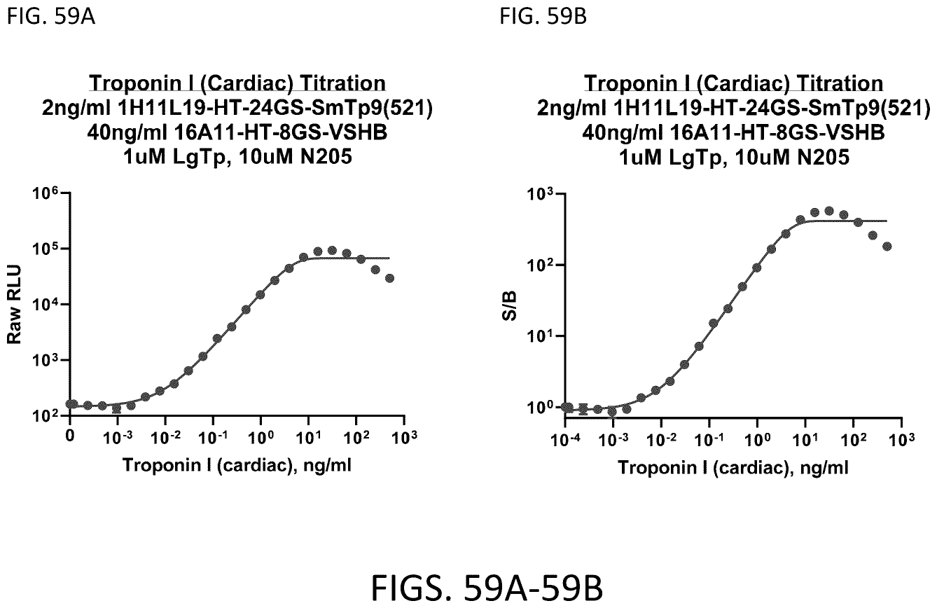

FIG. 59A (raw RLUs) and 59B (signal/background) display the dose response curve for the solution-based homogenous cardiac troponin I immunoassay performed in a standard assay buffer consisting of 0.01% BSA in PBS, pH 7.0. Purified recombinant human cardiac troponin I (Fitzgerald) was used to generate the dose response curve. For these experiments, 2 ng/ml of clone 1H11 μL19 labeled with HaloTag-24gly/ser-SmTrip9 Pep521 (SEQ ID NO: 16), 40 ng/ml of clone 16A11 labeled with HaloTag-8gly/ser-SmallTrip10 Pep289 (SEQ ID NO: 17), and 1 μM LgTrip 3546 (SEQ ID NO: 12) protein were incubated in the presence of recombinant human cardiac troponin I for 90 minutes. Furimazine (Promega Live Cell Substrate N205) was added according to the manufacturer's instructions, but using 0.01% BSA in PBS as the buffer. Luminescent signal was analyzed on a GLOMAX luminometer.

To evaluate the potential application of lyophilization for preservation of the entire cardiac troponin I tripartite immunoassay in a single vial, formulations containing the peptide labeled antibodies (SmTrip9 Pep521 (SEQ ID NO: 16) and SmTriplO Pep289 (SEQ ID NO: 17)), LgTrip 3546 (SEQ ID NO: 12), and furimazine were prepared. The 20× stock formulations are as follows:

Approximately, 5 mM azothiothymine, 5 mM ascorbic acid, 2.5% pullulan w/v, 20 mM HEPES buffer (pH 8.0), 90 mM glycine, 20 mM histidine, 25 mg/ml sucrose, 0.01% polysorbate 80, 0.08 ug/ml clone 1H11 μL19 antibody labeled with HaloTag-SmTrip9 Pep521 (SEQ ID NO: 16), 1.6 ug/ml of clone 16A11 antibody labeled with HaloTag-SmTriplO Pep289 (SEQ ID NO: 17), 20 μM LgTrip 3546 (SEQ ID NO: 12), and 200 μM furimazine (Promega NANOGLO substrate N113).

One mL aliquots of 20× stock solution were dispensed into 10 mL amber glass vials, and a runner stopper was partially inserted into the vial. Vials were loaded into the lyophilizer (Virtis Genesis 12EL lyophilizer) with shelves pre-chilled to 4.7° C. Product then underwent a freezing step with a shelf temperature of −50° C. for 2 hr after which time the condenser step started. During the run, the condenser temperature ran between −5° C. and −87° C. A vacuum pull down ran next at the pressure set-points of 75 and 200 mTorr. Sublimation lasted ˜7.5 hr, and desorption lasted ˜16.1 hr. At the end of the lyophilization process, the vials were back-filled with nitrogen and sealed with fully inserted stoppers at −600 Torr of pressure.

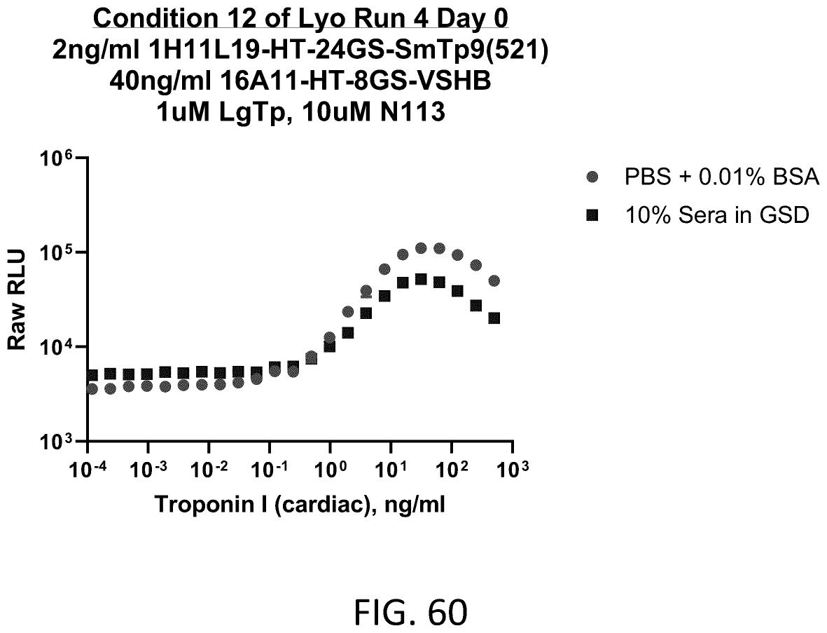

For activity-based assays, single-reagent cakes were reconstituted with 10 mL of PBS containing 0.01% BSA. The vials were shaken manually and allowed to equilibrate at room temperature for 5 minutes. 50 1 μl of the reconstituted single-reagent cardiac troponin I NanoTrip (tripartite NanoLuc) immunoassay was added to 50 μl of recombinant human cardiac troponin I (Fitzgerald) that was reconstituted in the same BSA buffer or with 20% human serum diluted in General Serum Diluent (Immunochemistry Technologies). Assays were performed in solid, white, nonbinding surface (NBS) plates (Costar) and analyzed on a GLOMAX Discover Multimode Microplate Reader (Promega) collecting total luminescence using an endpoint read. FIG. 60 shows the cardiac troponin I dose response curve of the resulting bioluminescence upon reconstitution of the single-reagent troponin NanoTrip immunoassay with the sample in 0.01% BSA in PBS buffer or in the presence of the complex matrix sample of human serum diluted in General Serum Diluent. Troponin was effectively detected even in the presence of serum using this immunoassay.

### Example 24

### Investigating and Mitigating the Effects of Complex Sample Matrices on Tripartite Immunoassay Performance

A solution-based, homogeneous IL-6 NanoTrip (tripartite NanoLuc) immunoassay was tested to determine if the assay was compatible with human sample types commonly analyzed for clinical biomarkers, and factors in the samples that might affect the performance of the assay and possible solutions to mitigate these effects were investigated. This is critical because sample matrix interference effects in immunoassays, defined as the effect of a substance present in the sample that alters the correct value of the result, are a common phenomenon especially in homogenous formats due to the removal of the wash steps.

Reagents used for the following experiments were the HaloTag-peptide labeled antibodies described in Example 19. 30 ng/ml clone 5IL6 antibody labeled with HaloTag-SmTrip9 Pep521 (SEQ ID NO: 16), 60 ng/ml 505E A12 A3 antibody labeled with HaloTag-SmTriplO Pep289 (SEQ ID NO: 17), 1 μM LgTrip 3546 (SEQ ID NO: 12), and NANOGLO Live Cell Substrate (Promega N205) or NANOGLO substrate (Promega N113), which were used according to the manufacturer's instructions, but were diluted in the given buffer for that experiment. Assays were performed +/−50 ng/ml recombinant human IL-6 (R&D Systems) with assay backgrounds, and Bmax analyzed. Assays were allowed to incubate on the bench for 90 minutes prior to addition of substrate. Assays were performed in solid, white, nonbinding surface (NBS) plates (Costar) and analyzed on a GLOMAX Discover Multimode Microplate Reader (Promega) collecting total luminescence using an endpoint read.

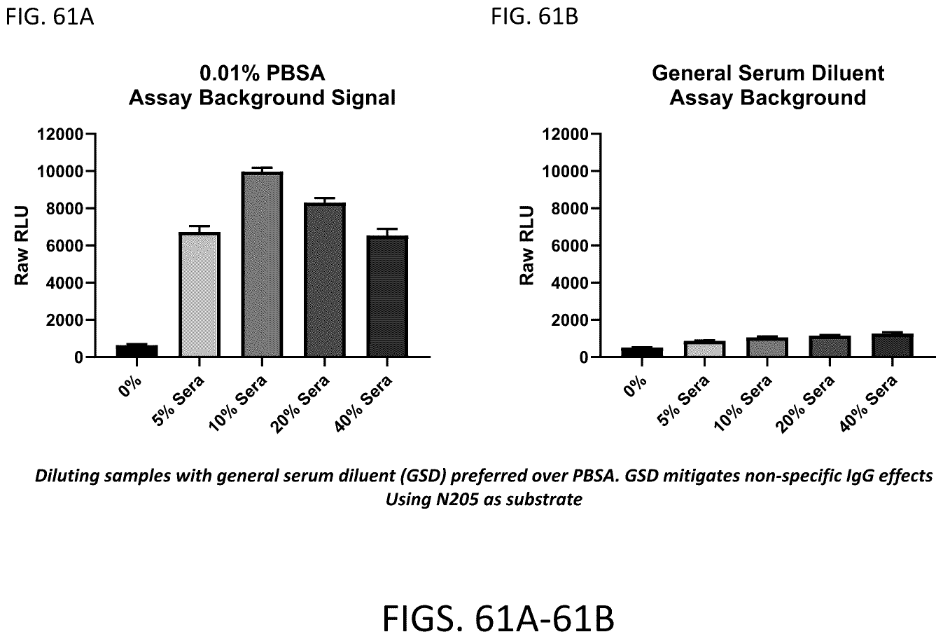

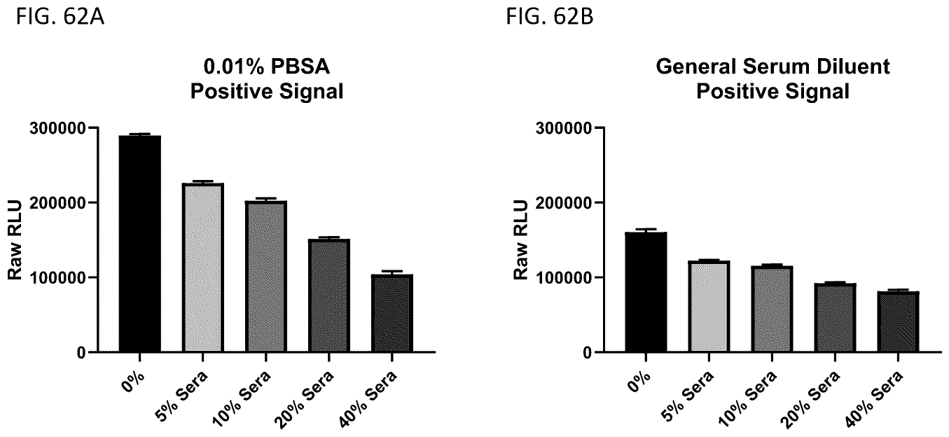

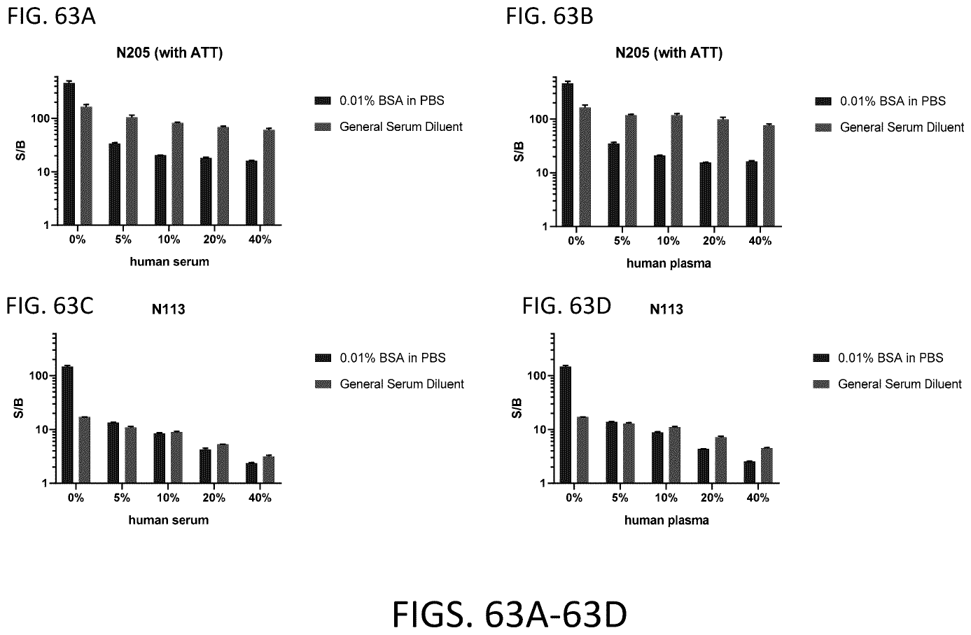

FIG. 61 shows the solution-based, homogeneous IL-6 NanoTrip (tripartite NanoLuc) assay background in the presence of increasing normal, pooled human serum when the assay was performed in (A) 0.01% BSA in PBS (pH 7.0) assay buffer or (B) in General Serum Diluent (Immunochemistry Technologies) and using NANOGLO Live Cell Substrate (Promega N205). General Serum Diluent mitigated non-specific IgG effects and had a positive effect by decreasing the assay background. FIG. 62 shows the bioluminescent response when in the presence of 50 ng/ml rhIL-6 and increasing human serum when the assay was performed in (A) 0.01% BSA in PBS (pH 7.0) assay buffer or (B) General Serum Diluent and using NANOGLO Live Cell Substrate (Promega N205). General Serum Diluent displayed a slightly lower Bmax overall, but less of a loss in signal with increasing human serum. FIG. 63A-D shows the fold response of results when the rhIL-6 screening assays were performed with 0.01% BSA in PBS (pH 7.0) or General Serum Diluent and using NANOGLO Live Cell Substrate (Promega N205) or NANOGLO substrate (Promega N113) and testing in increasing amounts of normal, pooled human serum or plasma. Overall, using General Serum Diluent paired with the NANOGLO Live Cell Substrate (Promega N205) provided the best assay results in these complex sample matrices.

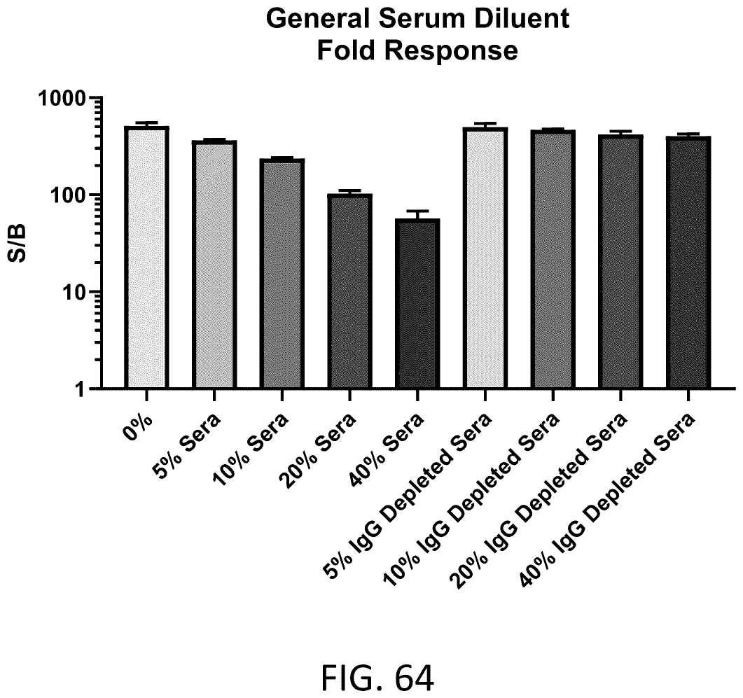

Next, the effects of endogenous IgG in human serum samples had on assay performance was determined. Using the solution-based, homogeneous IL-6 NanoTrip assay +/−50 ng/ml rhIL-6 in General Serum Diluent, the bioluminescent response when running the assay in normal, pooled human serum or in serum that had been depleted of endogenous IgG was analyzed. FIG. 64 shows the fold response of this experiment, which indicates that endogenous IgG is one of the components in serum that negatively effects the performance of the immunoassay.

Next, the effects of blood biochemistry on the solution-based, homogenous IL-6 tripartite immunoassay was investigated using the VeriChem reference plus chemistry kit, which contains the following:

| Analyte | Units | Level A | Level B | Level C | Level D | Level E |
|---|---|---|---|---|---|---|
| Glucose | mg/dL | 5 | 40 | 75 | 110 | 145 |
| Urea | mg/dL | 1.0 | 7.5 | 14.0 | 20.5 | 27.0 |
| Nitrogen | | | | | | |
| Creatinine | mg/dL | 0.04 | 1.24 | 2.44 | 3.64 | 4.84 |
| Calcium | mg/dL | 1.0 | 1.5 | 2.0 | 2.5 | 3.0 |
| Phosphorus | mg/dL | 0.2 | 0.7 | 1.2 | 1.7 | 2.2 |
| Magnesium | mg/dL | 0.16 | 0.46 | 0.76 | 1.06 | 1.36 |
| Magnesium | mEq/L | 0.132 | 0.38 | 0.63 | 0.87 | 1.12 |
| Triglyceride | mg/dL | 2 | 49 | 240 | 143 | 190 |

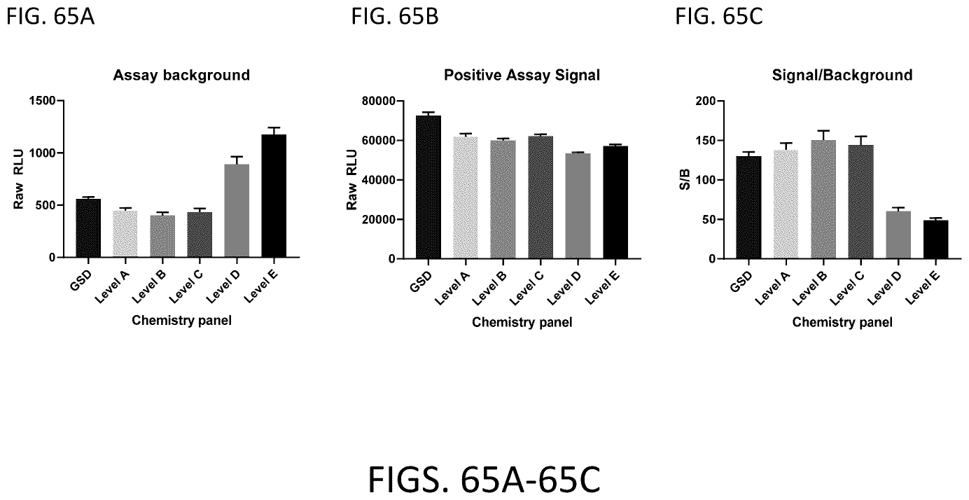

The IL-6 NanoTrip assay was run in the presence of Level A-E diluted in general serum diluent and using NANOGLO Live Cell Substrate (Promega N205) to determine the effects of increasing these blood chemistry components on assay performance. FIG. 65A shows the assay background in raw RLUs, FIG. 65B shows the Bmax signal when in the presence of 50 ng/ml rhIL-6, and FIG. 65C shows the signal over background results. The results indicate that increasing these chemistry components had an effect on increasing assay background as well as decreasing the Bmax impacting the overall signal to background of the assay performance.

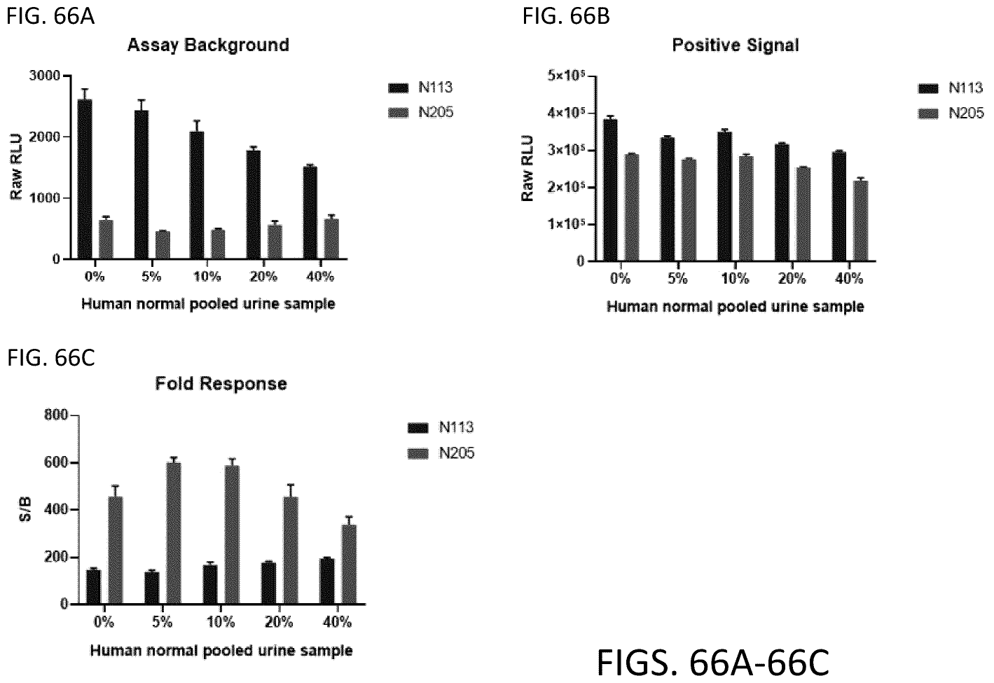

To determine the effects of urine on the solution-based, homogeneous IL-6 NanoTrip immunoassay performance, a IL-6 screening assay in the presence of increasing normal, pooled human urine diluted in General Serum Diluent and NANOGLO substrate (Promega Ni13) or NANOGLO Live Cell Substrate (Promega N205) was performed. FIG. 66A shows the assay background in raw RLUs, FIG. 66B shows the Bmax signal when in the presence of 50 ng/ml rhIL-6, and FIG. 66C shows the signal over background results. The results indicate that the IL-6 NanoTrip immunoassay was compatible with human urine when using the General Serum Diluent paired with the NANOGLO Live Cell Substrate (Promega N205).

### Example 25

### Creating a Stable, Lyophilized Substrate and LgTrip Cake Reagent in a Single Vial

To evaluate the potential application of lyophilization for preservation of furimazine, LgTrip and furimazine were paired with LgTrip 3546 used as a general detection reagent for tripartite applications and supplied in a single vial. Formulations containing furimazine, LgTrip 3546 (SEQ ID NO: 12), and furimazine with LgTrip 3546 were prepared. The 20× stock formulations are as follows:

Furimazine only formulation: 5 mM azothiothymine, 5 mM ascorbic acid, 2.75% pullulan w/v, 200 μM furimazine in ethanol, and ddH20 millipore

LgTrip 3546 only formulation: 5 mM azothiothymine, 5 mM ascorbic acid, 2.75% pullulan w/v, 20 μM LgTrip 3546 (SEQ ID NO: 12), and ddH₂0 (Millipore)

Furimazine with LgTrip 3546formulation: 5 mM azothiothymine, 5 mM ascorbic acid, 2.75% pullulan w/v, 200 μM furimazine in ethanol, 20 μM LgTrip 3546 (SEQ ID NO: 12) and ddH₂0 (Millipore).

One mL aliquots of 20× stock solution was dispensed into 10 mL amber glass vials, and a runner stopper was partially inserted into the vial. Vials were loaded into the lyophilizer (Virtis Genesis 12EL lyophilizer) with shelves pre-chilled to 4.7° C. Product then underwent a freezing step with a shelf temperature of −50° C. for 2 hr after when time the condenser step started. During the run, the condenser temperature ran between −5° C. and −87° C. A vacuum pull down ran next at the pressure set-points of 75 and 200 mTorr. Sublimation lasted ˜7.5 hr and desorption lasted ˜16.1 hr. At the end of the lyophilization process, the vials were back-filled with nitrogen and sealed with fully inserted stoppers at −600 Torr of pressure.

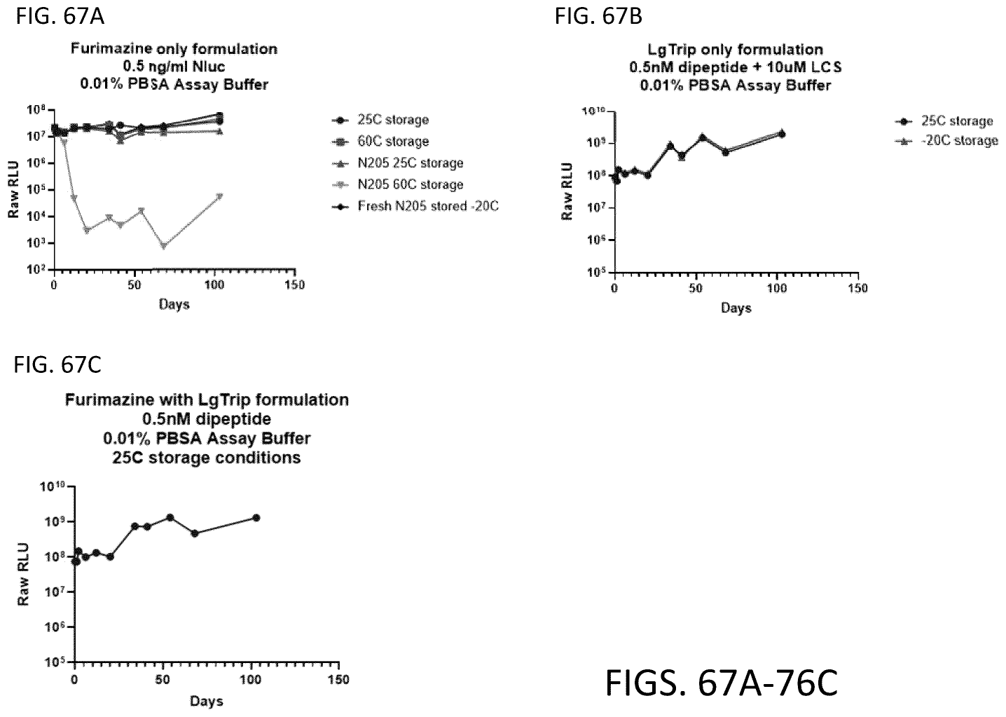

Vials were stored at 25° C. or 60° C. and tested at various time points post-lyophilization. For activity-based assays, lyophilized cakes were reconstituted with 10 mL of PBS containing 0.01% BSA. The vials were shaken manually and allowed to equilibrate at room temperature for 5 minutes. 50 μl of the reconstituted substrate was added to 50 μl of purified NANOLUC enzyme (Promega) or dipeptide (SEQ ID NO: 14) that was reconstituted in the same BSA buffer. LgTrip 3546 only formulations required the addition of furimazine in which NANOGLO Live Cell Substrate (Promega N205) was used. Assays were performed in solid, white, nonbinding surface (NBS) plates (Costar) and analyzed on a GLOMAX Discover Multimode Microplate Reader (Promega) collecting total luminescence using an endpoint read. FIG. 67 displays the Bmax signal produced for (A) furimazine only formulation when in the presence of NanoLuc, (B) LgTrip 3546 only formulation when in the presence of the dipeptide, and (C) furimazine with LgTrip 3546 formulation when in the presence of dipeptide. All formulations displayed thermal stability at all temperatures tested for the 100 day duration of the storage conditions, as opposed to the N205 substrate which is predissolved in organic solvent.

### Example 26

### Creating a Solution-Based and Lyophilized, Single-Reagent Tripartite Immunoassays in Vials for the Target Analytes Anti-TNFα Biologics

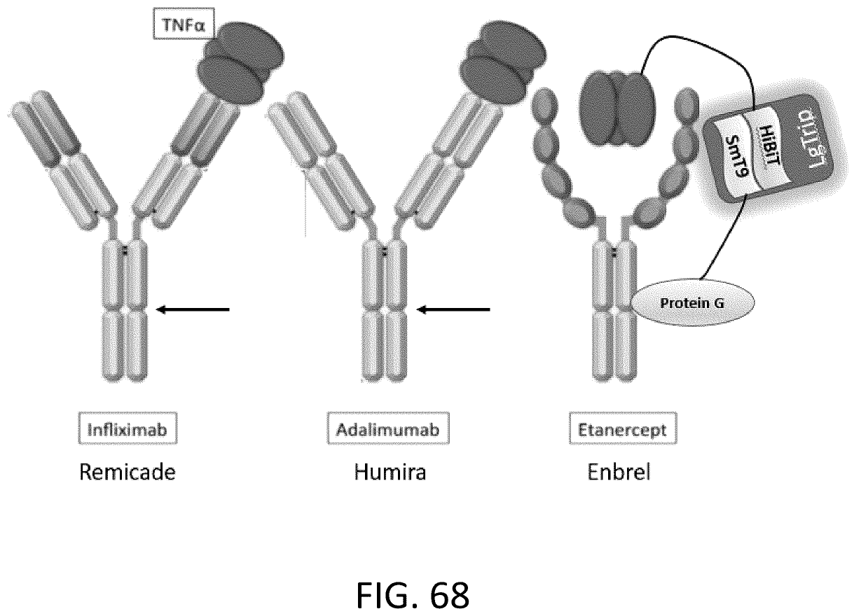

The basic principle of the homogeneous anti-TNFα biologics NanoTrip (tripartite NanoLuc) immunoassay is depicted in FIG. 68. In this model, protein G-SmTrip9 (or variants thereof) fusion proteins and TNFα-HiBiT (or variants thereof) fusion proteins were used. Protein G will bind the Fc region of the anti-TNFα biologic antibody analyte, and the analyte itself will bind the TNFα thus bringing the complementary subunits into proximity, thereby reconstituting a bright luciferase in the presence of the LgTrip 3546 protein and furimazine substrate. This assay is quantitative because the amount of luminescence generated by a standard plate-reading luminometer is directly proportional to the amount of target analyte present.

6xHis-TNFα-15GS-HiBiT (ATG-3998). Genetic fusions containing the SmTrip10 (SEQ ID NO: 15) separated by a 15GS linker (SSSGGGGSGGGSSGG) to the carboxyl-terminus of TNFα was achieved using the pF4Ag CMV Flexi Vector (Promega). Purified plasmid DNA of the TNFα-strand 10 fusion was transformed into Shuffle T7 E. coli K12 (New England Biolabs) and plated at a 1:100 dilution on an LB plate containing 100 g/ml ampicillin and incubated overnight at 37° C. A colony from this plate was used to inoculate 50 mL starter cultures, which were grown overnight at 37° C. in LB media containing 100 g/ml ampicillin. Starter cultures were diluted 1:100 into 500 mL fresh LB media containing 100 g/ml ampicillin and were incubated at 37° C. until it reached an OD of 0.6, at which time a final concentration of 1 mM IPTG was added to the sample. After IPTG inoculation, cultures were grown overnight at 25° C. Cells were pelleted by centrifugation (10,000 rpm) for 30 min at 4° C. and re-suspended in 50 mL TBS, 1 mL protease inhibitor cocktail (Promega), 0.5 mL RQ1 DNase (Promega), and 1 mL of 10 mg/mL lysozyme (Sigma), and the cell suspension was incubated on ice with mild agitation for 1 h. Cells were lysed by three freeze-thaw cycles from −80° C. freezer to a 37° C. water bath and subsequently centrifuged at 10,000 rpm for 30 min at 4° C. Supernatant was collected and protein was purified using Ni Sepharose 6 Fast Flow resin (GE), following manufacturer's recommended protocol. Protein was eluted using a step-wise imidazole elution starting at 100 mM imidazole and reaching up to 500 mM imidazole, dialyzed in TBS, characterized using SDS-PAGE gel and was >95% pure. Proteins were stored in 50% glycerol at −20° C.

SmTrip9(521)-15GS-PtnG-6xHis (ATG4002). Genetic fusions containing the SmTrip9 (SEQ ID NO: 13) separated by a linker (GSSGGGGSGGGGSSG) to the amino terminus of Protein G was achieved using the pF1A T7 Flexi Vector (Promega). Glycerol stocks of E. coli expressing SmTrip9(521)-PtnG fusion protein was used to inoculate 50 mL starter cultures, which were grown overnight at 37° C. in LB media containing 100 g/ml ampicillin. Starter cultures were diluted 1:100 into 500 mL fresh LB media, containing 100 g/mL ampicillin, 0.15% glucose, and 0.1% rhamnose. Cultures were grown for 16-24 h at 25° C. Cells were pelleted by centrifugation (10,000 rpm) for 30 min at 4° C. and re-suspended in 50 mL TBS. 1 mL protease inhibitor cocktail (Promega), 0.5 mL RQ1 DNase (Promega), and 1 mL of 10 mg/mL lysozyme (Sigma) were added, and the cell suspension was incubated on ice with mild agitation for 1 h. Cells were lysed by three freeze-thaw cycles from −80° C. freezer to a 37° C. water bath and subsequently centrifuged at 10,000 rpm for 30 min at 4° C. Supernatant was collected and protein purified using HisTag columns (GE), following manufacturer's recommended protocol. Protein was eluted using gradient elution with a 500 mM imidazole final concentration, dialyzed in TBS, characterized using SDS-PAGE gel and was >95% pure. Proteins were stored in 50% glycerol at −20° C.

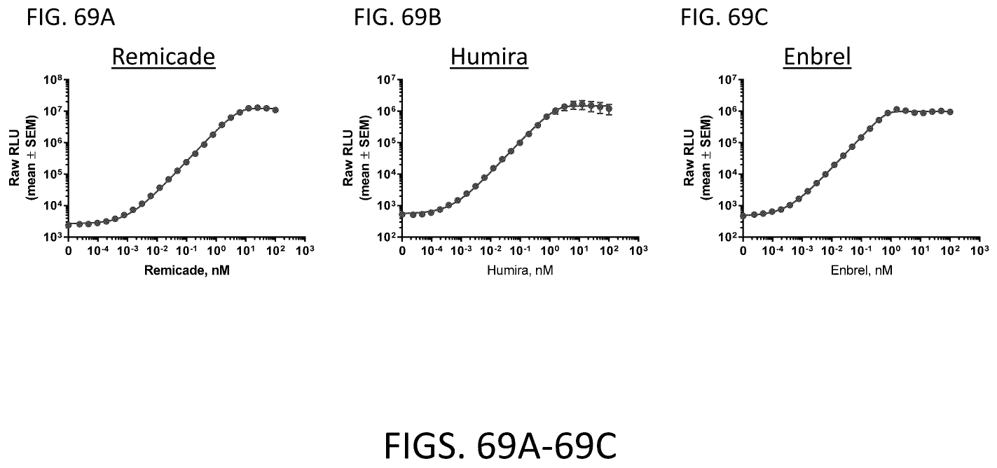

FIG. 69 displays the dose response curves for the solution-based homogenous anti-TNFα biologics immunoassay performed in a standard assay buffer consisting of 0.01% BSA in PBS, pH 7.0. For these experiments, 10 nM of protein G-15gly/ser-SmTrip9 Pep521 (SEQ ID NO: 16), 10 nM TNFα-15 gly/ser-SmTriplO Pep289 (SEQ ID NO: 17), and 1 μM LgTrip 3546 (SEQ ID NO: 12) protein were incubated in the presence of (A) Remicade, (B) Humira, and (C) Enbrel for 90 minutes. Furimazine (NANOGLO Live Cell Substrate; Promega N205) was added, and total luminescence signal was analyzed using a GLOMAX Discover.

To evaluate the potential application of lyophilization for preservation of the entire anti-TNFαTNFα biologics, NanoTrip and NanoBiT immunoassays in single vial formulations containing peptide-labeled fusion proteins and LgTrip 3546 (SEQ ID NO: 12; for NanoTrip assays) and furimazine were prepared. The 20× stock formulations are as follows:

NanoTrip anti-TNFα biologics immunoassay: 5 mM azothiothymine, 5 mM ascorbic acid, 2.75% w/v pullulan, ddH20 (Millipore), 200 μM furimazine in ethanol, 20 μM LgTrip 3546 protein (SEQ ID NO:12), 200 nM protein G-SmTrip9 Pep521 (SEQ ID NO: 16) fusion protein, and 200 nM TNFα-SmTrip10 Pep289 (SEQ ID NO:17) fusion protein.

NanoBiT anti-TNFα biologics immunoassay: 5 mM azothiothymine, 5 mM ascorbic acid, 2.75% w/v pullulan, ddH20 (Millipore), 200 μM furimazine in ethanol, 200 nM protein G-SmBiT (SEQ ID NO:10) fusion protein, and 200 nM TNFα-LgBiT (SEQ ID NO: 12) fusion protein.

One mL aliquots of 20× stock solution was dispensed into 10 mL amber glass vials, and a runner stopper was partially inserted into the vial. Vials were loaded into the lyophilizer (Virtis Genesis 12EL lyophilizer) with shelves pre-chilled to 4.7° C. Product then underwent a freezing step with a shelf temperature of −50° C. for 2 hr after which time the condenser step started. During the run, the condenser temperature ran between −5° C. and −87° C. A vacuum pull down ran next at the pressure set-points of 75 and 200 mTorr. Sublimation lasted ˜7.5 hr and desorption lasted ˜16.1 hr. At the end of the lyophilization process, the vials were back-filled with nitrogen and sealed with fully inserted stoppers at −600 Torr of pressure.

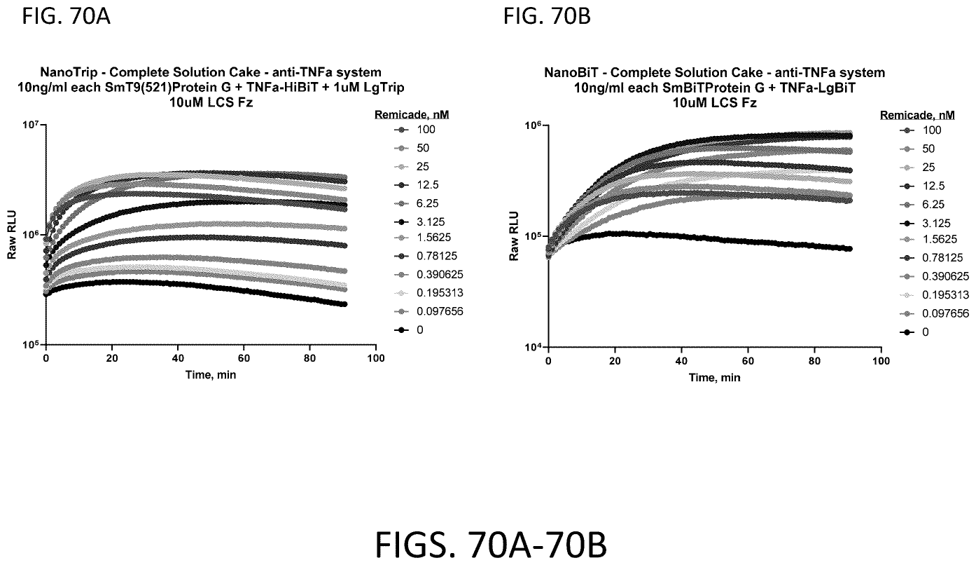

For activity-based assays, single-reagent cakes were reconstituted with 10 mL of PBS containing 0.01% BSA. The vials were shaken manually and allowed to equilibrate at room temperature for 5 minutes. 50 μl of the reconstituted single-reagent anti-TNFα biologics NanoTrip and NanoBiT immunoassays were added to 50 μl of Remicade in a titration that was reconstituted in the same BSA buffer. Assays were performed in solid, white, nonbinding surface (NBS) plates (Costar) and analyzed on a GLOMAX Discover Multimode Microplate Reader (Promega) collecting total luminescence using a kinetic read. FIG. 70 shows the Remicade dose response curves of the resulting bioluminescence upon reconstitution of the single-reagent Remicade (A) NanoTrip immunoassay or (B) NanoBiT immunoassay.

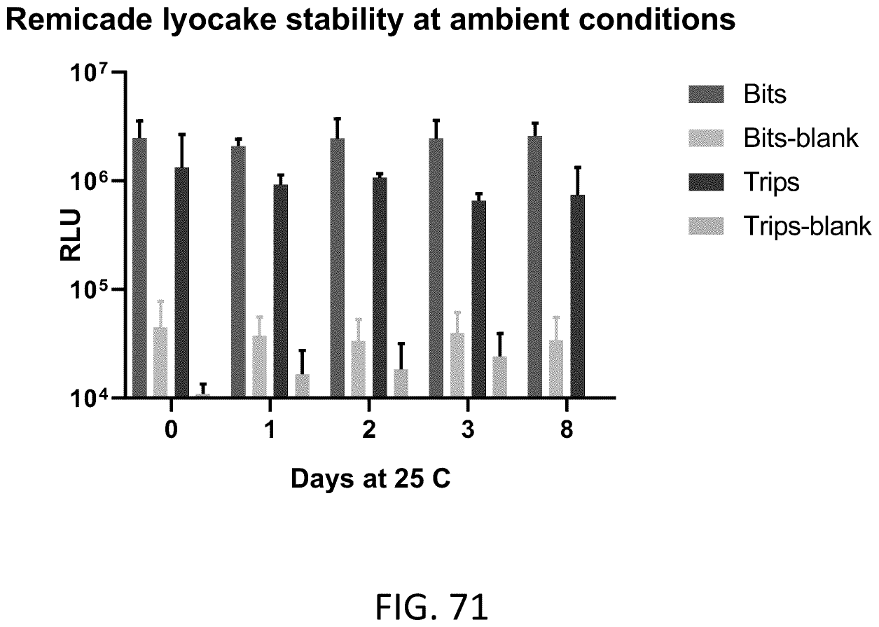

Testing the thermal stability of these lyophilized, single-reagent anti-TNFα biologics NanoTrip and NanoBiT immunoassays when stored at ambient temperatures indicated that both assays, when reconstituted in 0.01% BSA in PBS (pH 7.0) in the presence or absence of 100 nM Remicade, displayed shelf stability and a significant increase in signal when the analyte Remicade is present. Results are shown in FIG. 71.

### Example 27

### Developing Stable, Lyophilized Tripartite and NanoBiT Immunoassay Using a Split-Reagent Approach

To evaluate the potential application of lyophilization for preservation of separate components of the anti-TNFα biologics, NanoTrip and NanoBiT immunoassays that are then combined in a single vial formulations containing the peptide labeled fusion proteins and LgTrip 3546 (SEQ ID NO: 12; for NanoTrip assays) and furimazine were prepared. The 20× stock formulations are as follows:

NanoBiT anti-TNFα biologics immunoassay:

Furimazine with LgBiT-TNFα: 5 mM azothiothymine, 5 mM ascorbic acid, 2.75% w/v pullulan, ddH20 (Millipore), 200 μMfurimazine in ethanol, and 200 nM TNFα-LgBiT (SEQ ID NO: 12) fusion protein.

NanoBiT protein G: 5 mM azothiothymine, 5 mM ascorbic acid, 2.75% w/v pullulan, ddH20 millipore, 200 nM protein G-SmBiT (SEQ ID NO: 10) fusion protein

NanoTrip anti-TNFα biologics immunoassay:

Furimazine with LgTrip 3546: 5 mM azothiothymine, 5 mM ascorbic acid, 2.75% w/v pullulan, ddH₂0 (Millipore), 200 μMfurimazine in ethanol, 20 μM LgTrip 3546 protein (SEQ ID NO: 12),

Protein G with TNFα: 5 mM azothiothymine, 5 mM ascorbic acid, 2.75% w/v pullulan, ddH20 (Millipore), 200 nM protein G-SmTrip9 Pep521 (SEQ ID NO: 16) fusion protein, and 200 nM TNFα-SmTrip10 Pep289 (SEQ ID NO: 17) fusion protein.

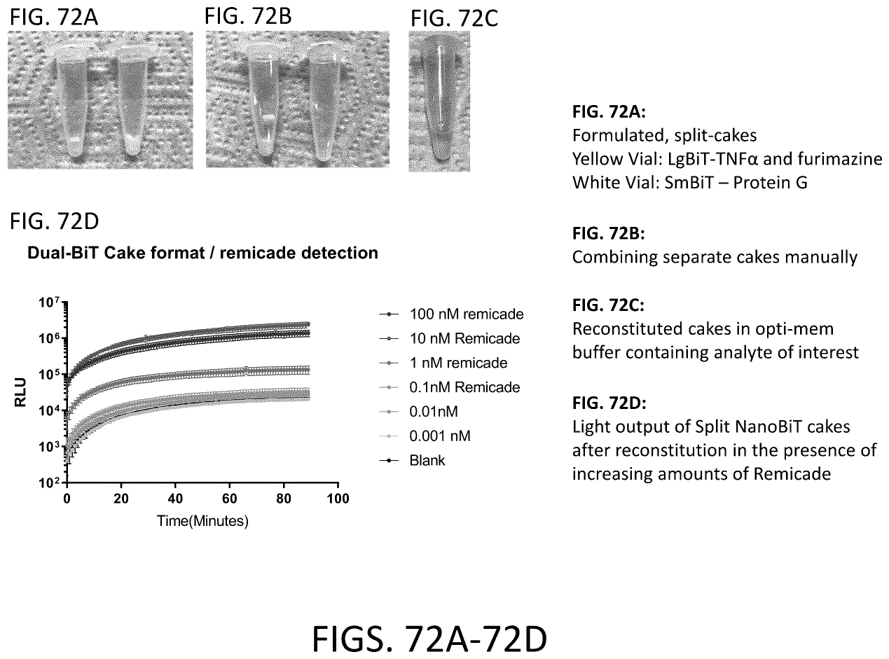

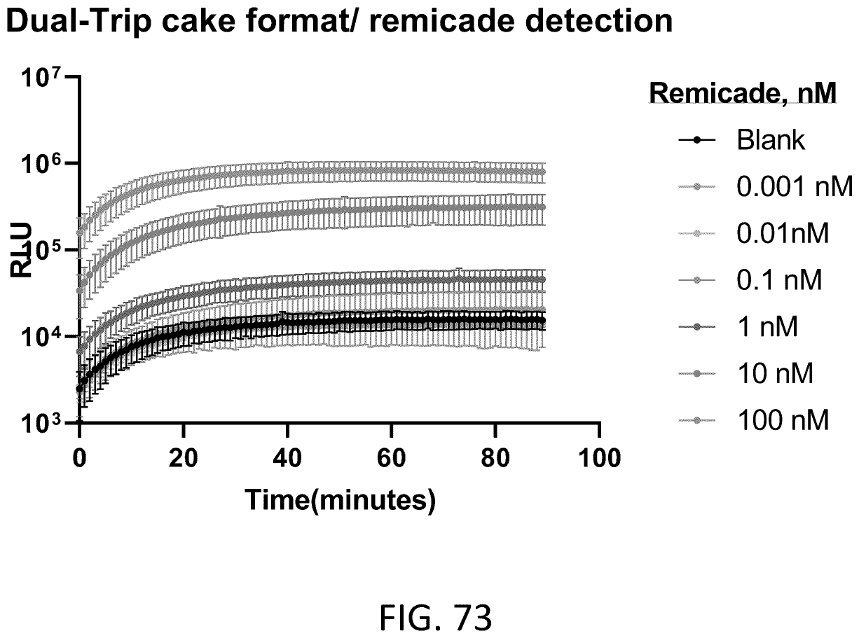

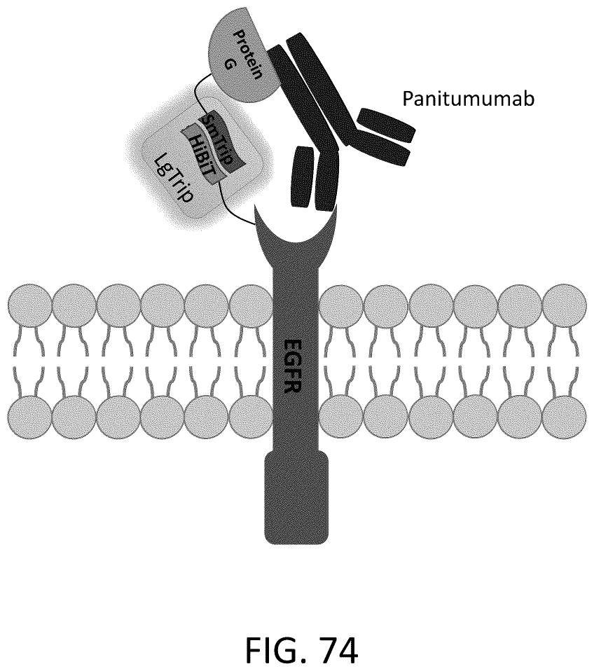

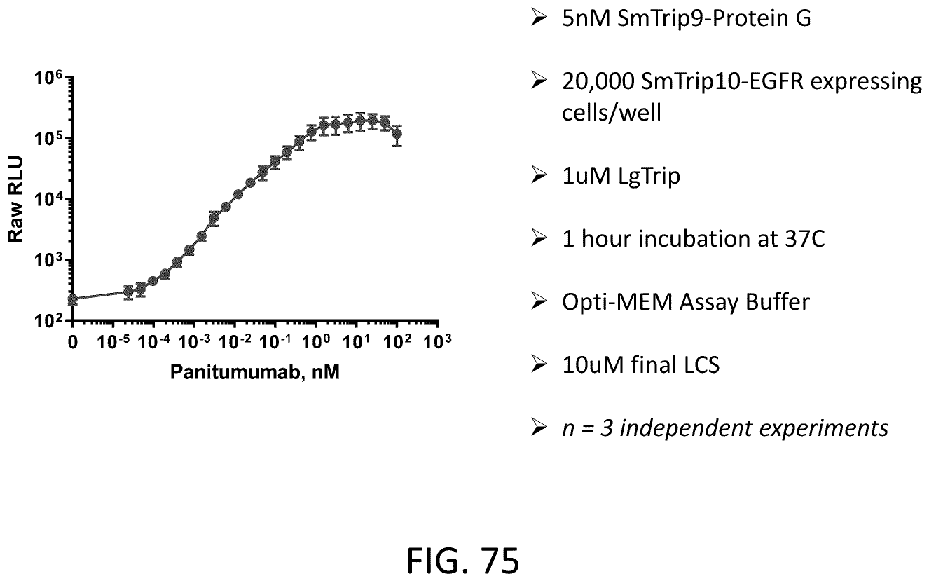

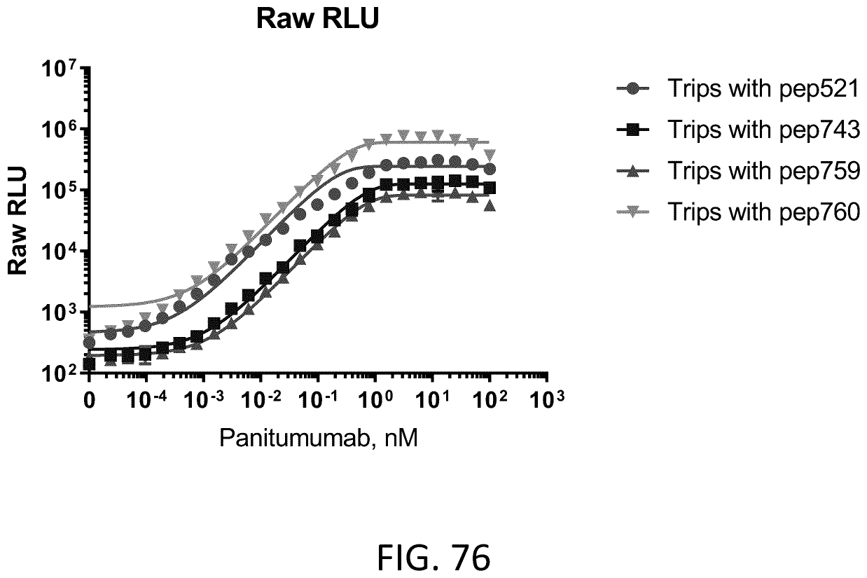

Formulations were lyophilized as separate components then manually combined to create the complete immunoassay. Cakes were reconstituted with Opti-MEM (Gibco), and 50 ul added to 50 μl of Remicade in a dose titration. Assays were performed in solid, white, nonbinding surface (NBS) plates (Costar) and analyzed on a GLOMAX Discover Multimode Microplate Reader (Promega) collecting total luminescence using a kinetic read. FIG. 72 displays the process and assay results for the NanoBiT anti-TNFα biologics “split-cake” lyophilized immunoassay. FIG. 72A depicts the independent lyophilized products. FIG. 72B depicts the results after manually combining the two separate cakes into one microcentrifuge tube. FIG. 72C depicts the lyophilized products after reconstitution with Opti-MEM buffer. FIG. 72D displays the kinetic bioluminescence results when in the presence of increasing amounts of Remicade. FIG. 73 displays the kinetic bioluminescence results for the anti-TNFα biologics NanoTrip assay using a kinetic read for bioluminescence in the presence of Remicade after following the same process laid out in FIG. 72. The dual cake format also created a successful immunoassay for Remicade.

### Example 28

### Developing a Cell-Based, Homogeneous Tripartite Assay for the Quantitation of Anti-EGFR Biologics

A bulk transfection was performed on HEK293 cells by preparing a 10 g/ml solution of DNA with a 1:10 dilution of IL6-VSHiBiT-15GS-EGFR (GSSGGGGSGGGGSS) (ATG-4288) and pGEM3Z carrier DNA (Promega). FuGENE HD was added to the DNA mixture to form a lipid:DNA complex. This complex was added to HEK293 cells with an adjusted cell density of 2×10⁵ cells/ml and incubated at 37° C. and 5% CO₂ overnight.

Transfected HEK293 cells were added to 96-well NBS plates (a separate plate for each SmTrip-15GS-G being tested) at a final concentration of 2×10⁵ cells/well. A reagent mixture of LgTrip 3546 and SmTrip9-G was added to the cells at a final concentration of 1 μM LgTrip 3546 and 10 nM SmTrip9-15GS-G. A 24-point panitumumab titration was added to each well with a final starting concentration of 100 nM and diluted 1:2 with a final ending concentration of 0 nM. All plates were covered and incubated for an hour at 37° C. and 5% CO₂. NANOLUC Live Cell Substrate was added to all wells at a final concentration of 10 μM, and luminescence of each plate was subsequently read on a luminometer. The following SmTrip9-G constructs were tested: ATG4002 SmTrip9(521)-15GS-G (SEQ ID NO: 724); ATG4496 SmTrip9(743)-15GS-G (SEQ ID NO: (726); ATG4558 SmTrip9(759)-15GS-G (SEQ ID NO: 728); and ATG4551 SmTrip9(760)-15GS-G (SEQ ID NO: 730). Each configuration was successful in quantitatively detecting panitumumab.

### Example 29

### Testing Various SmTrip9-Protein G Fusion Proteins in Solution-Based, Homogeneous Anti-TNFα Biologics Tripartite Immunoassays

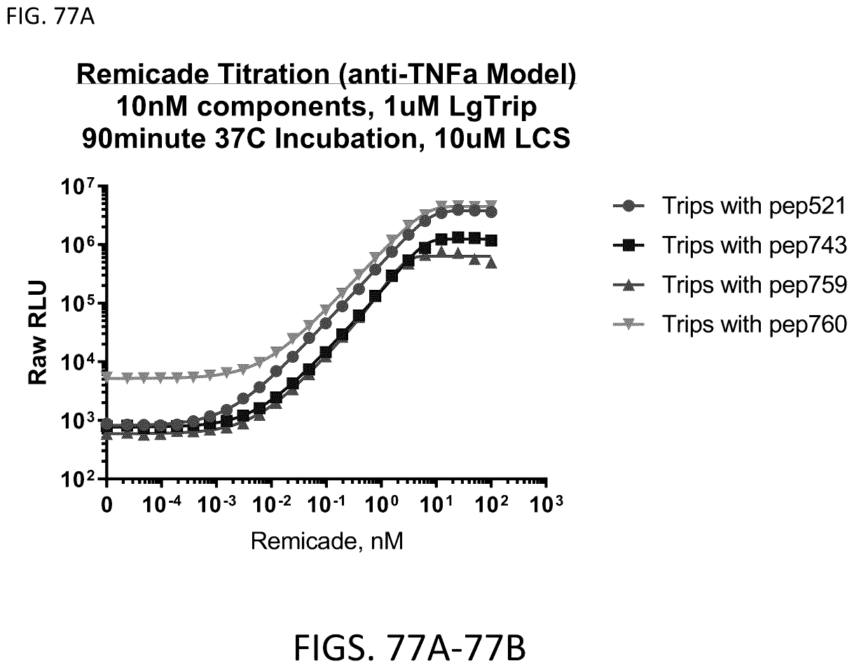

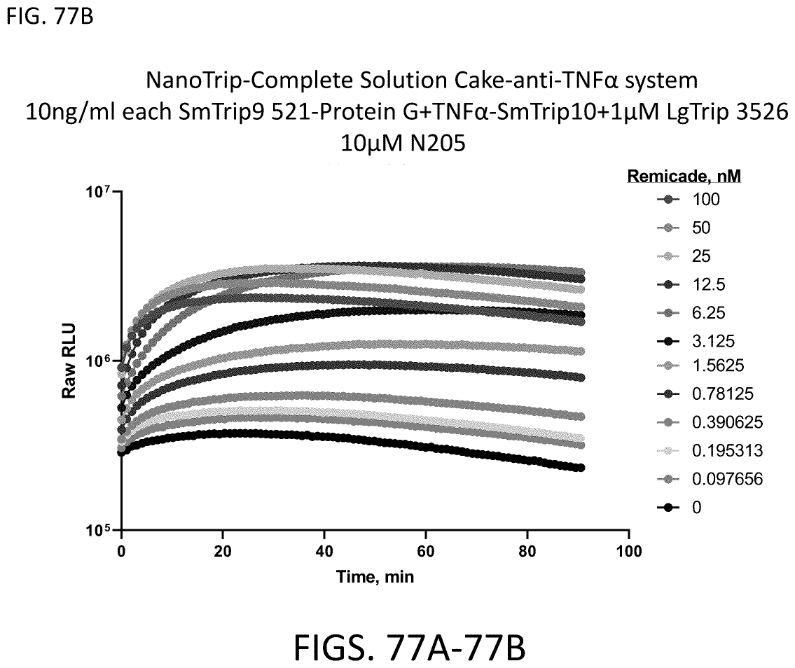

FIG. 77 displays the dose response curves for the solution-based homogenous anti-TNFα biologics immunoassay using SmTrip9 variants SmTrip9 pep521 (SEQ ID NO: 16), SmTrip9 pep743 (SEQ ID NO: 21), SmTrip9 pep759 (SEQ ID NO: 22), or SmTrip 9 pep760 (SEQ ID NO: 23) in a standard assay buffer consisting of 0.01% BSA in PBS, pH 7.0. For these experiments, 10 nM of protein G-15gly/ser-SmTrip9 variant, 10 nM TNFα-15 gly/ser-SmTriplO Pep289 (SEQ ID NO: 17), and 1 μM LgTrip 3546 (SEQ ID NO: 12) protein were incubated in the presence of Remicade for 90 minutes. Furimazine (NANOGLO Live Cell Substrate; Promega N205) was added, and total luminescence signal was analyzed using a GLOMAX Discover. All of the SmTrip9 variants were successful in the assay detecting Remicade, albeit with different levels of background and Bmax.

To evaluate the potential application of lyophilization for preservation of the entire anti-TNFα biologics, NanoTrip immunoassays in single vial formulations containing peptide-labeled fusion proteins and LgTrip 3546 (SEQ ID NO: 12) and furimazine were prepared. The 20× stock formulations are as follows:

NanoTrip anti-TNFα biologics immunoassay: 5 mM azothiothymine, 5 mM ascorbic acid, 2.75% w/v pullulan, ddH20 (Millipore), 200 μM furimazine in ethanol, 20 μM LgTrip 3546 protein (SEQ ID NO:12), 200 nM protein G-SmTrip9 variant fusion protein, and 200 nM TNFα-SmTriplO Pep289 (SEQ ID NO:17) fusion protein.

One mL aliquots of 20× stock solution was dispensed into 10 mL amber glass vials, and a runner stopper was partially inserted into the vial. Vials were loaded into the lyophilizer (Virtis Genesis 12EL lyophilizer) with shelves pre-chilled to 4.7° C. Product then underwent a freezing step with a shelf temperature of −50° C. for 2 hr after which time the condenser step started. During the run, the condenser temperature ran between −5° C. and −87° C. A vacuum pull down ran next at the pressure set-points of 75 and 200 mTorr. Sublimation lasted ˜7.5 hr and desorption lasted ˜16.1 hr. At the end of the lyophilization process, the vials were back-filled with nitrogen and sealed with fully inserted stoppers at −600 Torr of pressure. FIG. 77B provides the dose response curve for Remicade using the lyophilized anti-TNFα biologics immunoassay.

### Example 30

### Direct-Labeling of Antibodies Via Reactive Peptides for Development of Solution-Based, Homogenous IL-6 Immunoassays

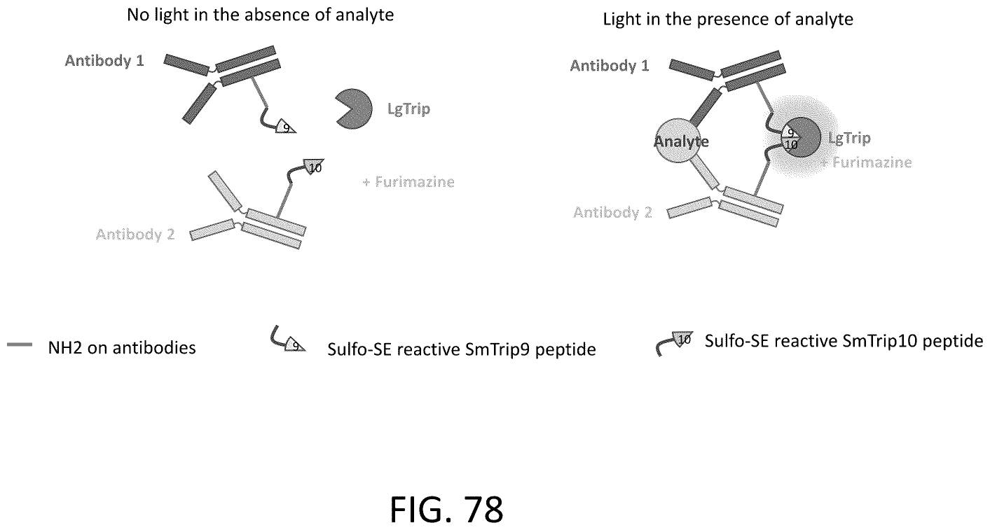

The basic principle of homogeneous NanoLuc tripartite immunoassays with directly-labeled antibodies is depicted in FIG. 78. First, a pair of antibodies that target non-overlapping epitopes on IL-6 are chemically conjugated to SmTrip9 or SmTrip10-based reactive peptides. When the labeled antibodies bind IL-6 analyte, the complementary subunits are brought into proximity, thereby reconstituting a bright luciferase that produces a bioluminescent signal in the presence of the LgTrip protein and furimazine substrate. The amount of luminescence generated by this assay configuration is directly proportional to the amount of target analyte.

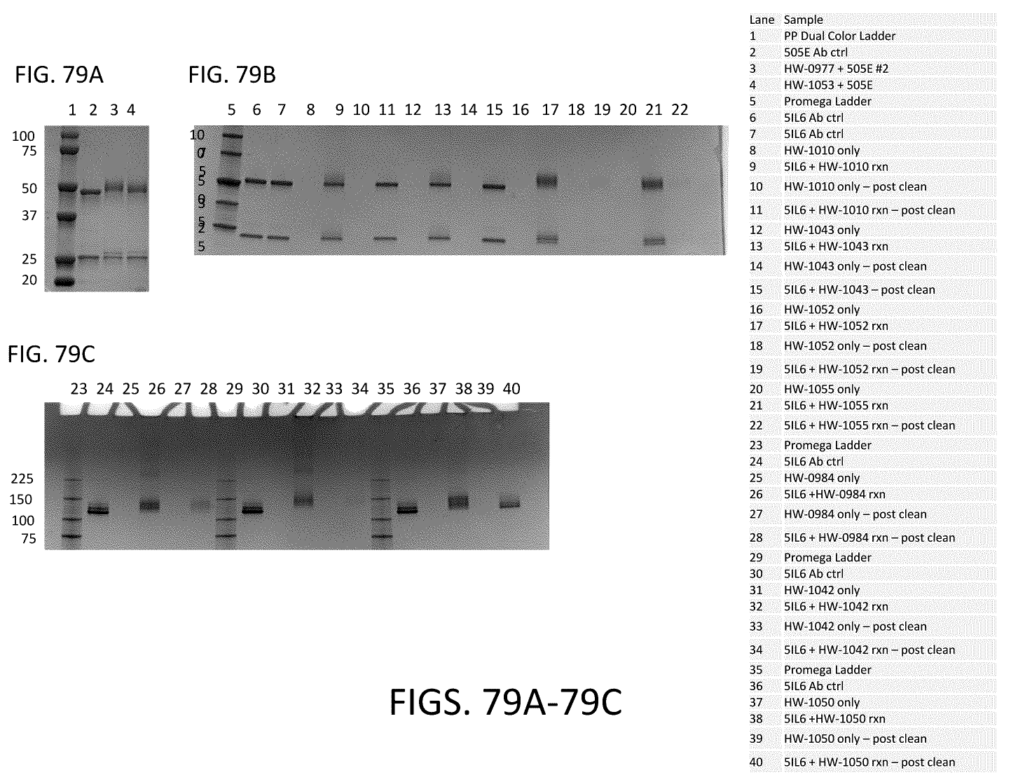

SmTrip9 variants such as Pep693 (SEQ ID NO: 20), Pep895 (SEQ ID NO: 24), and Pep929 (SEQ ID NO: 25) or SmTriplO variants such as Pep691 (SEQ ID NO: 18) and Pep692 (SEQ ID NO: 19) were individually dissolved in DMF to 5 mM. Antibodies were buffered exchanged 2× into 10 mM sodium bicarbonate buffer (pH 8.5) using Zeba spin desalting columns (ThermoFisher). Subsequently, these antibodies were combined with 20× molar excess of a reactive peptide for 1 hr at 4° C. while shaking in order to covalently label the proteins. Unreacted label was removed with two passes through Zeba spin columns in PBS buffer. To create the reagents for the exemplary human IL-6 immunoassay, the mouse anti-human IL-6 monoclonal antibodies clone 5IL6 (Thermo cat #M620) and clone 505E 9A12 A3 (Thermo cat #AHC0662) were used. SmTrip9 reactive peptides were used to label antibody 5IL6 while SmTriplO reactive peptides were used to label antibody 505E. The denaturing SDS-PAGE gel shown in FIG. 79 was used to characterize the conjugated antibodies. The gel revealed that the degree of antibody labeling was dependent on the peptide sequence and chemical structure of the label.

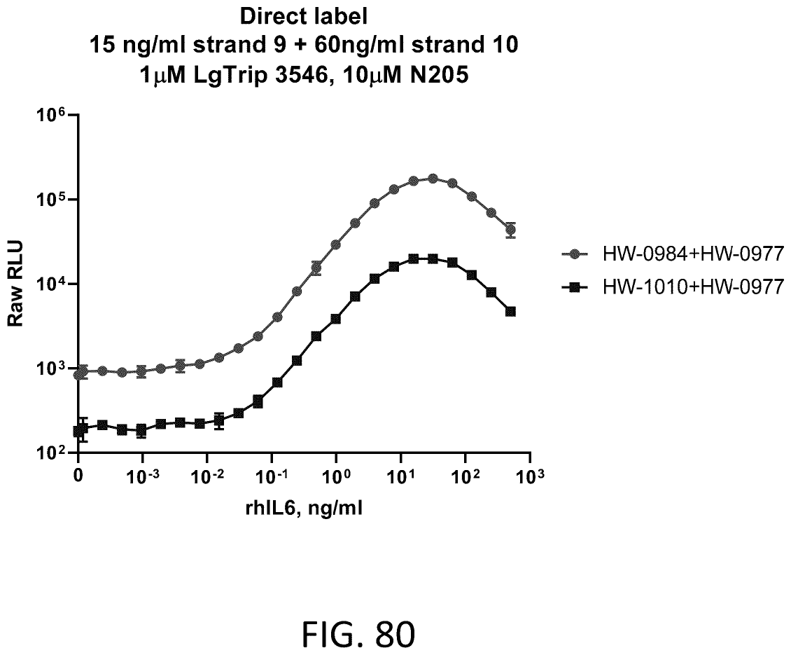

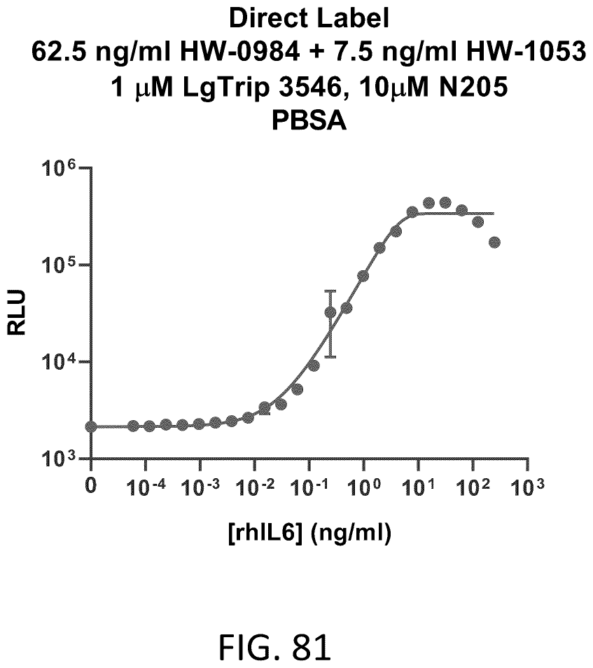

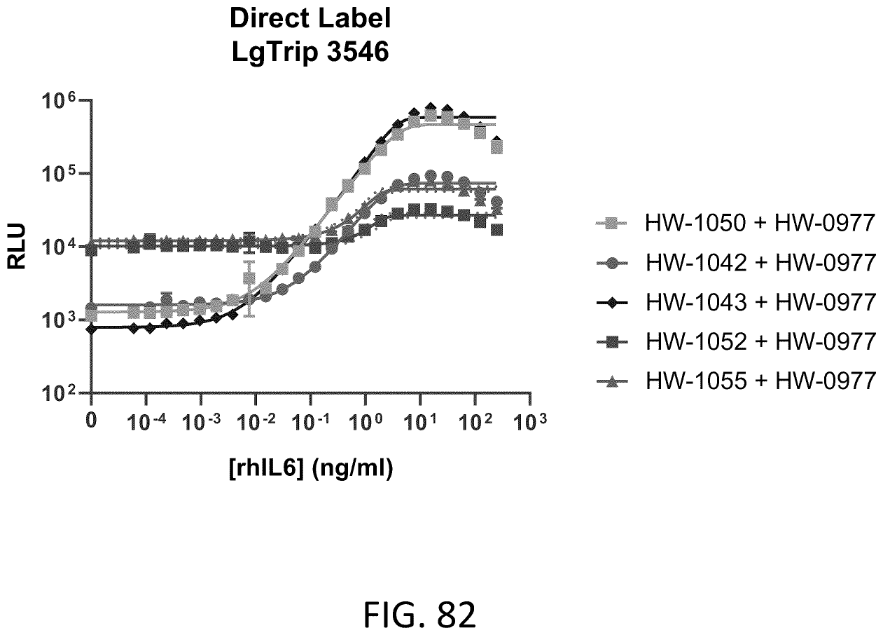

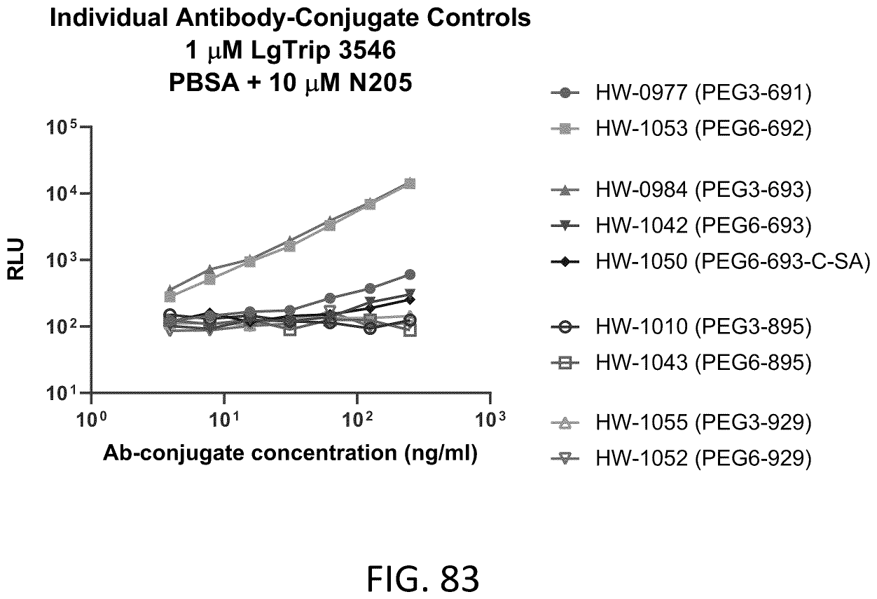

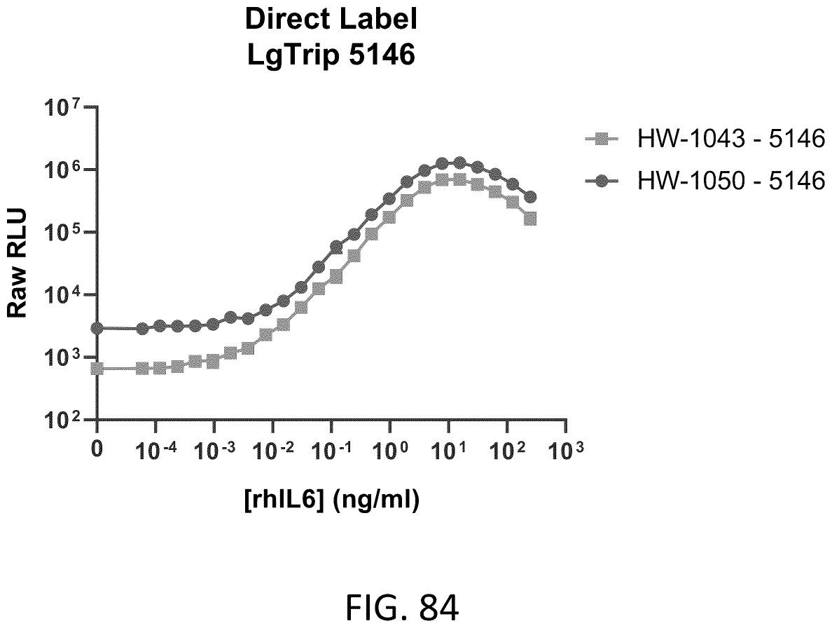

FIGS. 80-82 display raw RLU dose response curves for antibody conjugates in the presence of a rhIL-6 titration series. For these experiments, rhIL-6 and antibody conjugates were incubated for 90 minutes with 1 μM LgTrip 3546 (SEQ ID NO: 12) in PBS (pH 7.0) with 0.01% BSA. After addition of N205, luminescence signal was measured. Data in FIG. 80 were generated using 15 ng/ml of SmTrip9-labeled variant (HW-0984 or HW-1010) 5IL6 antibody and 60 ng/ml of SmTrip10-labeled variant (HW-0977) 505E antibody. Data in FIG. 81 were generated using 62.5 ng/ml of SmTrip9-labeled (HW-0984) 5IL6 antibody and 60 ng/ml of SmTrip10-labeled (HW-1053) 505E antibody. Data in FIG. 82 were generated using the following concentrations of antibody conjugates: 15 ng/ml HW-1043 (SEQ ID NO: 24)+30 ng/ml HW-1053 (SEQ ID NO: 18), 15 ng/ml HW-1052 (SEQ ID NO: 25)+15 ng/ml HW-1053, (SEQ ID NO: 18) 15 ng/ml HW-1055(SEQ ID NO: 25)+15 ng/ml HW-1053 (SEQ ID NO: 18), 60 ng/ml HW-1042 (SEQ ID NO: 20)+8 ng/ml HW-1053 (SEQ ID NO: 18), and 60 ng/ml HW-1050 (SEQ ID NO: 27)+8 ng/ml HW-1053 (SEQ ID NO: 18). In this experiment, SmTrip9 variant labels HW-1050 (SEQ ID NO: 27) and HW-1043 (SEQ ID NO: 24) gave the best signal to background displaying close to 10⁶ RLUs in the presence of high rhIL-6 concentrations and low light output in the absence of the analyte. In contrast, SmTrip9 variant labels HW-1055(SEQ ID NO: 25 (SulfoSE-PEG3)) and HW-1052 (SEQ ID NO: 25 (SulfoSE-PEG6)) had high signal even in the absence of rhIL-6 suggesting these labels spontaneously assemble into the reconstituted luciferase. FIG. 83 displays light output from titration of individual antibody conjugates in PBS (pH 7.0) with 0.01% BSA, 1 μM LgTrip 3546 (SEQ ID NO: 12), and N205. Most conjugates show RLUs equivalent to furimazine background (˜100 RLU), and no increase in RLU with increasing concentration of labeled antibodies. Conjugates HW-0984 (SEQ ID NO: 20) and HW-1053 (SEQ ID NO: 19) were exceptions, generating increasing RLUs with concentration and reaching over 1,000 at concentrations above 100 ng/ml. In FIG. 84, two SmTrip9 conjugates with high S/B (labeled with HW-1050 (SEQ ID NO: 27) and HW-1043 (SEQ ID NO: 24)) were assayed under conditions described for FIG. 82, but with 1 μM LgTrip 5146 (SEQ ID NO: 451), producing results similar to LgTrip 3546 (SEQ ID NO: 12), demonstrating the feasibility of using different LgTrp variants to construct these assays.

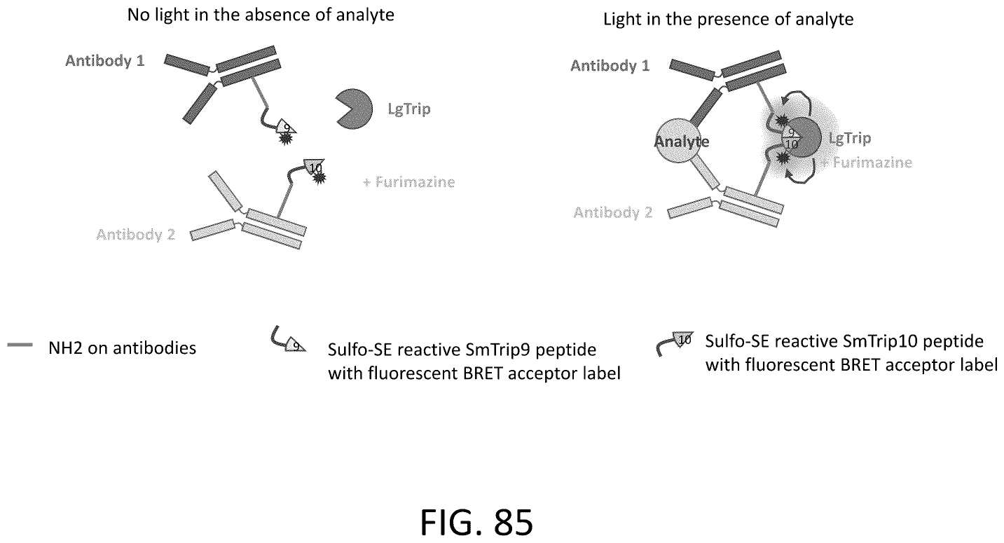

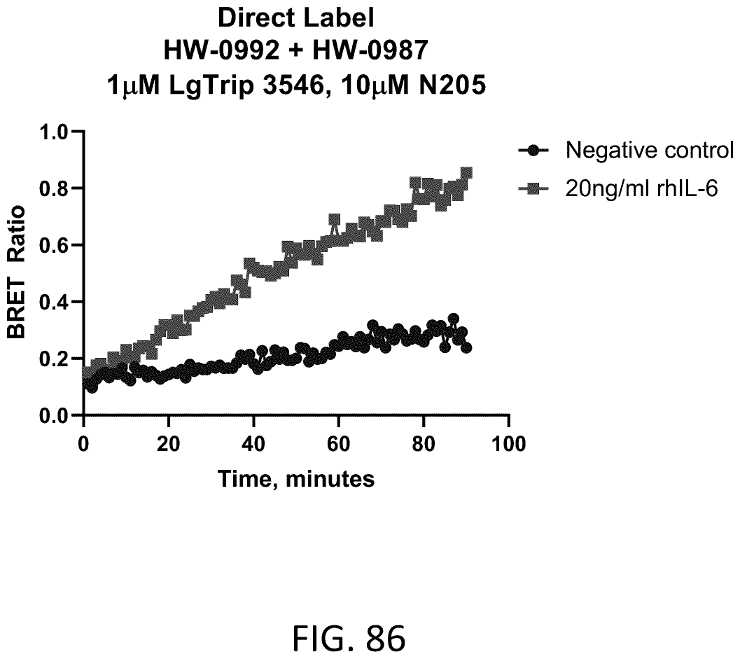

Components for homogeneous tripartite NanoLuc immunoassays can also be constructed by direct-labeling antibodies with SmTrip9 or SmTrip10 variants that contain a fluorophore such as tetramethylrhodamine (TMR). This is depicted schematically in FIG. 85 including the expected BRET from the luciferase to the fluorophore labels. Kinetic reads for BRET with labels HW-0987 (SmTrip9 variants with TMR) and HW-0992 (SmTriplO variants with TMR) in the IL-6 immunoassay are shown in FIG. 86. BRET was observed only in the presence of rhIL-6 analyte demonstrating the complementation and energy transfer are occurring when the analyte brings these components together.

### Example 31

### SulfoSE-PEG3-SmTrip9 Pep693 (HW-0984)

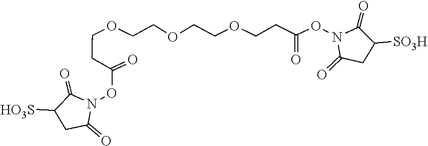 [SMILES: O=C(CCOCCOCCOCCC(=O)ON1C(=O)CC(S(=O)(=O)O)C1=O)ON1C(=O)CC(S(=O)(=O)O)C1=O]

3,3′-((oxybis(ethane-2,1-diyl))bis(oxy))dipropionic acid (55 mg, 0.22 mmol) was dissolved in anhydrous DMF, and then diisopropylethylamine (120 mg, 0.88 mmol) and HATU (176 mg, 0.45 mmol) added. The mixture was stirred for five minutes. Meanwhile, N-hydroxy-2,5-dioxopyrrolidine-3-sulfonic acid (90 mg, 0.46 mmol) was dissolved in 5 ml DMSO and then added to the previous solution dropwise. The mixture was stirred for another hour until LC-MS shows disappearance of acid. The solution was directly used in the next step. Calculated: m/z=603.05 [M⁻]; measured (ESI): m/z=603.04 [M⁻].

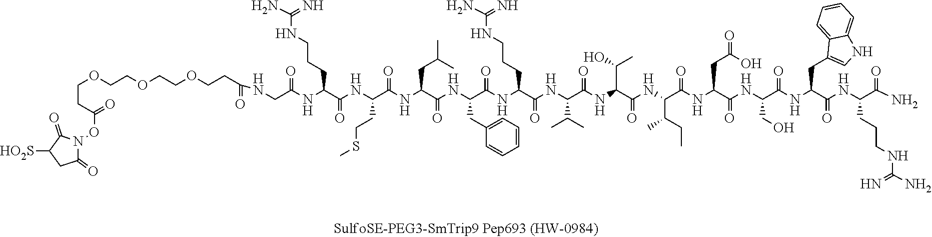

SmTrip9 Pep693 (GRMLFRVTINSWR, 27 mg, 0.045 mmol) was dissolved in DMF. The solution was then added to the previous PEG3 bis Sulfo-SE solution. The mixture was then stirred for another hour and directly purified by preparative HPLC. Calculated: m/z=1022.98 [M+2H]²⁺; measured (ESI): m/z=1023.09 [M+2H]²⁺.

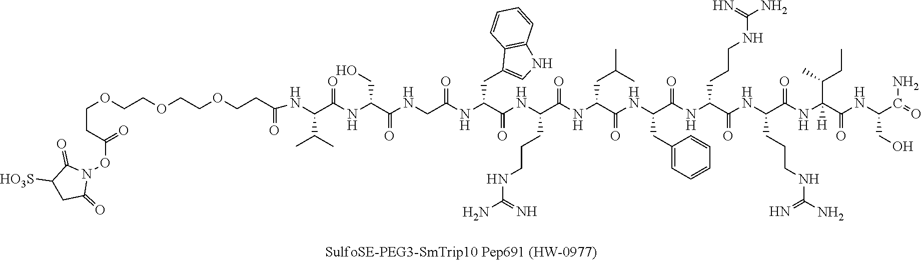 [SMILES: CC[C@@H](C)[C@H](NC(=O)[C@H](CCCNC(=N)N)NC(=O)[C@@H](CCCNC(=N)N)NC(=O)[C@H](Cc1ccccc1)NC(=O)[C@@H](CC(C)C)NC(=O)[C@H](CCCNC(=N)N)NC(=O)[C@@H](Cc1c[nH]c2ccccc12)NC(=O)CNC(=O)[C@@H](CO)NC(=O)[C@@H](NC(=O)CCOCCOCCOCCC(=O)ON1C(=O)CC(S(=O)(=O)O)C1=O)C(C)C)C(=O)N[C@@H](CO)C(N)=O]

### Example 32

### SulfoSE-PEG3-SmTriplO Pep691 (HW-0977)

HW-0977 was synthesized by the same method as HW-0984. Calculated: m/z=892.93 [M+2H]²⁺; measured (ESI): m/z=893.61 [M+2H]²⁺.

### Example 33

### SulfoSE-PEG3-SmTrip9 Pep895 (HW-1010)

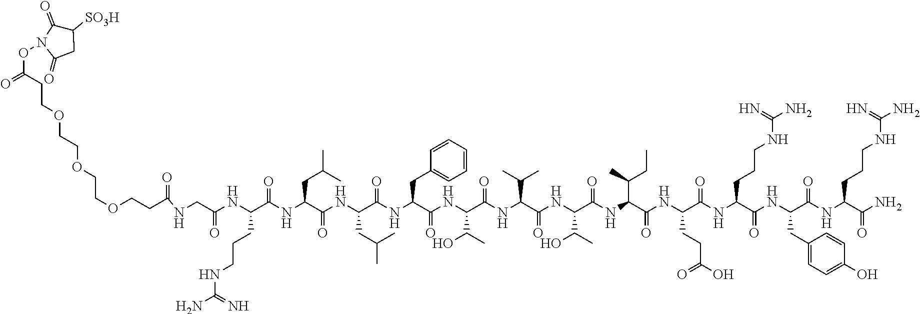 [SMILES: CC[C@H](C)[C@H](NC(=O)[C@@H](NC(=O)[C@@H](NC(=O)[C@@H](NC(=O)[C@H](Cc1ccccc1)NC(=O)[C@H](CC(C)C)NC(=O)[C@H](CC(C)C)NC(=O)[C@H](CCCNC(=N)N)NC(=O)CNC(=O)CCOCCOCCOCCC(=O)ON1C(=O)CC(S(=O)(=O)O)C1=O)C(C)O)C(C)C)C(C)O)C(=O)N[C@@H](CCC(=O)O)C(=O)N[C@@H](CCCNC(=N)N)C(=O)N[C@@H](Cc1ccc(O)cc1)C(=O)N[C@@H](CCCNC(=N)N)C(N)=O]

HW-1010 was synthesized by the same method as HW-0984. Calculated: m/z=1016.51 [M+2H]²⁺; measured (ESI): m/z=1016.92 [M+2H]²⁺.

### Example 34

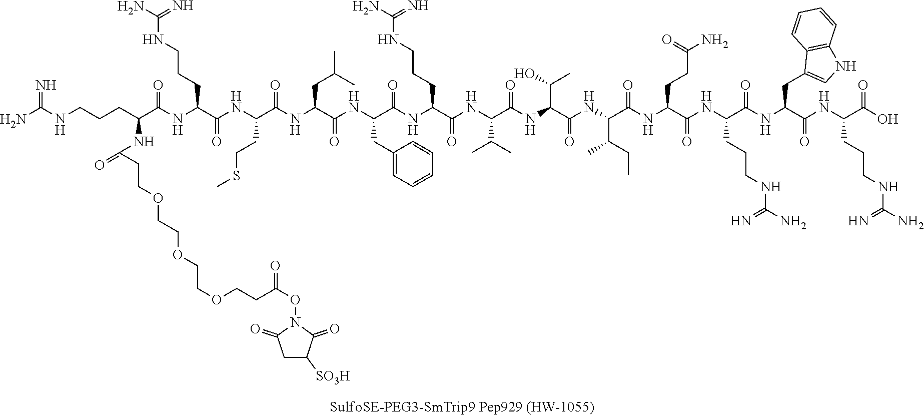 [SMILES: CC[C@H](C)[C@H](NC(=O)[C@@H](NC(=O)[C@@H](NC(=O)[C@H](CCCNC(=N)N)NC(=O)[C@H](Cc1ccccc1)NC(=O)[C@H](CC(C)C)NC(=O)[C@H](CCSC)NC(=O)[C@H](CCCNC(=N)N)NC(=O)[C@H](CCCNC(=N)N)NC(=O)CCOCCOCCOCCC(=O)ON1C(=O)CC(S(=O)(=O)O)C1=O)C(C)C)[C@@H](C)O)C(=O)N[C@@H](CCC(N)=O)C(=O)N[C@@H](CCCNC(=N)N)C(=O)N[C@@H](Cc1c[nH]c2ccccc12)C(=O)N[C@@H](CCCNC(=N)N)C(=O)O]

HW-1055 was synthesized by the same method as HW-0984. Calculated: m/z=1114.06 [M+2H]²⁺; measured (ESI): m/z=1113.95 [M+2H]²⁺.

### Example 35

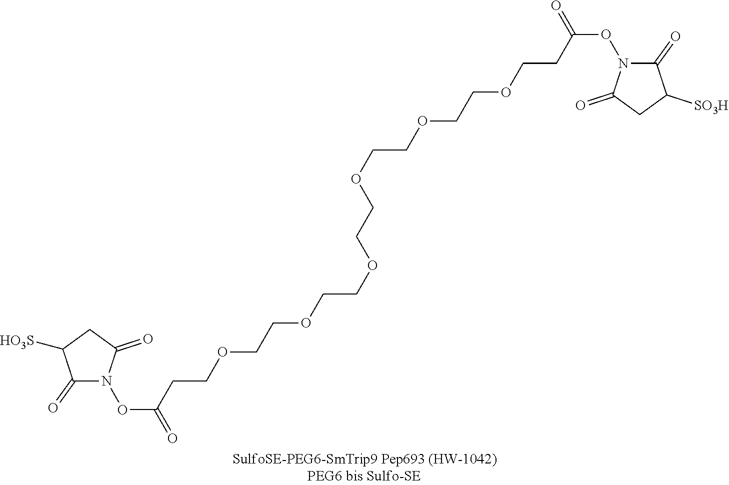 [SMILES: O=C(CCOCCOCCOCCOCCOCCOCCC(=O)ON1C(=O)CC(S(=O)(=O)O)C1=O)ON1C(=O)CC(S(=O)(=O)O)C1=O]

Bis PEG6-acid (39 mg, 0.10 mmol) was dissolved in anhydrous DMF and then diisopropylethylamine (53 mg, 0.4 mmol) and HATU (78 mg, 0.20 mmol) added. The mixture was stirred for five minutes. Meanwhile, N-hydroxy-2,5-dioxopyrrolidine-3-sulfonic acid (40 mg, 0.20 mmol) was dissolved in 5 ml DMSO and then added to the previous solution dropwise. The mixture was stirred for another hour until LC-MS shows disappearance of acid. The solution was directly used in the next step. Calculated: m/z=735.13 [M⁻]; measured (ESI): m/z=735.04 [M].

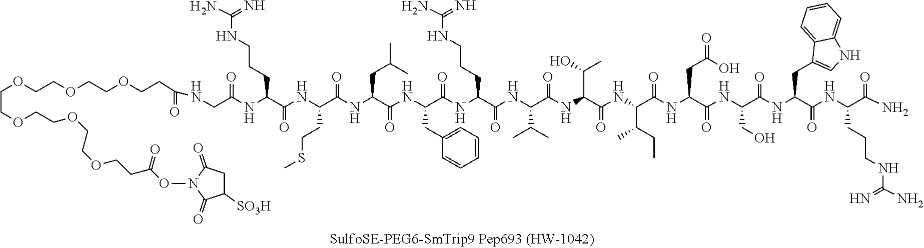 [SMILES: CC[C@H](C)[C@H](NC(=O)[C@@H](NC(=O)[C@@H](NC(=O)[C@H](CCCNC(=N)N)NC(=O)[C@H](Cc1ccccc1)NC(=O)[C@H](CC(C)C)NC(=O)[C@H](CCSC)NC(=O)[C@H](CCCNC(=N)N)NC(=O)CNC(=O)CCOCCOCCOCCOCCOCCOCCC(=O)ON1C(=O)CC(S(=O)(=O)O)C1=O)C(C)C)[C@@H](C)O)C(=O)N[C@@H](CC(=O)O)C(=O)N[C@@H](CO)C(=O)N[C@@H](Cc1c[nH]c2ccccc12)C(=O)N[C@@H](CCCNC(=N)N)C(N)=O]

SmTrip9 Pep693 (GRMLFRVTINSWR, 20 mg, 0.013 mmol) was dissolved in DMF. The solution was then added to the previous PEG6 bis Sulfo-SE solution. The mixture was then stirred for another hour and directly purified by preparative HPLC. Calculated: m/z=1089.02 [M+2H]²⁺; measured (ESI): m/z=1088.94 [M+2H]²⁺.

### Example 36

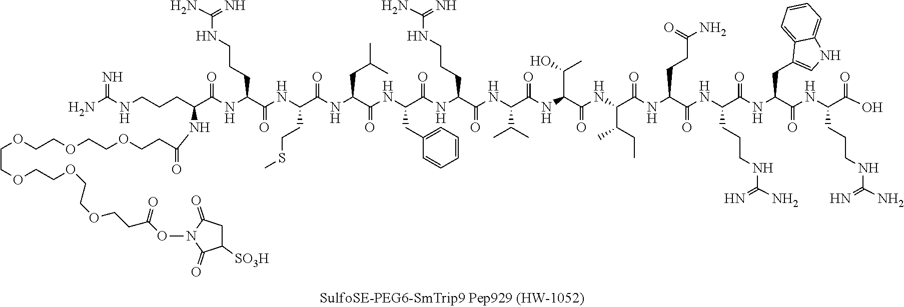 [SMILES: CC[C@H](C)[C@H](NC(=O)[C@@H](NC(=O)[C@@H](NC(=O)[C@H](CCCNC(=N)N)NC(=O)[C@H](Cc1ccccc1)NC(=O)[C@H](CC(C)C)NC(=O)[C@H](CCSC)NC(=O)[C@H](CCCNC(=N)N)NC(=O)[C@H](CCCNC(=N)N)NC(=O)CCOCCOCCOCCOCCOCCOCCC(=O)ON1C(=O)CC(S(=O)(=O)O)C1=O)C(C)C)[C@@H](C)O)C(=O)N[C@@H](CCC(N)=O)C(=O)N[C@@H](CCCNC(=N)N)C(=O)N[C@@H](Cc1c[nH]c2ccccc12)C(=O)N[C@@H](CCCNC(=N)N)C(=O)O]

HW-1052 was synthesized by the same method as HW-1042. Calculated: m/z=1180.10 [M+2H]²⁺; measured (ESI): m/z=1179.82 [M+2H]²⁺.

### Example 37

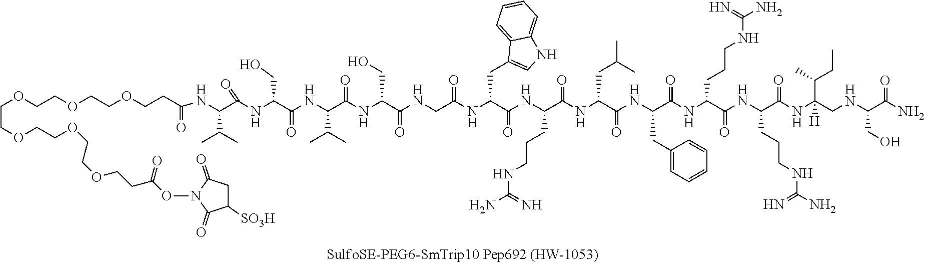 [SMILES: CC[C@@H](C)[C@@H](CN[C@@H](CO)C(N)=O)NC(=O)[C@H](CCCNC(=N)N)NC(=O)[C@@H](CCCNC(=N)N)NC(=O)[C@H](Cc1ccccc1)NC(=O)[C@@H](CC(C)C)NC(=O)[C@H](CCCNC(=N)N)NC(=O)[C@@H](Cc1c[nH]c2ccccc12)NC(=O)CNC(=O)[C@@H](CO)NC(=O)[C@@H](NC(=O)[C@@H](CO)NC(=O)[C@@H](NC(=O)CCOCCOCCOCCOCCOCCOCCC(=O)ON1C(=O)CC(S(=O)(=O)O)C1=O)C(C)C)C(C)C]

HW-1053 was synthesized by the same method as HW-1042. Calculated: m/z=1052.03 [M+2H]²⁺; measured (ESI): m/z=1051.92 [M+2H]²⁺.

### Example 38

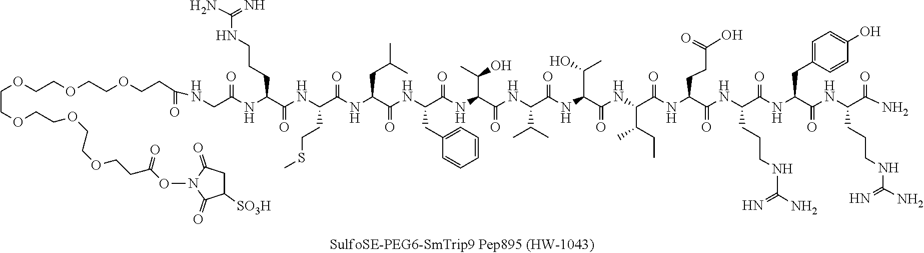 [SMILES: CC[C@H](C)[C@H](NC(=O)[C@@H](NC(=O)[C@@H](NC(=O)[C@@H](NC(=O)[C@H](Cc1ccccc1)NC(=O)[C@H](CC(C)C)NC(=O)[C@H](CCSC)NC(=O)[C@H](CCCNC(=N)N)NC(=O)CNC(=O)CCOCCOCCOCCOCCOCCOCCC(=O)ON1C(=O)CC(S(=O)(=O)O)C1=O)[C@@H](C)O)C(C)C)[C@@H](C)O)C(=O)N[C@@H](CCC(=O)O)C(=O)N[C@@H](CCCNC(=N)N)C(=O)N[C@@H](Cc1ccc(O)cc1)C(=O)N[C@@H](CCCNC(=N)N)C(N)=O]

HW-1043 was synthesized by the same method as HW-1042. Calculated: m/z=1082.55 [M+2H]²⁺; measured (ESI): m/z=1082.34 [M+2H]²⁺

### Example 39

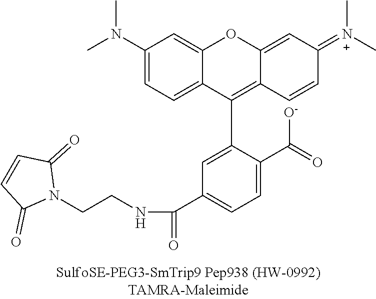 [SMILES: CN(C)c1ccc2c(-c3cc(C(=O)NCCN4C(=O)C=CC4=O)ccc3C(=O)[O-])c3ccc(=[N+](C)C)cc-3oc2c1]

5-TAMRA (50 mg, 0.116 mmol) was dissolved in DMF. Diisopropylethylamine (45 mg, 0.128 mmol) was added followed by TSTU (38 mg, 0.128 mmol). The mixture was stirred for 20 min, 1-(2-aminoethyl)-1H-pyrrole-2,5-dione (18 mg, 0.128 mmol) added, and the resulting reaction mixture was stirred for another hour and directly purified by preparative HPLC. Calculated: m/z=553.20 [M+H]+; measured (ESI): m/z=553.40 [M+H]⁺.

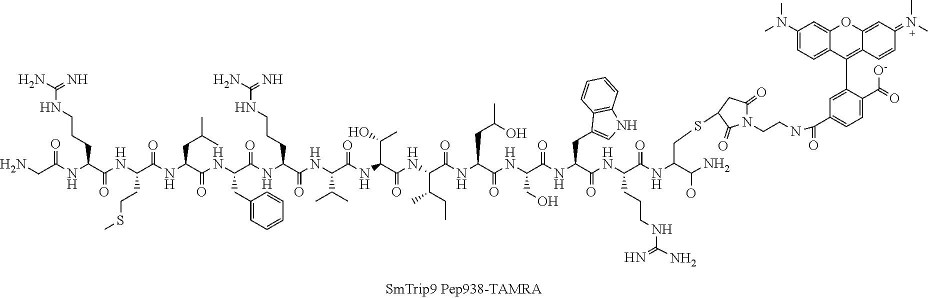

TAMRA-Maleimide (8 mg, 0.014 mmol) was dissolved in DMF. A solution of SmTrip9 (Pep938) (GRMLFRVTINSWRC, 25 mg, 0.014 mmol) in PBS buffer (pH 7.4, 200 mM) was added. The reaction mixture was stirred for two hours and directly purified by preparative HPLC. Calculated: m/z=1146.05 [M+2H]²⁺; measured (ESI): m/z=1146.33 [M+2H]²⁺

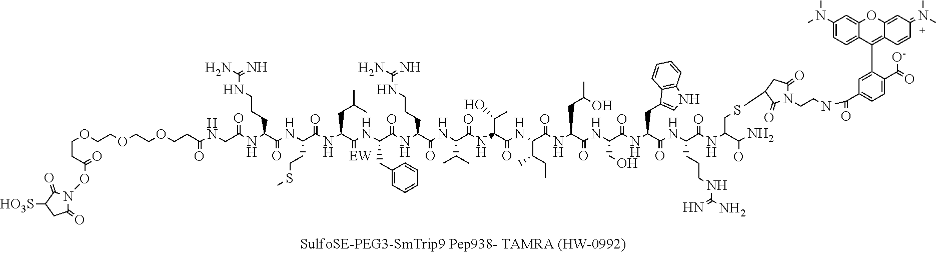

SmTrip9 Pep938-TAMRA (8.5 mg, 0.0038 mmol) was dissolved in DMF. The solution was then added to PEG3 bis Sulfo-SE prepared as shown in synthesis of HW-0984. The reaction mixture was stirred for two hours and directly purified by preparative HPLC. Calculated: m/z=901.05 [M+3H]³⁺; measured (ESI): m/z=901.20 [M+3H]³⁺.

### Example 40

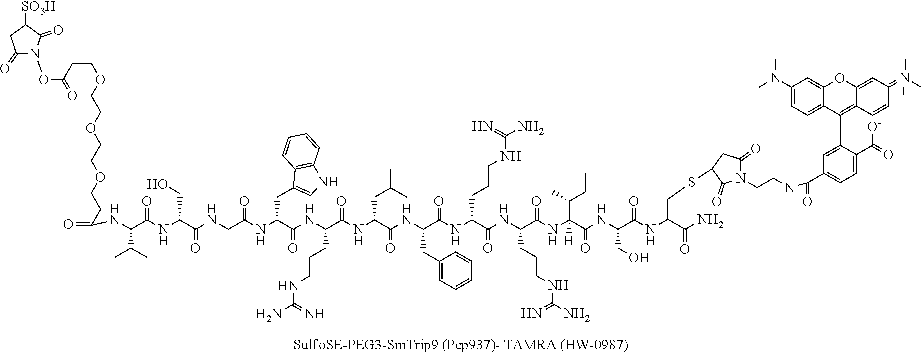

HW-0987 was synthesized by the same method as HW-0992. Calculated: m/z=814.03 [M+3H]³⁺; measured (ESI): m/z=814.40 [M+3H]³⁺.

### Example 41

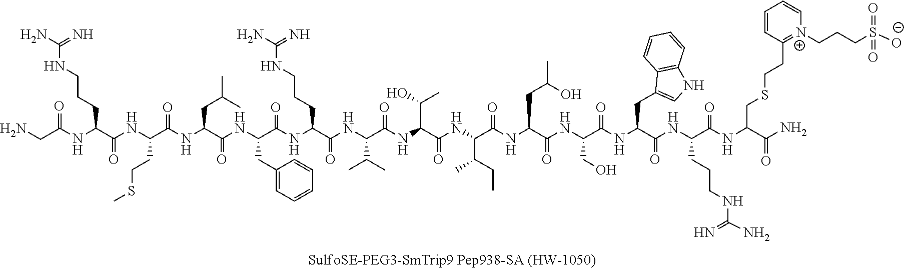 [SMILES: CC[C@H](C)[C@H](NC(=O)[C@@H](NC(=O)[C@@H](NC(=O)[C@H](CCCNC(=N)N)NC(=O)[C@H](Cc1ccccc1)NC(=O)[C@H](CC(C)C)NC(=O)[C@H](CCSC)NC(=O)[C@H](CCCNC(=N)N)NC(=O)CN)C(C)C)[C@@H](C)O)C(=O)N[C@@H](CC(C)O)C(=O)N[C@@H](CO)C(=O)N[C@@H](Cc1c[nH]c2ccccc12)C(=O)N[C@@H](CCCNC(=N)N)C(=O)NC(CSCCc1cccc[n+]1CCCS(=O)(=O)[O-])C(N)=O]

SmTrip9 Pep938 (GRMLFRVTINSWR, 26 mg, 0.015 mmol) was dissolved in DMSO. 1-(3-Sulfopropyl)-2-vinylpyridinium Hydroxide Inner Salt (3.40 mg 0.015 mmol) was dissolved in phosphate buffer (pH=7.4, 100 mM) and was added slowly to the peptide solution. The mixture was stirred for another three hours and directly purified by preparative HPLC. Calculated: m/z=983.48 [M+2H]²⁺; measured (ESI): m/z=983.39 [M+2H]²⁺.

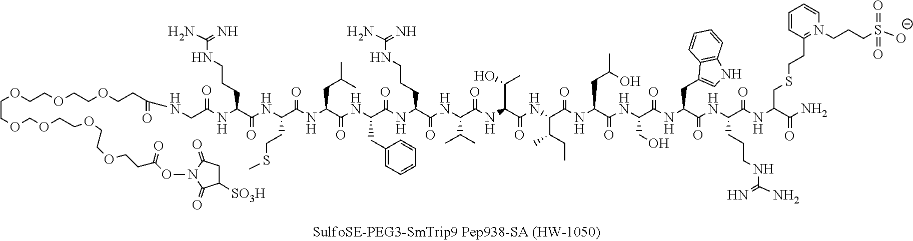

SmTrip9 Pep938-SA (10 mg, 0.005 mmol) was dissolved in DMF. The solution was then added to PEG6 bis Sulfo-SE prepared as shown in HW-0984. The reaction mixture was stirred for two hours and directly purified by preparative HPLC. Calculated: m/z=1254.05 [M+2H]²⁺; measured (ESI): m/z=1253.98 [M+2H]²⁺.

Shown below is a representative scheme for the synthesis of PEG-linked peptide SulfoSE.

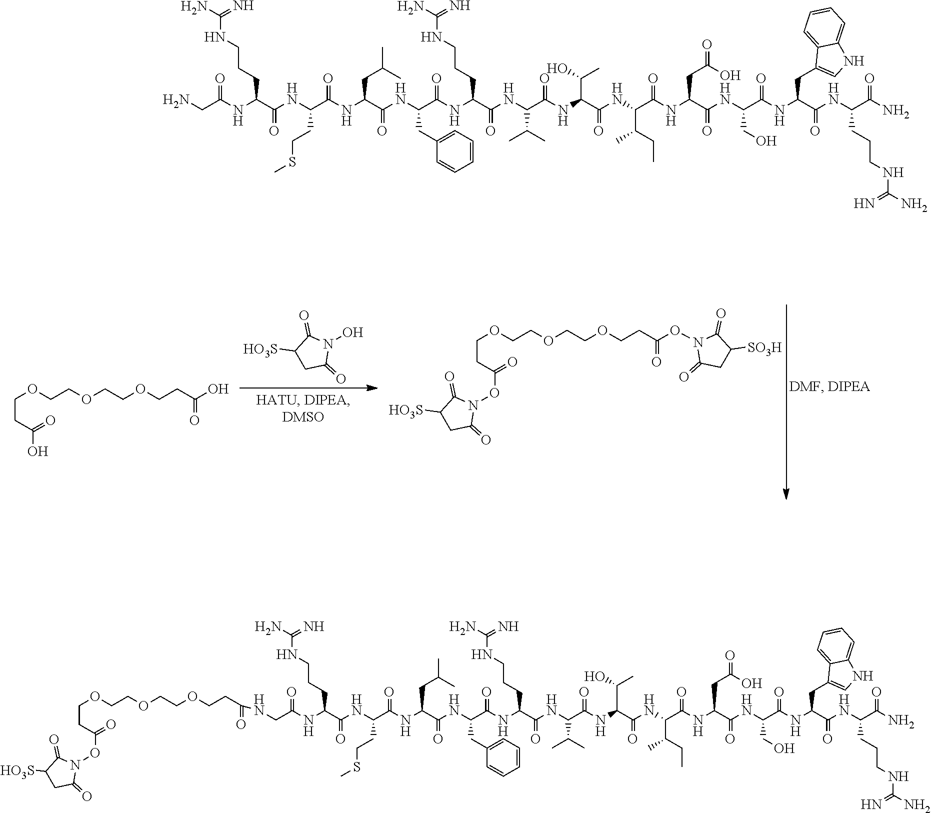

Shown below is a representative scheme for the synthesis of PEG-linked peptide SulfoSE linked to a fluorophore.

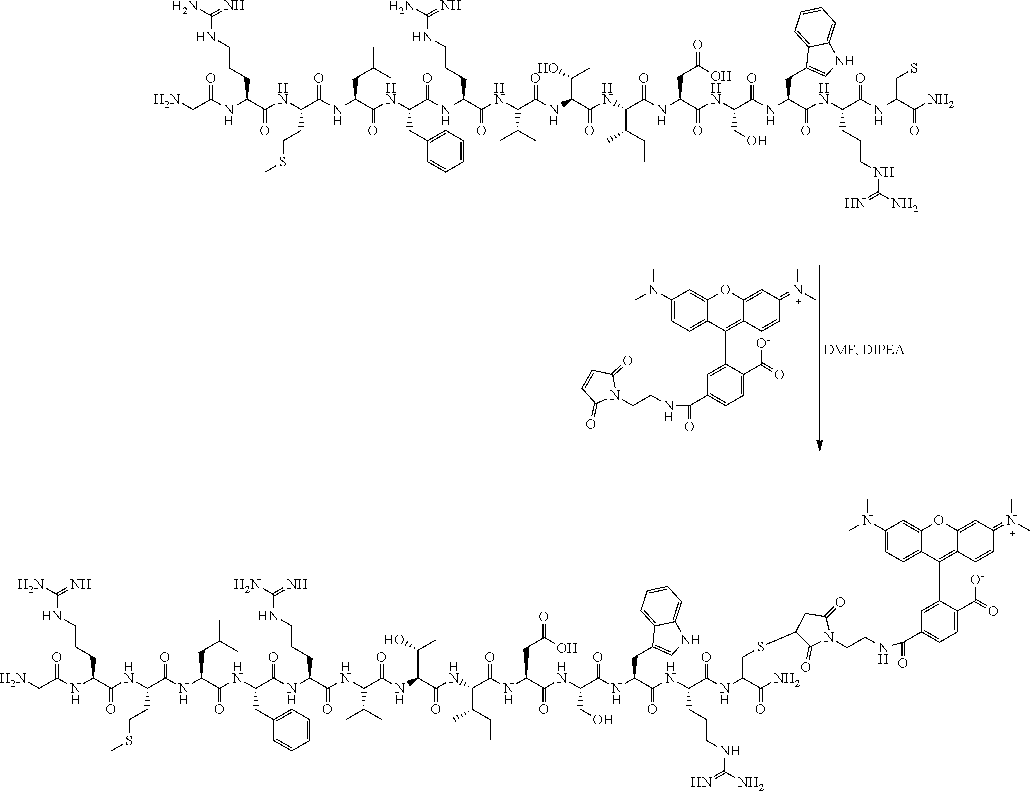

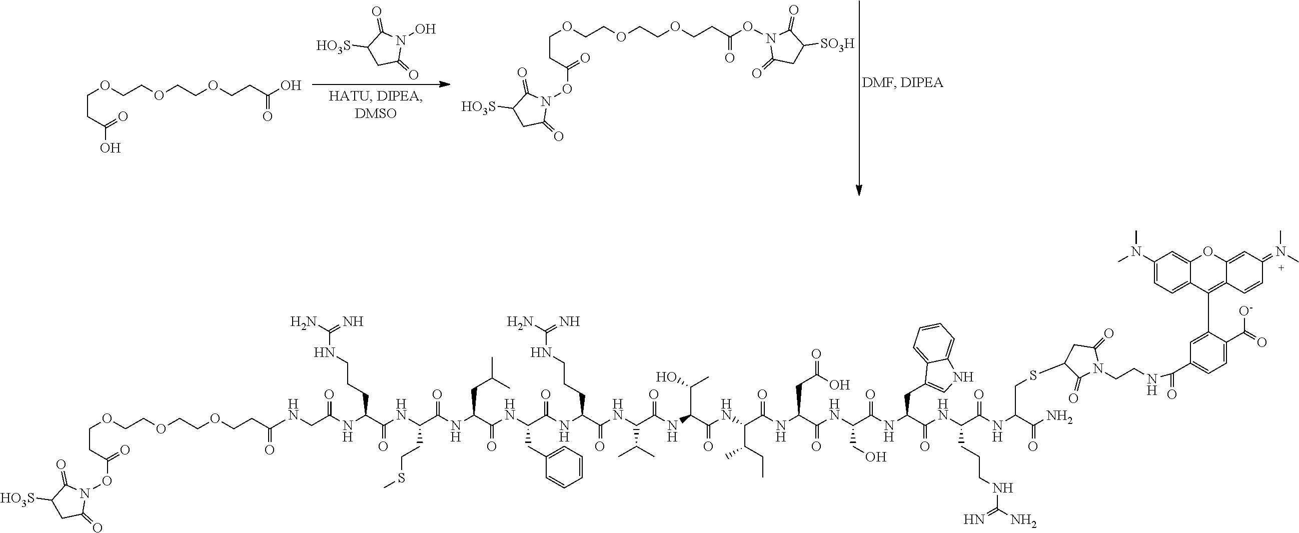

### Example 42

### Investigating Luminescence in Complex Sample Matrices on Performance of Coelenterazine Derivatives JRW-1404 and JRW-1482

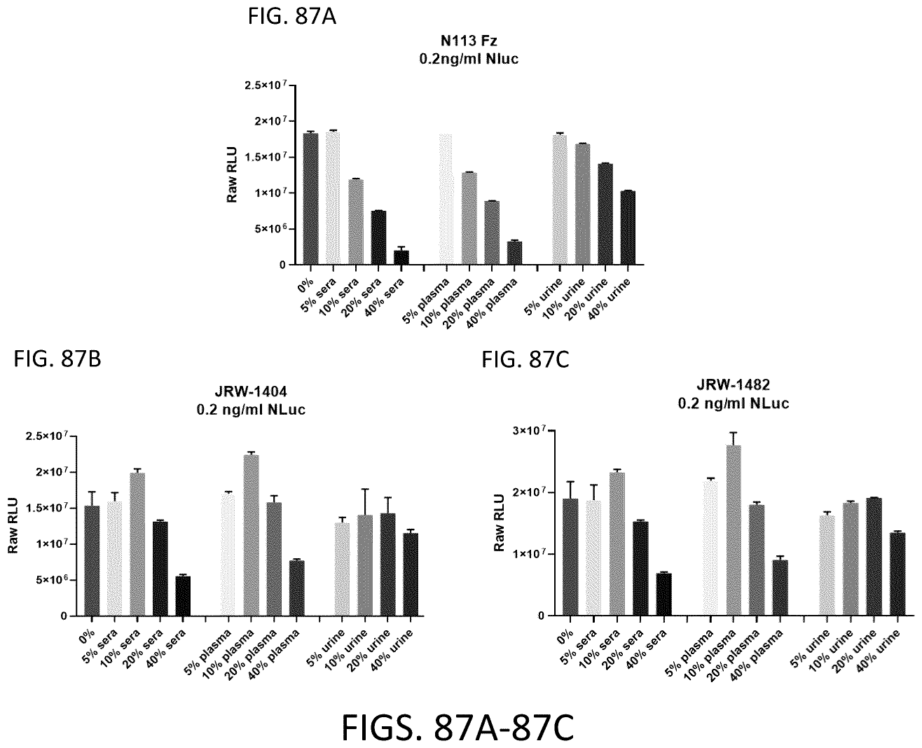

FIG. 87 displays the luminescence derived from coelenterazine derivative substrates JRW-1404 and JRW-1482 in complex sample matrices. 100% samples of plasma (12/28/18), urine (Innovative research 2/25/19), and Human-Sera (2/11/19) were diluted to 10%, 20%, 0%, and 80% in PBS. The sample with “0%” is PBS. In duplicate, 50 1a of each sample was combined with 50 1a NanoLuc diluted to 0.4 ng/ml in PBS. Each substrate was diluted to 20 μM PBS and then 1001 of each diluted substrate was added to the NanoLuc/sample mixtures. Luminescence was measured on a GloMax® Discover plate luminometer.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

### Sequences

The following polypeptide sequences each comprise an N-terminal methionine residue or corresponding ATG codon; polypeptide sequences lacking the N-terminal methionine residue or corresponding ATG codon are also within the scope herein and are incorporated herein by reference.

The following peptide sequences each lack an N-terminal methionine residue; peptide sequences comprising an N-terminal methionine residue are also within the scope herein and are incorporated herein by reference.

TABLE 2
Exemplary peptide, dipeptide, and polypeptide sequences.

| SEQ | | |
|---|---|---|
| ID | | |
| NO | Name | Sequence |
| 1 | WT OgLuc | MFTLADFVGDWQQTAGYNQDQVLEQGGLSSLFQALGVSVTPIQKVVLSGENGLKADIHVIIPYEGLSGFQMGL |
| | | IEMIFKVVYPVDDHHFKIILHYGTLVIDGVTPNMIDYFGRPYPGIAVFDGKQITVTGTLWNGNKIYDERLINP |
| | | DGSLLFRVTINGVTGWRLCENILA |
| 28 | WT OgLuc | atggtgtttaccttggcagatttcgttggagactggcaacagacagctggatacaaccaagatcaagtgttag |
| | | aacaaggaggattgtctagtctgttccaagccctgggagtgtcagtcaccccaatccagaaagttgtgctgtc |
| | | tggggagaatgggttaaaagctgatattcatgtcatcatcccttacgagggactcagtggttttcaaatgggt |
| | | ctgattgaaatgatcttcaaagttgtttacccagtggatgatcatcatttcaagattattctccattatggta |
| | | cactcgttattgacggtgtgacaccaaacatgattgactactttggacgcccttaccctggaattgctgtgtt |
| | | tgacggcaagcagatcacagttactggaactctgtggaacggcaacaagatctatgatgagcgcctgatcaac |
| | | ccagatggttcactcctcttccgcgttactatcaatggagtcaccggatggcgcctttgcgagaacattcttg |
| | | cc |
| 5 | NanoLuc | MVFTLEDFVGDWRQTAGYNLDQVLEQGGVSSLFQNLGVSVTPIQRIVLSGENGLKIDIHVIIPYEGLSGDQMG |
| | | QIEKIFKVVYPVDDHHFKVILHYGTLVIDGVTPNMIDYFGRPYEGIAVFDGKKITVTGTLWNGNKIIDERLIN |
| | | PDGSLLFRVTINGVTGWRLCERILA |
| 29 | NanoLuc | atgaaacatcaccatcaccatcatgcgatcgccatggtcttcacactcgaagatttcgttggggactggcgac |
| | | agacagccggctacaacctggaccaagtccttgaacagggaggtgtgtccagtttgtttcagaatctcggggt |
| | | gtccgtaactccgatccaaaggattgtcctgagcggtgaaaatgggctgaagatcgacatccatgtcatcatc |
| | | ccgtatgaaggtctgagcggcgaccaaatgggccagatcgaaaaaatttttaaggtggtgtaccctgtggatg |
| | | atcatcactttaaggtgatcctgcactatggcacactggtaatcgacggggttacgccgaacatgatcgacta |
| | | tttcggacggccgtatgaaggcatcgccgtgttcgacggcaaaaagatcactgtaacagggaccctgtggaac |
| | | ggcaacaaaattatcgacgagcgcctgatcaaccccgacggctccctgctgttccgagtaaccatcaacggag |
| | | tgaccggctggcggctgtgcgaacgcattctggcggtt |
| 2 | WT OgLuc Lg | MFTLADFVGDWQQTAGYNQDQVLEQGGLSSLFQALGVSVTPIQKVVLSGENGLKADIHVIIPYEGLSGFQMGL |
| | | IEMIFKVVYPVDDHHFKIILHYGTLVIDGVTPNMIDYFGRPYPGIAVFDGKQITVTGTLWNGNKIYDERLINP |
| | | D |
| 3 | WT OgLuc β9 | GSLLFRVTIN |
| 4 | WT OgLuc β10 | GVTGWRLCENILA |
| 6 | WT NanoLuc Lg | MVFTLEDFVGDWRQTAGYNLDQVLEQGGVSSLFQNLGVSVTPIQRIVLSGENGLKIDIHVIIPYEGLSGDQMG |
| | | QIEKIFKVVYPVDDHHFKVILHYGTLVIDGVTPNMIDYFGRPYEGIAVFDGKKITVTGTLWNGNKIIDERLIN |
| | | PD |
| 7 | WT NanoLuc β9 | GSLLFRVTINV |
| 8 | WT NanoLuc β10 | GVTGWRLCERILA |
| 9 | LgBit | MVFTLEDFVGDWEQTAAYNLDQVLEQGGVSSLLQNLAVSVTPIQRIVRSGENALKIDIHVIIPYEGLSADQMA |
| | | QIEEVFKVVYPVDDHHFKVILPYGTLVIDGVTPNMLNYFGRPYEGIAVFDGKKITVTGTLWNGNKIIDERLIT |
| | | PDGSMLFRVTIN |
| 30 | LgBit | atggtcttcacactcgaagatttcgttggggactgggaacagacagccgcctacaacctggaccaagtccttg |
| | | aacagggaggtgtgtccagtttgctgcagaatctcgccgtgtccgtaactccgatccaaaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacggcaacaaaattatcgacgagcgcctgatcacc |
| | | cccgacggctccatgctgttccgagtaaccatcaacagccatcatcaccatcaccac |
| 10 | SmBit | VTGYRLFEEIL |
| 31 | SmBit | gtgaccggctaccggctgttcgaggagattctg |
| 11 | HiBit | VSGWRLFKKIS |
| 32 | HiBit | gtgagcggctggcggctgttcaagaagattagc |
| 33 | LgTrip 2098 | MVFTLEDFVGDWEQTAAYNLDQVLEQGGVSSLLQNLAVSVTPIQRIVRSGENALKIDIHVIIPYEGLSADQMA |
| | | QIEEVFKVVYPVDDHHFKVILPYGTLVIDGVTPNMLNYFGRPYEGIAVFDGKKITVTGTLWNGNKIIDERLIT |
| | | PD |
| 34 | LgTrip 2098 | atggtcttcacactcgaagatttcgttggggactgggaacagacagccgcctacaacctggaccaagtccttg |
| | | aacagggaggtgtgtccagtttgctgcagaatctcgccgtgtccgtaactccgatccaaaggattgtccggag |
| | | cggtgaaaatgCcctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacggcaacaaaattatcgacgagcgcctgatcacc |
| | | cccgac |
| 35 | LgTrip 3092 His | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKITVTGTLWNGNKI |
| | | IDERLITPD |
| 36 | LgTrip 3092 His | atgaaacatcaccatcaccatcatgtcttcacactcgaagatttcgttggggactgggaacagacagccgcct |
| | | acaacctggaccaagtccttgaacagggaggtgtgtccagtttgctgcagaatctcgccgtgtccgtaactcc |
| | | gatccaaaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacatgctgaactatttcggacggcc |
| | | gtatgaaggcatcgccgtgttcgacggcaaaaagatcactgtaacagggaccctgtggaacggcaacaaaatt |
| | | atcgacgagcgcctgatcacccccgac |
| 37 | LgTrip 3092 | MVFTLDDFVGDWEQTAAYNLDQVLEQGGVSSLLQNLAVSVTPIMRIVRSGENALKIDIHVIIPYEGLSADQMA |
| | | QIEEVFKVVYPVDDHHFKVILPYGTLVIDGVTPNKLNYFGRPYEGIAVFDGKKITVTGTLWNGNKIIDERLIT |
| | | PD |
| 38 | LgTrip 3092 | atggtcttcacactcgacgatttcgttggggactgggaacagacagccgcctacaacctggaccaagtccttg |
| | | aacagggaggtgtgtccagtttgctgcagaatctcgccgtgtccgtaactccgatcatgaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacaagctgaactatttcggacggccgtatgaaggcatcgccgtgtt |
| | | cgacggcaaaaagatcactgtaacagggaccctgtggaacggcaacaaaattatcgacgagcgcctgatcacc |
| | | cccgac |
| 13 | SmTrip9 | GSMLFRVTINS |
| 39 | SmTrip9 | ggctccatgctgttccgagtaaccatcaacagc |
| 15 | SmTrip10 | VSGWRLFKKIS |
| 40 | SmTrip10 | gtgagcggctggcggctgttcaagaagattagc |
| 41 | 5P-B9 | MVFTLEDFVGDWEQTAAYNLDQVLEQGGVSSLFQNLAVSVTPIQRIVLSGENALKIDIHVIIPYEGLSADQMA |
| | | QIEKIFKVVYPVDDHHFKVILHYGTLVIDGVTPNMINYFGRPYEGIAVFDGKKITVTGTLWNGNKIIDERLIT |
| | | PD |
| 42 | 5P-B9 | atggtcttcacactcgaagatttcgttggggactgggaacagacagccgcctacaacctggaccaagtccttg |
| | | aacagggaggtgtgtccagtttgtttcagaatctcgccgtgtccgtaactccgatccaaaggattgtcctgag |
| | | cggtgaaaatgccctgaagatcgacatccatgtcatcatcccgtatgaaggtctgagcgccgaccaaatggcc |
| | | cagatcgaaaaaatttttaaggtggtgtaccctgtggatgatcatcactttaaggtgatcctgcactatggca |
| | | cactggtaatcgacggggttacgccgaacatgatcaactatttcggacggccgtatgaaggcatcgccgtgtt |
| | | cgacggcaaaaagatcactgtaacagggaccctgtggaacggcaacaaaattatcgacgagcgcctgatcacc |
| | | cccgac |
| 43 | 5P(147-157) | GSMLFRVTINV |
| 44 | 5P(147-157) | ggctccatgctgttccgagtaaccatcaac |
| 45 | LgTrip 2098 His | MKHHHHHHVFTLEDFVGDWEQTAAYNLDQVLEQGGVSSLLQNLAVSVTPIQRIVRSGENALKIDIHVIIPYEG |
| | | LSADQMAQIEEVFKVVYPVDDHHFKVILPYGTLVIDGVTPNMLNYFGRPYEGIAVFDGKKITVTGTLWNGNKI |
| | | IDERLITPD |
| 46 | LgTrip 2098 His | atgaaacatcaccatcaccatcatgtcttcacactcgaagatttcgttggggactgggaacagacagccgcct |
| | | acaacctggaccaagtccttgaacagggaggtgtgtccagtttgctgcagaatctcgccgtgtccgtaactcc |
| | | gatccaaaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacatgctgaactatttcggacggcc |
| | | gtatgaaggcatcgccgtgttcgacggcaaaaagatcactgtaacagggaccctgtggaacggcaacaaaatt |
| | | atcgacgagcgcctgatcacccccgac |
| 14 | SmTrip9/10 | GSMLFRVTINSVSGWRLFKKIS |
| | Dipeptide | |
| | (pep263) | |
| 47 | SmTrip9/10 | ggctccatgctgttccgagtaaccatcaacagcgtgagcggctggcggctgttcaagaagattagc |
| | Dipeptide | |
| | (Pep263) | |
| 48 | SmTrip9+ | SSWKRGSMLFRVTINS |
| | (pep286) | |
| 49 | SmTrip9+ | Agcagctggaagcgcggctccatgctgttccgagtaaccatcaacagc |
| | (pep286) | |
| 50 | LgTrip 3440 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGDTPNKLNYFGRPYDGIAVFDGKKITVTGTLWNGNKI |
| | | IDERLITPD |
| 51 | LgTrip 3440 | atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggatacgccgaacaagctgaactatttcggacggcc |
| | | gtatgatggcatcgccgtgttcgacggcaaaaagatcactgtaacagggaccctgtggaacggcaacaaaatt |
| | | atcgacgagcgcctgatcacccccgac |
| 52 | LgTrip 3121 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPSKLNYFGRPYEGIAVFDGKKITVTGTLWNGNKI |
| | | IDERLITPD |
| 53 | LgTrip 3121 | atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgagcaagctgaactatttcggacggcc |
| | | gtatgaaggcatcgccgtgttcgacggcaaaaagatcactgtaacagggaccctgtggaacggcaacaaaatt |
| | | atcgacgagcgcctgatcacccccgac |
| 54 | LgTrip 3482 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGFAVFDGKKITVTGTLWNGNKI |
| | | IDERLITPD |
| 55 | LgTrip 3482 | atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcttcgccgtgttcgacggcaaaaagatcactgtaacagggaccctgtggaacggcaacaaaatt |
| | | atcgacgagcgcctgatcacccccgac |
| 56 | LgTrip 3497 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVCDGKKITVTGTLWNGNKI |
| | | IDERLITPD |
| 57 | LgTrip 3497 | atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgtgcgacggcaaaaagatcactgtaacagggaccctgtggaacggcaacaaaatt |
| | | atcgacgagcgcctgatcacccccgac |
| 58 | LgTrip 3125 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKISVTGTLWNGNKI |
| | | IDERLITPD |
| 59 | LgTrip 3125 | atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcgggcggcc |
| | | gtatgaaggcatcgccgtgttcgacggcaaaaagatctctgtaacagggaccctgtggaacggcaacaaaatt |
| | | atcgacgagcgcctgatcacccccgac |
| 60 | LgTrip 3118 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKITATGTLWNGNKI |
| | | IDERLITPD |
| 61 | LgTrip 3118 | atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactgcaacagggaccctgtggaacggcaacaaaatt |
| | | atcgacgagcgcctgatcacccccgac |
| 12 | LgTrip 3546 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKITTTGTLWNGNKI |
| | | IDERLITPD |
| 62 | LgTrip 3546 | atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactaccacagggaccctgtggaacggcaacaaaatt |
| | | atcgacgagcgcctgatcacccccgac |
| 63 | LgTrip 3546 + G | MVFTLDDFVGDWEQTAAYNLDQVLEQGGVSSLLQNLAVSVTPIMRIVRSGENALKIDIHVIIPYEGLSADQMA |
| | (ATG 3572) | QIEEVFKVVYPVDDHHFKVILPYGTLVIDGVTPNKLNYFGRPYEGIAVFDGKKITTTGTLWNGNKIIDERLIT |
| | | PDG |
| 64 | LgTrip 3546 + G | atggtcttcacactcgacgatttcgttggggactgggaacagacagccgcctacaacctggaccaagtccttg |
| | (ATG 3572) | aacagggaggtgtgtccagtttgctgcagaatctcgccgtgtccgtaactccgatcatgaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacaagctgaactatttcggacggccgtatgaaggcatcgccgtgtt |
| | | cgacggcaaaaagatcactaccacagggaccctgtggaacggcaacaaaattatcgacgagcgcctgatcacc |
| | | cccgacggc |
| 65 | LgTrip 3546 − D | MVFTLDDFVGDWEQTAAYNLDQVLEQGGVSSLLQNLAVSVTPIMRIVRSGENALKIDIHVIIPYEGLSADQMA |
| | (ATG 3573) | QIEEVFKVVYPVDDHHFKVILPYGTLVIDGVTPNKLNYFGRPYEGIAVFDGKKITTTGTLWNGNKIIDERLIT |
| | | P |
| 66 | LgTrip 3546 − D | atggtcttcacactcgacgatttcgttggggactgggaacagacagccgcctacaacctggaccaagtccttg |
| | (ATG 3573) | aacagggaggtgtgtccagtttgctgcagaatctcgccgtgtccgtaactccgatcatgaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacaagctgaactatttcggacggccgtatgaaggcatcgccgtgtt |
| | | cgacggcaaaaagatcactaccacagggaccctgtggaacggcaacaaaattatcgacgagcgcctgatcacc |
| | | ccc |
| 67 | LgTrip 3546 − PD | MVFTLDDFVGDWEQTAAYNLDQVLEQGGVSSLLQNLAVSVTPIMRIVRSGENALKIDIHVIIPYEGLSADQMA |
| | (ATG 3574) | QIEEVFKVVYPVDDHHFKVILPYGTLVIDGVTPNKLNYFGRPYEGIAVFDGKKITTTGTLWNGNKIIDERLIT |
| 68 | LgTrip 3546 − PD | atggtcttcacactcgacgatttcgttggggactgggaacagacagccgcctacaacctggaccaagtccttg |
| | (ATG 3574) | aacagggaggtgtgtccagtttgctgcagaatctcgccgtgtccgtaactccgatcatgaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacaagctgaactatttcggacggccgtatgaaggcatcgccgtgtt |
| | | cgacggcaaaaagatcactaccacagggaccctgtggaacggcaacaaaattatcgacgagcgcctgatcacc |
| 69 | LgTrip 3546 + GS | MVFTLDDFVGDWEQTAAYNLDQVLEQGGVSSLLQNLAVSVTPIMRIVRSGENALKIDIHVIIPYEGLSADQMA |
| | (ATG 3575) | QIEEVFKVVYPVDDHHFKVILPYGTLVIDGVTPNKLNYFGRPYEGIAVFDGKKITTTGTLWNGNKIIDERLIT |
| | | PDGS |
| 70 | LgTrip 3546 + GS | atggtcttcacactcgacgatttcgttggggactgggaacagacagccgcctacaacctggaccaagtccttg |
| | (ATG 3575) | aacagggaggtgtgtccagtttgctgcagaatctcgccgtgtccgtaactccgatcatgaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacaagctgaactatttcggacggccgtatgaaggcatcgccgtgtt |
| | | cgacggcaaaaagatcactaccacagggaccctgtggaacggcaacaaaattatcgacgagcgcctgatcacc |
| | | cccgacggcagc |
| 71 | -V_LgBiT | MFTLEDFVGDWEQTAAYNLDQVLEQGGVSSLLQNLAVSVTPIQRIVRSGENALKIDIHVIIPYEGLSADQMAQ |
| | (ATG3618) | IEEVFKVVYPVDDHHFKVILPYGTLVIDGVTPNMLNYFGRPYEGIAVFDGKKITVTGTLWNGNKIIDERLITP |
| | | DGSMLFRVTINSHHHHHH |
| 72 | -V_LgBiT | atgttcacactcgaagatttcgttggggactgggaacagacagccgcctacaacctggaccaagtccttgaac |
| | (ATG3618) | agggaggtgtgtccagtttgctgcagaatctcgccgtgtccgtaactccgatccaaaggattgtccggagcgg |
| | | tgaaaatgccctgaagatcgacatccatgtcatcatcccgtatgaaggtctgagcgccgaccaaatggcccag |
| | | atcgaagaggtgtttaaggtggtgtaccctgtggatgatcatcactttaaggtgatcctgccctatggcacac |
| | | tggtaatcgacggggttacgccgaacatgctgaactatttcggacggccgtatgaaggcatcgccgtgttcga |
| | | cggcaaaaagatcactgtaacagggaccctgtggaacggcaacaaaattatcgacgagcgcctgatcaccccc |
| | | gacggctccatgctgttccgagtaaccatcaacagccatcatcaccatcaccactaa |
| 73 | -VF_LgBiT | MTLEDFVGDWEQTAAYNLDQVLEQGGVSSLLQNLAVSVTPIQRIVRSGENALKIDIHVIIPYEGLSADQMAQI |
| | (ATG3619) | EEVFKVVYPVDDHHFKVILPYGTLVIDGVTPNMLNYFGRPYEGIAVFDGKKITVTGTLWNGNKIIDERLITPD |
| | | GSMLFRVTINSHHHHHH |
| 74 | -VF_LgBiT | atgacactcgaagatttcgttggggactgggaacagacagccgcctacaacctggaccaagtccttgaacagg |
| | (ATG3619) | gaggtgtgtccagtttgctgcagaatctcgccgtgtccgtaactccgatccaaaggattgtccggagcggtga |
| | | aaatgccctgaagatcgacatccatgtcatcatcccgtatgaaggtctgagcgccgaccaaatggcccagatc |
| | | gaagaggtgtttaaggtggtgtaccctgtggatgatcatcactttaaggtgatcctgccctatggcacactgg |
| | | taatcgacggggttacgccgaacatgctgaactatttcggacggccgtatgaaggcatcgccgtgttcgacgg |
| | | caaaaagatcactgtaacagggaccctgtggaacggcaacaaaattatcgacgagcgcctgatcacccccgac |
| | | ggctccatgctgttccgagtaaccatcaacagccatcatcaccatcaccactaa |
| 75 | -VFT_LgBiT | MLEDFVGDWEQTAAYNLDQVLEQGGVSSLLQNLAVSVTPIQRIVRSGENALKIDIHVIIPYEGLSADQMAQIE |
| | (ATG3620) | EVFKVVYPVDDHHFKVILPYGTLVIDGVTPNMLNYFGRPYEGIAVFDGKKITVTGTLWNGNKIIDERLITPDG |
| | | SMLFRVTINSHHHHHH |
| 76 | -VFT_LgBiT | atgctcgaagatttcgttggggactgggaacagacagccgcctacaacctggaccaagtccttgaacagggag |
| | (ATG3620) | gtgtgtccagtttgctgcagaatctcgccgtgtccgtaactccgatccaaaggattgtccggagcggtgaaaa |
| | | tgccctgaagatcgacatccatgtcatcatcccgtatgaaggtctgagcgccgaccaaatggcccagatcgaa |
| | | gaggtgtttaaggtggtgtaccctgtggatgatcatcactttaaggtgatcctgccctatggcacactggtaa |
| | | tcgacggggttacgccgaacatgctgaactatttcggacggccgtatgaaggcatcgccgtgttcgacggcaa |
| | | aaagatcactgtaacagggaccctgtggaacggcaacaaaattatcgacgagcgcctgatcacccccgacggc |
| | | tccatgctgttccgagtaaccatcaacagccatcatcaccatcaccactaa |
| 77 | -VFTL_LgBiT | MEDFVGDWEQTAAYNLDQVLEQGGVSSLLQNLAVSVTPIQRIVRSGENALKIDIHVIIPYEGLSADQMAQIEE |
| | (ATG3621) | VFKVVYPVDDHHFKVILPYGTLVIDGVTPNMLNYFGRPYEGIAVFDGKKITVTGTLWNGNKIIDERLITPDGS |
| | | MLFRVTINSHHHHHH |
| 78 | -VFTL_LgBiT | atggaagatttcgttggggactgggaacagacagccgcctacaacctggaccaagtccttgaacagggaggtg |
| | (ATG3621) | tgtccagtttgctgcagaatctcgccgtgtccgtaactccgatccaaaggattgtccggagcggtgaaaatgc |
| | | cctgaagatcgacatccatgtcatcatcccgtatgaaggtctgagcgccgaccaaatggcccagatcgaagag |
| | | gtgtttaaggtggtgtaccctgtggatgatcatcactttaaggtgatcctgccctatggcacactggtaatcg |
| | | acggggttacgccgaacatgctgaactatttcggacggccgtatgaaggcatcgccgtgttcgacggcaaaaa |
| | | gatcactgtaacagggaccctgtggaacggcaacaaaattatcgacgagcgcctgatcacccccgacggctcc |
| | | atgctgttccgagtaaccatcaacagccatcatcaccatcaccactaa |
| 79 | (M)FKKIS- | MFKKISGSSGVFTLEDFVGDWEQTAAYNLDQVLEQGGVSSLLQNLAVSVTPIQRIVRSGENALKIDIHVIIPY |
| | GSSG-LgBiT | EGLSADQMAQIEEVFKVVYPVDDHHFKVILPYGTLVIDGVTPNMLNYFGRPYEGIAVFDGKKITVTGTLWNGN |
| | (ATG3632) | KIIDERLITPDGSMLFRVTINSHHHHHH |
| 80 | (M)FKKIS- | atgttcaagaagattagcggctcgagcggtgtcttcacactcgaagatttcgttggggactgggaacagacag |
| | GSSG-LgBiT | ccgcctacaacctggaccaagtccttgaacagggaggtgtgtccagtttgctgcagaatctcgccgtgtccgt |
| | (ATG3632) | aactccgatccaaaggattgtccggagcggtgaaaatgccctgaagatcgacatccatgtcatcatcccgtat |
| | | gaaggtctgagcgccgaccaaatggcccagatcgaagaggtgtttaaggtggtgtaccctgtggatgatcatc |
| | | actttaaggtgatcctgccctatggcacactggtaatcgacggggttacgccgaacatgctgaactatttcgg |
| | | acggccgtatgaaggcatcgccgtgttcgacggcaaaaagatcactgtaacagggaccctgtggaacggcaac |
| | | aaaattatcgacgagcgcctgatcacccccgacggctccatgctgttccgagtaaccatcaacagccatcatc |
| | | accatcaccactaa |
| 81 | (M)KKIS-GSSG- | MKKISGSSGVFTLEDFVGDWEQTAAYNLDQVLEQGGVSSLLQNLAVSVTPIQRIVRSGENALKIDIHVIIPYE |
| | LgBiT | GLSADQMAQIEEVFKVVYPVDDHHFKVILPYGTLVIDGVTPNMLNYFGRPYEGIAVFDGKKITVTGTLWNGNK |
| | (ATG3633) | IIDERLITPDGSMLFRVTINSHHHHHH |
| 82 | (M)KKIS-GSSG- | atgaagaagattagcggctcgagcggtgtcttcacactcgaagatttcgttggggactgggaacagacagccg |
| | LgBiT | cctacaacctggaccaagtccttgaacagggaggtgtgtccagtttgctgcagaatctcgccgtgtccgtaac |
| | (ATG3633) | tccgatccaaaggattgtccggagcggtgaaaatgccctgaagatcgacatccatgtcatcatcccgtatgaa |
| | | ggtctgagcgccgaccaaatggcccagatcgaagaggtgtttaaggtggtgtaccctgtggatgatcatcact |
| | | ttaaggtgatcctgccctatggcacactggtaatcgacggggttacgccgaacatgctgaactatttcggacg |
| | | gccgtatgaaggcatcgccgtgttcgacggcaaaaagatcactgtaacagggaccctgtggaacggcaacaaa |
| | | attatcgacgagcgcctgatcacccccgacggctccatgctgttccgagtaaccatcaacagccatcatcacc |
| | | atcaccactaa |
| 83 | (M)KIS-GSSG- | MKISGSSGVFTLEDFVGDWEQTAAYNLDQVLEQGGVSSLLQNLAVSVTPIQRIVRSGENALKIDIHVIIPYEG |
| | LgBiT | LSADQMAQIEEVFKVVYPVDDHHFKVILPYGTLVIDGVTPNMLNYFGRPYEGIAVFDGKKITVTGTLWNGNKI |
| | (ATG3634) | IDERLITPDGSMLFRVTINSHHHHHH |
| 84 | (M)KIS-GSSG- | atgaagattagcggctcgagcggtgtcttcacactcgaagatttcgttggggactgggaacagacagccgcct |
| | LgBiT | acaacctggaccaagtccttgaacagggaggtgtgtccagtttgctgcagaatctcgccgtgtccgtaactcc |
| | (ATG3634) | gatccaaaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacatgctgaactatttcggacggcc |
| | | gtatgaaggcatcgccgtgttcgacggcaaaaagatcactgtaacagggaccctgtggaacggcaacaaaatt |
| | | atcgacgagcgcctgatcacccccgacggctccatgctgttccgagtaaccatcaacagccatcatcaccatc |
| | | accactaa |
| 85 | (M)IS-GSSG- | MISGSSGVFTLEDFVGDWEQTAAYNLDQVLEQGGVSSLLQNLAVSVTPIQRIVRSGENALKIDIHVIIPYEGL |
| | LgBiT | SADQMAQIEEVFKVVYPVDDHHFKVILPYGTLVIDGVTPNMLNYFGRPYEGIAVFDGKKITVTGTLWNGNKII |
| | (ATG3635) | DERLITPDGSMLFRVTINSHHHHHH |
| 86 | (M)IS-GSSG- | atgattagcggctcgagcggtgtcttcacactcgaagatttcgttggggactgggaacagacagccgcctaca |
| | LgBiT | acctggaccaagtccttgaacagggaggtgtgtccagtttgctgcagaatctcgccgtgtccgtaactccgat |
| | (ATG3635) | ccaaaggattgtccggagcggtgaaaatgccctgaagatcgacatccatgtcatcatcccgtatgaaggtctg |
| | | agcgccgaccaaatggcccagatcgaagaggtgtttaaggtggtgtaccctgtggatgatcatcactttaagg |
| | | tgatcctgccctatggcacactggtaatcgacggggttacgccgaacatgctgaactatttcggacggccgta |
| | | tgaaggcatcgccgtgttcgacggcaaaaagatcactgtaacagggaccctgtggaacggcaacaaaattatc |
| | | gacgagcgcctgatcacccccgacggctccatgctgttccgagtaaccatcaacagccatcatcaccatcacc |
| | | actaa |
| 87 | (M)S-GSSG- | MSGSSGVFTLEDFVGDWEQTAAYNLDQVLEQGGVSSLLQNLAVSVTPIQRIVRSGENALKIDIHVIIPYEGLS |
| | LgBIT | ADQMAQIEEVFKVVYPVDDHHFKVILPYGTLVIDGVTPNMLNYFGRPYEGIAVFDGKKITVTGTLWNGNKIID |
| | (ATG3636) | ERLITPDGSMLFRVTINSHHHHHH |
| 88 | (M)S-GSSG- | atgagcggctcgagcggtgtcttcacactcgaagatttcgttggggactgggaacagacagccgcctacaacc |
| | LgBiT | tggaccaagtccttgaacagggaggtgtgtccagtttgctgcagaatctcgccgtgtccgtaactccgatcca |
| | (ATG3636) | aaggattgtccggagcggtgaaaatgccctgaagatcgacatccatgtcatcatcccgtatgaaggtctgagc |
| | | gccgaccaaatggcccagatcgaagaggtgtttaaggtggtgtaccctgtggatgatcatcactttaaggtga |
| | | tcctgccctatggcacactggtaatcgacggggttacgccgaacatgctgaactatttcggacggccgtatga |
| | | aggcatcgccgtgttcgacggcaaaaagatcactgtaacagggaccctgtggaacggcaacaaaattatcgac |
| | | gagcgcctgatcacccccgacggctccatgctgttccgagtaaccatcaacagccatcatcaccatcaccact |
| | | aa |
| 89 | LgTrip + GSM | MKHHHHHHVFTLDDFVGDWEQTAAYNLDQVLEQGGVSSLLQNLAVSVTPIMRIVRSGENALKIDIHVIIPYEG |
| | (ATG3722) | LSADQMAQIEEVFKVVYPVDDHHFKVILPYGTLVIDGVTPNKLNYFGRPYEGIAVFDGKKITTTGTLWNGNKI |
| | | IDERLITPDGSM |
| 90 | LgTrip + GSM | atgaaacatcaccatcaccatcatgtcttcacactcgacgatttcgttggggactgggaacagacagccgcct |
| | (ATG3722) | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactaccacagggaccctgtggaacggcaacaaaatt |
| | | atcgacgagcgcctgatcacccccgacggcagcatgtaa |
| 91 | LgTrip + GSML | MKHHHHHHVFTLDDFVGDWEQTAAYNLDQVLEQGGVSSLLQNLAVSVTPIMRIVRSGENALKIDIHVIIPYEG |
| | (ATG3723) | LSADQMAQIEEVFKVVYPVDDHHFKVILPYGTLVIDGVTPNKLNYFGRPYEGIAVFDGKKITTTGTLWNGNKI |
| | | IDERLITPDGSML |
| 92 | LgTrip + GSML | atgaaacatcaccatcaccatcatgtcttcacactcgacgatttcgttggggactgggaacagacagccgcct |
| | (ATG3723) | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactaccacagggaccctgtggaacggcaacaaaatt |
| | | atcgacgagcgcctgatcacccccgacggcagcatgctgtaa |
| 93 | LgTrip + GSMLF | MKHHHHHHVFTLDDFVGDWEQTAAYNLDQVLEQGGVSSLLQNLAVSVTPIMRIVRSGENALKIDIHVIIPYEG |
| | (ATG3724) | LSADQMAQIEEVFKVVYPVDDHHFKVILPYGTLVIDGVTPNKLNYFGRPYEGIAVFDGKKITTTGTLWNGNKI |
| | | IDERLITPDGSMLF |
| 94 | LgTrip + GSMLF | atgaaacatcaccatcaccatcatgtcttcacactcgacgatttcgttggggactgggaacagacagccgcct |
| | (ATG3724) | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactaccacagggaccctgtggaacggcaacaaaatt |
| | | atcgacgagcgcctgatcacccccgacggcagcatgctgttctaa |
| 95 | LgTrip - TPD | MKHHHHHHVFTLDDFVGDWEQTAAYNLDQVLEQGGVSSLLQNLAVSVTPIMRIVRSGENALKIDIHVIIPYEG |
| | (ATG3725) | LSADQMAQIEEVFKVVYPVDDHHFKVILPYGTLVIDGVTPNKLNYFGRPYEGIAVFDGKKITTTGTLWNGNKI |
| | | IDERLI |
| 96 | LgTrip - TPD | atgaaacatcaccatcaccatcatgtcttcacactcgacgatttcgttggggactgggaacagacagccgcct |
| | (ATG3725) | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactaccacagggaccctgtggaacggcaacaaaatt |
| | | atcgacgagcgcctgatctaa |
| 97 | LgTrip - ITPD | MKHHHHHHVFTLDDFVGDWEQTAAYNLDQVLEQGGVSSLLQNLAVSVTPIMRIVRSGENALKIDIHVIIPYEG |
| | (ATG3726) | LSADQMAQIEEVFKVVYPVDDHHFKVILPYGTLVIDGVTPNKLNYFGRPYEGIAVFDGKKITTTGTLWNGNKI |
| | | IDERL |
| 98 | LgTrip - ITPD | atgaaacatcaccatcaccatcatgtcttcacactcgacgatttcgttggggactgggaacagacagccgcct |
| | (ATG3726) | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactaccacagggaccctgtggaacggcaacaaaatt |
| | | atcgacgagcgcctgtaa |
| 99 | LgTrip - LITPD | MKHHHHHHVFTLDDFVGDWEQTAAYNLDQVLEQGGVSSLLQNLAVSVTPIMRIVRSGENALKIDIHVIIPYEG |
| | (ATG3727) | LSADQMAQIEEVFKVVYPVDDHHFKVILPYGTLVIDGVTPNKLNYFGRPYEGIAVFDGKKITTTGTLWNGNKI |
| | | IDER |
| 100 | LgTrip - LITPD | atgaaacatcaccatcaccatcatgtcttcacactcgacgatttcgttggggactgggaacagacagccgcct |
| | (ATG3727) | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactaccacagggaccctgtggaacggcaacaaaatt |
| | | atcgacgagcgctaa |
| 101 | FRB-15GS-AI-86 | MVAILWHEMWHEGLEEASRLYFGERNVKGMFEVLEPLHAMMERGPQTLKETSFNQAYGRDLMEAQEWCRKYMK |
| | (ATG1634) | SGNVKDLTQAWDLYYHVFRRISGGSGGGGSGGSSSGGAIVSGWRLFKKIS |
| 102 | FRB-15GS-AI-86 | atggtggccatcctctggcatgagatgtggcatgaaggcctggaagaggcatctcgtttgtactttggggaaa |
| | (ATG1634) | ggaacgtgaaaggcatgtttgaggtgctggagcccttgcatgctatgatggaacggggcccccagactctgaa |
| | | ggaaacatcctttaatcaggcctatggtcgagatttaatggaggcccaagagtggtgcaggaagtacatgaaa |
| | | tcagggaatgtcaaggacctcacccaagcctgggacctctattatcatgtgttccgacgaatcagtggtggtt |
| | | caggtggtggcgggagcggtggctcgagcagcggtggagcgatcgtgagcggctggcggctgttcaagaagat |
| | | tagctaa |
| 103 | FRB-15GS-AI- | MVAILWHEMWHEGLEEASRLYFGERNVKGMFEVLEPLHAMMERGPQTLKETSFNQAYGRDLMEAQEWCRKYMK |
| | 289 (ATG3586) | SGNVKDLTQAWDLYYHVFRRISGGSGGGGSGGSSSGGAIVSVSGWRLFKKIS |
| 104 | FRB-15GS-AI- | atggtggccatcctctggcatgagatgtggcatgaaggcctggaagaggcatctcgtttgtactttggggaaa |
| | 289 (ATG3586) | ggaacgtgaaaggcatgtttgaggtgctggagcccttgcatgctatgatggaacggggcccccagactctgaa |
| | | ggaaacatcctttaatcaggcctatggtcgagatttaatggaggcccaagagtggtgcaggaagtacatgaaa |
| | | tcagggaatgtcaaggacctcacccaagcctgggacctctattatcatgtgttccgacgaatcagtggtggtt |
| | | caggtggtggcgggagcggtggctcgagcagcggtggagcgatcgttagcgttagcggctggcgcctgttcaa |
| | | gaagatcagctaa |
| 105 | FRB-15GS-AI- | MVAILWHEMWHEGLEEASRLYFGERNVKGMFEVLEPLHAMMERGPQTLKETSFNQAYGRDLMEAQEWCRKYMK |
| | 86 − His6 | SGNVKDLTQAWDLYYHVFRRISGGSGGGGSGGSSSGGAIVSGWRLFKKISHHHHHH |
| | (ATG3743) | |
| 106 | FRB-15GS-AI- | atggtggccatcctctggcatgagatgtggcatgaaggcctggaagaggcatctcgtttgtactttggggaaa |
| | 86 − His6 | ggaacgtgaaaggcatgtttgaggtgctggagcccttgcatgctatgatggaacggggcccccagactctgaa |
| | (ATG3743) | ggaaacatcctttaatcaggcctatggtcgagatttaatggaggcccaagagtggtgcaggaagtacatgaaa |
| | | tcagggaatgtcaaggacctcacccaagcctgggacctctattatcatgtgttccgacgaatcagtggtggtt |
| | | caggtggtggcgggagcggtggctcgagcagcggtggagcgatcgtgagcggctggcggctgttcaagaagat |
| | | tagccatcatcaccatcaccactaa |
| 107 | FRB-15GS-AI- | MVAILWHEMWHEGLEEASRLYFGERNVKGMFEVLEPLHAMMERGPQTLKETSFNQAYGRDLMEAQEWCRKYMK |
| | 289-His6 | SGNVKDLTQAWDLYYHVFRRISGGSGGGGSGGSSSGGAIVSVSGWRLFKKISHHHHHH |
| | (ATG3744) | |
| 108 | FRB-15GS-AI- | atggtggccatcctctggcatgagatgtggcatgaaggcctggaagaggcatctcgtttgtactttggggaaa |
| | 289-His6 | ggaacgtgaaaggcatgtttgaggtgctggagcccttgcatgctatgatggaacggggcccccagactctgaa |
| | (ATG3744) | ggaaacatcctttaatcaggcctatggtcgagatttaatggaggcccaagagtggtgcaggaagtacatgaaa |
| | | tcagggaatgtcaaggacctcacccaagcctgggacctctattatcatgtgttccgacgaatcagtggtggtt |
| | | caggtggtggcgggagcggtggctcgagcagcggtggagcgatcgttagcgtgagcggctggcggctgttcaa |
| | | gaagattagccatcatcaccatcaccactaa |
| 109 | His6-FRB-5GS- | MKHHHHHHVAILWHEMWHEGLEEASRLYFGERNVKGMFEVLEPLHAMMERGPQTLKETSFNQAYGRDLMEAQE |
| | 86 (ATG3760) | WCRKYMKSGNVKDLTQAWDLYYHVFRRISGGSGGVSGWRLFKKIS |
| 110 | His6-FRB-5GS- | atgaaacatcaccatcaccatcatgtggccatcctctggcatgagatgtggcatgaaggcctggaagaggcat |
| | 86 (ATG3760) | ctcgtttgtactttggggaaaggaacgtgaaaggcatgtttgaggtgctggagcccttgcatgctatgatgga |
| | | acggggcccccagactctgaaggaaacatcctttaatcaggcctatggtcgagatttaatggaggcccaagag |
| | | tggtgcaggaagtacatgaaatcagggaatgtcaaggacctcacccaagcctgggacctctattatcatgtgt |
| | | tccgacgaatcagtggtggttcaggtggtgtgagcggctggcggctgttcaagaagattagctaa |
| 111 | His6-FRB-10GS- | MKHHHHHHVAILWHEMWHEGLEEASRLYFGERNVKGMFEVLEPLHAMMERGPQTLKETSFNQAYGRDLMEAQE |
| | 86 (ATG3761) | WCRKYMKSGNVKDLTQAWDLYYHVFRRISGGSGGGGSGGVSGWRLFKKIS |
| 112 | His6-FRB-10GS- | atgaaacatcaccatcaccatcatgtggccatcctctggcatgagatgtggcatgaaggcctggaagaggcat |
| | 86 (ATG3761) | ctcgtttgtactttggggaaaggaacgtgaaaggcatgtttgaggtgctggagcccttgcatgctatgatgga |
| | | acggggcccccagactctgaaggaaacatcctttaatcaggcctatggtcgagatttaatggaggcccaagag |
| | | tggtgcaggaagtacatgaaatcagggaatgtcaaggacctcacccaagcctgggacctctattatcatgtgt |
| | | tccgacgaatcagtggtggttcaggtggtggcgggagcggtggcgtgagcggctggcggctgttcaagaagat |
| | | tagctaa |
| 113 | His6-FRB-15GS- | MKHHHHHHVAILWHEMWHEGLEEASRLYFGERNVKGMFEVLEPLHAMMERGPQTLKETSFNQAYGRDLMEAQE |
| | 86 (ATG3762) | WCRKYMKSGNVKDLTQAWDLYYHVFRRISGGSGGGGSGGSSSGGVSGWRLFKKIS |
| 114 | His6-FRB-15GS- | atgaaacatcaccatcaccatcatgtggccatcctctggcatgagatgtggcatgaaggcctggaagaggcat |
| | 86 (ATG3762) | ctcgtttgtactttggggaaaggaacgtgaaaggcatgtttgaggtgctggagcccttgcatgctatgatgga |
| | | acggggcccccagactctgaaggaaacatcctttaatcaggcctatggtcgagatttaatggaggcccaagag |
| | | tggtgcaggaagtacatgaaatcagggaatgtcaaggacctcacccaagcctgggacctctattatcatgtgt |
| | | tccgacgaatcagtggtggttcaggtggtggcgggagcggtggctcgagcagcggtggagtgagcggctggcg |
| | | gctgttcaagaagattagctaa |
| 115 | His6-FRB-5GS- | MKHHHHHHVAILWHEMWHEGLEEASRLYFGERNVKGMFEVLEPLHAMMERGPQTLKETSFNQAYGRDLMEAQE |
| | 289 (ATG3763) | WCRKYMKSGNVKDLTQAWDLYYHVFRRISGGSGGVSVSGWRLFKKIS |
| 116 | His6-FRB-5GS- | atgaaacatcaccatcaccatcatgtggccatcctctggcatgagatgtggcatgaaggcctggaagaggcat |
| | 289 (ATG3763) | ctcgtttgtactttggggaaaggaacgtgaaaggcatgtttgaggtgctggagcccttgcatgctatgatgga |
| | | acggggcccccagactctgaaggaaacatcctttaatcaggcctatggtcgagatttaatggaggcccaagag |
| | | tggtgcaggaagtacatgaaatcagggaatgtcaaggacctcacccaagcctgggacctctattatcatgtgt |
| | | tccgacgaatcagtggtggttcaggtggtgttagcgttagcggctggcgcctgttcaagaagatcagctaa |
| 117 | His6-FRB-10GS- | MKHHHHHHVAILWHEMWHEGLEEASRLYFGERNVKGMFEVLEPLHAMMERGPQTLKETSFNQAYGRDLMEAQE |
| | 289 (ATG3764) | WCRKYMKSGNVKDLTQAWDLYYHVFRRISGGSGGGGSGGVSVSGWRLFKKIS |
| 118 | His6-FRB-10GS- | atgaaacatcaccatcaccatcatgtggccatcctctggcatgagatgtggcatgaaggcctggaagaggcat |
| | 289 (ATG3764) | ctcgtttgtactttggggaaaggaacgtgaaaggcatgtttgaggtgctggagcccttgcatgctatgatgga |
| | | acggggcccccagactctgaaggaaacatcctttaatcaggcctatggtcgagatttaatggaggcccaagag |
| | | tggtgcaggaagtacatgaaatcagggaatgtcaaggacctcacccaagcctgggacctctattatcatgtgt |
| | | tccgacgaatcagtggtggttcaggtggtggcgggagcggtggcgttagcgttagcggctggcgcctgttcaa |
| | | gaagatcagctaa |
| 119 | His6-FRB-15GS- | MKHHHHHHVAILWHEMWHEGLEEASRLYFGERNVKGMFEVLEPLHAMMERGPQTLKETSFNQAYGRDLMEAQE |
| | 289 (ATG3765) | WCRKYMKSGNVKDLTQAWDLYYHVFRRISGGSGGGGSGGSSSGGVSVSGWRLFKKIS |
| 120 | His6-FRB-15GS- | atgaaacatcaccatcaccatcatgtggccatcctctggcatgagatgtggcatgaaggcctggaagaggcat |
| | 289 (ATG3765) | ctcgtttgtactttggggaaaggaacgtgaaaggcatgtttgaggtgctggagcccttgcatgctatgatgga |
| | | acggggcccccagactctgaaggaaacatcctttaatcaggcctatggtcgagatttaatggaggcccaagag |
| | | tggtgcaggaagtacatgaaatcagggaatgtcaaggacctcacccaagcctgggacctctattatcatgtgt |
| | | tccgacgaatcagtggtggttcaggtggtggcgggagcggtggctcgagcagcggtggagttagcgttagcgg |
| | | ctggcgcctgttcaagaagatcagctaa |
| 121 | SmTrip9-FKBP | M-GSMLFRVTINS-SSSGGGGSGGGSSGGGVQVETISPGDGRTFPKRGQTCVVHYTGMLEDGKKFDSSRDRNK |
| | fusion template | PFKFMLGKQEVIRGWEEGVAQMSVGQRAKLTISPDYAYGATGHPGIIPPHATLVFDVELLKLE |
| | (ATG780) | |
| 122 | SmTrip9-FKBP | atgggctccatgctgttccgagtaaccatcaacagctcgagttcaggtggtggcgggagcggtggagggagca |
| | fusion template | gcggtggaggagtgcaggtggaaaccatctccccaggagacgggcgcaccttccccaagcgcggccagacctg |
| | (ATG780) | cgtggtgcactacaccgggatgcttgaagatggaaagaaatttgattcctcccgggacagaaacaagcccttt |
| | | aagtttatgctaggcaagcaggaggtgatccgaggctgggaagaaggggttgcccagatgagtgtgggtcaga |
| | | gagccaaactgactatatctccagattatgcctatggtgccactgggcacccaggcatcatcccaccacatgc |
| | | cactctcgtcttcgatgtggagcttctaaaactggaataa |
| 123 | FKBP-SmTrip9 | MGVQVETISPGDGRTFPKRGQTCVVHYTGMLEDGKKFDSSRDRNKPFKFMLGKQEVIRGWEEGVAQMSVGQRA |
| | fusion template | KLTISPDYAYGATGHPGIIPPHATLVFDVELLKLEGGSGGGGSGGSSSGGAI-GSMLFRVTINS |
| | (ATG777) | |
| 124 | FKBP-SmTrip9 | Atgggagtgcaggtggaaaccatctccccaggagacgggcgcaccttccccaagcgcggccagacctgcgtgg |
| | fusion template | tgcactacaccgggatgcttgaagatggaaagaaatttgattcctcccgggacagaaacaagccctttaagtt |
| | (ATG777) | tatgctaggcaagcaggaggtgatccgaggctgggaagaaggggttgcccagatgagtgtgggtcagagagcc |
| | | aaactgactatatctccagattatgcctatggtgccactgggcacccaggcatcatcccaccacatgccactc |
| | | tcgtcttcgatgtggagcttctaaaactggaaggtggttcaggtggtggcgggagcggtggctcgagcagcgg |
| | | tggagcgatcggctccatgctgttccgagtaaccatcaacagc |
| 125 | LgBiT | MVFTLEDFVGDWEQTAAYNLDQVLEQGGVSSLLQNLAVSVTPIQRIVRSGENALKIDIHVIIPYEGLSADQMA |
| | (ATG2623) | QIEEVFKVVYPVDDHHFKVILPYGTLVIDGVTPNMLNYFGRPYEGIAVFDGKKITVTGTLWNGNKIIDERLIT |
| | | PDGSMLFRVTINSHHHHHH |
| 126 | LgBiT | atggtcttcacactcgaagatttcgttggggactgggaacagacagccgcctacaacctggaccaagtccttg |
| | (ATG2623) | aacagggaggtgtgtccagtttgctgcagaatctcgccgtgtccgtaactccgatccaaaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacggcaacaaaattatcgacgagcgcctgatcacc |
| | | cccgacggctccatgctgttccgagtaaccatcaacagccatcatcaccatcaccactaa |
| 127 | pep78 | NVSGWRLFKKISN |
| 128 | pep79 | NVTGYRLFKKISN |
| 129 | pep80 | VSGWRLFKKISN |
| 130 | pep81 | SGWRLFKKISN |
| 131 | pep82 | GWRLFKKISN |
| 132 | pep99 | VTGYRLFEKIS |
| 133 | pep219 | SGWRLFKKIS |
| 134 | pep225 | VSGWRL |
| 135 | pep226 | VSGWRLF |
| 136 | pep227 | VSGWRLFK |
| 137 | pep228 | VSGWRLFKK |
| 138 | pep229 | VSGWRLFKKI |
| 139 | pep243 | VSGWRLYKKIS |
| 140 | pep272 | GSMLFRVTINSVSGWALFKKIS |
| 141 | pep274 | GSMLFRVTINSVTGYRLFEEIL |
| 142 | pep287 (WT | GSMLFRVTINSSSWKR |
| | SmTrip9) + Cterm | |
| | solubility tag | |
| 143 | pep288 | VSGVSGWRLFKKIS |
| 144 | pep290 | VVSGWRLFKKIS |
| 145 | pep291 | SSWKRSMLFRVTINS |
| 146 | pep292 | SSWKRMLFRVTINS |
| 147 | pep293 | SSWKRDGSMLFRVTINS |
| 148 | pep294 | SSWKRPDGSMLFRVTINS |
| 149 | pep296 | SSWKRSMLFRVTINSV |
| 150 | pep297 | SSWKRMLFRVTINSV |
| 151 | pep298 | SSWKRDGSMLFRVTINSV |
| 152 | pep299 | SSWKRPDGSMLFRVTINSV |
| 153 | pep301 | SSWKRSMLFRVTINSVS |
| 154 | pep302 | SSWKRMLFRVTINSVS |
| 155 | pep303 | SSWKRDGSMLFRVTINSVS |
| 156 | pep304 | SSWKRPDGSMLFRVTINSVS |
| 157 | pep305 | SSWKRGSMLFRVTIN |
| 158 | pep306 | SSWKRGSMLFRVTI |
| 159 | pep307 | SSWKRSMLFRVTIN |
| 160 | pep308 | SSWKRMLFRVTIN |
| 161 | pep309 | SSWKRDGSMLFRVTIN |
| 162 | pep310 | SSWKRPDGSMLFRVTIN |
| 163 | pep311 | SSWKRSMLFRVTI |
| 164 | pep312 | SSWKRMLFRVTI |
| 165 | pep313 | SSWKRDGSMLFRVTI |
| 166 | pep314 | SSWKRPDGSMLFRVTI |
| 167 | pep316 | VSGWRLFKKISVFTL |
| 168 | pep317 | VSGWRLFKKISVFT |
| 169 | pep318 | VSGWRLFKKISVF |
| 170 | pep319 | VSGWRLFKKISV |
| 171 | pep320 | VSGWRLCKKIS |
| 172 | pep326 | VSGWRLFKKISGSMLFRVTINS |
| 173 | pep380 | SSWKRLFRVTINS |
| 174 | pep383 | SSWKRFRVTINS |
| 175 | pep386 | SSWKRRVTINS |
| 176 | pep389 | SSWKRTPDGSMLFRVTINS |
| 177 | pep392 | SSWKRITPDGSMLFRVTINS |
| 178 | pep395 | SSWKRLITPDGSMLFRVTINS |
| 179 | pep396 | SSRGSMLFRVTINSWK |
| 180 | pep397 | SKRGSMLFRVTINSWS |
| 181 | pep398 | SWRGSMLFRVTINS |
| 182 | pep400 | SSRGSMLFRVTIWK |
| 183 | pep401 | SSWKRGSMLYRVTINS |
| 184 | pep402 | SSWKRGSMLWRVTINS |
| 185 | pep403 | SSWKRGSMLHRVTINS |
| 186 | pep404 | SSWKRGSLLFRVTINS |
| 187 | pep405 | SSWKRGSKLFRVTINS |
| 188 | pep406 | SSWKRGSRLFRVTINS |
| 189 | pep407 | SSWKRGSFLFRVTINS |
| 190 | pep408 | SSWKRGSWLFRVTINS |
| 191 | pep409 | SSWKRGSMLFRVSINS |
| 192 | pep410 | SSWKRGSMLFRVQINS |
| 193 | pep411 | SSWKRGSMLFRVNINS |
| 194 | SmTrip9-286 with | SSWKRGSMLFRVTINSC |
| | cysteine | |
| 195 | HiBit with | CVSGWRLFKKIS |
| | cysteine | |
| 196 | SmTrip9-286 with | SSWKRGSMLFRVTINSK(Aza) |
| | azide | |
| 197 | HiBit with azide | (aza)KVSGWRLFKKIS |
| 198 | WT OgLuc | GSLLFRVTINGVTGWRLCENILA |
| | dipeptide | |
| 199 | WT NanoLuc | GSLLFRVTINVGVTGWRLCERILA |
| | dipeptide | |
| 200 | pep157 | SVSGWRLFKKIS |
| 201 | pep158 | NSVSGWRLFKKIS |
| 202 | pep206 | GWRLFKKIS |
| 203 | HiBiT-His- | Atggtgagcggctggcggctgttcaagaagattagccaccatcaccatcaccatcatcacttcacactcgacg |
| | LgTrip3546 | atttcgttggggactgggaacagacagccgcctacaacctggaccaagtccttgaacagggaggtgtgtccag |
| | (ATG 3745) | tttgctgcagaatctcgccgtgtccgtaactccgatcatgaggattgtccggagcggtgaaaatgccctgaag |
| | | atcgacatccatgtcatcatcccgtatgaaggtctgagcgccgaccaaatggcccagatcgaagaggtgttta |
| | | aggtggtgtaccctgtggatgatcatcactttaaggtgatcctgccctatggcacactggtaatcgacggggt |
| | | tacgccgaacaagctgaactatttcggacggccgtatgaaggcatcgccgtgttcgacggcaaaaagatcact |
| | | accacagggaccctgtggaacggcaacaaaattatcgacgagcgcctgatcacccccgactaa |
| 204 | HiBiT-His- | MVSGWRLFKKISHHHHHHHHFTLDDFVGDWEQTAAYNLDQVLEQGGVSSLLQNLAVSVTPIMRIVRSGENALK |
| | LgTrip3546 | IDIHVIIPYEGLSADQMAQIEEVFKVVYPVDDHHFKVILPYGTLVIDGVTPNKLNYFGRPYEGIAVFDGKKIT |
| | (ATG 3745) | TTGTLWNGNKIIDERLITPD |
| 205 | His-HiBiT- | Atgaaacatcaccatcaccatcatgtgagcggctggcggctgttcaagaagattagcggcagctccggtttca |
| | GSSG- | cactcgacgatttcgttggggactgggaacagacagccgcctacaacctggaccaagtccttgaacagggagg |
| | LgTrip3546 | tgtgtccagtttgctgcagaatctcgccgtgtccgtaactccgatcatgaggattgtccggagcggtgaaaat |
| | (ATG 3746) | gccctgaagatcgacatccatgtcatcatcccgtatgaaggtctgagcgccgaccaaatggcccagatcgaag |
| | | aggtgtttaaggtggtgtaccctgtggatgatcatcactttaaggtgatcctgccctatggcacactggtaat |
| | | cgacggggttacgccgaacaagctgaactatttcggacggccgtatgaaggcatcgccgtgttcgacggcaaa |
| | | aagatcactaccacagggaccctgtggaacggcaacaaaattatcgacgagcgcctgatcacccccgactaa |
| 206 | His-HiBiT- | MKHHHHHHVSGWRLFKKISGSSGFTLDDFVGDWEQTAAYNLDQVLEQGGVSSLLQNLAVSVTPIMRIVRSGEN |
| | GSSG- | ALKIDIHVIIPYEGLSADQMAQIEEVFKVVYPVDDHHFKVILPYGTLVIDGVTPNKLNYFGRPYEGIAVFDGK |
| | LgTrip3546 | KITTTGTLWNGNKIIDERLITPD |
| | (ATG 3746) | |
| 207 | FRB-15GS-86, no | Atggtggccatcctctggcatgagatgtggcatgaaggcctggaagaggcatctcgtttgtactttggggaaa |
| | AI in linker | ggaacgtgaaaggcatgtttgaggtgctggagcccttgcatgctatgatggaacggggcccccagactctgaa |
| | (ATG3768) | ggaaacatcctttaatcaggcctatggtcgagatttaatggaggcccaagagtggtgcaggaagtacatgaaa |
| | | tcagggaatgtcaaggacctcacccaagcctgggacctctattatcatgtgttccgacgaatcagtggtggtt |
| | | caggtggtggcgggagcggtggctcgagcagcggtggagtgagcggctggcggctgttcaagaagattagcta |
| | | a |
| 208 | FRB-15GS-86, no | MVAILWHEMWHEGLEEASRLYFGERNVKGMFEVLEPLHAMMERGPQTLKETSFNQAYGRDLMEAQEWCRKYMK |
| | AI in linker | SGNVKDLTQAWDLYYHVFRRISGGSGGGGSGGSSSGGVSGWRLFKKIS |
| | (ATG3768) | |
| 209 | FRB-15GS-289 | Atggtggccatcctctggcatgagatgtggcatgaaggcctggaagaggcatctcgtttgtactttggggaaa |
| | (ATG3769) | ggaacgtgaaaggcatgtttgaggtgctggagcccttgcatgctatgatggaacggggcccccagactctgaa |
| | | ggaaacatcctttaatcaggcctatggtcgagatttaatggaggcccaagagtggtgcaggaagtacatgaaa |
| | | tcagggaatgtcaaggacctcacccaagcctgggacctctattatcatgtgttccgacgaatcagtggtggtt |
| | | caggtggtggcgggagcggtggctcgagcagcggtggagttagcgttagcggctggcgcctgttcaagaagat |
| | | cagctaa |
| 210 | FRB-15GS-289 | MVAILWHEMWHEGLEEASRLYFGERNVKGMFEVLEPLHAMMERGPQTLKETSFNQAYGRDLMEAQEWCRKYMK |
| | (ATG3769) | SGNVKDLTQAWDLYYHVFRRISGGSGGGGSGGSSSGGVSVSGWRLFKKIS |
| 211 | FKBP-SmTrip9 | atgggagtgcaggtggaaaccatctccccaggagacgggcgcaccttccccaagcgcggccagacctgcgtgg |
| | fusion template, | tgcactacaccgggatgcttgaagatggaaagaaatttgattcctcccgggacagaaacaagccctttaagtt |
| | no AI in linker | tatgctaggcaagcaggaggtgatccgaggctgggaagaaggggttgcccagatgagtgtgggtcagagagcc |
| | (ATG3770) | aaactgactatatctccagattatgcctatggtgccactgggcacccaggcatcatcccaccacatgccactc |
| | | tcgtcttcgatgtggagcttctaaaactggaaggtggttcaggtggtggcgggagcggtggctcgagcagcgg |
| | | tgga |
| 212 | FKBP-SmTrip9 | MGVQVETISPGDGRTFPKRGQTCVVHYTGMLEDGKKFDSSRDRNKPFKFMLGKQEVIRGWEEGVAQMSVGQRA |
| | fusion template, | KLTISPDYAYGATGHPGIIPPHATLVFDVELLKLEGGSGGGGSGGSSSGG |
| | no AI in linker | |
| | (ATG3770) | |
| 213 | 295 | GSMLFRVTINSV |
| 214 | 300 | GSMLFRVTINSVS |
| 215 | 412 | MLFRVTINSVSG |
| 216 | 413 | MLFRVTINSVSGW |
| 217 | 415 | MLFRVTINSVSGWK |
| 218 | 416 | MLFRVTINSVSGWR |
| 219 | 418 | GSMLFRVTINSVSG |
| 220 | 419 | GSMLFRVTINSVSGW |
| 221 | 422 | GSMLFRVTINSVSGWR |
| 222 | 423 | GSMLFRVTINSVSGWK |
| 223 | 434 | GSMLFRVTIWK |
| 224 | 435 | GSMLFRVTINSWK |
| 225 | 477 | MLFRVTINSWK |
| 226 | 478 | MLFRVTINSWS |
| 227 | 479 | MLFRVTIWS |
| 228 | 480 | MLFRVTIWK |
| 229 | 481 | MLFRVKINS |
| 230 | 482 | GSMLFRVTINSWS |
| 231 | 483 | GSMLFRVKINS |
| 232 | 484 | GSMLFRVTIWS |
| 233 | 485 | MLFRVNINS |
| 234 | 486 | MLFRVWINS |
| 235 | 487 | LLFRVKINS |
| 236 | 488 | FLFRVTINS |
| 237 | 295 | SSWKRGSMLFRVTINSV |
| 238 | 300 | SSWKRGSMLFRVTINSVS |
| 239 | 412 | SSWKRMLFRVTINSVSG |
| 240 | 413 | SSWKRMLFRVTINSVSGW |
| 241 | 414 | SSWKRMLFRVTINSVSGWR |
| 242 | 415 | SSWKRMLFRVTINSVSGWK |
| 243 | 417 | MLFRVTINSVSGWK |
| 244 | 418 | SSWKRGSMLFRVTINSVSG |
| 245 | 419 | SSWKRGSMLFRVTINSVSGW |
| 246 | 420 | SSWKRGSMLFRVTINSVSGWR |
| 247 | 421 | SSWKRGSMLFRVTINSVSGWK |
| 248 | 424 | SSWKRGSYLFRVTINS |
| 249 | 425 | SSWKRGSMLFRVKINS |
| 250 | 426 | SSWKRGSMLFRVRINS |
| 251 | 427 | SSWKRGSMLFRVWINS |
| 252 | 428 | SSKRGSMLFRVTIWSV |
| 253 | 429 | SSKRGSMLFRVTIWSVS |
| 254 | 430 | SSWRGSMLFRVTIKS |
| 255 | 431 | KRSSGSMLFRVTIWS |
| 256 | 432 | SSKRMLFRVTIWS |
| 257 | 433 | KRSSMLFRVTIWS |
| 258 | 445 | GSMKFRVTINSWK |
| 259 | 450 | GSMLFRKTINSWK |
| 260 | 455 | GSMLFRVTKNSWK |
| 261 | 522 | GKMLFRVTIWK |
| 262 | 523 | GSMKFRVTINSWK |
| 263 | 524 | GSMKFRVTIWK |
| 264 | 525 | GRMLFRVTINSWK |
| 265 | 526 | GRMLFRVTIWK |
| 266 | 527 | GSMRFRVTINSWK |
| 267 | 528 | GSMRFRVTIWK |
| 268 | 529 | GDMLFRVTINSWK |
| 269 | 530 | GDMLFRVTIWK |
| 270 | 531 | GSMDFRVTINSWK |
| 271 | 532 | GSMDFRVTIWK |
| 272 | 533 | GEMLFRVTINSWK |
| 273 | 535 | GSMEFRVTINSWK |
| 274 | 536 | GSMEFRVTIWK |
| 275 | 538 | GSMLFRVTIWKVK |
| 276 | 539 | GSMLFRVTIWSVK |
| 277 | 540 | GSMLFRVTIWSK |
| 278 | 541 | GSMLFRVTIWKWK |
| 279 | 542 | GSMLFRVTIWKK |
| 280 | 245 | GSMLFRVTINS |
| 281 | 292.x | MLFRVTINS |
| 282 | 297.x | MLFVTINSV |
| 283 | 302.x | MLFRVTINSVS |
| 284 | 305.x | GSMLFRVTIN |
| 285 | 306.x | GSMLFRVTI |
| 286 | 307.x | SMLFRVTIN |
| 287 | 308.x | MLFRVTIN |
| 288 | 312.x | MLFRVTI |
| 289 | 399 | SSKRGSMLFRVTIWS |
| 290 | 273 | GSMLFRVTINSGVSGWALFKKIS |
| 291 | 264 | GSMLFRVTINSGVSGWRLFKKIS |
| 292 | 167 | VSGWALFKKIS |
| 293 | 331 | GSMLFRVTINSVSGVSGWRLFKKIS |
| 294 | LgTrip 3546 (no | MVFTLDDFVGDWEQTAAYNLDQVLEQGGVSSLLQNLAVSVTPIMRIVRSGENALKIDIHVIIPYEGLSADQMA |
| | His6) | QIEEVFKVVYPVDDHHFKVILPYGTLVIDGVTPNKLNYFGRPYEGIAVFDGKKITTTGTLWNGNKIIDERLIT |
| | | PD |
| 295 | LgTrip 3546 (no | atggtcttcacactcgacgatttcgttggggactgggaacagacagccgcctacaacctggaccaagtccttg |
| | His6) | aacagggaggtgtgtccagtttgctgcagaatctcgccgtgtccgtaactccgatcatgaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacaagctgaactatttcggacggccgtatgaaggcatcgccgtgtt |
| | | cgacggcaaaaagatcactaccacagggaccctgtggaacggcaacaaaattatcgacgagcgcctgatcacc |
| | | cccgac |
| 296 | LgTrip 2098 (no | MVFTLDDFVGDWEQTAAYNLDQVLEQGGVSSLLQNLAVSVTPIMRIVRSGENALKIDIHVIIPYEGLSADQMA |
| | His6) | QIEEVFKVVYPVDDHHFKVILPYGTLVIDGVTPNKLNYFGRPYEGIAVFDGKKITVTGTLWNGNKIIDERLIT |
| | | PD |
| 297 | LgTrip 2098 (no | atggtcttcacactcgaagatttcgttggggactgggaacagacagccgcctacaacctggaccaagtccttg |
| | His6) | aacagggaggtgtgtccagtttgctgcagaatctcgccgtgtccgtaactccgatccaaaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttoggacggccgtatgaaggcatcgccgtgtt |
| | | cgacggcaaaaagatcactgtaacagggaccctgtggaacggcaacaaaattatcgacgagcgcctgatcacc |
| | | cccgac |
| 298 | 157 | SVSGWRLFKKIS |
| 299 | 158 | NSVSGWRLFKKIS |
| 300 | 206 | GWRLFKKIS |
| 301 | 264 | GSMLFRVTINSGVSGWRLFKKIS |
| 302 | 489 | GSMLFRVTINSWK (N-term unblocked) |
| 303 | 490 | GSMLFRVTINSWK (C-term unblocked) |
| 304 | 491 | GSMLFRVTINSWK (Both unblocked) |
| 305 | 492 | GSMLFRVTINKWK |
| 306 | 493 | GSMLFRVTIKSWK |
| 307 | 494 | GSMLFRVTINRWK |
| 308 | 495 | GSMLFRVTIRSWK |
| 309 | 496 | GSMLFRVTINDWK |
| 310 | 497 | GSMLFRVTIDSWK |
| 311 | 498 | GSMLFRVTINEWK |
| 312 | 499 | GSMLFRVTIESWK |
| 313 | 465 | GSMRFRVTINSWK (Both termini unblocked) |
| 314 | 466 | GSMDFRVTINSWK (Both termini unblocked) |
| 315 | 467 | GSMEFRVTINSWK (Both termini unblocked) |
| 316 | 468 | GSMLFRRTINSWK (Both termini unblocked) |
| 317 | 469 | GSMLFRDTINSWK (Both termini unblocked) |
| 318 | 470 | GSMLFRETINSWK (Both termini unblocked) |
| 319 | 472 | GSMLFRVTDNSWK (Both termini unblocked) |
| 320 | 473 | GSMLFRVTENSWK (Both termini unblocked) |
| 321 | 474 | GSMKFRVTINSWK (Both termini unblocked) |
| 322 | 475 | GSMLFRKTINSWK (Both termini unblocked) |
| 323 | 476 | GSMLFRVTKNSWK (Both termini unblocked) |
| 324 | 436 | GSMLFRVTINS (N-term unblocked) |
| 325 | 437 | GSMLFRVSINS (N-term unblocked) |
| 326 | 438 | GSMLFRVNINS (N-term unblocked) |
| 327 | 439 | GSKLFRVTINS (N-term unblocked) |
| 328 | 440 | GSRLFRVTINS (N-term unblocked) |
| 329 | 441 | GSMWFRVTINS (N-term unblocked) |
| 330 | 442 | GSMSFRVTINS (N-term unblocked) |
| 331 | 443 | GSMNERVTINS (N-term unblocked) |
| 332 | 444 | GSMKFRVTINS (N-term unblocked) |
| 333 | 446 | GSMLFRWTINS (N-term unblocked) |
| 334 | 447 | GSMLFRSTINS (N-term unblocked) |
| 335 | 448 | GSMLFRNTINS (N-term unblocked) |
| 336 | 449 | GSMLFRKTINS (N-term unblocked) |
| 337 | 451 | GSMLFRVTWNS (N-term unblocked) |
| 338 | 452 | GSMLFRVTSNS (N-term unblocked) |
| 339 | 453 | GSMLFRVTNNS (N-term unblocked) |
| 340 | 454 | GSMLFRVTKNS (N-term unblocked) |
| 341 | 456 | GSMLFRVTIKS (N-term unblocked) |
| 342 | Antares ATG | MKHHHHHHVSKGEELIKENMRSKLYLEGSVNGHQFKCTHEGEGKPYEGKQTNRIKVVEGGPLPFAFDILATHF |
| | 3802 | MYGSKVFIKYPADLPDYFKQSFPEGFTWERVMVFEDGGVLTATQDTSLQDGELIYNVKVRGVNFPANGPVMQK |
| | | KTLGWEPSTETMYPADGGLEGRCDKALKLVGGGHLHVNFKTTYKSKKPVKMPGVHYVDRRLERIKEADNETYV |
| | | EQYEHAVARYSNLGGGFTLEDFVGDWRQTAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGVTPNMIDYFGRPYEGIAVFDGKKITVTGT |
| | | LWNGNKIIDERLINPDGSLLFRVTINGVTGWRLCERILARHELIKENMRSKLYLEGSVNGHQFKCTHEGEGKP |
| | | YEGKQTNRIKVVEGGPLPFAFDILATHFMYGSKVFIKYPADLPDYFKQSFPEGFTWERVMVFEDGGVLTATQD |
| | | TSLQDGELIYNVKVRGVNFPANGPVMQKKTLGWEPSTETMYPADGGLEGRCDKALKLVGGGHLHVNFKTTYKS |
| | | KKPVKMPGVHYVDRRLERIKEADNETYVEQYEHAVARYSNLGGGMDELYK |
| 343 | Antares ATG | atgaaacatcaccatcaccatcatgtgagcaagggagaagaacttataaaagaaaacatgcggtctaaactgt |
| | 3802 | acctcgagggctccgtcaatgggcaccagtttaagtgtacccacgagggtgagggaaagccctatgaggggaa |
| | | gcagacaaaccgcatcaaggtcgtcgaagggggacccctcccgtttgcctttgatatcttggctactcacttt |
| | | atgtacggaagcaaagttttcataaagtatcctgccgaccttcctgattattttaaacagtcatttcccgagg |
| | | gtttcacatgggaaagggtcatggtgtttgaggatggaggcgtgctcactgcaactcaggacacctcactgca |
| | | ggacggcgagctgatctacaatgtgaaggtccggggtgtaaacttccctgccaacgggcctgtaatgcagaag |
| | | aagaccctgggatgggagccgtccaccgaaaccatgtaccctgctgatggtgggctggagggccgatgtgaca |
| | | aggctctgaagctcgttggaggtggtcatttgcacgtaaatttcaagacaacttacaagagcaaaaaacccgt |
| | | aaaaatgcccggggttcattacgttgacagaaggcttgaacgcataaaggaagctgataacgagacatacgtg |
| | | gagcagtacgagcacgccgttgcccggtactcaaacctggggggggctttacactggaggattttgtgggaga |
| | | ttggagacagacagccggctacaatctggatcaggtgctggaacaaggaggagtgtcttctctgtttcagaat |
| | | ctgggagtgagcgtgacacctatccagaggatcgtgctgtctggcgagaatggactgaagatcgatattcacg |
| | | tgatcatcccctacgaaggcctgtctggagaccagatgggccagattgagaagatcttcaaagtggtgtatcc |
| | | tgtggacgatcaccacttcaaggtgatcctgcactacggcaccctggtgattgatggagtgacacctaacatg |
| | | atcgactacttcggaagaccttacgagggaatcgccgtgttcgacggaaagaagatcaccgtgacaggaacac |
| | | tgtggaatggaaacaagatcatcgacgagcggctgatcaaccctgatggatctctgctgttcagagtgaccat |
| | | caacggagtgacaggatggagactgtgcgagagaattctggctagacatgagctaatcaaggaaaatatgaga |
| | | agtaagctatacttagaggggtccgtcaacggtcaccagtttaaatgcactcatgaaggtgaggggaaacctt |
| | | atgaaggtaagcagactaatcgaataaaagtggtcgagggcggtcctctgccattcgctttcgatattctggc |
| | | cactcactttatgtatgggtctaaggtctttattaaataccccgctgatttgccagactactttaaacagtcc |
| | | ttccctgaaggattcacatgggagcgggtgatggtgttcgaggatggaggcgttcttactgcaactcaggata |
| | | cttccttgcaagacggggaactgatctacaacgttaaggtccgcggcgtcaatttcccagccaatggtccagt |
| | | gatgcagaagaaaaccttgggggggagccctcaacggagacaatgtaccctgcggacggcgggcttgagggta |
| | | gatgtgataaggcattgaaactcgtcgggggcggccaccttcatgtgaatttcaagactacatataaaagtaa |
| | | aaaaccagtcaagatgcctggagtgcactacgtggatcgtaggttggagaggataaaagaagccgacaacgaa |
| | | acttatgtagagcaatatgagcacgccgtggctcgttattccaacttgggcggaggaatggatgaactgtaca |
| | | ag |
| 344 | Antares (LgBiT) | MKHHHHHHVSKGEELIKENMRSKLYLEGSVNGHQFKCTHEGEGKPYEGKQTNRIKVVEGGPLPFAFDILATHF |
| | ATG 3803 | MYGSKVFIKYPADLPDYFKQSFPEGFTWERVMVFEDGGVLTATQDTSLQDGELIYNVKVRGVNFPANGPVMQK |
| | | KTLGWEPSTETMYPADGGLEGRCDKALKLVGGGHLHVNFKTTYKSKKPVKMPGVHYVDRRLERIKEADNETYV |
| | | EQYEHAVARYSNLGGGFTLEDFVGDWEQTAAYNLDQVLEQGGVSSLLQNLAVSVTPIQRIVRSGENALKIDIH |
| | | VIIPYEGLSADQMAQIEEVFKVVYPVDDHHFKVILPYGTLVIDGVTPNMLNYFGRPYEGIAVFDGKKITVTGT |
| | | LWNGNKIIDERLITPDGSMLFRVTINSRHELIKENMRSKLYLEGSVNGHQFKCTHEGEGKPYEGKQTNRIKVV |
| | | EGGPLPFAFDILATHFMYGSKVFIKYPADLPDYFKQSFPEGFTWERVMVFEDGGVLTATQDTSLQDGELIYNV |
| | | KVRGVNFPANGPVMQKKTLGWEPSTETMYPADGGLEGRCDKALKLVGGGHLHVNFKTTYKSKKPVKMPGVHYV |
| | | DRRLERIKEADNETYVEQYEHAVARYSNLGGGMDELYK |
| 345 | Antares (LgBiT) | atgaaacatcaccatcaccatcatgtgagcaagggagaagaacttataaaagaaaacatgcggtctaaactgt |
| | ATG 3803 | acctcgagggctccgtcaatgggcaccagtttaagtgtacccacgagggtgagggaaagccctatgaggggaa |
| | | gcagacaaaccgcatcaaggtcgtcgaagggggacccctcccgtttgcctttgatatettggctactcacttt |
| | | atgtacggaagcaaagttttcataaagtatcctgccgaccttcctgattattttaaacagtcatttcccgagg |
| | | gtttcacatgggaaagggtcatggtgtttgaggatggaggcgtgctcactgcaactcaggacacctcactgca |
| | | ggacggcgagctgatctacaatgtgaaggtccggggtgtaaacttccctgccaacgggcctgtaatgcagaag |
| | | aagaccctgggatgggagccgtccaccgaaaccatgtaccctgctgatggtgggctggagggccgatgtgaca |
| | | aggctctgaagctcgttggaggtggtcatttgcacgtaaatttcaagacaacttacaagagcaaaaaacccgt |
| | | aaaaatgcccggggttcattacgttgacagaaggcttgaacgcataaaggaagctgataacgagacatacgtg |
| | | gagcagtacgagcacgccgttgcccggtactcaaacctggggggggcttcacactcgaagatttcgttgggga |
| | | ctgggaacagacagccgcctacaacctggaccaagtccttgaacagggaggtgtgtccagtttgctgcagaat |
| | | ctcgccgtgtccgtaactccgatccaaaggattgtccggagcggtgaaaatgccctgaagatcgacatccatg |
| | | tcatcatcccgtatgaaggtctgagcgccgaccaaatggcccagatcgaagaggtgtttaaggtggtgtaccc |
| | | tgtggatgatcatcactttaaggtgatcctgccctatggcacactggtaatcgacggggttacgccgaacatg |
| | | ctgaactatttcggacggccgtatgaaggcatcgccgtgttcgacggcaaaaagatcactgtaacagggaccc |
| | | tgtggaacggcaacaaaattatcgacgagcgcctgatcacccccgacggctccatgctgttccgagtaaccat |
| | | caacagcagacatgagctaatcaaggaaaatatgagaagtaagctatacttagaggggtccgtcaacggtcac |
| | | cagtttaaatgcactcatgaaggtgaggggaaaccttatgaaggtaagcagactaatcgaataaaagtggtcg |
| | | agggcggtcctctgccattcgctttcgatattctggccactcactttatgtatgggtctaaggtctttattaa |
| | | ataccccgctgatttgccagactactttaaacagtccttccctgaaggattcacatgggagcgggtgatggtg |
| | | ttcgaggatggaggcgttcttactgcaactcaggatacttccttgcaagacggggaactgatctacaacgtta |
| | | aggtccgcggcgtcaatttcccagccaatggtccagtgatgcagaagaaaaccttggggtgggagccctcaac |
| | | ggagacaatgtaccctgcggacggcgggcttgagggtagatgtgataaggcattgaaactcgtcgggggggcc |
| | | accttcatgtgaatttcaagactacatataaaagtaaaaaaccagtcaagatgcctggagtgcactacgtgga |
| | | tcgtaggttggagaggataaaagaagccgacaacgaaacttatgtagagcaatatgagcacgccgtggctcgt |
| | | tattccaacttgggcggaggaatggatgaactgtacaag |
| 346 | Antares (LgTrip | MKHHHHHHVSKGEELIKENMRSKLYLEGSVNGHQFKCTHEGEGKPYEGKQTNRIKVVEGGPLPFAFDILATHF |
| | 3546) ATG 3804 | MYGSKVFIKYPADLPDYFKQSFPEGFTWERVMVFEDGGVLTATQDTSLQDGELIYNVKVRGVNFPANGPVMQK |
| | | KTLGWEPSTETMYPADGGLEGRCDKALKLVGGGHLHVNFKTTYKSKKPVKMPGVHYVDRRLERIKEADNETYV |
| | | EQYEHAVARYSNLGGGFTLDDFVGDWEQTAAYNLDQVLEQGGVSSLLQNLAVSVTPIMRIVRSGENALKIDIH |
| | | VIIPYEGLSADQMAQIEEVFKVVYPVDDHHFKVILPYGTLVIDGVTPNKLNYFGRPYEGIAVFDGKKITTTGT |
| | | LWNGNKIIDERLITPDRHELIKENMRSKLYLEGSVNGHQFKCTHEGEGKPYEGKQTNRIKVVEGGPLPFAFDI |
| | | LATHFMYGSKVFIKYPADLPDYFKQSFPEGFTWERVMVFEDGGVLTATQDTSLQDGELIYNVKVRGVNFPANG |
| | | PVMQKKTLGWEPSTETMYPADGGLEGRCDKALKLVGGGHLHVNFKTTYKSKKPVKMPGVHYVDRRLERIKEAD |
| | | NETYVEQYEHAVARYSNLGGGMDELYK |
| 347 | Antares (LgTrip | atgaaacatcaccatcaccatcatgtgagcaagggagaagaacttataaaagaaaacatgcggtctaaactgt |
| | 3546) ATG 3804 | acctcgagggctccgtcaatgggcaccagtttaagtgtacccacgagggtgagggaaagccctatgaggggaa |
| | | gcagacaaaccgcatcaaggtcgtcgaagggggacccctcccgtttgcctttgatatcttggctactcacttt |
| | | atgtacggaagcaaagttttcataaagtatcctgccgaccttcctgattattttaaacagtcatttcccgagg |
| | | gtttcacatgggaaagggtcatggtgtttgaggatggaggcgtgctcactgcaactcaggacacctcactgca |
| | | ggacggcgagctgatctacaatgtgaaggtccggggtgtaaacttccctgccaacgggcctgtaatgcagaag |
| | | aagaccctgggatgggagccgtccaccgaaaccatgtaccctgctgatggtgggctggagggccgatgtgaca |
| | | aggctctgaagctcgttggaggtggtcatttgcacgtaaatttcaagacaacttacaagagcaaaaaacccgt |
| | | aaaaatgcccggggttcattacgttgacagaaggcttgaacgcataaaggaagctgataacgagacatacgtg |
| | | gagcagtacgagcacgccgttgcccggtactcaaacctggggggggcttcacactcgacgatttcgttgggga |
| | | ctgggaacagacagccgcctacaacctggaccaagtccttgaacagggaggtgtgtccagtttgctgcagaat |
| | | ctcgccgtgtccgtaactccgatcatgaggattgtccggagcggtgaaaatgccctgaagatcgacatccatg |
| | | tcatcatcccgtatgaaggtctgagcgccgaccaaatggcccagatcgaagaggtgtttaaggtggtgtaccc |
| | | tgtggatgatcatcactttaaggtgatcctgccctatggcacactggtaatcgacggggttacgccgaacaag |
| | | ctgaactatttcggacggccgtatgaaggcatcgccgtgttcgacggcaaaaagatcactaccacagggaccc |
| | | tgtggaacggcaacaaaattatcgacgagcgcctgatcacccccgacagacatgagctaatcaaggaaaatat |
| | | gagaagtaagctatacttagaggggtccgtcaacggtcaccagtttaaatgcactcatgaaggtgaggggaaa |
| | | ccttatgaaggtaagcagactaatcgaataaaagtggtcgagggcggtcctctgccattcgctttcgatattc |
| | | tggccactcactttatgtatgggtctaaggtctttattaaataccccgctgatttgccagactactttaaaca |
| | | gtccttccctgaaggattcacatgggagcgggtgatggtgttcgaggatggaggcgttcttactgcaactcag |
| | | gatacttccttgcaagacggggaactgatctacaacgttaaggtccgeggegtcaatttcccagccaatggtc |
| | | cagtgatgcagaagaaaaccttgggggggagccctcaacggagacaatgtaccctgcggacggcgggcttgag |
| | | ggtagatgtgataaggcattgaaactcgtcgggggcggccaccttcatgtgaatttcaagactacatataaaa |
| | | gtaaaaaaccagtcaagatgcctggagtgcactacgtggatcgtaggttggagaggataaaagaagccgacaa |
| | | cgaaacttatgtagagcaatatgagcacgccgtggctcgttattccaacttgggcggaggaatggatgaactg |
| | | tacaag |
| 348 | ATG 3815 | MKHHHHHHFTLEDFVGDWEQTAAYNLDQVLEQGGVSSLLQNLAVSVTPIQRIVRSGENALKIDIHVIIPYEGL |
| | | SADQMAQIEEVFKVVYPVDDHHFKVILPYGTLVIDGVTPNMLNYFGRPYEGIAVFDGKKITVTGTLWNGNKII |
| | | DERLITPDGSMLFRVTINSGGSGGSSGELIKENMRSKLYLEGSVNGHQFKCTHEGEGKPYEGKQTNRIKVVEG |
| | | GPLPFAFDILATHFMYGSKVFIKYPADLPDYFKQSFPEGFTWERVMVFEDGGVLTATQDTSLQDGELIYNVKV |
| | | RGVNFPANGPVMQKKTLGWEPSTETMYPADGGLEGRCDKALKLVGGGHLHVNFKTTYKSKKPVKMPGVHYVDR |
| | | RLERIKEADNETYVEQYEHAVARYSNLGGGMDELYK |
| 349 | ATG 3815 | atgaaacatcaccatcaccatcatttcacactcgaagatttcgttggggactgggaacagacagccgcctaca |
| | | acctggaccaagtccttgaacagggaggtgtgtccagtttgctgcagaatctcgccgtgtccgtaactccgat |
| | | ccaaaggattgtccggagcggtgaaaatgccctgaagatcgacatccatgtcatcatcccgtatgaaggtctg |
| | | agcgccgaccaaatggcccagatcgaagaggtgtttaaggtggtgtaccctgtggatgatcatcactttaagg |
| | | tgatcctgccctatggcacactggtaatcgacggggttacgccgaacatgctgaactatttcggacggccgta |
| | | tgaaggcatcgccgtgttcgacggcaaaaagatcactgtaacagggaccctgtggaacggcaacaaaattatc |
| | | gacgagcgcctgatcacccccgacggctccatgctgttccgagtaaccatcaacagcggaggctcaggtggat |
| | | cctcaggtgagctaatcaaggaaaatatgagaagtaagctatacttagaggggtccgtcaacggtcaccagtt |
| | | taaatgcactcatgaaggtgaggggaaaccttatgaaggtaagcagactaatcgaataaaagtggtcgagggc |
| | | ggtcctctgccattcgctttcgatattctggccactcactttatgtatgggtctaaggtctttattaaatacc |
| | | ccgctgatttgccagactactttaaacagtccttccctgaaggattcacatgggagcgggtgatggtgttcga |
| | | ggatggaggcgttcttactgcaactcaggatacttccttgcaagacggggaactgatctacaacgttaaggtc |
| | | cgcggcgtcaatttcccagccaatggtccagtgatgcagaagaaaaccttgggggggagccctcaacggagac |
| | | aatgtaccctgcggacgggggcttgagggtagatgtgataaggcattgaaactcgtcgggggcggccaccttc |
| | | atgtgaatttcaagactacatataaaagtaaaaaaccagtcaagatgcctggagtgcactacgtggatcgtag |
| | | gttggagaggataaaagaagccgacaacgaaacttatgtagagcaatatgagcacgccgtggctcgttattcc |
| | | aacttgggcggaggaatggatgaactgtacaag |
| 350 | ATG 3816 | MKHHHHHHFTLEDFVGDWEQTAAYNLDQVLEQGGVSSLLQNLAVSVTPIQRIVRSGENALKIDIHVIIPYEGL |
| | | SADQMAQIEEVFKVVYPVDDHHFKVILPYGTLVIDGVTPNMLNYFGRPYEGIAVFDGKKITVTGTLWNGNKII |
| | | DERLITPDGSMLFRVTINSRHELIKENMRSKLYLEGSVNGHQFKCTHEGEGKPYEGKQTNRIKVVEGGPLPFA |
| | | FDILATHFMYGSKVFIKYPADLPDYFKQSFPEGFTWERVMVFEDGGVLTATQDTSLODGELIYNVKVRGVNFP |
| | | ANGPVMQKKTLGWEPSTETMYPADGGLEGRCDKALKLVGGGHLHVNFKTTYKSKKPVKMPGVHYVDRRLERIK |
| | | EADNETYVEQYEHAVARYSNLGGGMDELYK |
| 351 | ATG 3816 | Atgaaacatcaccatcaccatcatttcacactcgaagatttcgttggggactgggaacagacagccgcctaca |
| | | acctggaccaagtccttgaacagggaggtgtgtccagtttgctgcagaatctcgccgtgtccgtaactccgat |
| | | ccaaaggattgtccggagcggtgaaaatgccctgaagatcgacatccatgtcatcatcccgtatgaaggtctg |
| | | agcgccgaccaaatggcccagatcgaagaggtgtttaaggtggtgtaccctgtggatgatcatcactttaagg |
| | | tgatcctgccctatggcacactggtaatcgacggggttacgccgaacatgctgaactatttcggacggccgta |
| | | tgaaggcatcgccgtgttcgacggcaaaaagatcactgtaacagggaccctgtggaacggcaacaaaattatc |
| | | gacgagcgcctgatcacccccgacggctccatgctgttccgagtaaccatcaacagcagacatgagctaatca |
| | | aggaaaatatgagaagtaagctatacttagaggggtccgtcaacggtcaccagtttaaatgcactcatgaagg |
| | | tgaggggaaaccttatgaaggtaagcagactaatcgaataaaagtggtcgagggggtcctctgccattcgctt |
| | | tcgatattctggccactcactttatgtatgggtctaaggtctttattaaataccccgctgatttgccagacta |
| | | ctttaaacagtccttccctgaaggattcacatgggagcgggtgatggtgttcgaggatggaggcgttcttact |
| | | gcaactcaggatacttccttgcaagacggggaactgatctacaacgttaaggtccgcggcgtcaatttcccag |
| | | ccaatggtccagtgatgcagaagaaaaccttgggggggagccctcaacggagacaatgtaccctgcggacggc |
| | | gggcttgagggtagatgtgataaggcattgaaactcgtcgggggcggccaccttcatgtgaatttcaagacta |
| | | catataaaagtaaaaaaccagtcaagatgcctggagtgcactacgtggatcgtaggttggagaggataaaaga |
| | | agccgacaacgaaacttatgtagagcaatatgagcacgccgtggctcgttattccaacttgggcggaggaatg |
| | | gatgaactgtacaag |
| 352 | ATG 3817 | MKHHHHHHFTLDDFVGDWEQTAAYNLDQVLEQGGVSSLLQNLAVSVTPIMRIVRSGENALKIDIHVIIPYEGL |
| | | SADQMAQIEEVFKVVYPVDDHHFKVILPYGTLVIDGVTPNKLNYFGRPYEGIAVFDGKKITTTGTLWNGNKII |
| | | DERLITPDGGSGGSSGELIKENMRSKLYLEGSVNGHQFKCTHEGEGKPYEGKQTNRIKVVEGGPLPFAFDILA |
| | | THFMYGSKVFIKYPADLPDYFKQSFPEGFTWERVMVFEDGGVLTATQDTSLQDGELIYNVKVRGVNFPANGPV |
| | | MQKKTLGWEPSTETMYPADGGLEGRCDKALKLVGGGHLHVNFKTTYKSKKPVKMPGVHYVDRRLERIKEADNE |
| | | TYVEQYEHAVARYSNLGGGMDELYK |
| 353 | ATG 3817 | Atgaaacatcaccatcaccatcatttcacactcgacgatttcgttggggactgggaacagacagccgcctaca |
| | | acctggaccaagtccttgaacagggaggtgtgtccagtttgctgcagaatctcgccgtgtccgtaactccgat |
| | | catgaggattgtccggagcggtgaaaatgccctgaagatcgacatccatgtcatcatcccgtatgaaggtctg |
| | | agcgccgaccaaatggcccagatcgaagaggtgtttaaggtggtgtaccctgtggatgatcatcactttaagg |
| | | tgatcctgccctatggcacactggtaatcgacggggttacgccgaacaagctgaactatttcggacggccgta |
| | | tgaaggcatcgccgtgttcgacggcaaaaagatcactaccacagggaccctgtggaacggcaacaaaattatc |
| | | gacgagcgcctgatcacccccgacggaggctcaggtggatcctcaggtgagctaatcaaggaaaatatgagaa |
| | | gtaagctatacttagaggggtccgtcaacggtcaccagtttaaatgcactcatgaaggtgaggggaaacctta |
| | | tgaaggtaagcagactaatcgaataaaagtggtcgagggcggtcctctgccattcgctttcgatattctggcc |
| | | actcactttatgtatgggtctaaggtctttattaaataccccgctgatttgccagactactttaaacagtcct |
| | | tccctgaaggattcacatgggagcgggtgatggtgttcgaggatggaggcgttcttactgcaactcaggatac |
| | | ttccttgcaagacggggaactgatctacaacgttaaggtccgcggcgtcaatttcccagccaatggtccagtg |
| | | atgcagaagaaaaccttgggggggagccctcaacggagacaatgtaccctgcggacggcgggcttgagggtag |
| | | atgtgataaggcattgaaactcgtcgggggcggccaccttcatgtgaatttcaagactacatataaaagtaaa |
| | | aaaccagtcaagatgcctggagtgcactacgtggatcgtaggttggagaggataaaagaagccgacaacgaaa |
| | | cttatgtagagcaatatgagcacgccgtggctcgttattccaacttgggcggaggaatggatgaactgtacaa |
| | | g |
| 354 | ATG 3818 | MKHHHHHHFTLDDFVGDWEQTAAYNLDQVLEQGGVSSLLQNLAVSVTPIMRIVRSGENALKIDIHVIIPYEGL |
| | | SADQMAQIEEVFKVVYPVDDHHFKVILPYGTLVIDGVTPNKLNYFGRPYEGIAVFDGKKITTTGTLWNGNKII |
| | | DERLITPDRHELIKENMRSKLYLEGSVNGHQFKCTHEGEGKPYEGKQTNRIKVVEGGPLPFAFDILATHFMYG |
| | | SKVFIKYPADLPDYFKQSFPEGFTWERVMVFEDGGVLTATQDTSLQDGELIYNVKVRGVNFPANGPVMQKKTL |
| | | GWEPSTETMYPADGGLEGRCDKALKLVGGGHLHVNFKTTYKSKKPVKMPGVHYVDRRLERIKEADNETYVEQY |
| | | EHAVARYSNLGGGMDELYK |
| 355 | ATG 3818 | Atgaaacatcaccatcaccatcatttcacactcgacgatttcgttggggactgggaacagacagccgcctaca |
| | | acctggaccaagtccttgaacagggaggtgtgtccagtttgctgcagaatctcgccgtgtccgtaactccgat |
| | | catgaggattgtccggagcggtgaaaatgccctgaagatcgacatccatgtcatcatcccgtatgaaggtctg |
| | | agcgccgaccaaatggcccagatcgaagaggtgtttaaggtggtgtaccctgtggatgatcatcactttaagg |
| | | tgatcctgccctatggcacactggtaatcgacggggttacgccgaacaagctgaactatttcggacggccgta |
| | | tgaaggcatcgccgtgttcgacggcaaaaagatcactaccacagggaccctgtggaacggcaacaaaattatc |
| | | gacgagcgcctgatcacccccgacagacatgagctaatcaaggaaaatatgagaagtaagctatacttagagg |
| | | ggtccgtcaacggtcaccagtttaaatgcactcatgaaggtgaggggaaaccttatgaaggtaagcagactaa |
| | | tcgaataaaagtggtcgagggcggtcctctgccattcgctttcgatattctggccactcactttatgtatggg |
| | | tctaaggtctttattaaataccccgctgatttgccagactactttaaacagtccttccctgaaggattcacat |
| | | gggagcgggtgatggtgttcgaggatggaggcgttcttactgcaactcaggatacttccttgcaagacgggga |
| | | actgatctacaacgttaaggtccgcggcgtcaatttcccagccaatggtccagtgatgcagaagaaaaccttg |
| | | ggggggagccctcaacggagacaatgtaccctgcggacggcgggcttgagggtagatgtgataaggcattgaa |
| | | actcgtcgggggcggccaccttcatgtgaatttcaagactacatataaaagtaaaaaaccagtcaagatgcct |
| | | ggagtgcactacgtggatcgtaggttggagaggataaaagaagccgacaacgaaacttatgtagagcaatatg |
| | | agcacgccgtggctcgttattccaacttgggcggaggaatggatgaactgtacaag |
| 356 | LgTrip 2899 | MKHHHHHHVFTLEDFVGDWEQTAAYNLDQVLEQGGVSSLLQNLAVSVTPILRIVRSGENALKIDIHVIIPYEG |
| | (LgTrip | LSADQMAQIEEVFKVVYPVDDHHFKVILPYGTLVIDGVTPNMLNYFGRPYEGIAVFDGKKITVTGTLWNGNKI |
| | 2098 + Q42L) | IDERLITPD |
| 357 | LgTrip 2899 | atgaaacatcaccatcaccatcatgtcttcacactcgaagatttcgttggggactgggaacagaccgccgcct |
| | (LgTrip | acaacctggaccaagtccttgaacagggaggtgtgtccagtttgctgcagaatctegccgtgtccgtaactcc |
| | 2098 + Q42L) | gatcctaaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacatgctgaactatttcggacggcc |
| | | gtatgaaggcatcgccgtgttcgacggcaaaaagatcactgtaacagggaccctgtggaacggcaacaaaatt |
| | | atcgacgagcgcctgatcacccccgac |
| 358 | ATG-3930 | atgAAACATCACCATCACCATCATgtcTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACGGCC |
| | | GTATGAAGGCATCGCCGTGTTCGACGGCTAA |
| 359 | ATG-3930 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVEDG |
| 360 | SmTrip9-15GS- | gggagctccGGTGGTGGCGGGAGCGGAGGTGGAGGctcgAGCGGTATGACGTATAAGTTAATCCTTAATGGTA |
| | ProteinG (ATG | AAACATTGAAAGGCGAGACAACTACTGAAGCTGTTGATGCTGCTACTGCAGAAAAAGTCTTCAAACAATACGC |
| | 4002) | TAACGACAACGGTGTTGACGGTGAATGGACTTACGACGATGCGACGAAAACCTTTACGGTCACCGAAAAACCA |
| | | GAAGTGATCGATGCGTCTGAATTAACACCAGCCGTGACAACTTACAAACTTGTTATTAATGGTAAAACATTGA |
| | | AAGGCGAAACAACTACTGAGGCTGTTGATGCTGCTACTGCAGAGAAGGTGTTCAAACAATATGCGAATGACAA |
| | | CGGTGTTGACGGTGAGTGGACTTACGACGATGCGACTAAGACCTTTACAGTTACTGAAAAACCAGAAGTGATC |
| | | GATGCGTCTGAGTTAACACCAGCCGTGACAACTTACAAACTTGTTATTAATGGTAAAACATTGAAAGGCGAAA |
| | | CAACTACTAAAGCAGTAGACGCAGAAACTGCGGAGAAGGCCTTCAAACAATACGCTAACGACAACGGTGTTGA |
| | | TGGTGTTTGGACTTATGATGATGCCACAAAAACCTTTACGGTAACTGAGCATCATCACCATCACCACTAA |
| 361 | SmTrip9-15GS- | GSSGGGGSGGGGSSGMTYKLILNGKTLKGETTTEAVDAATAEKVFKQYANDNGVDGEWTYDDATKTFTVTEKP |
| | ProteinG (ATG | EVIDASELTPAVTTYKLVINGKTLKGETTTEAVDAATAEKVFKQYANDNGVDGEWTYDDATKTFTVTEKPEVI |
| | 4002) | DASELTPAVTTYKLVINGKTLKGETTTKAVDAETAEKAFKQYANDNGVDGVWTYDDATKTFTVTEHHHHHH |
| 362 | ATG-3929 | atgAAACATCACCATCACCATCATgtcTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGATAA |
| 363 | ATG-3929 | Mkhhhhhhvftlddfvgdweqtaaynldqvleqggvssllqnlavsvtpimrivrsgenalkidihviipyeg |
| | | lsadqmaqieevfkvvypvddhhfkvilpygtlvidgvtpnklnyfg |
| 364 | ATG-3930 | atgAAACATCACCATCACCATCATgtcTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACGGCC |
| | | GTATGAAGGCATCGCCGTGTTCGACGGCTAA |
| 365 | ATG-3930 | Mkhhhhhhvftlddfvgdweqtaaynldqvleqggvssllqnlavsvtpimrivrsgenalkidihviipyeg |
| | | lsadqmagieevfkvvypvddhhfkvilpygtlvidgvtpnklnyfgrpyegiavfdg |
| 366 | ATG-3931 | atgAAACATCACCATCACCATCATgtcTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACGGCC |
| | | GTATGAAGGCATCGCCGTGTTCGACGGCAAAAAGATCACTACCACAGGGACCCTGTAA |
| 367 | ATG-3931 | Mkhhhhhhvftlddfvgdweqtaaynldqvleqggvssllqnlavsvtpimrivrsgenalkidihviipyeg |
| | | lsadqmagieevfkvvypvddhhfkvilpygtlvidgvtpnklnyfgrpyegiavfdgkkitttgtl |
| 368 | ATG-3932 | atgAAACATCACCATCACCATCATgtcTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACGGCC |
| | | GTATGAAGGCATCGCCGTGTTCGACGGCAAAAAGATCACTACCACAGGGACCCTGTGGAACGGCTAA |
| 369 | ATG-3932 | Mkhhhhhhvftlddfvgdweqtaaynldqvleqggvssllqnlavsvtpimrivrsgenalkidihviipyeg |
| | | lsadqmaqieevfkvvypvddhhfkvilpygtlvidgvtpnklnyfgrpyegiavfdgkkitttgtlwng |
| 370 | ATG-4808 | Atggtttccgtgagcggctggcggctgttcaagaagattagcttcacactcgacgatttcgttggggactggg |
| | | aacagacagccgcctacaacctggaccaagtccttgaacagggaggtgtgtccagtttgctgcagaatctcgc |
| | | cgtgtccgtaactccgatcatgaggattgtccggagcggtgaaaatgccctgaagatcgacatccatgtcatc |
| | | atcccgtatgaaggtctgagcgccgaccaaatggcccagatcgaagaggtgtttaaggtggtgtaccctgtgg |
| | | atgatcatcactttaaggtgatcctgccctatggcacactggtaatcgacggggttacgccgaacaagctgaa |
| | | ctatttcggacggccgtatgaaggcatcgccgtgttcgacggcaaaaagatcactaccacagggaccctgtgg |
| | | aacggcaacaaaattatcgacgagcgcctgatcacccccgactaa |
| 371 | ATG-4808 | Mvsvsgwrlfkkisftlddfvgdweqtaaynldqvleqggvssllqnlavsvtpimrivrsgenalkidihvi |
| | | ipyeglsadqmagieevfkvvypvddhhfkvilpygtlvidgvtpnklnyfgrpyegiavfdgkkitttgtlw |
| | | ngnkiiderlitpd |
| 372 | ATG-4809 | Atggtttccgtgagcggctggcggctgttcaagaagattagcggcagctccggtttcacactcgacgatttcg |
| | | ttggggactgggaacagacagccgcctacaacctggaccaagtccttgaacagggaggtgtgtccagtttgct |
| | | gcagaatctcgccgtgtccgtaactccgatcatgaggattgtccggagcggtgaaaatgccctgaagatcgac |
| | | atccatgtcatcatcccgtatgaaggtctgagcgccgaccaaatggcccagatcgaagaggtgtttaaggtgg |
| | | tgtaccctgtggatgatcatcactttaaggtgatcctgccctatggcacactggtaatcgacggggttacgcc |
| | | gaacaagctgaactatttcggacggccgtatgaaggcatcgccgtgttcgacggcaaaaagatcactaccaca |
| | | gggaccctgtggaacggcaacaaaattatcgacgagcgcctgatcacccccgactaa |
| 373 | ATG-4809 | MVSVSGWRLFKKISGSSGFTLDDFVGDWEQTAAYNLDQVLEQGGVSSLLQNLAVSVTPIMRIVRSGENALKID |
| | | IHVIIPYEGLSADQMAQIEEVFKVVYPVDDHHFKVILPYGTLVIDGVTPNKLNYFGRPYEGIAVFDGKKITTT |
| | | GTLWNGNKIIDERLITPD |
| 374 | ATG-4810 | Atggtttccgtgagcggctggcggctgttcaagaagattagcggctcgagcggtggctcgagcggtttcacac |
| | | tcgacgatttcgttggggactgggaacagacagccgcctacaacctggaccaagtccttgaacagggaggtgt |
| | | gtccagtttgctgcagaatctcgccgtgtccgtaactccgatcatgaggattgtccggagcggtgaaaatgcc |
| | | ctgaagatcgacatccatgtcatcatcccgtatgaaggtctgagcgccgaccaaatggcccagatcgaagagg |
| | | tgtttaaggtggtgtaccctgtggatgatcatcactttaaggtgatcctgccctatggcacactggtaatcga |
| | | cggggttacgccgaacaagctgaactatttcggacggccgtatgaaggcatcgccgtgttcgacggcaaaaag |
| | | atcactaccacagggaccctgtggaacggcaacaaaattatcgacgagcgcctgatcacccccgactaa |
| 375 | ATG-4810 | MVSVSGWRLFKKISGSSGGSSGFTLDDFVGDWEQTAAYNLDQVLEQGGVSSLLQNLAVSVTPIMRIVRSGENA |
| | | LKIDIHVIIPYEGLSADQMAQIEEVFKVVYPVDDHHFKVILPYGTLVIDGVTPNKLNYFGRPYEGIAVFDGKK |
| | | ITTTGTLWNGNKIIDERLITPD |
| 376 | ATG-4811 | Atggtttccgtgagcggctggcggctgttcaagaagattagcggctcgagcggtggctcgagcggtggctcga |
| | | gcggtttcacactcgacgatttcgttggggactgggaacagacagccgcctacaacctggaccaagtccttga |
| | | acagggaggtgtgtccagtttgctgcagaatctcgccgtgtccgtaactccgatcatgaggattgtccggagc |
| | | ggtgaaaatgccctgaagatcgacatccatgtcatcatcccgtatgaaggtctgagcgccgaccaaatggccc |
| | | agatcgaagaggtgtttaaggtggtgtaccctgtggatgatcatcactttaaggtgatcctgccctatggcac |
| | | actggtaatcgacggggttacgccgaacaagctgaactatttcggacggccgtatgaaggcatcgccgtgttc |
| | | gacggcaaaaagatcactaccacagggaccctgtggaacggcaacaaaattatcgacgagcgcctgatcaccc |
| | | ccgactaa |
| 377 | ATG-4811 | MVSVSGWRLFKKISGSSGGSSGGSSGFTLDDFVGDWEQTAAYNLDQVLEQGGVSSLLQNLAVSVTPIMRIVRS |
| | | GENALKIDIHVIIPYEGLSADQMAQIEEVFKVVYPVDDHHFKVILPYGTLVIDGVTPNKLNYFGRPYEGIAVF |
| | | DGKKITTTGTLWNGNKIIDERLITPD |
| 378 | ATG-4812 | Atggtttccgtgagcggctggcggctgttcaagaagattagcggctcgagcggtggctcgagcggtggctcga |
| | | gcggtggctcgagcggtttcacactcgacgatttcgttggggactgggaacagacagccgcctacaacctgga |
| | | ccaagtccttgaacagggaggtgtgtccagtttgctgcagaatctcgccgtgtccgtaactccgatcatgagg |
| | | attgtccggageggtgaaaatgccctgaagatcgacatccatgtcatcatcccgtatgaaggtctgagcgccg |
| | | accaaatggcccagatcgaagaggtgtttaaggtggtgtaccctgtggatgatcatcactttaaggtgatcct |
| | | gccctatggcacactggtaatcgacggggttacgccgaacaagctgaactatttcggacggccgtatgaaggc |
| | | atcgccgtgttcgacggcaaaaagatcactaccacagggaccctgtggaacggcaacaaaattatcgacgagc |
| | | gcctgatcacccccgactaa |
| 379 | ATG-4812 | MVSVSGWRLFKKISGSSGGSSGGSSGGSSGFTLDDFVGDWEQTAAYNLDQVLEQGGVSSLLQNLAVSVTPIMR |
| | | IVRSGENALKIDIHVIIPYEGLSADQMAQIEEVFKVVYPVDDHHFKVILPYGTLVIDGVTPNKLNYFGRPYEG |
| | | IAVFDGKKITTTGTLWNGNKIIDERLITPD |
| 380 | ATG-4813 | Atggtttccgtgagcggctggcggctgttcaagaagattagcggctcgagcggtggctcgagcggtggctcga |
| | | gcggtggctcgagcggtggctcgagcggtttcacactcgacgatttcgttggggactgggaacagacagccgc |
| | | ctacaacctggaccaagtccttgaacagggaggtgtgtccagtttgctgcagaatctcgccgtgtccgtaact |
| | | ccgatcatgaggattgtccggagcggtgaaaatgccctgaagatcgacatccatgtcatcatcccgtatgaag |
| | | gtctgagcgccgaccaaatggcccagatcgaagaggtgtttaaggtggtgtaccctgtggatgatcatcactt |
| | | taaggtgatcctgccctatggcacactggtaatcgacggggttacgccgaacaagctgaactatttcggacgg |
| | | ccgtatgaaggcatcgccgtgttcgacggcaaaaagatcactaccacagggaccctgtggaacggcaacaaaa |
| | | ttatcgacgagegcctgatcacccccgactaa |
| 381 | ATG-4813 | MVSVSGWRLFKKISGSSGGSSGGSSGGSSGGSSGFTLDDFVGDWEQTAAYNLDQVLEQGGVSSLLQNLAVSVT |
| | | PIMRIVRSGENALKIDIHVIIPYEGLSADQMAQIEEVFKVVYPVDDHHFKVILPYGTLVIDGVTPNKLNYFGR |
| | | PYEGIAVFDGKKITTTGTLWNGNKIIDERLITPD |
| 382 | ATG-4814 | Atggtgagcggctggcggctgttcaagaagattagcggctcgagcggtggctcgagcggtggctcgagcggtg |
| | | gctcgagcggtggctcgagcggtttcacactcgacgatttcgttggggactgggaacagacagccgcctacaa |
| | | cctggaccaagtccttgaacagggaggtgtgtccagtttgctgcagaatctcgccgtgtccgtaactccgatc |
| | | atgaggattgtccggagcggtgaaaatgccctgaagatcgacatccatgtcatcatcccgtatgaaggtctga |
| | | gcgccgaccaaatggcccagatcgaagaggtgtttaaggtggtgtaccctgtggatgatcatcactttaaggt |
| | | gatcctgccctatggcacactggtaatcgacggggttacgccgaacaagctgaactatttcggacggccgtat |
| | | gaaggcatcgccgtgttcgacggcaaaaagatcactaccacagggaccctgtggaacggcaacaaaattatcg |
| | | acgagcgcctgatcacccccgactaa |
| 383 | ATG-4814 | MVSGWRLFKKISGSSGGSSGGSSGGSSGGSSGFTLDDFVGDWEQTAAYNLDQVLEQGGVSSLLQNLAVSVTPI |
| | | MRIVRSGENALKIDIHVIIPYEGLSADQMAQIEEVFKVVYPVDDHHFKVILPYGTLVIDGVTPNKLNYFGRPY |
| | | EGIAVFDGKKITTTGTLWNGNKIIDERLITPD |
| 384 | ATG-4815 | Atggtcttcacactcgacgatttcgttggggactgggaacagacagccgcctacaacctggaccaagtccttg |
| | | aacagggaggtgtgtccagtttgctgcagaatctcgccgtgtccgtaactccgatcatgaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacaagctgaactatttcggacggccgtatgaaggcatcgccgtgtt |
| | | cgacggcaaaaagatcactaccacagggaccctgtggaacggcaacaaaattatcgacgagcgcctgatcacc |
| | | cccgacgtttccgtgagcggctggcggctgttcaagaagattagctaa |
| 385 | ATG-4815 | MVFTLDDFVGDWEQTAAYNLDQVLEQGGVSSLLQNLAVSVTPIMRIVRSGENALKIDIHVIIPYEGLSADQMA |
| | | QIEEVFKVVYPVDDHHFKVILPYGTLVIDGVTPNKLNYFGRPYEGIAVFDGKKITTTGTLWNGNKIIDERLIT |
| | | PDVSVSGWRLFKKIS |
| 386 | ATG-4816 | Atggtcttcacactcgacgatttcgttggggactgggaacagacagccgcctacaacctggaccaagtccttg |
| | | aacagggaggtgtgtccagtttgctgcagaatctcgccgtgtccgtaactccgatcatgaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacaagctgaactatttcggacggccgtatgaaggcatcgccgtgtt |
| | | cgacggcaaaaagatcactaccacagggaccctgtggaacggcaacaaaattatcgacgagcgcctgatcacc |
| | | cccgacggctcgagcggtgtttccgtgagcggctggcggctgttcaagaagattagctaa |
| 387 | ATG-4816 | MVFTLDDFVGDWEQTAAYNLDQVLEQGGVSSLLQNLAVSVTPIMRIVRSGENALKIDIHVIIPYEGLSADQMA |
| | | QIEEVFKVVYPVDDHHFKVILPYGTLVIDGVTPNKLNYFGRPYEGIAVFDGKKITTTGTLWNGNKIIDERLIT |
| | | PDGSSGVSVSGWRLFKKIS |
| 388 | ATG-4817 | Atggtcttcacactcgacgatttcgttggggactgggaacagacagccgcctacaacctggaccaagtccttg |
| | | aacagggaggtgtgtccagtttgctgcagaatctcgccgtgtccgtaactccgatcatgaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacaagctgaactatttcggacggccgtatgaaggcatcgccgtgtt |
| | | cgacggcaaaaagatcactaccacagggaccctgtggaacggcaacaaaattatcgacgagcgcctgatcacc |
| | | cccgacggctcgagcggtggctcgagcggtgtttccgtgagcggctggcggctgttcaagaagattagctaa |
| 389 | ATG-4817 | MVFTLDDFVGDWEQTAAYNLDQVLEQGGVSSLLQNLAVSVTPIMRIVRSGENALKIDIHVIIPYEGLSADQMA |
| | | QIEEVFKVVYPVDDHHFKVILPYGTLVIDGVTPNKLNYFGRPYEGIAVFDGKKITTTGTLWNGNKIIDERLIT |
| | | PDGSSGGSSGVSVSGWRLFKKIS |
| 390 | ATG-4818 | Atggtcttcacactcgacgatttcgttggggactgggaacagacagccgcctacaacctggaccaagtccttg |
| | | aacagggaggtgtgtccagtttgctgcagaatctcgccgtgtccgtaactccgatcatgaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacaagctgaactatttcggacggccgtatgaaggcatcgccgtgtt |
| | | cgacggcaaaaagatcactaccacagggaccctgtggaacggcaacaaaattatcgacgagcgcctgatcacc |
| | | cccgacggctcgagcggtggctcgagcggtgtgagcggctggcggctgttcaagaagattagctaa |
| 391 | ATG-4818 | MVFTLDDFVGDWEQTAAYNLDQVLEQGGVSSLLQNLAVSVTPIMRIVRSGENALKIDIHVIIPYEGLSADQMA |
| | | QIEEVFKVVYPVDDHHFKVILPYGTLVIDGVTPNKLNYFGRPYEGIAVFDGKKITTTGTLWNGNKIIDERLIT |
| | | PDGSSGGSSGVSGWRLFKKIS |
| 392 | ATG-4819 | Atggtttccgtgagcggctggcggctgttcaagaagattagcttcacactcgacgatttcgttggggactggg |
| | | aacagacagccgcctacaacctggaccaagtccttgaacagggaggtgtgtccagtttgctgcagaatctcgc |
| | | cgtgtccgtaactccgatcatgaggattgtccggagcggtgaaaatgccctgaagatcgacatccatgtcatc |
| | | atcccgtatgaaggtctgagcgccgaccaaatggcccagatcgaagaggtgtttaaggtggtgtaccctgtgg |
| | | atgatcatcactttaaggtgatcctgccctatggcacactggtaatcgacggggttacgccgaacaagctgaa |
| | | ctatttcggacggccgtatgaaggcatcgccgtgttcgacggcaaaaagatcactaccacagggaccctgtgg |
| | | aacggcaacaaaattatcgacgagcgcctgatcacccccgaccatcaccatcaccatcattaa |
| 393 | ATG-4819 | MVSVSGWRLFKKISFTLDDFVGDWEQTAAYNLDQVLEQGGVSSLLQNLAVSVTPIMRIVRSGENALKIDIHVI |
| | | IPYEGLSADQMAQIEEVFKVVYPVDDHHFKVILPYGTLVIDGVTPNKLNYFGRPYEGIAVFDGKKITTTGTLW |
| | | NGNKIIDERLITPDHHHHHH |
| 394 | ATG-4820 | Atggtttccgtgagcggctggcggctgttcaagaagattagcggcagctccggtttcacactcgacgatttcg |
| | | ttggggactgggaacagacagccgcctacaacctggaccaagtccttgaacagggaggtgtgtccagtttgct |
| | | gcagaatctcgccgtgtccgtaactccgatcatgaggattgtccggagcggtgaaaatgccctgaagatcgac |
| | | atccatgtcatcatcccgtatgaaggtctgagcgccgaccaaatggcccagatcgaagaggtgtttaaggtgg |
| | | tgtaccctgtggatgatcatcactttaaggtgatcctgccctatggcacactggtaatcgacggggttacgcc |
| | | gaacaagctgaactatttcggacggccgtatgaaggcatcgccgtgttcgacggcaaaaagatcactaccaca |
| | | gggaccctgtggaacggcaacaaaattatcgacgagegcctgatcacccccgaccatcaccatcaccatcatt |
| | | aa |
| 395 | ATG-4820 | MVSVSGWRLFKKISGSSGFTLDDFVGDWEQTAAYNLDQVLEQGGVSSLLQNLAVSVTPIMRIVRSGENALKID |
| | | IHVIIPYEGLSADQMAQIEEVFKVVYPVDDHHFKVILPYGTLVIDGVTPNKLNYFGRPYEGIAVFDGKKITTT |
| | | GTLWNGNKIIDERLITPDHHHHHH |
| 396 | ATG-4821 | Atggtttccgtgagcggctggcggctgttcaagaagattagcggctcgagcggtggctcgagcggtttcacac |
| | | tcgacgatttcgttggggactgggaacagacagccgcctacaacctggaccaagtccttgaacagggaggtgt |
| | | gtccagtttgctgcagaatctcgccgtgtccgtaactccgatcatgaggattgtccggagcggtgaaaatgcc |
| | | ctgaagatcgacatccatgtcatcatcccgtatgaaggtctgagcgccgaccaaatggcccagatcgaagagg |
| | | tgtttaaggtggtgtaccctgtggatgatcatcactttaaggtgatcctgccctatggcacactggtaatcga |
| | | cggggttacgccgaacaagctgaactatttcggacggccgtatgaaggcatcgccgtgttcgacggcaaaaag |
| | | atcactaccacagggaccctgtggaacggcaacaaaattatcgacgagegcctgatcacccccgaccatcacc |
| | | atcaccatcattaa |
| 397 | ATG-4821 | MVSVSGWRLFKKISGSSGGSSGFTLDDFVGDWEQTAAYNLDQVLEQGGVSSLLQNLAVSVTPIMRIVRSGENA |
| | | LKIDIHVIIPYEGLSADQMAQIEEVFKVVYPVDDHHFKVILPYGTLVIDGVTPNKLNYFGRPYEGIAVFDGKK |
| | | ITTTGTLWNGNKIIDERLITPDHHHHHH |
| 398 | ATG-4822 | Atggtttccgtgagcggctggcggctgttcaagaagattagcggctcgagcggtggctcgagcggtggctcga |
| | | gcggtttcacactcgacgatttcgttggggactgggaacagacagccgcctacaacctggaccaagtccttga |
| | | acagggaggtgtgtccagtttgctgcagaatctcgccgtgtccgtaactccgatcatgaggattgtccggagc |
| | | ggtgaaaatgccctgaagatcgacatccatgtcatcatcccgtatgaaggtctgagcgccgaccaaatggccc |
| | | agatcgaagaggtgtttaaggtggtgtaccctgtggatgatcatcactttaaggtgatcctgccctatggcac |
| | | actggtaatcgacggggttacgccgaacaagctgaactatttcggacggccgtatgaaggcatcgccgtgttc |
| | | gacggcaaaaagatcactaccacagggaccctgtggaacggcaacaaaattatcgacgagcgcctgatcaccc |
| | | ccgaccatcaccatcaccatcattaa |
| 399 | ATG-4822 | MVSVSGWRLFKKISGSSGGSSGGSSGFTLDDFVGDWEQTAAYNLDQVLEQGGVSSLLQNLAVSVTPIMRIVRS |
| | | GENALKIDIHVIIPYEGLSADQMAQIEEVFKVVYPVDDHHFKVILPYGTLVIDGVTPNKLNYFGRPYEGIAVF |
| | | DGKKITTTGTLWNGNKIIDERLITPDHHHHHH |
| 400 | ATG-4823 | Atggtttccgtgagcggctggcggctgttcaagaagattagcggctcgagcggtggctcgagcggtggctcga |
| | | gcggtggctcgagcggtttcacactcgacgatttcgttggggactgggaacagacagccgcctacaacctgga |
| | | ccaagtccttgaacagggaggtgtgtccagtttgctgcagaatctcgccgtgtccgtaactccgatcatgagg |
| | | attgtccggagcggtgaaaatgccctgaagatcgacatccatgtcatcatcccgtatgaaggtctgagcgccg |
| | | accaaatggcccagatcgaagaggtgtttaaggtggtgtaccctgtggatgatcatcactttaaggtgatcct |
| | | gccctatggcacactggtaatcgacggggttacgccgaacaagctgaactatttcggacggccgtatgaaggc |
| | | atcgccgtgttcgacggcaaaaagatcactaccacagggaccctgtggaacggcaacaaaattatcgacgagc |
| | | gcctgatcacccccgaccatcaccatcaccatcattaa |
| 401 | ATG-4823 | MVSVSGWRLFKKISGSSGGSSGGSSGGSSGFTLDDFVGDWEQTAAYNLDQVLEQGGVSSLLQNLAVSVTPIMR |
| | | IVRSGENALKIDIHVIIPYEGLSADQMAQIEEVFKVVYPVDDHHFKVILPYGTLVIDGVTPNKLNYFGRPYEG |
| | | IAVFDGKKITTTGTLWNGNKIIDERLITPDHHHHHH |
| 402 | ATG-4824 | Atggtgagcggctggcggctgttcaagaagattagcggctcgagcggtggctcgagcggtggctcgagcggtg |
| | | gctcgagcggtggctcgagcggtttcacactcgacgatttcgttggggactgggaacagacagccgcctacaa |
| | | cctggaccaagtccttgaacagggaggtgtgtccagtttgctgcagaatctcgccgtgtccgtaactccgatc |
| | | atgaggattgtccggagcggtgaaaatgccctgaagatcgacatccatgtcatcatcccgtatgaaggtctga |
| | | gcgccgaccaaatggcccagatcgaagaggtgtttaaggtggtgtaccctgtggatgatcatcactttaaggt |
| | | gatcctgccctatggcacactggtaatcgacggggttacgccgaacaagctgaactatttcggacggccgtat |
| | | gaaggcatcgccgtgttcgacggcaaaaagatcactaccacagggaccctgtggaacggcaacaaaattatcg |
| | | acgagcgcctgatcacccccgaccatcaccatcaccatcattaa |
| 403 | ATG-4824 | MVSGWRLFKKISGSSGGSSGGSSGGSSGGSSGFTLDDFVGDWEQTAAYNLDQVLEQGGVSSLLQNLAVSVTPI |
| | | MRIVRSGENALKIDIHVIIPYEGLSADQMAQIEEVFKVVYPVDDHHFKVILPYGTLVIDGVTPNKLNYFGRPY |
| | | EGIAVFDGKKITTTGTLWNGNKIIDERLITPDHHHHHH |
| 404 | ATG-4825 | Atggtttccgtgagcggctggcggctgttcaagaagattagcggctcgagcggtggctcgagcggtggctcga |
| | | gcggtggctcgagcggtggctcgagcggtttcacactcgacgatttcgttggggactgggaacagacagccgc |
| | | ctacaacctggaccaagtccttgaacagggaggtgtgtccagtttgctgcagaatctcgccgtgtccgtaact |
| | | ccgatcatgaggattgtccggagcggtgaaaatgccctgaagatcgacatccatgtcatcatcccgtatgaag |
| | | gtctgagcgccgaccaaatggcccagatcgaagaggtgtttaaggtggtgtaccctgtggatgatcatcactt |
| | | taaggtgatcctgccctatggcacactggtaatcgacggggttacgccgaacaagctgaactatttcggacgg |
| | | ccgtatgaaggcatcgccgtgttcgacggcaaaaagatcactaccacagggaccctgtggaacggcaacaaaa |
| | | ttatcgacgagcgcctgatcacccccgaccatcaccatcaccatcattaa |
| 405 | ATG-4825 | MVSVSGWRLFKKISGSSGGSSGGSSGGSSGGSSGFTLDDFVGDWEQTAAYNLDQVLEQGGVSSLLQNLAVSVT |
| | | PIMRIVRSGENALKIDIHVIIPYEGLSADQMAQIEEVFKVVYPVDDHHFKVILPYGTLVIDGVTPNKLNYFGR |
| | | PYEGIAVFDGKKITTTGTLWNGNKIIDERLITPDHHHHHH |
| 406 | ATG-4826 | Atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcategccgtgttcgacggcaaaaagatcactaccacagggaccctgtggaacggcaacaaaatt |
| | | atcgacgagcgcctgatcacccccgacgtttccgtgagcggctggcggctgttcaagaagattagctaa |
| 407 | ATG-4826 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKITTTGTLWNGNKI |
| | | IDERLITPDVSVSGWRLFKKIS |
| 408 | ATG-4827 | Atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactaccacagggaccctgtggaacggcaacaaaatt |
| | | atcgacgagcgcctgatcacccccgacggctcgagcggtgtttccgtgagcggctggcggctgttcaagaaga |
| | | ttagctaa |
| 409 | ATG-4827 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKITTTGTLWNGNKI |
| | | IDERLITPDGSSGVSVSGWRLFKKIS |
| 410 | ATG-4828 | Atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactaccacagggaccctgtggaacggcaacaaaatt |
| | | atcgacgagcgcctgatcacccccgacggctcgagcggtggctcgagcggtgtgagcggctggcggctgttca |
| | | agaagattagctaa |
| 411 | ATG-4828 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKITTTGTLWNGNKI |
| | | IDERLITPDGSSGGSSGVSGWRLFKKIS |
| 412 | ATG-4829 | Atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgacggggttacgccgaacaagctgaactatttcggacggcc |
| | | gtatgaaggcatcgccgtgttcgacggcaaaaagatcactaccacagggaccctgtggaacggcaacaaaatt |
| | | atcgacgagcgcctgatcacccccgacggctcgagcggtggctcgagcggtgtttccgtgagcggctggcggc |
| | | tgttcaagaagattagctaa |
| 413 | ATG-4829 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKITTTGTLWNGNKI |
| | | IDERLITPDGSSGGSSGVSVSGWRLFKKIS |
| 414 | ATG-2623 | atggtcttcacactcgaagatttcgttggggactgggaacagacagccgcctacaacctggaccaagtccttg |
| | | aacagggaggtgtgtccagtttgctgcagaatctcgccgtgtccgtaactccgatccaaaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacggcaacaaaattatcgacgagcgcctgatcacc |
| | | cccgacggctccatgctgttccgagtaaccatcaacagccatcatcaccatcaccactaa |
| 415 | ATG-2623 | MVFTLEDFVGDWEQTAAYNLDQVLEQGGVSSLLQNLAVSVTPIQRIVRSGENALKIDIHVIIPYEGLSADQMA |
| | | QIEEVFKVVYPVDDHHFKVILPYGTLVIDGVTPNMLNYFGRPYEGIAVFDGKKITVTGTLWNGNKIIDERLIT |
| | | PDGSMLFRVTINSHHHHHH |
| 416 | ATG-3745 | atggtgagcggctggcggctgttcaagaagattagccaccatcaccatcaccatcatcacttcacactcgacg |
| | | atttcgttggggactgggaacagacagccgcctacaacctggaccaagtccttgaacagggaggtgtgtccag |
| | | tttgctgcagaatctcgccgtgtccgtaactccgatcatgaggattgtccggagcggtgaaaatgccctgaag |
| | | atcgacatccatgtcatcatcccgtatgaaggtctgagcgccgaccaaatggcccagatcgaagaggtgttta |
| | | aggtggtgtaccctgtggatgatcatcactttaaggtgatcctgccctatggcacactggtaatcgacggggt |
| | | tacgccgaacaagctgaactatttoggacggccgtatgaaggcatcgccgtgttcgacggcaaaaagatcact |
| | | accacagggaccctgtggaacggcaacaaaattatcgacgagcgcctgatcacccccgactaa |
| 417 | ATG-3745 | MVSGWRLFKKISHHHHHHHHFTLDDFVGDWEQTAAYNLDQVLEQGGVSSLLQNLAVSVTPIMRIVRSGENALK |
| | | IDIHVIIPYEGLSADQMAQIEEVFKVVYPVDDHHFKVILPYGTLVIDGVTPNKLNYFGRPYEGIAVFDGKKIT |
| | | TTGTLWNGNKIIDERLITPD |
| 418 | ATG-3746 | atgaaacatcaccatcaccatcatgtgagcggctggcggctgttcaagaagattagcggcagctccggtttca |
| | | cactcgacgatttcgttggggactgggaacagacagccgcctacaacctggaccaagtccttgaacagggagg |
| | | tgtgtccagtttgctgcagaatctcgccgtgtccgtaactccgatcatgaggattgtccggagcggtgaaaat |
| | | gccctgaagatcgacatccatgtcatcatcccgtatgaaggtctgagcgccgaccaaatggcccagatcgaag |
| | | aggtgtttaaggtggtgtaccctgtggatgatcatcactttaaggtgatcctgccctatggcacactggtaat |
| | | cgacggggttacgccgaacaagctgaactatttoggacggccgtatgaaggcatcgccgtgttcgacggcaaa |
| | | aagatcactaccacagggaccctgtggaacggcaacaaaattatcgacgagcgcctgatcacccccgactaa |
| 419 | ATG-3746 | MKHHHHHHVSGWRLFKKISGSSGFTLDDFVGDWEQTAAYNLDQVLEQGGVSSLLQNLAVSVTPIMRIVRSGEN |
| | | ALKIDIHVIIPYEGLSADQMAQIEEVFKVVYPVDDHHFKVILPYGTLVIDGVTPNKLNYFGRPYEGIAVFDGK |
| | | KITTTGTLWNGNKIIDERLITPD |
| 420 | ATG-4632 | atggtgagcggctggcggctgttcaagaagattagcggcagctccggtttcacactcgacgatttcgttgggg |
| | | actgggaacagacagccgcctacaacctggaccaagtccttgaacagggaggtgtgtccagtttgctgcagaa |
| | | tctcgccgtgtccgtaactccgatcatgaggattgtccggageggtgaaaatgccctgaagatcgacatccat |
| | | gtcatcatcccgtatgaaggtctgagcgccgaccaaatggcccagatcgaagaggtgtttaaggtggtgtacc |
| | | ctgtggatgatcatcactttaaggtgatcctgccctatggcacactggtaatcgacggggttacgccgaacaa |
| | | gctgaactatttcggacggccgtatgaaggcatcgccgtgttcgacggcaaaaagatcactaccacagggacc |
| | | ctgtggaacggcaacaaaattatcgacgagcgcctgatcacccccgaccatcaccatcaccatcattaa |
| 421 | ATG-4632 | MVSGWRLFKKISGSSGFTLDDFVGDWEQTAAYNLDQVLEQGGVSSLLQNLAVSVTPIMRIVRSGENALKIDIH |
| | | VIIPYEGLSADQMAQIEEVFKVVYPVDDHHFKVILPYGTLVIDGVTPNKLNYFGRPYEGIAVFDGKKITTTGT |
| | | LWNGNKIIDERLITPDHHHHHH |
| 422 | ATG-2757 | atggtcttcacactcgaagatttcgttggggactgggaacagacagccgcctacaacctggaccaagtccttg |
| | | aacagggaggtgtgtccagtttgctgcagaatctcgccgtgtccgtaactccgatccaaaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacgagaacaaaattatcgacgagcgcctgatcacc |
| | | cccgacggctccatgctgttccgagtaaccatcaacagccatcatcaccatcaccactaa |
| 423 | ATG-2757 | MVFTLEDFVGDWEQTAAYNLDQVLEQGGVSSLLQNLAVSVTPIQRIVRSGENALKIDIHVIIPYEGLSADQMA |
| | | QIEEVFKVVYPVDDHHFKVILPYGTLVIDGVTPNMLNYFGRPYEGIAVFDGKKITVTGTLWNENKIIDERLIT |
| | | PDGSMLFRVTINSHHHHHH |
| 424 | ATG-2760 | atggtcttcacactcgaagatttcgttggggactgggaacagacagccgcctacaacctggaccaagtccttg |
| | | aacagggaggtgtgtccagtttgctgcagaatctcgccgtgtccgtaactccgatccaaaggattgtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacggcgttaaaattatcgacgagcgcctgatcacc |
| | | cccgacggctccatgctgttccgagtaaccatcaacagccatcatcaccatcaccactaa |
| 425 | ATG-2760 | MVFTLEDFVGDWEQTAAYNLDQVLEQGGVSSLLQNLAVSVTPIQRIVRSGENALKIDIHVIIPYEGLSADQMA |
| | | QIEEVFKVVYPVDDHHFKVILPYGTLVIDGVTPNMLNYFGRPYEGIAVFDGKKITVTGTLWNGVKIIDERLIT |
| | | PDGSMLFRVTINSHHHHHH |
| 426 | ATG-3882 | atggtcttcacactcgaagatttcgttggggactgggaacagacagccgcctacaacctggaccaagtccttg |
| | | aacagggaggtgtgtccagtttgctgcagaatctcgccgtgtccgtaactccgatccaaaggatggtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacggcaacaaaattatcgacgagcgcctgatcacc |
| | | cccgacggctccatgctgttccgagtaaccatcaacagccatcatcaccatcaccactaa |
| 427 | ATG-3882 | MVFTLEDFVGDWEQTAAYNLDQVLEQGGVSSLLQNLAVSVTPIQRMVRSGENALKIDIHVIIPYEGLSADQMA |
| | | QIEEVFKVVYPVDDHHFKVILPYGTLVIDGVTPNMLNYFGRPYEGIAVFDGKKITVTGTLWNGNKIIDERLIT |
| | | PDGSMLFRVTINSHHHHHH |
| 428 | ATG-3901 | atggtcttcacactcgaagatttcgttggggactggaagcagacagccgcctacaacctggaccaagtccttg |
| | | aacagggaggtgtgtccagtttgctgcagaatctcgccgtgtccgtaactccgatccaaaggatggtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacggcaacaaaattatcgacgagcgcctgatcacc |
| | | cccgacggctccatgctgttccgagtaaccatcaacagccatcatcaccatcaccactaa |
| 429 | ATG-3901 | MVFTLEDFVGDWKQTAAYNLDQVLEQGGVSSLLQNLAVSVTPIQRMVRSGENALKIDIHVIIPYEGLSADQMA |
| | | QIEEVFKVVYPVDDHHFKVILPYGTLVIDGVTPNMLNYFGRPYEGIAVFDGKKITVTGTLWNGNKIIDERLIT |
| | | PDGSMLFRVTINSHHHHHH |
| 430 | ATG-3945 | atggtcttcacactcgaagatttcgttggggactggaagcagacagccgcctacaacctggaccaagtccttg |
| | | aacagggaggtgtgtccagtttgctgcagaatctcgccgtgtccgtaactccgatccaaaggatggtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacgacgtcaaaattatcgacgagcgcctgatcacc |
| | | cccgacggctccatgctgttccgagtaaccatcaacagccatcatcaccatcaccactaa |
| 431 | ATG-3945 | MVFTLEDFVGDWKQTAAYNLDQVLEQGGVSSLLQNLAVSVTPIQRMVRSGENALKIDIHVIIPYEGLSADQMA |
| | | QIEEVFKVVYPVDDHHFKVILPYGTLVIDGVTPNMLNYFGRPYEGIAVFDGKKITVTGTLWNDVKIIDERLIT |
| | | PDGSMLFRVTINSHHHHHH |
| 432 | ATG-3984 | atggtcttcacactcgaagatttcgttggggactggaagcagacagccgcctacaacctggaccaagtccttg |
| | | aacagggaggtgtgtccagtttgctgcagaatctcgccgtgtccgtaactccgatccaaaggatggtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacgacgtcaaaattatcgacgagegcctgatcacc |
| | | cccgacggctccatgtccttccgagtaaccatcaacagccatcatcaccatcaccactaa |
| 433 | ATG-3984 | MVFTLEDFVGDWKQTAAYNLDQVLEQGGVSSLLQNLAVSVTPIQRMVRSGENALKIDIHVIIPYEGLSADQMA |
| | | QIEEVFKVVYPVDDHHFKVILPYGTLVIDGVTPNMLNYFGRPYEGIAVFDGKKITVTGTLWNDVKIIDERLIT |
| | | PDGSMSFRVTINSHHHHHH |
| 434 | ATG-4147 | atggtcttcacactcgaagatttcgttggggactggaagcagacagccgcctacaacctggaccaagtccttg |
| | | aacagggaggtgtgtccagtttgctgcagaatctcgccgtgtccgtaactccgatccaaaggatggtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacggcaacaaaattatcgacgagcgcctgatcacc |
| | | cccgacggctccatgtccttccgagtaaccatcaacagccatcatcaccatcaccactaa |
| 435 | ATG-4147 | MVFTLEDFVGDWKQTAAYNLDQVLEQGGVSSLLQNLAVSVTPIQRMVRSGENALKIDIHVIIPYEGLSADQMA |
| | | QIEEVFKVVYPVDDHHFKVILPYGTLVIDGVTPNMLNYFGRPYEGIAVFDGKKITVTGTLWNGNKIIDERLIT |
| | | PDGSMSFRVTINSHHHHHH |
| 436 | ATG-4166 | atggtcttcacactcgaagatttcgttggggactggaagcagacagccgcctacaacctggaccaagtccttg |
| | | aacagggaggtgtgtccagtttgctgcagaatctcgccgtgtccgtaactccgatccaaaggatggtccggag |
| | | cggtgaaaatgccctgaagatcgacatccatgtcatcatcccgtatgaaggtctgagcgccgaccaaatggcc |
| | | cagatcgaagaggtgtttaaggtggtgtaccctgtggatgatcatcactttaaggtgatcctgccctatggca |
| | | cactggtaatcgacggggttacgccgaacatgctgaactatttcggacggccgtatgaaggcatcgccgtgtt |
| | | cgacggcaaaaagatcactgtaacagggaccctgtggaacggcgtcaaaattatcgacgagcgcctgatcacc |
| | | cccgacggctccatgtccttccgagtaaccatcaacagccatcatcaccatcaccactaa |
| 437 | ATG-4166 | MVFTLEDFVGDWKQTAAYNLDQVLEQGGVSSLLQNLAVSVTPIQRMVRSGENALKIDIHVIIPYEGLSADQMA |
| | | QIEEVFKVVYPVDDHHFKVILPYGTLVIDGVTPNMLNYFGRPYEGIAVFDGKKITVTGTLWNGVKIIDERLIT |
| | | PDGSMSFRVTINSHHHHHH |
| 438 | ATG-5037 | ATGAAACATCACCATCACCATCATGTCTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACACCC |
| | | GTATGAAGGCATCGCCGTGTTCGACGGCAAAAAGATCACTACCACAGGGACCCTGTGGAACGGCAACAAAATT |
| | | ATCGACGAGCGCCTGATCACCCCCGACTAA |
| 439 | ATG-5037 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGHPYEGIAVFDGKKITTTGTLWNGNKI |
| | | IDERLITPD |
| 440 | ATG-5038 | ATGAAACATCACCATCACCATCATGTCTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACGGCC |
| | | GTATGAAGGCATCGCCGTGTTCGACGGCGAGAAGATCACTACCACAGGGACCCTGTGGAACGGCAACAAAATT |
| | | ATCGACGAGCGCCTGATCACCCCCGACTAA |
| 441 | ATG-5038 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVEDGEKITTTGTLWNGNKI |
| | | IDERLITPD |
| 442 | ATG-5039 | ATGAAACATCACCATCACCATCATGTCTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACGGCC |
| | | GTATGAAGGCATCGCCGTGTTCGACGGCAAAAAGATCACTACCACAGGGACCCTGCCTAACGGCAACAAAATT |
| | | ATCGACGAGCGCCTGATCACCCCCGACTAA |
| 443 | ATG-5039 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKITTTGTLPNGNKI |
| | | IDERLITPD |
| 444 | ATG-5040 | ATGAAACATCACCATCACCATCATGTCTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACGGCC |
| | | GTATGAAGGCATCGCCGTGTTCGACGGCAAAAAGATCACTACCACAGGGACCCTGTGGAACGGCAACAAAATT |
| | | ATCGACGAGCGCCTGATCGATCCCGACTAA |
| 445 | ATG-5040 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKITTTGTLWNGNKI |
| | | IDERLIDPD |
| 446 | ATG-5041 | ATGAAACATCACCATCACCATCATGTCTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACGGCC |
| | | GTATGAAGGCATCGCCGTGTTCGACGGCAAAAAGATCACTACCACAGGGACCCTGTGGAACGGCAACAAAATT |
| | | ATCGACGAGCGCCTGATCACCGATGACTAA |
| 447 | ATG-5041 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGRPYEGIAVFDGKKITTTGTLWNGNKI |
| | | IDERLITDD |
| 448 | ATG-5135 | ATGAAACATCACCATCACCATCATGTCTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACACCC |
| | | GTATGAAGGCATCGCCGTGTTCGACGGCGAGAAGATCACTACCACAGGGACCCTGTGGAACGGCAACAAAATT |
| | | ATCGACGAGCGCCTGATCACCCCCGACTAA |
| 449 | ATG-5135 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGHPYEGIAVEDGEKITTTGTLWNGNKI |
| | | IDERLITPD |
| 450 | ATG-5146 | ATGAAACATCACCATCACCATCATGTCTTCACACTCGACGATTTCGTTGGGGACTGGGAACAGACAGCCGCCT |
| | (LgTrip 5146) | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACACCC |
| | | GTATGAAGGCATCGCCGTGTTCGACGGCGAGAAGATCACTACCACAGGGACCCTGTGGAACGGCAACAAAATT |
| | | ATCGACGAGCGCCTGATCGATCCCGACTAA |
| 451 | ATG-5146 | MKHHHHHHVFTLDDFVGDWEQTAAYNLDQVLEQGGVSSLLQNLAVSVTPIMRIVRSGENALKIDIHVIIPYEG |
| | (LgTrip 5146) | LSADQMAQIEEVFKVVYPVDDHHFKVILPYGTLVIDGVTPNKLNYFGHPYEGIAVFDGEKITTTGTLWNGNKI |
| | | IDERLIDPD |
| 452 | ATG-5158 | ATGAAACATCACCATCACCATCATGTCTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCTATGGCACACTGGTAATCGACGGGGTTACGCCGAACAAGCTGAACTATTTCGGACACCC |
| | | GTATGAAGGCATCGCCGTGTTCGACGGCGAGAAGATCACTACCACAGGGACCCTGTGGAACGGCAACAAAATT |
| | | ATCGACGAGCGCCTGATCGATGATGACTAA |
| 453 | ATG-5158 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVIDGVTPNKLNYFGHPYEGIAVEDGEKITTTGTLWNGNKI |
| | | IDERLIDDD |
| 454 | ATG-5260 | ATGAAACATCACCATCACCATCATGATTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCATCGGCACACTGGTAATCGACGGGGTTACGCCGAACAAGCTGAACTATTTCGGACACCC |
| | | GTATGAAGGCATCGCCGTGTTCGACGGCGAGAAGATCACTACCACAGGGACCCTGTGGAACGGCAACAAAATT |
| | | ATCGACGAGCGCCTGATCGATCCCGACTAA |
| 455 | ATG-5260 | MKHHHHHHDFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPIGTLVIDGVTPNKLNYFGHPYEGIAVFDGEKITTTGTLWNGNKI |
| | | IDERLIDPD |
| 456 | ATG-5266 | ATGAAACATCACCATCACCATCATGTCTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCATCGGCACACTGGTAATCGACGGGGAGACGCCGAACAAGCTGAACTATTTCGGACACCC |
| | | GTATGAAGGCATCGCCGTGTTCGACGGCGAGAAGATCACTACCACAGGGACCCTGTGGAACGGCAACAAAATT |
| | | ATCGACGAGCGCCTGATCGATCCCGACTAA |
| 457 | ATG-5266 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPIGTLVIDGETPNKLNYFGHPYEGIAVEDGEKITTTGTLWNGNKI |
| | | IDERLIDPD |
| 458 | ATG-5267 | ATGAAACATCACCATCACCATCATGTCTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCATCGGCACACTGGTAATCGACGGGGTTACGCCGAACAAGCTGAACTATTTCGGACACCC |
| | | GTATGAAGGCATCGCCGATTTCGACGGCGAGAAGATCACTACCACAGGGACCCTGTGGAACGGCAACAAAATT |
| | | ATCGACGAGCGCCTGATCGATCCCGACTAA |
| 459 | ATG-5267 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPIGTLVIDGVTPNKLNYFGHPYEGIADFDGEKITTTGTLWNGNKI |
| | | IDERLIDPD |
| 460 | ATG-5278 | ATGAAACATCACCATCACCATCATGTCTTCACACTCGACGATTTCGTTGGGGACTGGGAACAGACAGCCGCCT |
| | | ACAACCTGGACCAAGTCCTTGAACAGGGAGGTGTGTCCAGTTTGCTGCAGAATCTCGCCGTGTCCGTAACTCC |
| | | GATCATGAGGATTGTCCGGAGCGGTGAAAATGCCCTGAAGATCGACATCCATGTCATCATCCCGTATGAAGGT |
| | | CTGAGCGCCGACCAAATGGCCCAGATCGAAGAGGTGTTTAAGGTGGTGTACCCTGTGGATGATCATCACTTTA |
| | | AGGTGATCCTGCCCATCGGCACACTGGTAATCGACGGGGAGACGCCGAACAAGCTGAACTATTTCGGACACCC |
| | | GTATGAAGGCATCGCCGATTTCGACGGCGAGAAGATCACTACCACAGGGACCCTGTGGAACGGCAACAAAATT |
| | | ATCGACGAGCGCCTGATCGATCCCGACTAA |
| 461 | ATG-5278 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPIGTLVIDGETPNKLNYFGHPYEGIADFDGEKITTTGTLWNGNKI |
| | | IDERLIDPD |
| 462 | ATG-4794 | atgaaacatcaccatcaccatcatgtcttcacactcgacgatttcgttggggactgggaacagacagccgcct |
| | | acaacctggaccaagtccttgaacagggaggtgtgtccagtttgctgcagaatctcgccgtgtccgtaactcc |
| | | gatcatgaggattgtccggagcggtgaaaatgccctgaagatcgacatccatgtcatcatcccgtatgaaggt |
| | | ctgagcgccgaccaaatggcccagatcgaagaggtgtttaaggtggtgtaccctgtggatgatcatcacttta |
| | | aggtgatcctgccctatggcacactggtaatcgac |
| 463 | ATG-4794 | MKHHHHHHVFTLDDFVGDWEQTAAYNLDQVLEQGGVSSLLQNLAVSVTPIMRIVRSGENALKIDIHVIIPYEG |
| | | LSADQMAQIEEVFKVVYPVDDHHFKVILPYGTLVID |
| 720 | HALOTAG | MAEIGTGFPFDPHYVEVLGERMHYVDVGPRDGTPVLFLHGNPTSSYVWRNIIPHVAPTHRCIAPDLIGMGKSD |
| | | KPDLGYFFDDHVRFMDAFIEALGLEEVVLVIHDWGSALGFHWAKRNPERVKGIAFMEFIRPIPTWDEWPEFAR |
| | | ETFQAFRTTDVGRKLIIDQNVFIEGTLPMGVVRPLTEVEMDHYREPFLNPVDREPLWRFPNELPIAGEPANIV |
| | | ALVEEYMDWLHQSPVPKLLFWGTPGVLIPPAEAARLAKSLPNCKAVDIGPGLNLLQEDNPDLIGSEIARWLST |
| | | LEISG |
| 721 | ATG3998 [6xHis- | atgaaacatcaccatcaccatcatgtcagatcatcttctcgaaccccgagtgacaagcctgtagcccatgttg |
| | TNFa(sol)-VS- | tagcaaaccctcaagctgaggggcagctccagtggctgaaccgccgggccaatgccctcctggccaatggcgt |
| | HiBiT] | ggagctgagagataaccagctggtggtgccatcagagggcctgtacctcatctactcccaggtcctcttcaag |
| | | ggccaaggctgcccctccacccatgtgctcctcacccacaccatcagccgcatcgccgtctcctaccagacca |
| | | aggtcaacctcctctctgccatcaagagcccctgccagagggagaccccagagggggctgaggccaagccctg |
| | | gtatgagcccatctatctgggaggggtcttccagctggagaagggtgaccgactcagcgctgagatcaatcgg |
| | | cccgactatctcgactttgccgagtctgggcaggtctactttgggatcattgccctgtcgagttcaggtggtg |
| | | gcgggagcggtggagggagcagcggtggagtttccgtgagcggctggcggctgttcaagaagattagctaa |
| 722 | ATG3998 [6xHis- | MKHHHHHHVRSSSRTPSDKPVAHVVANPQAEGQLQWLNRRANALLANGVELRDNQLVVPSEGLYLIYSQVLFK |
| | TNFa(sol)-VS- | GQGCPSTHVLLTHTISRIAVSYQTKVNLLSAIKSPCQRETPEGAEAKPWYEPIYLGGVFQLEKGDRLSAEINR |
| | HiBiT] | PDYLDFAESGQVYFGIIALSSSGGGGSGGGSSGGVSVSGWRLFKKIS. |
| 723 | ATG4002 | ATGGgcaagatgctgttccgagtaaccatcaacagctggaaggggagctccGGTGGTGGCGGGAGCGGAGGTG |
| | [smTrip9(521)- | GAGGctcgAGCGGTATGACGTATAAGTTAATCCTTAATGGTAAAACATTGAAAGGCGAGACAACTACTGAAGC |
| | 15GS-protein G- | TGTTGATGCTGCTACTGCAGAAAAAGTCTTCAAACAATACGCTAACGACAACGGTGTTGACGGTGAATGGACT |
| | 6xHis] | TACGACGATGCGACGAAAACCTTTACGGTCACCGAAAAACCAGAAGTGATCGATGCGTCTGAATTAACACCAG |
| | | CCGTGACAACTTACAAACTTGTTATTAATGGTAAAACATTGAAAGGCGAAACAACTACTGAGGCTGTTGATGC |
| | | TGCTACTGCAGAGAAGGTGTTCAAACAATATGCGAATGACAACGGTGTTGACGGTGAGTGGACTTACGACGAT |
| | | GCGACTAAGACCTTTACAGTTACTGAAAAACCAGAAGTGATCGATGCGTCTGAGTTAACACCAGCCGTGACAA |
| | | CTTACAAACTTGTTATTAATGGTAAAACATTGAAAGGCGAAACAACTACTAAAGCAGTAGACGCAGAAACTGC |
| | | GGAGAAGGCCTTCAAACAATACGCTAACGACAACGGTGTTGATGGTGTTTGGACTTATGATGATGCCACAAAA |
| | | ACCTTTACGGTAACTGAGCATCATCACCATCACCACTAA |
| 724 | ATG4002 | MGKMLFRVTINSWKGSSGGGGSGGGGSSGMTYKLILNGKTLKGETTTEAVDAATAEKVFKQYANDNGVDGEWT |
| | [smTrip9(521)- | YDDATKTFTVTEKPEVIDASELTPAVTTYKLVINGKTLKGETTTEAVDAATAEKVFKQYANDNGVDGEWTYDD |
| | 15GS-protein G- | ATKTFTVTEKPEVIDASELTPAVTTYKLVINGKTLKGETTTKAVDAETAEKAFKQYANDNGVDGVWTYDDATK |
| | 6xHis] | TFTVTEHHHHHH. |
| 725 | ATG4496 | atggacaagatgctgttccgagtaaccatcaacaagtggaaggggagctccggtggtggcgggagcggaggtg |
| | SmTrip9(743)- | gaggctcgagcggtatgacgtataagttaatccttaatggtaaaacattgaaaggcgagacaactactgaagc |
| | 15GS-G | tgttgatgctgctactgcagaaaaagtcttcaaacaatacgctaacgacaacggtgttgacggtgaatggact |
| | | tacgacgatgcgacgaaaacctttacggtcaccgaaaaaccagaagtgatcgatgcgtctgaattaacaccag |
| | | ccgtgacaacttacaaacttgttattaatggtaaaacattgaaaggcgaaacaactactgaggctgttgatgc |
| | | tgctactgcagagaaggtgttcaaacaatatgcgaatgacaacggtgttgacggtgagtggacttacgacgat |
| | | gcgactaagacctttacagttactgaaaaaccagaagtgatcgatgcgtctgagttaacaccagccgtgacaa |
| | | cttacaaacttgttattaatggtaaaacattgaaaggcgaaacaactactaaagcagtagacgcagaaactgc |
| | | ggagaaggccttcaaacaatacgctaacgacaacggtgttgatggtgtttggacttatgatgatgccacaaaa |
| | | acctttacggtaactgagcatcatcaccatcaccac |
| 726 | ATG4496 | MDKMLFRVTINKWKGSSGGGGSGGGGSSGMTYKLILNGKTLKGETTTEAVDAATAEKVFKQYANDNGVDGEWT |
| | SmTrip9(743)- | YDDATKTFTVTEKPEVIDASELTPAVTTYKLVINGKTLKGETTTEAVDAATAEKVFKQYANDNGVDGEWTYDD |
| | 15GS-G | ATKTFTVTEKPEVIDASELTPAVTTYKLVINGKTLKGETTTKAVDAETAEKAFKQYANDNGVDGVWTYDDATK |
| | | TFTVTEHHHHHH |
| 727 | ATG4558 | atggacaagctcctgttcacggtaaccatcgagaagtataaggggagctccggtggtggcgggagcggaggtg |
| | SmTrip9(759)- | gaggctcgagcggtatgacgtataagttaatccttaatggtaaaacattgaaaggcgagacaactactgaagc |
| | 15GS-G | tgttgatgctgctactgcagaaaaagtcttcaaacaatacgctaacgacaacggtgttgacggtgaatggact |
| | | tacgacgatgcgacgaaaacctttacggtcaccgaaaaaccagaagtgatcgatgcgtctgaattaacaccag |
| | | ccgtgacaacttacaaacttgttattaatggtaaaacattgaaaggcgaaacaactactgaggctgttgatgc |
| | | tgctactgcagagaaggtgttcaaacaatatgcgaatgacaacggtgttgacggtgagtggacttacgacgat |
| | | gcgactaagacctttacagttactgaaaaaccagaagtgatcgatgcgtctgagttaacaccagccgtgacaa |
| | | cttacaaacttgttattaatggtaaaacattgaaaggcgaaacaactactaaagcagtagacgcagaaactgc |
| | | ggagaaggccttcaaacaatacgctaacgacaacggtgttgatggtgtttggacttatgatgatgccacaaaa |
| | | acctttacggtaactgagcatcatcaccatcaccac |
| 728 | ATG4558 | MDKLLFTVTIEKYKGSSGGGGSGGGGSSGMTYKLILNGKTLKGETTTEAVDAATAEKVFKQYANDNGVDGEWT |
| | SmTrip9(759)- | YDDATKTFTVTEKPEVIDASELTPAVTTYKLVINGKTLKGETTTEAVDAATAEKVFKQYANDNGVDGEWTYDD |
| | 15GS-G | ATKTFTVTEKPEVIDASELTPAVTTYKLVINGKTLKGETTTKAVDAETAEKAFKQYANDNGVDGVWTYDDATK |
| | | TFTVTEHHHHHH |
| 729 | ATG4551 | atgaagaagatgctgttccgagtaaccatccagaagtggaaggggagctccggtggtggcgggagcggaggtg |
| | SmTrip9(760)- | gaggctcgagcggtatgacgtataagttaatccttaatggtaaaacattgaaaggcgagacaactactgaagc |
| | 15GS-G | tgttgatgctgctactgcagaaaaagtcttcaaacaatacgctaacgacaacggtgttgacggtgaatggact |
| | | tacgacgatgcgacgaaaacctttacggtcaccgaaaaaccagaagtgatcgatgcgtctgaattaacaccag |
| | | ccgtgacaacttacaaacttgttattaatggtaaaacattgaaaggcgaaacaactactgaggctgttgatgc |
| | | tgctactgcagagaaggtgttcaaacaatatgcgaatgacaacggtgttgacggtgagtggacttacgacgat |
| | | gcgactaagacctttacagttactgaaaaaccagaagtgatcgatgcgtctgagttaacaccagccgtgacaa |
| | | cttacaaacttgttattaatggtaaaacattgaaaggcgaaacaactactaaagcagtagacgcagaaactgc |
| | | ggagaaggccttcaaacaatacgctaacgacaacggtgttgatggtgtttggacttatgatgatgccacaaaa |
| | | acctttacggtaactgagcatcatcaccatcaccac |
| 730 | ATG4551 | MKKMLFRVTIQKWKGSSGGGGSGGGGSSGMTYKLILNGKTLKGETTTEAVDAATAEKVFKQYANDNGVDGEWT |
| | SmTrip9(760)- | YDDATKTFTVTEKPEVIDASELTPAVTTYKLVINGKTLKGETTTEAVDAATAEKVFKQYANDNGVDGEWTYDD |
| | 15GS-G | ATKTFTVTEKPEVIDASELTPAVTTYKLVINGKTLKGETTTKAVDAETAEKAFKQYANDNGVDGVWTYDDATK |
| | | TFTVTEHHHHHH |

TABLE 3
Exemplary peptide sequences.

| | SEQ | |
|---|---|---|
| | ID | |
| Pep ID | NO. | Sequence |
| 521 | 16 | GKMLFRVTINSWK |
| (SmTrip9 Pep521) | | |
| 289 | 17 | VSVSGWRLFKKIS |
| (SmTrip10 Pep289; | | |
| VSHiBiT) | | |
| 691 | 18 | VSGWRLFRRIS |
| (SmTrip10 Pep691; | | |
| HW-0977) | | |
| 692 | 19 | VSVSGWRLFRRIS |
| (SmTrip10 Pep692; | | |
| HW-1053) | | |
| 693 | 20 | GRMLFRVTINSWR |
| (SmTrip9 Pep693; HW- | | |
| 0984 (SulfoSE-PEG3); | | |
| HW-1042 (SulfoSE-PEG6)) | | |
| 743 | 21 | GKMLFRVTINKWK |
| (SmTrip9 Pep743) | | |
| 759 | 22 | DKLLFTVTIEKYK |
| (SmTrip9 Pep759) | | |
| 760 | 23 | KKMLFRVTIQKWK |
| (SmTrip9 Pep760) | | |
| 895 | 24 | GRLLFVVVIERYR |
| (SmTrip9 Pep895; HW- | | |
| 1010 (SulfoSE-PEG3); | | |
| HW-1043 (SulfoSE-PEG6)) | | |
| 929 | 25 | RRMLFRVTIQRWR |
| (SmTrip9 Pep929; HW- | | |
| 1055 (SulfoSE-PEG3); | | |
| HW-1052 (SulfoSE-PEG6)) | | |
| 937 | 26 | VSGWRLFRRISC |
| (SmTrip9 Pep937; | | |
| HW-0987) | | |
| 938 | 27 | GRMLFRVTINSWRC |
| (SmTrip9 Pep938; | | |
| HW-0992 (TAMRA); | | |
| HW-1050 (SA)) | | |
| 86 | 464 | VSGWRLFKKIS |
| 229 | 465 | VSGWRLFKKI |
| 543 | 466 | WNGNKIIDERLITPD |
| 544 | 467 | KKITTTGTLWNGR |
| 545 | 468 | RPYEGIAVFDGK |
| 591 | 469 | GKMLFRVTIWKVSVSGWRLFKKIS |
| 592 | 470 | GKMLFRVTIWKVSGWRLFKKIS |
| 593 | 471 | GSMKFRVTINSWKVSVSGWRLFKKIS |
| 594 | 472 | GSMKFRVTINSWKVSGWRLFKKIS |
| 595 | 473 | GSMKFRVTINSWKNVTGYRLFKKISN |
| 596 | 474 | GSMKFRVTINSWKVTGYRLFEKIS |
| 597 | 475 | GSMKFRVTIWKVSVSGWRLFKKIS |
| 598 | 476 | GSMKFRVTIWKVSGWRLFKKIS |
| 599 | 477 | GRMLFRVTINSWKVSVSGWRLFKKIS |
| 600 | 478 | GRMLFRVTINSWKVSGWRLFKKIS |
| 601 | 479 | GRMLFRVTIWKVSVSGWRLFKKIS |
| 602 | 480 | GRMLFRVTIWKVSGWRLFKKIS |
| 603 | 481 | GSMLFRVTINSVSVSGWRLFKKIS |
| 604 | 482 | GSMLFKVTINSVSGWRLFKKIS |
| 605 | 483 | GSMLFQVTINSVSGWRLFKKIS |
| 606 | 484 | GSMLFEVTINSVSGWRLFKKIS |
| 607 | 485 | GSMLFNVTINSVSGWRLFKKIS |
| 608 | 486 | GRPYEGIAVFDGKKITTTGTL |
| 609 | 487 | GSMKFRVTINSWKVTGYRLFEKES |
| 610 | 488 | GSMKFRVTINSWKVEGYRLFEKIS |
| 611 | 489 | KKITTTGTLWNGNKIIDERLITPD |
| 612 | 490 | WNGNKIIDERLITPDGSMLFRVTINS |
| 671 | 491 | GKMLFRVTIQKWK |
| 668 | 492 | GKMLFRVTIGKWK |
| 727 | 493 | GKMLFRVTIGRWK |
| 669 | 494 | GKMLFRVTIGNWK |
| 674 | 495 | GKMLFRVTIQNWK |
| 702 | 496 | GKMLFRVTIDKWK |
| 703 | 497 | GKMLFRVTIEKWK |
| 705 | 498 | EKMLFRVTIESWK |
| 724 | 499 | EKLLFRVTIESWK |
| 725 | 500 | EKLLFRVTIESYK |
| 730 | 501 | GKMLFRVTIERWK |
| 731 | 502 | GKMLFRVTIDRWK |
| 738 | 503 | DKMLFRVTIQKWK |
| 739 | 504 | DKMLFRVTIGKWK |
| 848 | 505 | DKMLFRVTIGRWK |
| 740 | 506 | DKMLFRVTIGNWK |
| 741 | 507 | DKMLFRVTIQNWK |
| 732 | 508 | DKMLFRVTIDKWK |
| 742 | 509 | DKMLFRVTIEKWK |
| 735 | 510 | DKMLFRVTIERWK |
| 733 | 511 | DKMLFRVTIDRWK |
| 798 | 512 | RPYEGIAVFDGKKITVTGTLWNGNKIIDER |
| | | LITPD |
| 849 | 513 | EKMLFRVTIQKWK |
| 708 | 514 | EKMLFRVTIGKWK |
| 709 | 515 | EKMLFRVTIGRWK |
| 775 | 516 | DKMLFTVTIQKVSGWRLFKKIS |
| 788 | 517 | DKLLFTVTIEKVSGWRLFKKIS |
| 789 | 518 | DKLLFTVTIEKWKVSGWRLFKKIS |
| 790 | 519 | DKLLFTVTIEKYKVSGWRLFKKIS |
| 792 | 520 | DKLLFTVTIEKYKVSVSGWRLFKKIS |
| 795 | 521 | KKMLFRVTIQKVSGWRLFKKIS |
| 797 | 522 | KKMLFRVTIQKWKVSVSGWRLFKKIS |
| 796 | 523 | KKMLFRVTIQKWKVSGWRLFKKIS |
| 804 | 524 | DKLLFTVTIGKVSGWRLFKKIS |
| 805 | 525 | DKLLFTVTIGKYKVSGWRLFKKIS |
| 806 | 526 | DKLLFTVTIGKYKVSVSGWRLFKKIS |
| 807 | 527 | DKLLFTVTIGKWKVSVSGWRLFKKIS |
| 808 | 528 | DKLLFTVTIQKVSGWRLFKKIS |
| 813 | 529 | KKMLFTVTIQKVSGWRLFKKIS |
| 816 | 530 | KKLLFRVTIQKVSGWRLFKKIS |
| 825 | 531 | DKLLFTVTIEKVSGWRLFKKI |
| 826 | 532 | DKLLFTVTIEKYKVSVSGWRLFKKI |
| 827 | 533 | DRLLFTVTIERVSGWRLFKKIS |
| 831 | 534 | EKLLFTVTIEKVSGWRLFKKIS |
| 832 | 535 | KKLLFTVTIGKVSGWRLFKKIS |
| 833 | 536 | GSMRFRVTINSWRVTGYRLFERES |
| 834 | 537 | GSMKFRVTINSVTGYRLFEKES |
| 844 | 538 | KKITTTGTLWNGNKIID |
| 845 | 539 | ERLITPDGSMLFRVTINSVSGWRLFKKIS |
| 846 | 540 | GRPYEGIAVDFGKKITTTGTLWNGNKIIDE |
| | | RLITPDGSMLFRVTINSVSGWRLFKKIS |
| 847 | 541 | GVTPNKLNYFGRPYEGIAVDFGKKITTTGT |
| | | LWNGNKIIDERLITPDGSMLFRVTINSVSG |
| | | WRLFKKIS |
| 850 | 542 | EKMLFRVTIGNWK |
| 851 | 543 | EKMLFRVTIQNWK |
| 706 | 544 | EKMLFRVTIDKWK |
| 707 | 545 | EKMLFRVTIEKWK |
| 737 | 546 | EKMLFRVTIERWK |
| 736 | 547 | EKMLFRVTIDRWK |
| 852 | 548 | KKMLFRVTIGKWK |
| 853 | 549 | KKMLFRVTIGRWK |
| 854 | 550 | KKMLFRVTIGNWK |
| 855 | 551 | KKMLFRVTIQNWK |
| 856 | 552 | KKMLFRVTIDKWK |
| 857 | 553 | KKMLFRVTIEKWK |
| 858 | 554 | KKMLFRVTIERWK |
| 859 | 555 | KKMLFRVTIDRWK |
| 860 | 556 | RKMLFRVTIQKWK |
| 861 | 557 | RKMLFRVTIGKWK |
| 862 | 558 | RKMLFRVTIGRWK |
| 863 | 559 | RKMLFRVTIGNWK |
| 864 | 560 | RKMLFRVTIQNWK |
| 865 | 561 | RKMLFRVTIDKWK |
| 866 | 562 | RKMLFRVTIEKWK |
| 867 | 563 | RKMLFRVTIERWK |
| 868 | 564 | RKMLFRVTIDRWK |
| 656 | 565 | EQMLFRVTINSWK |
| 869 | 566 | SRMLFRVTINSWK |
| 533 | 567 | GEMLFRVTINSWK |
| 690 | 568 | GKMKFRVTINSWK |
| 678 | 569 | GKMLFRVKINSWK |
| 679 | 570 | GKMLFRVRINSWK |
| 681 | 571 | GKMLFRVDINSWK |
| 663 | 572 | GKMLFRVTIDSWK |
| 714 | 573 | EKMLFKVTIQKWK |
| 870 | 574 | EKMLFTVTIQKWK |
| 871 | 575 | EKMLFKVTIDKWK |
| 872 | 576 | EKMLFTVTIDKWK |
| 873 | 577 | EKMLFKVTIGRWK |
| 744 | 578 | DKMLFKVTIQKWK |
| 745 | 579 | DKMLFTVTIQKWK |
| 874 | 580 | DKMLFKVTIDKWK |
| 875 | 581 | DKMLFTVTIDKWK |
| 876 | 582 | GKMLFKVTIEKWK |
| 877 | 583 | GKMLFTVTIEKWK |
| 748 | 584 | DKMLFKVTIGKWK |
| 749 | 585 | DKMLFTVTIGKWK |
| 878 | 586 | DKMLFKVTIGNWK |
| 879 | 587 | DKMLFKVTIQNWK |
| 781 | 588 | GKMLFKVTINKWK |
| 782 | 589 | GKMLFTVTINKWK |
| 752 | 590 | DKMLFKVTIEKWK |
| 753 | 591 | DKMLFTVTIEKWK |
| 750 | 592 | DKLLFKVTIGKWK |
| 786 | 593 | DKMLFTVTINKWK |
| 756 | 594 | DKLLFTVTIQKWK |
| 757 | 595 | DKLLFTVTIQKYK |
| 758 | 596 | DKLLFTVTIEKWK |
| 793 | 597 | DKLLFTVTIGKWK |
| 794 | 598 | DKLLFTVTIGKYK |
| 799 | 599 | DKLLFTVTINKWK |
| 800 | 600 | DKLLFTVTINKYK |
| 780 | 601 | GKMLFRVTINS |
| 765 | 602 | DKMLFTVTIQK |
| 779 | 603 | DKMLFKVTIQK |
| 820 | 604 | DKLLFTVTIGK |
| 819 | 605 | DKMLFTVTIGK |
| 822 | 606 | DKMLFTVTIEK |
| 821 | 607 | DKLLFTVTIEK |
| 627 | 608 | *DKMLFRVTINSWK |
| 628 | 609 | *EKMLFRVTINSWK |
| 629 | 610 | *RKMLFRVTINSWK |
| 630 | 611 | *KKMLFRVTINSWK |
| 631 | 612 | *HKMLFRVTINSWK |
| 632 | 613 | *GLMLFRVTINSWK |
| 633 | 614 | *GQMLFRVTINSWK |
| 634 | 615 | *GTMLFRVTINSWK |
| 635 | 616 | *GKLLFRVTINSWK |
| 636 | 617 | *GKMLFKVTINSWK |
| 637 | 618 | *GKMLFRVTIQSWK |
| 638 | 619 | *GKMLFRVTIDSWK |
| 639 | 620 | *GKMLFRVTIGSWK |
| 640 | 621 | *GKMLFRVTINTWK |
| 641 | 622 | *GKMLFRVTINNWK |
| 642 | 623 | *GKMLFRVTINQWK |
| 643 | 624 | *GKMLFRVTINPWK |
| 644 | 625 | *GKMLFRVTINKWK |
| 645 | 626 | *GKMLFRVTINSWQ |
| 646 | 627 | *GKMLFRVTINSWN |
| 647 | 628 | *GKMLFRVTINSWT |
| 648 | 629 | *GKMLFRVTINSWH |
| 649 | 630 | *GKMLFRVTINSWP |
| 650 | 631 | *GKMLFRVTINSWR |
| 677 | 632 | GKMKFRVTIDSWK |
| 680 | 633 | GKMLFRVEINSWK |
| 682 | 634 | GKMLFRVQINSWK |
| 683 | 635 | GKMKFRVKINSWK |
| 684 | 636 | GKMKFRVRINSWK |
| 685 | 637 | GKMKFRVEINSWK |
| 686 | 638 | GKMKFRVDINSWK |
| 687 | 639 | GKMKFRVQINSWK |
| 688 | 640 | GKMKFRVNINSWK |
| 689 | 641 | GKMKFRVSINSWK |
| 613 | 642 | GKMLFRVNINSWK |
| 614 | 643 | GKMLFRVSINSWK |
| 615 | 644 | GKMLFRVWINSWK |
| 616 | 645 | GKMSFRVTINSWK |
| 617 | 646 | GKMWFRVTINSWK |
| 618 | 647 | GKMNFRVTINSWK |
| 619 | 648 | GSMLFRVTINSYK |
| 620 | 649 | GKMLFRVTINSYK |
| 621 | 650 | GKMLFRVTIKSWK |
| 622 | 651 | GKMLFRVTIESWK |
| 716 | 652 | GKMKFRVTIQSWK |
| 717 | 653 | GKMKFRVTIESWK |
| 718 | 654 | GKMKFRVTIKSWK |
| 719 | 655 | GKMKFRVTIRSWK |
| 651 | 656 | RLMLFRVTINSWK |
| 652 | 657 | RQMLFRVTINSWK |
| 653 | 658 | KLMLFRVTINSWK |
| 654 | 659 | KQMLFRVTINSWK |
| 655 | 660 | ELMLFRVTINSWK |
| 657 | 661 | DLMLFRVTINSWK |
| 658 | 662 | DQMLFRVTINSWK |
| 659 | 663 | DKMLFRVTINSWK |
| 660 | 664 | EKMLFRVTINSWK |
| 661 | 665 | RKMLFRVTINSWK |
| 662 | 666 | KKMLFRVTINSWK |
| 665 | 667 | GKMLFRVTIGSWK |
| 667 | 668 | GKMLFRVTINKWK |
| 670 | 669 | GKMLFRVTISKWK |
| 671 | 670 | GKMLFRVTIQKWK |
| 672 | 671 | GKMLFRVTITKWK |
| 673 | 672 | GKMLFRVTIKKWK |
| 675 | 673 | GKMLFKVTINSWK |
| 676 | 674 | RLMLFRVTIGKWK |
| 701 | 675 | GKMLFRVTINRWK |
| 710 | 676 | EKMLFTVTIGKWK |
| 711 | 677 | EKLLFTVTIGKWK |
| 712 | 678 | EKMLFTVTIGRWK |
| 720 | 679 | EKMLFTVTIEKWK |
| 722 | 680 | DKMLFRVTIESWK |
| 726 | 681 | EKLLFRVTIGKYK |
| 746 | 682 | DKLLFKVTIQKWK |
| 747 | 683 | DKLLFKVTIQKYK |
| 751 | 684 | DKLLFKVTIGKYK |
| 754 | 685 | DKLLFKVTIEKWK |
| 755 | 686 | DKLLFKVTIEKYK |
| 761 | 687 | KKLLFRVTIQKWK |
| 762 | 688 | DRMLFRVTIQRWR |
| 766 | 689 | ERMLFRVTIGRWR |
| 768 | 690 | GRMLFRVTINRWR |
| 770 | 691 | DRMLFRVTIERWR |
| 783 | 692 | DKMLFKVTIQKYK |
| 784 | 693 | DKMLFRVTINKWK |
| 785 | 694 | DKMLFKVTIEKYK |
| 787 | 695 | DKMLFKVTINKWK |
| 693 | 696 | GRMLFRVTINSWR |
| 895 | 697 | GRLLFVVVIERYR |
| 937 | 698 | VSGWRLFRRISC |
| 938 | 699 | GRMLFRVTINSWRC |
| 939 | 700 | GRLLFTVTIERYRC |
| 840 | 701 | GKLLFVVVIEKYK |
| 900 | 702 | GKLLFVTIEKVSGWRLFKKIS |

*Terminus unblocked

TABLE 4
Exemplary luciferase base sequences.

| | SEQ ID | |
|---|---|---|
| Pep ID | NO. | Sequence |
| LgTrip 3546 | 703 | MVFTLDDFVGDWEQTAAYNLDQVLEQGGVSSLLONLAVSVTPIMRIVRSGENAL |
| -WT strand | | KIDIHVIIPYEGLSADQMAQIEEVFKVVYPVDDHHFKVILPYGTLVIDGVTPNK |
| 9-HiBiT | | LNYFGRPYEGIAVFDGKKITTTGTLWNGNKIIDERLITPDGSMLFRVTINSVSG |
| | | WRLFKKIS |
| LgTrip 3546 | 704 | MVFTLDDFVGDWEQTAAYNLDQVLEQGGVSSLLQNLAVSVTPIMRIVRSGENAL |
| -WT strand | | KIDIHVIIPYEGLSADQMAQIEEVFKVVYPVDDHHFKVILPYGTLVIDGVTPNK |
| 9-SmBiT | | LNYFGRPYEGIAVFDGKKITTTGTLWNGNKIIDERLITPDGSMLFRVTINSVTG |
| | | YRLFEEIL |
| LgTrip 3546 | 705 | MVFTLDDFVGDWEQTAAYNLDQVLEQGGVSSLLQNLAVSVTPIMRIVRSGENA |
| (1-5) | | LKIDIHVIIPYEGLSADQMAQIEEVFKVVYPVDDHHFKVILPYGTLVID |
| LgTrip 3546 | 706 | MVFTLDDFVGDWEQTAAYNLDQVLEQGGVSSLLQNLAVSVTPIMRIVRSGENA |
| (1-6) | | LKIDIHVIIPYEGLSADQMAQIEEVFKVVYPVDDHHFKVILPYGTLVIDGVTP |
| | | NKLNYFGRPYEGIAVEDG |
| LgTrip 3546 | 707 | MVFTLDDFVGDWEQTAAYNLDQVLEQGGVSSLLQNLAVSVTPIMRIVRSGENA |
| (1-7) | | LKIDIHVIIPYEGLSADQMAQIEEVFKVVYPVDDHHFKVILPYGTLVIDGVTP |
| | | NKLNYFGRPYEGIAVFDGKKITTTGTL |
| LgTrip 3546 | 708 | MVFTLDDFVGDWEQTAAYNLDQVLEQGGVSSLLQNLAVSVTPIMRIVRSGENA |
| (1-8) | | LKIDIHVIIPYEGLSADQMAQIEEVFKVVYPVDDHHFKVILPYGTLVIDGVTP |
| | | NKLNYFGRPYEGIAVFDGKKITTTGTLWNGNKIIDERLITPD |
| LgTrip 3546 | 709 | GVTPNKLNYFGRPYEGIAVFDGKKITTTGTLWNGNKIIDERLITPDGSMLFRV |
| (strands 6-8) | | TINSVSGWRLFKKIS |
| -WT strand | | |
| 9-HiBiT | | |
| LgTrip 3546 | 710 | KKITTTGTLWNGNKIIDERLITPDGSMLFRVTINSVSGWRLFKKIS |
| (strands 7-8) | | |
| -WT strand | | |
| 9-HiBiT | | |
| LgTrip 3546 | 711 | WNGNKIIDERLITPDGSMLFRVTINSVSGWRLFKKIS |
| (strand 8)- | | |
| WT strand 9 | | |
| -HiBiT | | |
| WT strand 9 | 712 | GSMLFRVTINSVSGWRLFKKIS |
| -HiBiT | | |
| LgTrip 3546 | 713 | GVTPNKLNYFGRPYEGIAVFDGKKITTTGTLWNGNKIIDERLITPDGSMLFRV |
| (strands 6-8) | | TINSVTGYRLFEEIL |
| -WT strand | | |
| 9-SmBiT | | |
| LgTrip 3546 | 714 | KKITTTGTLWNGNKIIDERLITPDGSMLFRVTINSVTGYRLFEEIL |
| (strands 7-8) | | |
| -WT strand | | |
| 9-SmBiT | | |
| LgTrip 3546 | 715 | WNGNKIIDERLITPDGSMLFRVTINSVTGYRLFEEIL |
| (strand 8)- | | |
| WT strand 9 | | |
| -SmBiT | | |
| WT strand 9 | 716 | GSMLFRVTINSVTGYRLFEEIL |
| -SmBiT | | |
| β6-like | 717 | GVTPNKLNYFGRPYEGIAVEDG |
| β7-like | 718 | KKITTTGTL |
| β8-like | 719 | WNGNKIIDERLITPD |
| ATG3998 | 721 | atgaaacatcaccatcaccatcatgtcagatcatcttctcgaaccccgagtg |
| [6xHis- | | acaagcctgtagcccatgttgtagcaaaccctcaagctgaggggcagctcca |
| TNFa(sol)- | | gtggctgaaccgccgggccaatgccctcctggccaatggcgtggagctgaga |
| VS-HiBiT] | | gataaccagctggtggtgccatcagagggcctgtacctcatctactcccagg |
| | | tcctcttcaagggccaaggctgcccctccacccatgtgctcctcacccacac |
| | | catcagccgcatcgccgtctcctaccagaccaaggtcaacctcctctctgcc |
| | | atcaagagcccctgccagagggagaccccagagggggctgaggccaagccct |
| | | ggtatgagcccatctatctgggaggggtcttccagctggagaagggtgaccg |
| | | actcagcgctgagatcaatcggcccgactatctcgactttgccgagtctggg |
| | | caggtctactttgggatcattgccctgtcgagttcaggtggtggcgggagcg |
| | | gtggagggagcagcggtggagtttccgtgagcggctggcggctgttcaagaa |
| | | gattagctaa |
| ATG3998 | 722 | MKHHHHHHVRSSSRTPSDKPVAHVVANPQAEGQLQWLNRRANALLANGVELR |
| [6xHis- | | DNQLVVPSEGLYLIYSQVLFKGQGCPSTHVLLTHTISRIAVSYQTKVNLLSA |
| TNFa(sol)- | | IKSPCQRETPEGAEAKPWYEPIYLGGVFQLEKGDRLSAEINRPDYLDFAESG |
| VS-HiBiT] | | QVYFGIIALSSSGGGGSGGGSSGGVSVSGWRLFKKIS. |
| ATG4002 | 723 | ATGGgcaagatgctgttccgagtaaccatcaacagctggaaggggagctccG |
| [smTrip9 | | GTGGTGGCGGGAGCGGAGGTGGAGGctcgAGCGGTATGACGTATAAGTTAAT |
| (521)-15GS- | | CCTTAATGGTAAAACATTGAAAGGCGAGACAACTACTGAAGCTGTTGATGCT |
| protein G- | | GCTACTGCAGAAAAAGTCTTCAAACAATACGCTAACGACAACGGTGTTGACG |
| 6xHis] | | GTGAATGGACTTACGACGATGCGACGAAAACCTTTACGGTCACCGAAAAACC |
| | | AGAAGTGATCGATGCGTCTGAATTAACACCAGCCGTGACAACTTACAAACTT |
| | | GTTATTAATGGTAAAACATTGAAAGGCGAAACAACTACTGAGGCTGTTGATG |
| | | CTGCTACTGCAGAGAAGGTGTTCAAACAATATGCGAATGACAACGGTGTTGA |
| | | CGGTGAGTGGACTTACGACGATGCGACTAAGACCTTTACAGTTACTGAAAAA |
| | | CCAGAAGTGATCGATGCGTCTGAGTTAACACCAGCCGTGACAACTTACAAAC |
| | | TTGTTATTAATGGTAAAACATTGAAAGGCGAAACAACTACTAAAGCAGTAGA |
| | | CGCAGAAACTGCGGAGAAGGCCTTCAAACAATACGCTAACGACAACGGTGTT |
| | | GATGGTGTTTGGACTTATGATGATGCCACAAAAACCTTTACGGTAACTGAGC |
| | | ATCATCACCATCACCACTAA |
| ATG4002 | 724 | MGKMLFRVTINSWKGSSGGGGSGGGGSSGMTYKLILNGKTLKGETTTEAVDA |
| [smTrip9(52 | | ATAEKVFKQYANDNGVDGEWTYDDATKTFTVTEKPEVIDASELTPAVTTYKL |
| 1)-15GS- | | VINGKTLKGETTTEAVDAATAEKVFKQYANDNGVDGEWTYDDATKTFTVTEK |
| protein G- | | PEVIDASELTPAVTTYKLVINGKTLKGETTTKAVDAETAEKAFKQYANDNGV |
| 6xHis] | | DGVWTYDDATKTFTVTEHHHHHH. |

What is claimed is:

1-68. (canceled)

69. A lateral flow detection system comprising:
an analytical membrane comprising at least one detection region and a control region, wherein the at least one detection region comprises a first target analyte binding agent immobilized to the detection region;
a conjugate pad comprising a second target analyte binding agent; and
a sample pad;
wherein a liquid sample added to the sample pad will flow from the sample pad through the conjugate pad to the at least one detection region and the control region on the analytical membrane;
wherein the first target analyte binding agent and the second target analyte binding agent form a bioluminescent analyte detection complex in the at least one detection region when a target analyte is detected in a sample
wherein the bioluminescent analyte detection complex is capable of emitting a bioluminescent signal in the presence of a luminogenic substrate.

70. The system of claim 69, wherein the first target analyte binding agent comprises a first target analyte binding element and is non-luminescent, and wherein the second target analyte binding agent comprises a second target analyte binding element and a bioluminescent polypeptide.

71. The system of claim 70, wherein the bioluminescent polypeptide has at least 60% sequence identity with SEQ ID NO: 5.

72. The system of claim 69, wherein the first target analyte binding agent comprises a target first analyte binding element and a polypeptide component of a bioluminescent complex, and the second target analyte binding agent comprises a second target analyte binding element and a peptide component of a bioluminescent complex, wherein the bioluminescent signal produced in the presence of the luminogenic substrate is substantially increased when the first target analyte binding agent contacts the second target analyte binding agent, as compared to a bioluminescent signal produced by the first target analyte binding agent and the luminogenic substrate alone.

73. The system of claim 69, wherein the first target analyte binding agent comprises a first target analyte binding element and a peptide component of a bioluminescent complex, and the second target analyte binding agent comprises a second target analyte binding element and a polypeptide component of a bioluminescent complex, wherein a bioluminescent signal produced in the presence of a luminogenic substrate is substantially increased when the first target analyte binding agent contacts the second target analyte binding agent, as compared to a bioluminescent signal produced by the first target analyte binding agent and the luminogenic substrate alone.

74. The system of claim 72, wherein the polypeptide component of a bioluminescent complex has at least 60% sequence identity with SEQ ID NO: 6, and wherein the peptide component of a bioluminescent complex has at least 60% sequence identity with SEQ ID NO: 10.

75. The system of claim 72, wherein the polypeptide component of a bioluminescent complex has at least 60% sequence identity with SEQ ID NO: 12, and wherein the peptide component of a bioluminescent complex has at least 60% sequence identity with SEQ ID NO: 14.

76. The system of claim 69, wherein the conjugate pad further comprises a polypeptide component of a tripartite bioluminescent complex, wherein the first target analyte binding agent comprises a first target analyte binding element and a first peptide component of the tripartite bioluminescent complex, and the second target analyte binding agent comprises a second target analyte binding element and a second peptide component of the tripartite bioluminescent complex, wherein a bioluminescent signal produced in the presence of a luminogenic substrate is substantially increased when the first target analyte binding agent contacts the second target analyte binding agent and the polypeptide component of the tripartite bioluminescent complex, as compared to a bioluminescent signal produced by (i) the first target analyte binding agent, the second target analyte binding agent, and/or the polypeptide component and (ii) the luminogenic substrate alone.

77. The system of claim 76, wherein the first peptide component of the tripartite bioluminescent complex has at least 60% sequence identity with SEQ ID NO: 11, wherein the first peptide component of the tripartite bioluminescent complex has at least 60% sequence identity with SEQ ID NO: 13, and wherein the polypeptide component of the tripartite bioluminescent complex has at least 60% sequence identity with SEQ ID NO: 12.

78. The system of claim 69, wherein the target analyte is a target antibody.

79. The system of claim 78, wherein the first target analyte binding agent comprises an element that binds non-specifically to antibodies.

80. The system of claim 79, wherein the second target analyte binding agent comprises an element that binds specifically to the target antibody.

81. The system of claim 78, wherein the target antibody is an antibody against a pathogen, toxin, or therapeutic biologic.

82. The system of claim 69, wherein a target analyte binding element is selected from the group consisting of an antibody, a polyclonal antibody, a monoclonal antibody, a recombinant antibody, an antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, an oligonucleotide probe, a peptide nucleic acid, a DARPin, an aptamer, an affimer, a protein domain, and a purified protein.

83. The system of claim 69, further comprising a luminogenic substrate.

84. The system of claim 83, wherein the luminogenic substrate is selected from coelenterazine, coelenterazine-h, coelenterazine-h-h, furimazine, and other coelenterazine analogs or derivatives.

85. The system of claim 84, wherein the luminogenic substrate is applied to the system as part of a composition comprising the luminogenic substrate and a polymer selected from pullulan, trehalose, maltose, cellulose, dextran, polystyrene, poly(meth)acrylate, and a combination of any thereof.

86. The system of claim 85, wherein the composition is applied to at least one of the sample pad, the conjugation pad, the at least one detection region, and the control region.

87. The system of claim 69, wherein the analytical membrane comprises a plurality of detection regions with each detection region comprising a distinct target analyte binding agent comprising distinct target analyte binding elements.

88. The system of claim 69, wherein the system further comprises a device for detecting or quantifying bioluminescent signals from the analyte detection complex.

89-133. (canceled)

134. A method of detecting an analyte in a sample using the lateral flow assay system of claim 69, the method comprising:
applying a sample to the sample pad;
facilitating flow of the sample from the sample pad to the conjugate pad, and then from the conjugate pad to the detection region and the control region on the analytical membrane;
wherein the first target analyte binding agent, the second target analyte binding agent, and the target analyte form the analyte detection complex in the at least one detection region.

135. The method of claim 134, wherein the sample is selected from blood, serum, plasma, urine, stool, cerebral spinal fluid, interstitial fluid, tissue, and saliva.

136. The method of claim 134, wherein the sample is selected from a water sample, a soil sample, a plant sample, a food sample, a beverage sample, an oil, and an industrial fluid sample.

137. The method of claim 134, wherein detecting the target analyte in the sample comprises detecting a bioluminescent signal generated from the analyte detection complex.

138. The method of claim 134, wherein the method further comprises quantifying a bioluminescent signal generated from the analyte detection complex.

139. The method of claim 134, wherein the method further comprises diagnosing a subject from which the sample was obtained as having or not having a disease based on the detection of the analyte.

140-177. (canceled)


SEQUENCE LISTING (730 sequences)

SEQ ID NO: 1 - protein, 170 aa, synthetic construct
  REGION 1..170: note = synthetic
MFTLADFVGDWQQTAGYNQDQVLEQGGLSSLFQALGVSVTPIQKVVLSGENGLKADIHVIIPYEGLSGFQMGLIEMIFKVVYPVDDHHFKIILHYGTLVIDGVTPNMIDYFGRPYPGIAVFDGKQITVTGTLWNGNKIYDERLINPDGSLLFRVTINGVTGWRLCENILA

SEQ ID NO: 2 - protein, 147 aa, synthetic construct
  REGION 1..147: note = synthetic
MFTLADFVGDWQQTAGYNQDQVLEQGGLSSLFQALGVSVTPIQKVVLSGENGLKADIHVIIPYEGLSGFQMGLIEMIFKVVYPVDDHHFKIILHYGTLVIDGVTPNMIDYFGRPYPGIAVFDGKQITVTGTLWNGNKIYDERLINPD

SEQ ID NO: 3 - protein, 10 aa, synthetic construct
  REGION 1..10: note = synthetic
GSLLFRVTIN

SEQ ID NO: 4 - protein, 13 aa, synthetic construct
  REGION 1..13: note = synthetic
GVTGWRLCENILA

SEQ ID NO: 5 - protein, 171 aa, synthetic construct
  REGION 1..171: note = synthetic
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 6 - protein, 148 aa, synthetic construct
  REGION 1..148: note = synthetic
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPD

SEQ ID NO: 7 - protein, 11 aa, synthetic construct
  REGION 1..11: note = synthetic
GSLLFRVTINV

SEQ ID NO: 8 - protein, 13 aa, synthetic construct
  REGION 1..13: note = synthetic
GVTGWRLCERILA

SEQ ID NO: 9 - protein, 158 aa, synthetic construct
  REGION 1..158: note = synthetic
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTIN

SEQ ID NO: 10 - protein, 11 aa, synthetic construct
  REGION 1..11: note = synthetic
VTGYRLFEEIL

SEQ ID NO: 11 - protein, 11 aa, synthetic construct
  REGION 1..11: note = synthetic
VSGWRLFKKIS

SEQ ID NO: 12 - protein, 155 aa, synthetic construct [sequence omitted]

SEQ ID NO: 13 - protein, 11 aa, synthetic construct
  REGION 1..11: note = synthetic
GSMLFRVTINS

SEQ ID NO: 14 - protein, 22 aa, synthetic construct
  REGION 1..22: note = synthetic
GSMLFRVTINSVSGWRLFKKIS

SEQ ID NO: 15 - protein, 11 aa, synthetic construct
  REGION 1..11: note = synthetic
VSGWRLFKKIS

SEQ ID NO: 16 - protein, 13 aa, synthetic construct
  REGION 1..13: note = synthetic
GKMLFRVTINSWK

SEQ ID NO: 17 - protein, 13 aa, synthetic construct
  REGION 1..13: note = synthetic
VSVSGWRLFKKIS

SEQ ID NO: 18 - protein, 11 aa, synthetic construct
  REGION 1..11: note = synthetic
VSGWRLFRRIS

SEQ ID NO: 19 - protein, 13 aa, synthetic construct
  REGION 1..13: note = synthetic
VSVSGWRLFRRIS

SEQ ID NO: 20 - protein, 13 aa, synthetic construct
  REGION 1..13: note = synthetic
GRMLFRVTINSWR

SEQ ID NO: 21 - protein, 13 aa, synthetic construct
  REGION 1..13: note = synthetic
GKMLFRVTINKWK

SEQ ID NO: 22 - protein, 13 aa, synthetic construct
  REGION 1..13: note = synthetic
DKLLFTVTIEKYK

SEQ ID NO: 23 - protein, 13 aa, synthetic construct
  REGION 1..13: note = synthetic
KKMLFRVTIQKWK

SEQ ID NO: 24 - protein, 13 aa, synthetic construct
  REGION 1..13: note = synthetic
GRLLFVVVIERYR

SEQ ID NO: 25 - protein, 13 aa, synthetic construct
  REGION 1..13: note = synthetic
RRMLFRVTIQRWR

SEQ ID NO: 26 - protein, 12 aa, synthetic construct
  REGION 1..12: note = synthetic
VSGWRLFRRISC

SEQ ID NO: 27 - protein, 14 aa, synthetic construct
  REGION 1..14: note = synthetic
GRMLFRVTINSWRC

SEQ ID NO: 28 - DNA, 513 bp, synthetic construct
  misc_feature 1..513: note = synthetic
  source 1..513: mol_type = other DNA
atggtgtttaccttggcagatttcgttggagactggcaacagacagctggatacaaccaagatcaagtgttagaacaaggaggattgtctagtctgttccaagccctgggagtgtcagtcaccccaatccagaaagttgtgctgtctggggagaatgggttaaaagctgatattcatgtcatcatcccttacgagggactcagtggttttcaaatgggtctgattgaaatgatcttcaaagttgtttacccagtggatgatcatcatttcaagattattctccattatggtacactcgttattgacggtgtgacaccaaacatgattgactactttggacgcccttaccctggaattgctgtgtttgacggcaagcagatcacagttactggaactctgtggaacggcaacaagatctatgatgagcgcctgatcaacccagatggttcactcctcttccgcgttactatcaatggagtcaccggatggcgcctttgcgagaacattcttgcc

SEQ ID NO: 29 - DNA, 549 bp, synthetic construct
  misc_feature 1..549: note = synthetic
  source 1..549: mol_type = other DNA
atgaaacatcaccatcaccatcatgcgatcgccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggtt

SEQ ID NO: 30 - DNA, 495 bp, synthetic construct
  misc_feature 1..495: note = synthetic
  source 1..495: mol_type = other DNA
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccac

SEQ ID NO: 31 - DNA, 33 bp, synthetic construct
  misc_feature 1..33: note = synthetic
  source 1..33: mol_type = other DNA
gtgaccggctaccggctgttcgaggagattctg

SEQ ID NO: 32 - DNA, 33 bp, synthetic construct
  misc_feature 1..33: note = synthetic
  source 1..33: mol_type = other DNA
gtgagcggctggcggctgttcaagaagattagc

SEQ ID NO: 33 - protein, 148 aa, synthetic construct
  REGION 1..148: note = synthetic
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPD

SEQ ID NO: 34 - DNA, 444 bp, synthetic construct
  misc_feature 1..444: note = synthetic
  source 1..444: mol_type = other DNA
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgac

SEQ ID NO: 35 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITVTGTLWNGNKIIDERLITPD

SEQ ID NO: 36 - DNA, 465 bp, synthetic construct
  misc_feature 1..465: note = synthetic
  source 1..465: mol_type = other DNA
atgaaacatcaccatcaccatcatgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgac

SEQ ID NO: 37 - protein, 148 aa, synthetic construct
  REGION 1..148: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITVTGTLWNGNKIIDERLITPD

SEQ ID NO: 38 - DNA, 444 bp, synthetic construct
  misc_feature 1..444: note = synthetic
  source 1..444: mol_type = other DNA
atggtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactgtaacagggaccctgtggaacggcaacaaaattatcgacgagcgcctgatcacccccgac

SEQ ID NO: 39 - DNA, 33 bp, synthetic construct
  misc_feature 1..33: note = synthetic
  source 1..33: mol_type = other DNA
ggctccatgctgttccgagtaaccatcaacagc

SEQ ID NO: 40 - DNA, 33 bp, synthetic construct; identical to SEQ ID NO: 32
  misc_feature 1..33: note = synthetic
  source 1..33: mol_type = other DNA

SEQ ID NO: 41 - protein, 148 aa, synthetic construct
  REGION 1..148: note = synthetic
MVFTLEDFVGDWEQTAAYNLDQVLEQGGVSSLFQNLAVSVTPIQRIVLSGENALKIDIHVIIPYEGLSADQMAQIEKIFKVVYPVDDHHFKVILHYGTLVIDGVTPNMINYFGRPYEGIAVFDGKKITVTGTLWNGNKIIDERLITPD

SEQ ID NO: 42 - DNA, 444 bp, synthetic construct
  misc_feature 1..444: note = synthetic
  source 1..444: mol_type = other DNA
atggtcttcacactcgaagatttcgttggggactgggaacagacagccgcctacaacctggaccaagtccttgaacagggaggtgtgtccagtttgtttcagaatctcgccgtgtccgtaactccgatccaaaggattgtcctgagcggtgaaaatgccctgaagatcgacatccatgtcatcatcccgtatgaaggtctgagcgccgaccaaatggcccagatcgaaaaaatttttaaggtggtgtaccctgtggatgatcatcactttaaggtgatcctgcactatggcacactggtaatcgacggggttacgccgaacatgatcaactatttcggacggccgtatgaaggcatcgccgtgttcgacggcaaaaagatcactgtaacagggaccctgtggaacggcaacaaaattatcgacgagcgcctgatcacccccgac

SEQ ID NO: 43 - protein, 11 aa, synthetic construct
  REGION 1..11: note = synthetic
GSMLFRVTINV

SEQ ID NO: 44 - DNA, 30 bp, synthetic construct
  misc_feature 1..30: note = synthetic
  source 1..30: mol_type = other DNA
ggctccatgctgttccgagtaaccatcaac

SEQ ID NO: 45 - protein, 155 aa, synthetic construct
  REGION 1..155: note = synthetic
MKHHHHHHVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPD

SEQ ID NO: 46 - DNA, 465 bp, synthetic construct; identical to SEQ ID NO: 36
  misc_feature 1..465: note = synthetic
  source 1..465: mol_type = other DNA

SEQ ID NO: 47 - DNA, 66 bp, synthetic construct
  misc_feature 1..66: note = synthetic
  source 1..66: mol_type = other DNA
ggctccatgctgttccgagtaaccatcaacagcgtgagcggctggcggctgttcaagaagattagc

SEQ ID NO: 48 - protein, 16 aa, synthetic construct
  REGION 1..16: note = synthetic
SSWKRGSMLFRVTINS

SEQ ID NO: 49 - DNA, 48 bp, synthetic construct
  misc_feature 1..48: note = synthetic
  source 1..48: mol_type = other DNA
agcagctggaagcgcggctccatgctgttccgagtaaccatcaacagc

SEQ ID NO: 50 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGDTPNKLNYFGRPYDGIAVFDGKKITVTGTLWNGNKIIDERLITPD

SEQ ID NO: 51 - DNA, 465 bp, synthetic construct
  misc_feature 1..465: note = synthetic
  source 1..465: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggatacgccgaacaagctgaactatttcggacggccgtatgatggcatcgccgtgttcgacggcaaaaagatcactgtaacagggaccctgtggaacggcaacaaaattatcgacgagcgcctgatcacccccgac

SEQ ID NO: 52 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPSKLNYFGRPYEGIAVFDGKKITVTGTLWNGNKIIDERLITPD

SEQ ID NO: 53 - DNA, 465 bp, synthetic construct
  misc_feature 1..465: note = synthetic
  source 1..465: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgagcaagctgaactatttcggacggccgtatgaaggcatcgccgtgttcgacggcaaaaagatcactgtaacagggaccctgtggaacggcaacaaaattatcgacgagcgcctgatcacccccgac

SEQ ID NO: 54 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGFAVFDGKKITVTGTLWNGNKIIDERLITPD

SEQ ID NO: 55 - DNA, 465 bp, synthetic construct
  misc_feature 1..465: note = synthetic
  source 1..465: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcttcgccgtgttcgacggcaaaaagatcactgtaacagggaccctgtggaacggcaacaaaattatcgacgagcgcctgatcacccccgac

SEQ ID NO: 56 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVCDGKKITVTGTLWNGNKIIDERLITPD

SEQ ID NO: 57 - DNA, 465 bp, synthetic construct
  misc_feature 1..465: note = synthetic
  source 1..465: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgtgcgacggcaaaaagatcactgtaacagggaccctgtggaacggcaacaaaattatcgacgagcgcctgatcacccccgac

SEQ ID NO: 58 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKISVTGTLWNGNKIIDERLITPD

SEQ ID NO: 59 - DNA, 465 bp, synthetic construct
  misc_feature 1..465: note = synthetic
  source 1..465: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcgggcggccgtatgaaggcatcgccgtgttcgacggcaaaaagatctctgtaacagggaccctgtggaacggcaacaaaattatcgacgagcgcctgatcacccccgac

SEQ ID NO: 60 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITATGTLWNGNKIIDERLITPD

SEQ ID NO: 61 - DNA, 465 bp, synthetic construct
  misc_feature 1..465: note = synthetic
  source 1..465: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactgcaacagggaccctgtggaacggcaacaaaattatcgacgagcgcctgatcacccccgac

SEQ ID NO: 62 - DNA, 465 bp, synthetic construct
  misc_feature 1..465: note = synthetic
  source 1..465: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgac

SEQ ID NO: 63 - protein, 149 aa, synthetic construct
  REGION 1..149: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDG

SEQ ID NO: 64 - DNA, 447 bp, synthetic construct
  misc_feature 1..447: note = synthetic
  source 1..447: mol_type = other DNA
atggtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggc

SEQ ID NO: 65 - protein, 147 aa, synthetic construct
  REGION 1..147: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITP

SEQ ID NO: 66 - DNA, 441 bp, synthetic construct
  misc_feature 1..441: note = synthetic
  source 1..441: mol_type = other DNA
atggtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcaccccc

SEQ ID NO: 67 - protein, 146 aa, synthetic construct
  REGION 1..146: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLIT

SEQ ID NO: 68 - DNA, 438 bp, synthetic construct
  misc_feature 1..438: note = synthetic
  source 1..438: mol_type = other DNA
atggtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacc

SEQ ID NO: 69 - protein, 150 aa, synthetic construct
  REGION 1..150: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGS

SEQ ID NO: 70 - DNA, 450 bp, synthetic construct
  misc_feature 1..450: note = synthetic
  source 1..450: mol_type = other DNA
atggtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggcagc

SEQ ID NO: 71 - protein, 164 aa, synthetic construct
  REGION 1..164: note = synthetic
MFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 72 - DNA, 495 bp, synthetic construct
  misc_feature 1..495: note = synthetic
  source 1..495: mol_type = other DNA
atgttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 73 - protein, 163 aa, synthetic construct
  REGION 1..163: note = synthetic
MTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 74 - DNA, 492 bp, synthetic construct
  misc_feature 1..492: note = synthetic
  source 1..492: mol_type = other DNA
atgacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 75 - protein, 162 aa, synthetic construct
  REGION 1..162: note = synthetic
MLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 76 - DNA, 489 bp, synthetic construct
  misc_feature 1..489: note = synthetic
  source 1..489: mol_type = other DNA
atgctcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 77 - protein, 161 aa, synthetic construct
  REGION 1..161: note = synthetic
MEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 78 - DNA, 486 bp, synthetic construct
  misc_feature 1..486: note = synthetic
  source 1..486: mol_type = other DNA
atggaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 79 - protein, 174 aa, synthetic construct
  REGION 1..174: note = synthetic
MFKKISGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 80 - DNA, 525 bp, synthetic construct
  misc_feature 1..525: note = synthetic
  source 1..525: mol_type = other DNA
atgttcaagaagattagcggctcgagcggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 81 - protein, 173 aa, synthetic construct
  REGION 1..173: note = synthetic
MKKISGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 82 - DNA, 522 bp, synthetic construct
  misc_feature 1..522: note = synthetic
  source 1..522: mol_type = other DNA
atgaagaagattagcggctcgagcggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 83 - protein, 172 aa, synthetic construct
  REGION 1..172: note = synthetic
MKISGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 84 - DNA, 519 bp, synthetic construct
  misc_feature 1..519: note = synthetic
  source 1..519: mol_type = other DNA
atgaagattagcggctcgagcggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 85 - protein, 171 aa, synthetic construct
  REGION 1..171: note = synthetic
MISGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 86 - DNA, 516 bp, synthetic construct
  misc_feature 1..516: note = synthetic
  source 1..516: mol_type = other DNA
atgattagcggctcgagcggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 87 - protein, 170 aa, synthetic construct
  REGION 1..170: note = synthetic
MSGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 88 - DNA, 513 bp, synthetic construct
  misc_feature 1..513: note = synthetic
  source 1..513: mol_type = other DNA
atgagcggctcgagcggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 89 - protein, 158 aa, synthetic construct
  REGION 1..158: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSM

SEQ ID NO: 90 - DNA, 477 bp, synthetic construct
  misc_feature 1..477: note = synthetic
  source 1..477: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggcagcatgtaa

SEQ ID NO: 91 - protein, 159 aa, synthetic construct
  REGION 1..159: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSML

SEQ ID NO: 92 - DNA, 480 bp, synthetic construct
  misc_feature 1..480: note = synthetic
  source 1..480: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggcagcatgctgtaa

SEQ ID NO: 93 - protein, 160 aa, synthetic construct
  REGION 1..160: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSMLF

SEQ ID NO: 94 - DNA, 483 bp, synthetic construct
  misc_feature 1..483: note = synthetic
  source 1..483: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggcagcatgctgttctaa

SEQ ID NO: 95 - protein, 152 aa, synthetic construct
  REGION 1..152: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLI

SEQ ID NO: 96 - DNA, 459 bp, synthetic construct
  misc_feature 1..459: note = synthetic
  source 1..459: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatctaa

SEQ ID NO: 97 - protein, 151 aa, synthetic construct
  REGION 1..151: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERL

SEQ ID NO: 98 - DNA, 456 bp, synthetic construct
  misc_feature 1..456: note = synthetic
  source 1..456: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgtaa

SEQ ID NO: 99 - protein, 150 aa, synthetic construct
  REGION 1..150: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDER

SEQ ID NO: 100 - DNA, 453 bp, synthetic construct
  misc_feature 1..453: note = synthetic
  source 1..453: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgctaa

SEQ ID NO: 101 - protein, 123 aa, synthetic construct
  REGION 1..123: note = synthetic
MVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGGGSGGSSSGGAIVSGWRLFKKIS

SEQ ID NO: 102 - DNA, 372 bp, synthetic construct
  misc_feature 1..372: note = synthetic
  source 1..372: mol_type = other DNA
atggtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtggcgggagcggtggctcgagcagcggtggagcgatcgtgagcggctggcggctgttcaagaagattagctaa

SEQ ID NO: 103 - protein, 125 aa, synthetic construct
  REGION 1..125: note = synthetic
MVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGGGSGGSSSGGAIVSVSGWRLFKKIS

SEQ ID NO: 104 - DNA, 378 bp, synthetic construct
  misc_feature 1..378: note = synthetic
  source 1..378: mol_type = other DNA
atggtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtggcgggagcggtggctcgagcagcggtggagcgatcgttagcgttagcggctggcgcctgttcaagaagatcagctaa

SEQ ID NO: 105 - protein, 129 aa, synthetic construct
  REGION 1..129: note = synthetic
MVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGGGSGGSSSGGAIVSGWRLFKKISHHHHHH

SEQ ID NO: 106 - DNA, 390 bp, synthetic construct
  misc_feature 1..390: note = synthetic
  source 1..390: mol_type = other DNA
atggtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtggcgggagcggtggctcgagcagcggtggagcgatcgtgagcggctggcggctgttcaagaagattagccatcatcaccatcaccactaa

SEQ ID NO: 107 - protein, 131 aa, synthetic construct
  REGION 1..131: note = synthetic
MVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGGGSGGSSSGGAIVSVSGWRLFKKISHHHHHH

SEQ ID NO: 108 - DNA, 396 bp, synthetic construct
  misc_feature 1..396: note = synthetic
  source 1..396: mol_type = other DNA
atggtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtggcgggagcggtggctcgagcagcggtggagcgatcgttagcgtgagcggctggcggctgttcaagaagattagccatcatcaccatcaccactaa

SEQ ID NO: 109 - protein, 118 aa, synthetic construct
  REGION 1..118: note = synthetic
MKHHHHHHVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGVSGWRLFKKIS

SEQ ID NO: 110 - DNA, 357 bp, synthetic construct
  misc_feature 1..357: note = synthetic
  source 1..357: mol_type = other DNA
atgaaacatcaccatcaccatcatgtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtgtgagcggctggcggctgttcaagaagattagctaa

SEQ ID NO: 111 - protein, 123 aa, synthetic construct
  REGION 1..123: note = synthetic
MKHHHHHHVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGGGSGGVSGWRLFKKIS

SEQ ID NO: 112 - DNA, 372 bp, synthetic construct
  misc_feature 1..372: note = synthetic
  source 1..372: mol_type = other DNA
atgaaacatcaccatcaccatcatgtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtggcgggagcggtggcgtgagcggctggcggctgttcaagaagattagctaa

SEQ ID NO: 113 - protein, 128 aa, synthetic construct
  REGION 1..128: note = synthetic
MKHHHHHHVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGGGSGGSSSGGVSGWRLFKKIS

SEQ ID NO: 114 - DNA, 387 bp, synthetic construct
  misc_feature 1..387: note = synthetic
  source 1..387: mol_type = other DNA
atgaaacatcaccatcaccatcatgtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtggcgggagcggtggctcgagcagcggtggagtgagcggctggcggctgttcaagaagattagctaa

SEQ ID NO: 115 - protein, 120 aa, synthetic construct
  REGION 1..120: note = synthetic
MKHHHHHHVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGVSVSGWRLFKKIS

SEQ ID NO: 116 - DNA, 363 bp, synthetic construct
  misc_feature 1..363: note = synthetic
  source 1..363: mol_type = other DNA
atgaaacatcaccatcaccatcatgtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtgttagcgttagcggctggcgcctgttcaagaagatcagctaa

SEQ ID NO: 117 - protein, 125 aa, synthetic construct
  REGION 1..125: note = synthetic
MKHHHHHHVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGGGSGGVSVSGWRLFKKIS

SEQ ID NO: 118 - DNA, 378 bp, synthetic construct
  misc_feature 1..378: note = synthetic
  source 1..378: mol_type = other DNA
atgaaacatcaccatcaccatcatgtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtggcgggagcggtggcgttagcgttagcggctggcgcctgttcaagaagatcagctaa

SEQ ID NO: 119 - protein, 130 aa, synthetic construct
  REGION 1..130: note = synthetic
MKHHHHHHVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGGGSGGSSSGGVSVSGWRLFKKIS

SEQ ID NO: 120 - DNA, 393 bp, synthetic construct
  misc_feature 1..393: note = synthetic
  source 1..393: mol_type = other DNA
atgaaacatcaccatcaccatcatgtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtggcgggagcggtggctcgagcagcggtggagttagcgttagcggctggcgcctgttcaagaagatcagctaa

SEQ ID NO: 121 - protein, 134 aa, synthetic construct
  REGION 1..134: note = synthetic
MGSMLFRVTINSSSSGGGGSGGGSSGGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 122 - DNA, 405 bp, synthetic construct
  misc_feature 1..405: note = synthetic
  source 1..405: mol_type = other DNA
atgggctccatgctgttccgagtaaccatcaacagctcgagttcaggtggtggcgggagcggtggagggagcagcggtggaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaataa

SEQ ID NO: 123 - protein, 136 aa, synthetic construct
  REGION 1..136: note = synthetic
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGGGSGGSSSGGAIGSMLFRVTINS

SEQ ID NO: 124 - DNA, 408 bp, synthetic construct
  misc_feature 1..408: note = synthetic
  source 1..408: mol_type = other DNA
atgggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggtggttcaggtggtggcgggagcggtggctcgagcagcggtggagcgatcggctccatgctgttccgagtaaccatcaacagc

SEQ ID NO: 125 - protein, 165 aa, synthetic construct
  REGION 1..165: note = synthetic
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 126 - DNA, 498 bp, synthetic construct
  misc_feature 1..498: note = synthetic
  source 1..498: mol_type = other DNA
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 127 - protein, 13 aa, synthetic construct
  REGION 1..13: note = synthetic
NVSGWRLFKKISN

SEQ ID NO: 128 - protein, 13 aa, synthetic construct
  REGION 1..13: note = synthetic
NVTGYRLFKKISN

SEQ ID NO: 129 - protein, 12 aa, synthetic construct
  REGION 1..12: note = synthetic
VSGWRLFKKISN

SEQ ID NO: 130 - protein, 11 aa, synthetic construct
  REGION 1..11: note = synthetic
SGWRLFKKISN

SEQ ID NO: 131 - protein, 10 aa, synthetic construct
  REGION 1..10: note = synthetic
GWRLFKKISN

SEQ ID NO: 132 - protein, 11 aa, synthetic construct
  REGION 1..11: note = synthetic
VTGYRLFEKIS

SEQ ID NO: 133 - protein, 10 aa, synthetic construct
  REGION 1..10: note = synthetic
SGWRLFKKIS

SEQ ID NO: 134 - protein, 6 aa, synthetic construct
  REGION 1..6: note = synthetic
VSGWRL

SEQ ID NO: 135 - protein, 7 aa, synthetic construct
  REGION 1..7: note = synthetic
VSGWRLF

SEQ ID NO: 136 - protein, 8 aa, synthetic construct
  REGION 1..8: note = synthetic
VSGWRLFK

SEQ ID NO: 137 - protein, 9 aa, synthetic construct
  REGION 1..9: note = synthetic
VSGWRLFKK

SEQ ID NO: 138 - protein, 10 aa, synthetic construct
  REGION 1..10: note = synthetic
VSGWRLFKKI

SEQ ID NO: 139 - protein, 11 aa, synthetic construct
  REGION 1..11: note = synthetic
VSGWRLYKKIS

SEQ ID NO: 140 - protein, 22 aa, synthetic construct
  REGION 1..22: note = synthetic
GSMLFRVTINSVSGWALFKKIS

SEQ ID NO: 141 - protein, 22 aa, synthetic construct
  REGION 1..22: note = synthetic
GSMLFRVTINSVTGYRLFEEIL

SEQ ID NO: 142 - protein, 16 aa, synthetic construct
  REGION 1..16: note = synthetic
GSMLFRVTINSSSWKR

SEQ ID NO: 143 - protein, 14 aa, synthetic construct
  REGION 1..14: note = synthetic
VSGVSGWRLFKKIS

SEQ ID NO: 144 - protein, 12 aa, synthetic construct
  REGION 1..12: note = synthetic
VVSGWRLFKKIS

SEQ ID NO: 145 - protein, 15 aa, synthetic construct
  REGION 1..15: note = synthetic
SSWKRSMLFRVTINS

SEQ ID NO: 146 - protein, 14 aa, synthetic construct
  REGION 1..14: note = synthetic
SSWKRMLFRVTINS

SEQ ID NO: 147 - protein, 17 aa, synthetic construct
  REGION 1..17: note = synthetic
SSWKRDGSMLFRVTINS

SEQ ID NO: 148 - protein, 18 aa, synthetic construct
  REGION 1..18: note = synthetic
SSWKRPDGSMLFRVTINS

SEQ ID NO: 149 - protein, 16 aa, synthetic construct
  REGION 1..16: note = synthetic
SSWKRSMLFRVTINSV

SEQ ID NO: 150 - protein, 15 aa, synthetic construct
  REGION 1..15: note = synthetic
SSWKRMLFRVTINSV

SEQ ID NO: 151 - protein, 18 aa, synthetic construct
  REGION 1..18: note = synthetic
SSWKRDGSMLFRVTINSV

SEQ ID NO: 152 - protein, 19 aa, synthetic construct
  REGION 1..19: note = synthetic
SSWKRPDGSMLFRVTINSV

SEQ ID NO: 153 - protein, 17 aa, synthetic construct
  REGION 1..17: note = synthetic
SSWKRSMLFRVTINSVS

SEQ ID NO: 154 - protein, 16 aa, synthetic construct
  REGION 1..16: note = synthetic
SSWKRMLFRVTINSVS

SEQ ID NO: 155 - protein, 19 aa, synthetic construct
  REGION 1..19: note = synthetic
SSWKRDGSMLFRVTINSVS

SEQ ID NO: 156 - protein, 20 aa, synthetic construct
  REGION 1..20: note = synthetic
SSWKRPDGSMLFRVTINSVS

SEQ ID NO: 157 - protein, 15 aa, synthetic construct
  REGION 1..15: note = synthetic
SSWKRGSMLFRVTIN

SEQ ID NO: 158 - protein, 14 aa, synthetic construct
  REGION 1..14: note = synthetic
SSWKRGSMLFRVTI

SEQ ID NO: 159 - protein, 14 aa, synthetic construct
  REGION 1..14: note = synthetic
SSWKRSMLFRVTIN

SEQ ID NO: 160 - protein, 13 aa, synthetic construct
  REGION 1..13: note = synthetic
SSWKRMLFRVTIN

SEQ ID NO: 161 - protein, 16 aa, synthetic construct
  REGION 1..16: note = synthetic
SSWKRDGSMLFRVTIN

SEQ ID NO: 162 - protein, 17 aa, synthetic construct
  REGION 1..17: note = synthetic
SSWKRPDGSMLFRVTIN

SEQ ID NO: 163 - protein, 13 aa, synthetic construct
  REGION 1..13: note = synthetic
SSWKRSMLFRVTI

SEQ ID NO: 164 - protein, 12 aa, synthetic construct
  REGION 1..12: note = synthetic
SSWKRMLFRVTI

SEQ ID NO: 165 - protein, 15 aa, synthetic construct
  REGION 1..15: note = synthetic
SSWKRDGSMLFRVTI

SEQ ID NO: 166 - protein, 16 aa, synthetic construct
  REGION 1..16: note = synthetic
SSWKRPDGSMLFRVTI

SEQ ID NO: 167 - protein, 15 aa, synthetic construct
  REGION 1..15: note = synthetic
VSGWRLFKKISVFTL

SEQ ID NO: 168 - protein, 14 aa, synthetic construct
  REGION 1..14: note = synthetic
VSGWRLFKKISVFT

SEQ ID NO: 169 - protein, 13 aa, synthetic construct
  REGION 1..13: note = synthetic
VSGWRLFKKISVF

SEQ ID NO: 170 - protein, 12 aa, synthetic construct
  REGION 1..12: note = synthetic
VSGWRLFKKISV

SEQ ID NO: 171 - protein, 11 aa, synthetic construct
  REGION 1..11: note = synthetic
VSGWRLCKKIS

SEQ ID NO: 172 - protein, 22 aa, synthetic construct
  REGION 1..22: note = synthetic
VSGWRLFKKISGSMLFRVTINS

SEQ ID NO: 173 - protein, 13 aa, synthetic construct
  REGION 1..13: note = synthetic
SSWKRLFRVTINS

SEQ ID NO: 174 - protein, 12 aa, synthetic construct
  REGION 1..12: note = synthetic
SSWKRFRVTINS

SEQ ID NO: 175 - protein, 11 aa, synthetic construct
  REGION 1..11: note = synthetic
SSWKRRVTINS

SEQ ID NO: 176 - protein, 19 aa, synthetic construct
  REGION 1..19: note = synthetic
SSWKRTPDGSMLFRVTINS

SEQ ID NO: 177 - protein, 20 aa, synthetic construct
  REGION 1..20: note = synthetic
SSWKRITPDGSMLFRVTINS

SEQ ID NO: 178 - protein, 21 aa, synthetic construct
  REGION 1..21: note = synthetic
SSWKRLITPDGSMLFRVTINS

SEQ ID NO: 179 - protein, 16 aa, synthetic construct
  REGION 1..16: note = synthetic
SSRGSMLFRVTINSWK

SEQ ID NO: 180 - protein, 16 aa, synthetic construct
  REGION 1..16: note = synthetic
SKRGSMLFRVTINSWS

SEQ ID NO: 181 - protein, 14 aa, synthetic construct
  REGION 1..14: note = synthetic
SWRGSMLFRVTINS

SEQ ID NO: 182 - protein, 14 aa, synthetic construct
  REGION 1..14: note = synthetic
SSRGSMLFRVTIWK

SEQ ID NO: 183 - protein, 16 aa, synthetic construct
  REGION 1..16: note = synthetic
SSWKRGSMLYRVTINS

SEQ ID NO: 184 - protein, 16 aa, synthetic construct
  REGION 1..16: note = synthetic
SSWKRGSMLWRVTINS

SEQ ID NO: 185 - protein, 16 aa, synthetic construct
  REGION 1..16: note = synthetic
SSWKRGSMLHRVTINS

SEQ ID NO: 186 - protein, 16 aa, synthetic construct
  REGION 1..16: note = synthetic
SSWKRGSLLFRVTINS

SEQ ID NO: 187 - protein, 16 aa, synthetic construct
  REGION 1..16: note = synthetic
SSWKRGSKLFRVTINS

SEQ ID NO: 188 - protein, 16 aa, synthetic construct
  REGION 1..16: note = synthetic
SSWKRGSRLFRVTINS

SEQ ID NO: 189 - protein, 16 aa, synthetic construct
  REGION 1..16: note = synthetic
SSWKRGSFLFRVTINS

SEQ ID NO: 190 - protein, 16 aa, synthetic construct
  REGION 1..16: note = synthetic
SSWKRGSWLFRVTINS

SEQ ID NO: 191 - protein, 16 aa, synthetic construct
  REGION 1..16: note = synthetic
SSWKRGSMLFRVSINS

SEQ ID NO: 192 - protein, 16 aa, synthetic construct
  REGION 1..16: note = synthetic
SSWKRGSMLFRVQINS

SEQ ID NO: 193 - protein, 16 aa, synthetic construct
  REGION 1..16: note = synthetic
SSWKRGSMLFRVNINS

SEQ ID NO: 194 - protein, 17 aa, synthetic construct
  REGION 1..17: note = synthetic
SSWKRGSMLFRVTINSC

SEQ ID NO: 195 - protein, 12 aa, synthetic construct
  REGION 1..12: note = synthetic
CVSGWRLFKKIS

SEQ ID NO: 196 - protein, 17 aa, synthetic construct
  REGION 1..17: note = synthetic
SSWKRGSMLFRVTINSK

SEQ ID NO: 197 - protein, 12 aa, synthetic construct
  REGION 1..12: note = synthetic
KVSGWRLFKKIS

SEQ ID NO: 198 - protein, 23 aa, synthetic construct
  REGION 1..23: note = synthetic
GSLLFRVTINGVTGWRLCENILA

SEQ ID NO: 199 - protein, 24 aa, synthetic construct
  REGION 1..24: note = synthetic
GSLLFRVTINVGVTGWRLCERILA

SEQ ID NO: 200 - protein, 12 aa, synthetic construct
  REGION 1..12: note = synthetic
SVSGWRLFKKIS

SEQ ID NO: 201 - protein, 13 aa, synthetic construct
  REGION 1..13: note = synthetic
NSVSGWRLFKKIS

SEQ ID NO: 202 - protein, 9 aa, synthetic construct
  REGION 1..9: note = synthetic
GWRLFKKIS

SEQ ID NO: 203 - DNA, 501 bp, synthetic construct
  misc_feature 1..501: note = synthetic
  source 1..501: mol_type = other DNA
atggtgagcggctggcggctgttcaagaagattagccaccatcaccatcaccatcatcacttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgactaa

SEQ ID NO: 204 - protein, 166 aa, synthetic construct
  REGION 1..166: note = synthetic
MVSGWRLFKKISHHHHHHHHFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPD

SEQ ID NO: 205 - DNA, 510 bp, synthetic construct
  misc_feature 1..510: note = synthetic
  source 1..510: mol_type = other DNA
atgaaacatcaccatcaccatcatgtgagcggctggcggctgttcaagaagattagcggcagctccggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgactaa

SEQ ID NO: 206 - protein, 169 aa, synthetic construct
  REGION 1..169: note = synthetic
MKHHHHHHVSGWRLFKKISGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPD

SEQ ID NO: 207 - DNA, 366 bp, synthetic construct
  misc_feature 1..366: note = synthetic
  source 1..366: mol_type = other DNA
atggtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtggcgggagcggtggctcgagcagcggtggagtgagcggctggcggctgttcaagaagattagctaa

SEQ ID NO: 208 - protein, 121 aa, synthetic construct
  REGION 1..121: note = synthetic
MVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGGGSGGSSSGGVSGWRLFKKIS

SEQ ID NO: 209 - DNA, 372 bp, synthetic construct
  misc_feature 1..372: note = synthetic
  source 1..372: mol_type = other DNA
atggtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatcagtggtggttcaggtggtggcgggagcggtggctcgagcagcggtggagttagcgttagcggctggcgcctgttcaagaagatcagctaa

SEQ ID NO: 210 - protein, 123 aa, synthetic construct
  REGION 1..123: note = synthetic
MVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGGGSGGSSSGGVSVSGWRLFKKIS

SEQ ID NO: 211 - DNA, 369 bp, synthetic construct
  misc_feature 1..369: note = synthetic
  source 1..369: mol_type = other DNA
atgggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggtggttcaggtggtggcgggagcggtggctcgagcagcggtgga

SEQ ID NO: 212 - protein, 123 aa, synthetic construct
  REGION 1..123: note = synthetic
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGGGSGGSSSGG

SEQ ID NO: 213 - protein, 12 aa, synthetic construct
  REGION 1..12: note = synthetic
GSMLFRVTINSV

SEQ ID NO: 214 - protein, 13 aa, synthetic construct
  REGION 1..13: note = synthetic
GSMLFRVTINSVS

SEQ ID NO: 215 - protein, 12 aa, synthetic construct
  REGION 1..12: note = synthetic
MLFRVTINSVSG

SEQ ID NO: 216 - protein, 13 aa, synthetic construct
  REGION 1..13: note = synthetic
MLFRVTINSVSGW

SEQ ID NO: 217 - protein, 14 aa, synthetic construct
  REGION 1..14: note = synthetic
MLFRVTINSVSGWK

SEQ ID NO: 218 - protein, 14 aa, synthetic construct
  REGION 1..14: note = synthetic
MLFRVTINSVSGWR

SEQ ID NO: 219 - protein, 14 aa, synthetic construct
  REGION 1..14: note = synthetic
GSMLFRVTINSVSG

SEQ ID NO: 220 - protein, 15 aa, synthetic construct
  REGION 1..15: note = synthetic
GSMLFRVTINSVSGW

SEQ ID NO: 221 - protein, 16 aa, synthetic construct
  REGION 1..16: note = synthetic
GSMLFRVTINSVSGWR

SEQ ID NO: 222 - protein, 16 aa, synthetic construct
  REGION 1..16: note = synthetic
GSMLFRVTINSVSGWK

SEQ ID NO: 223 - protein, 11 aa, synthetic construct
  REGION 1..11: note = synthetic
GSMLFRVTIWK

SEQ ID NO: 224 - protein, 13 aa, synthetic construct
  REGION 1..13: note = synthetic
GSMLFRVTINSWK

SEQ ID NO: 225 - protein, 11 aa, synthetic construct
  REGION 1..11: note = synthetic
MLFRVTINSWK

SEQ ID NO: 226 - protein, 11 aa, synthetic construct
  REGION 1..11: note = synthetic
MLFRVTINSWS

SEQ ID NO: 227 - protein, 9 aa, synthetic construct
  REGION 1..9: note = synthetic
MLFRVTIWS

SEQ ID NO: 228 - protein, 9 aa, synthetic construct
  REGION 1..9: note = synthetic
MLFRVTIWK

SEQ ID NO: 229 - protein, 9 aa, synthetic construct
  REGION 1..9: note = synthetic
MLFRVKINS

SEQ ID NO: 230 - protein, 13 aa, synthetic construct
  REGION 1..13: note = synthetic
GSMLFRVTINSWS

SEQ ID NO: 231 - protein, 11 aa, synthetic construct
  REGION 1..11: note = synthetic
GSMLFRVKINS

SEQ ID NO: 232 - protein, 11 aa, synthetic construct
  REGION 1..11: note = synthetic
GSMLFRVTIWS

SEQ ID NO: 233 - protein, 9 aa, synthetic construct
  REGION 1..9: note = synthetic
MLFRVNINS

SEQ ID NO: 234 - protein, 9 aa, synthetic construct
  REGION 1..9: note = synthetic
MLFRVWINS

SEQ ID NO: 235 - protein, 9 aa, synthetic construct
  REGION 1..9: note = synthetic
LLFRVKINS

SEQ ID NO: 236 - protein, 9 aa, synthetic construct
  REGION 1..9: note = synthetic
FLFRVTINS

SEQ ID NO: 237 - protein, 17 aa, synthetic construct
  REGION 1..17: note = synthetic
SSWKRGSMLFRVTINSV

SEQ ID NO: 238 - protein, 18 aa, synthetic construct
  REGION 1..18: note = synthetic
SSWKRGSMLFRVTINSVS

SEQ ID NO: 239 - protein, 17 aa, synthetic construct
  REGION 1..17: note = synthetic
SSWKRMLFRVTINSVSG

SEQ ID NO: 240 - protein, 18 aa, synthetic construct
  REGION 1..18: note = synthetic
SSWKRMLFRVTINSVSGW

SEQ ID NO: 241 - protein, 19 aa, synthetic construct
  REGION 1..19: note = synthetic
SSWKRMLFRVTINSVSGWR

SEQ ID NO: 242 - protein, 19 aa, synthetic construct
  REGION 1..19: note = synthetic
SSWKRMLFRVTINSVSGWK

SEQ ID NO: 243 - protein, 14 aa, synthetic construct
  REGION 1..14: note = synthetic
MLFRVTINSVSGWK

SEQ ID NO: 244 - protein, 19 aa, synthetic construct
  REGION 1..19: note = synthetic
SSWKRGSMLFRVTINSVSG

SEQ ID NO: 245 - protein, 20 aa, synthetic construct
  REGION 1..20: note = synthetic
SSWKRGSMLFRVTINSVSGW

SEQ ID NO: 246 - protein, 21 aa, synthetic construct
  REGION 1..21: note = synthetic
SSWKRGSMLFRVTINSVSGWR

SEQ ID NO: 247 - protein, 21 aa, synthetic construct
  REGION 1..21: note = synthetic
SSWKRGSMLFRVTINSVSGWK

SEQ ID NO: 248 - protein, 16 aa, synthetic construct
  REGION 1..16: note = synthetic
SSWKRGSYLFRVTINS

SEQ ID NO: 249 - protein, 16 aa, synthetic construct
  REGION 1..16: note = synthetic
SSWKRGSMLFRVKINS

SEQ ID NO: 250 - protein, 16 aa, synthetic construct
  REGION 1..16: note = synthetic
SSWKRGSMLFRVRINS

SEQ ID NO: 251 - protein, 16 aa, synthetic construct
  REGION 1..16: note = synthetic
SSWKRGSMLFRVWINS

SEQ ID NO: 252 - protein, 16 aa, synthetic construct
  REGION 1..16: note = synthetic
SSKRGSMLFRVTIWSV

SEQ ID NO: 253 - protein, 17 aa, synthetic construct
  REGION 1..17: note = synthetic
SSKRGSMLFRVTIWSVS

SEQ ID NO: 254 - protein, 15 aa, synthetic construct
  REGION 1..15: note = synthetic
SSWRGSMLFRVTIKS

SEQ ID NO: 255 - protein, 15 aa, synthetic construct
  REGION 1..15: note = synthetic
KRSSGSMLFRVTIWS

SEQ ID NO: 256 - protein, 13 aa, synthetic construct
  REGION 1..13: note = synthetic
SSKRMLFRVTIWS

SEQ ID NO: 257 - protein, 13 aa, synthetic construct
  REGION 1..13: note = synthetic
KRSSMLFRVTIWS

SEQ ID NO: 258 - protein, 13 aa, synthetic construct
  REGION 1..13: note = synthetic
GSMKFRVTINSWK

SEQ ID NO: 259 - protein, 13 aa, synthetic construct
  REGION 1..13: note = synthetic
GSMLFRKTINSWK

SEQ ID NO: 260 - protein, 13 aa, synthetic construct
  REGION 1..13: note = synthetic
GSMLFRVTKNSWK

SEQ ID NO: 261 - protein, 11 aa, synthetic construct
  REGION 1..11: note = synthetic
GKMLFRVTIWK

SEQ ID NO: 262 - protein, 13 aa, synthetic construct
  REGION 1..13: note = synthetic
GSMKFRVTINSWK

SEQ ID NO: 263 - protein, 11 aa, synthetic construct
  REGION 1..11: note = synthetic
GSMKFRVTIWK

SEQ ID NO: 264 - protein, 13 aa, synthetic construct
  REGION 1..13: note = synthetic
GRMLFRVTINSWK

SEQ ID NO: 265 - protein, 11 aa, synthetic construct
  REGION 1..11: note = synthetic
GRMLFRVTIWK

SEQ ID NO: 266 - protein, 13 aa, synthetic construct
  REGION 1..13: note = synthetic
GSMRFRVTINSWK

SEQ ID NO: 267 - protein, 11 aa, synthetic construct
  REGION 1..11: note = synthetic
GSMRFRVTIWK

SEQ ID NO: 268 - protein, 13 aa, synthetic construct
  REGION 1..13: note = synthetic
GDMLFRVTINSWK

SEQ ID NO: 269 - protein, 11 aa, synthetic construct
  REGION 1..11: note = synthetic
GDMLFRVTIWK

SEQ ID NO: 270 - protein, 13 aa, synthetic construct
  REGION 1..13: note = synthetic
GSMDFRVTINSWK

SEQ ID NO: 271 - protein, 11 aa, synthetic construct
  REGION 1..11: note = synthetic
GSMDFRVTIWK

SEQ ID NO: 272 - protein, 13 aa, synthetic construct
  REGION 1..13: note = synthetic
GEMLFRVTINSWK

SEQ ID NO: 273 - protein, 13 aa, synthetic construct
  REGION 1..13: note = synthetic
GSMEFRVTINSWK

SEQ ID NO: 274 - protein, 11 aa, synthetic construct
  REGION 1..11: note = synthetic
GSMEFRVTIWK

SEQ ID NO: 275 - protein, 13 aa, synthetic construct
  REGION 1..13: note = synthetic
GSMLFRVTIWKVK

SEQ ID NO: 276 - protein, 13 aa, synthetic construct
  REGION 1..13: note = synthetic
GSMLFRVTIWSVK

SEQ ID NO: 277 - protein, 12 aa, synthetic construct
  REGION 1..12: note = synthetic
GSMLFRVTIWSK

SEQ ID NO: 278 - protein, 13 aa, synthetic construct
  REGION 1..13: note = synthetic
GSMLFRVTIWKWK

SEQ ID NO: 279 - protein, 12 aa, synthetic construct
  REGION 1..12: note = synthetic
GSMLFRVTIWKK

SEQ ID NO: 280 - protein, 11 aa, synthetic construct
  REGION 1..11: note = synthetic
GSMLFRVTINS

SEQ ID NO: 281 - protein, 9 aa, synthetic construct
  REGION 1..9: note = synthetic
MLFRVTINS

SEQ ID NO: 282 - protein, 9 aa, synthetic construct
  REGION 1..9: note = synthetic
MLFVTINSV

SEQ ID NO: 283 - protein, 11 aa, synthetic construct
  REGION 1..11: note = synthetic
MLFRVTINSVS

SEQ ID NO: 284 - protein, 10 aa, synthetic construct
  REGION 1..10: note = synthetic
GSMLFRVTIN

SEQ ID NO: 285 - protein, 9 aa, synthetic construct
  REGION 1..9: note = synthetic
GSMLFRVTI

SEQ ID NO: 286 - protein, 9 aa, synthetic construct
  REGION 1..9: note = synthetic
SMLFRVTIN

SEQ ID NO: 287 - protein, 8 aa, synthetic construct
  REGION 1..8: note = synthetic
MLFRVTIN

SEQ ID NO: 288 - protein, 7 aa, synthetic construct
  REGION 1..7: note = synthetic
MLFRVTI

SEQ ID NO: 289 - protein, 15 aa, synthetic construct
  REGION 1..15: note = synthetic
SSKRGSMLFRVTIWS

SEQ ID NO: 290 - protein, 23 aa, synthetic construct
  REGION 1..23: note = synthetic
GSMLFRVTINSGVSGWALFKKIS

SEQ ID NO: 291 - protein, 23 aa, synthetic construct
  REGION 1..23: note = synthetic
GSMLFRVTINSGVSGWRLFKKIS

SEQ ID NO: 292 - protein, 11 aa, synthetic construct
  REGION 1..11: note = synthetic
VSGWALFKKIS

SEQ ID NO: 293 - protein, 25 aa, synthetic construct
  REGION 1..25: note = synthetic
GSMLFRVTINSVSGVSGWRLFKKIS

SEQ ID NO: 294 - protein, 148 aa, synthetic construct
  REGION 1..148: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPD

SEQ ID NO: 295 - DNA, 444 bp, synthetic construct
  misc_feature 1..444: note = synthetic
  source 1..444: mol_type = other DNA
atggtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgac

SEQ ID NO: 296 - protein, 148 aa, synthetic construct; identical to SEQ ID NO: 37
  REGION 1..148: note = synthetic

SEQ ID NO: 297 - DNA, 444 bp, synthetic construct; identical to SEQ ID NO: 34
  misc_feature 1..444: note = synthetic
  source 1..444: mol_type = other DNA

SEQ ID NO: 298 - protein, 12 aa, synthetic construct
  REGION 1..12: note = synthetic
SVSGWRLFKKIS

SEQ ID NO: 299 - protein, 13 aa, synthetic construct
  REGION 1..13: note = synthetic
NSVSGWRLFKKIS

SEQ ID NO: 300 - protein, 9 aa, synthetic construct
  REGION 1..9: note = synthetic
GWRLFKKIS

SEQ ID NO: 301 - protein, 23 aa, synthetic construct
  REGION 1..23: note = synthetic
GSMLFRVTINSGVSGWRLFKKIS

SEQ ID NO: 302 - protein, 13 aa, synthetic construct
  REGION 1..13: note = synthetic
GSMLFRVTINSWK

SEQ ID NO: 303 - protein, 13 aa, synthetic construct
  REGION 1..13: note = synthetic
GSMLFRVTINSWK

SEQ ID NO: 304 - protein, 13 aa, synthetic construct
  REGION 1..13: note = synthetic
GSMLFRVTINSWK

SEQ ID NO: 305 - protein, 13 aa, synthetic construct
  REGION 1..13: note = synthetic
GSMLFRVTINKWK

SEQ ID NO: 306 - protein, 13 aa, synthetic construct
  REGION 1..13: note = synthetic
GSMLFRVTIKSWK

SEQ ID NO: 307 - protein, 13 aa, synthetic construct
  REGION 1..13: note = synthetic
GSMLFRVTINRWK

SEQ ID NO: 308 - protein, 13 aa, synthetic construct
  REGION 1..13: note = synthetic
GSMLFRVTIRSWK

SEQ ID NO: 309 - protein, 13 aa, synthetic construct
  REGION 1..13: note = synthetic
GSMLFRVTINDWK

SEQ ID NO: 310 - protein, 13 aa, synthetic construct
  REGION 1..13: note = synthetic
GSMLFRVTIDSWK

SEQ ID NO: 311 - protein, 13 aa, synthetic construct
  REGION 1..13: note = synthetic
GSMLFRVTINEWK

SEQ ID NO: 312 - protein, 13 aa, synthetic construct
  REGION 1..13: note = synthetic
GSMLFRVTIESWK

SEQ ID NO: 313 - protein, 13 aa, synthetic construct
  REGION 1..13: note = synthetic
GSMRFRVTINSWK

SEQ ID NO: 314 - protein, 13 aa, synthetic construct
  REGION 1..13: note = synthetic
GSMDFRVTINSWK

SEQ ID NO: 315 - protein, 13 aa, synthetic construct
  REGION 1..13: note = synthetic
GSMEFRVTINSWK

SEQ ID NO: 316 - protein, 13 aa, synthetic construct
  REGION 1..13: note = synthetic
GSMLFRRTINSWK

SEQ ID NO: 317 - protein, 13 aa, synthetic construct
  REGION 1..13: note = synthetic
GSMLFRDTINSWK

SEQ ID NO: 318 - protein, 13 aa, synthetic construct
  REGION 1..13: note = synthetic
GSMLFRETINSWK

SEQ ID NO: 319 - protein, 13 aa, synthetic construct
  REGION 1..13: note = synthetic
GSMLFRVTDNSWK

SEQ ID NO: 320 - protein, 13 aa, synthetic construct
  REGION 1..13: note = synthetic
GSMLFRVTENSWK

SEQ ID NO: 321 - protein, 13 aa, synthetic construct
  REGION 1..13: note = synthetic
GSMKFRVTINSWK

SEQ ID NO: 322 - protein, 13 aa, synthetic construct
  REGION 1..13: note = synthetic
GSMLFRKTINSWK

SEQ ID NO: 323 - protein, 13 aa, synthetic construct
  REGION 1..13: note = synthetic
GSMLFRVTKNSWK

SEQ ID NO: 324 - protein, 11 aa, synthetic construct
  REGION 1..11: note = synthetic
GSMLFRVTINS

SEQ ID NO: 325 - protein, 11 aa, synthetic construct
  REGION 1..11: note = synthetic
GSMLFRVSINS

SEQ ID NO: 326 - protein, 11 aa, synthetic construct
  REGION 1..11: note = synthetic
GSMLFRVNINS

SEQ ID NO: 327 - protein, 11 aa, synthetic construct
  REGION 1..11: note = synthetic
GSKLFRVTINS

SEQ ID NO: 328 - protein, 11 aa, synthetic construct
  REGION 1..11: note = synthetic
GSRLFRVTINS

SEQ ID NO: 329 - protein, 11 aa, synthetic construct
  REGION 1..11: note = synthetic
GSMWFRVTINS

SEQ ID NO: 330 - protein, 11 aa, synthetic construct
  REGION 1..11: note = synthetic
GSMSFRVTINS

SEQ ID NO: 331 - protein, 11 aa, synthetic construct
  REGION 1..11: note = synthetic
GSMNFRVTINS

SEQ ID NO: 332 - protein, 11 aa, synthetic construct
  REGION 1..11: note = synthetic
GSMKFRVTINS

SEQ ID NO: 333 - protein, 11 aa, synthetic construct
  REGION 1..11: note = synthetic
GSMLFRWTINS

SEQ ID NO: 334 - protein, 11 aa, synthetic construct
  REGION 1..11: note = synthetic
GSMLFRSTINS

SEQ ID NO: 335 - protein, 11 aa, synthetic construct
  REGION 1..11: note = synthetic
GSMLFRNTINS

SEQ ID NO: 336 - protein, 11 aa, synthetic construct
  REGION 1..11: note = synthetic
GSMLFRKTINS

SEQ ID NO: 337 - protein, 11 aa, synthetic construct
  REGION 1..11: note = synthetic
GSMLFRVTWNS

SEQ ID NO: 338 - protein, 11 aa, synthetic construct
  REGION 1..11: note = synthetic
GSMLFRVTSNS

SEQ ID NO: 339 - protein, 11 aa, synthetic construct
  REGION 1..11: note = synthetic
GSMLFRVTNNS

SEQ ID NO: 340 - protein, 11 aa, synthetic construct
  REGION 1..11: note = synthetic
GSMLFRVTKNS

SEQ ID NO: 341 - protein, 11 aa, synthetic construct
  REGION 1..11: note = synthetic
GSMLFRVTIKS

SEQ ID NO: 342 - protein, 634 aa, synthetic construct
  REGION 1..634: note = synthetic
MKHHHHHHVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILARHELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 343 - DNA, 1902 bp, synthetic construct
  misc_feature 1..1902: note = synthetic
  source 1..1902: mol_type = other DNA
atgaaacatcaccatcaccatcatgtgagcaagggagaagaacttataaaagaaaacatgcggtctaaactgtacctcgagggctccgtcaatgggcaccagtttaagtgtacccacgagggtgagggaaagccctatgaggggaagcagacaaaccgcatcaaggtcgtcgaagggggacccctcccgtttgcctttgatatcttggctactcactttatgtacggaagcaaagttttcataaagtatcctgccgaccttcctgattattttaaacagtcatttcccgagggtttcacatgggaaagggtcatggtgtttgaggatggaggcgtgctcactgcaactcaggacacctcactgcaggacggcgagctgatctacaatgtgaaggtccggggtgtaaacttccctgccaacgggcctgtaatgcagaagaagaccctgggatgggagccgtccaccgaaaccatgtaccctgctgatggtgggctggagggccgatgtgacaaggctctgaagctcgttggaggtggtcatttgcacgtaaatttcaagacaacttacaagagcaaaaaacccgtaaaaatgcccggggttcattacgttgacagaaggcttgaacgcataaaggaagctgataacgagacatacgtggagcagtacgagcacgccgttgcccggtactcaaacctggggggtggctttacactggaggattttgtgggagattggagacagacagccggctacaatctggatcaggtgctggaacaaggaggagtgtcttctctgtttcagaatctgggagtgagcgtgacacctatccagaggatcgtgctgtctggcgagaatggactgaagatcgatattcacgtgatcatcccctacgaaggcctgtctggagaccagatgggccagattgagaagatcttcaaagtggtgtatcctgtggacgatcaccacttcaaggtgatcctgcactacggcaccctggtgattgatggagtgacacctaacatgatcgactacttcggaagaccttacgagggaatcgccgtgttcgacggaaagaagatcaccgtgacaggaacactgtggaatggaaacaagatcatcgacgagcggctgatcaaccctgatggatctctgctgttcagagtgaccatcaacggagtgacaggatggagactgtgcgagagaattctggctagacatgagctaatcaaggaaaatatgagaagtaagctatacttagaggggtccgtcaacggtcaccagtttaaatgcactcatgaaggtgaggggaaaccttatgaaggtaagcagactaatcgaataaaagtggtcgagggcggtcctctgccattcgctttcgatattctggccactcactttatgtatgggtctaaggtctttattaaataccccgctgatttgccagactactttaaacagtccttccctgaaggattcacatgggagcgggtgatggtgttcgaggatggaggcgttcttactgcaactcaggatacttccttgcaagacggggaactgatctacaacgttaaggtccgcggcgtcaatttcccagccaatggtccagtgatgcagaagaaaaccttggggtgggagccctcaacggagacaatgtaccctgcggacggcgggcttgagggtagatgtgataaggcattgaaactcgtcgggggcggccaccttcatgtgaatttcaagactacatataaaagtaaaaaaccagtcaagatgcctggagtgcactacgtggatcgtaggttggagaggataaaagaagccgacaacgaaacttatgtagagcaatatgagcacgccgtggctcgttattccaacttgggcggaggaatggatgaactgtacaag

SEQ ID NO: 344 - protein, 622 aa, synthetic construct
  REGION 1..622: note = synthetic
MKHHHHHHVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSRHELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 345 - DNA, 1866 bp, synthetic construct
  misc_feature 1..1866: note = synthetic
  source 1..1866: mol_type = other DNA
atgaaacatcaccatcaccatcatgtgagcaagggagaagaacttataaaagaaaacatgcggtctaaactgtacctcgagggctccgtcaatgggcaccagtttaagtgtacccacgagggtgagggaaagccctatgaggggaagcagacaaaccgcatcaaggtcgtcgaagggggacccctcccgtttgcctttgatatcttggctactcactttatgtacggaagcaaagttttcataaagtatcctgccgaccttcctgattattttaaacagtcatttcccgagggtttcacatgggaaagggtcatggtgtttgaggatggaggcgtgctcactgcaactcaggacacctcactgcaggacggcgagctgatctacaatgtgaaggtccggggtgtaaacttccctgccaacgggcctgtaatgcagaagaagaccctgggatgggagccgtccaccgaaaccatgtaccctgctgatggtgggctggagggccgatgtgacaaggctctgaagctcgttggaggtggtcatttgcacgtaaatttcaagacaacttacaagagcaaaaaacccgtaaaaatgcccggggttcattacgttgacagaaggcttgaacgcataaaggaagctgataacgagacatacgtggagcagtacgagcacgccgttgcccggtactcaaacctggggggtggcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcagacatgagctaatcaaggaaaatatgagaagtaagctatacttagaggggtccgtcaacggtcaccagtttaaatgcactcatgaaggtgaggggaaaccttatgaaggtaagcagactaatcgaataaaagtggtcgagggcggtcctctgccattcgctttcgatattctggccactcactttatgtatgggtctaaggtctttattaaataccccgctgatttgccagactactttaaacagtccttccctgaaggattcacatgggagcgggtgatggtgttcgaggatggaggcgttcttactgcaactcaggatacttccttgcaagacggggaactgatctacaacgttaaggtccgcggcgtcaatttcccagccaatggtccagtgatgcagaagaaaaccttggggtgggagccctcaacggagacaatgtaccctgcggacggcgggcttgagggtagatgtgataaggcattgaaactcgtcgggggcggccaccttcatgtgaatttcaagactacatataaaagtaaaaaaccagtcaagatgcctggagtgcactacgtggatcgtaggttggagaggataaaagaagccgacaacgaaacttatgtagagcaatatgagcacgccgtggctcgttattccaacttgggcggaggaatggatgaactgtacaag

SEQ ID NO: 346 - protein, 611 aa, synthetic construct
  REGION 1..611: note = synthetic
MKHHHHHHVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDRHELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 347 - DNA, 1833 bp, synthetic construct
  misc_feature 1..1833: note = synthetic
  source 1..1833: mol_type = other DNA
atgaaacatcaccatcaccatcatgtgagcaagggagaagaacttataaaagaaaacatgcggtctaaactgtacctcgagggctccgtcaatgggcaccagtttaagtgtacccacgagggtgagggaaagccctatgaggggaagcagacaaaccgcatcaaggtcgtcgaagggggacccctcccgtttgcctttgatatcttggctactcactttatgtacggaagcaaagttttcataaagtatcctgccgaccttcctgattattttaaacagtcatttcccgagggtttcacatgggaaagggtcatggtgtttgaggatggaggcgtgctcactgcaactcaggacacctcactgcaggacggcgagctgatctacaatgtgaaggtccggggtgtaaacttccctgccaacgggcctgtaatgcagaagaagaccctgggatgggagccgtccaccgaaaccatgtaccctgctgatggtgggctggagggccgatgtgacaaggctctgaagctcgttggaggtggtcatttgcacgtaaatttcaagacaacttacaagagcaaaaaacccgtaaaaatgcccggggttcattacgttgacagaaggcttgaacgcataaaggaagctgataacgagacatacgtggagcagtacgagcacgccgttgcccggtactcaaacctggggggtggcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacagacatgagctaatcaaggaaaatatgagaagtaagctatacttagaggggtccgtcaacggtcaccagtttaaatgcactcatgaaggtgaggggaaaccttatgaaggtaagcagactaatcgaataaaagtggtcgagggcggtcctctgccattcgctttcgatattctggccactcactttatgtatgggtctaaggtctttattaaataccccgctgatttgccagactactttaaacagtccttccctgaaggattcacatgggagcgggtgatggtgttcgaggatggaggcgttcttactgcaactcaggatacttccttgcaagacggggaactgatctacaacgttaaggtccgcggcgtcaatttcccagccaatggtccagtgatgcagaagaaaaccttggggtgggagccctcaacggagacaatgtaccctgcggacggcgggcttgagggtagatgtgataaggcattgaaactcgtcgggggcggccaccttcatgtgaatttcaagactacatataaaagtaaaaaaccagtcaagatgcctggagtgcactacgtggatcgtaggttggagaggataaaagaagccgacaacgaaacttatgtagagcaatatgagcacgccgtggctcgttattccaacttgggcggaggaatggatgaactgtacaag

SEQ ID NO: 348 - protein, 401 aa, synthetic construct
  REGION 1..401: note = synthetic
MKHHHHHHFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGSSGELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 349 - DNA, 1203 bp, synthetic construct
  misc_feature 1..1203: note = synthetic
  source 1..1203: mol_type = other DNA
atgaaacatcaccatcaccatcatttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcggaggctcaggtggatcctcaggtgagctaatcaaggaaaatatgagaagtaagctatacttagaggggtccgtcaacggtcaccagtttaaatgcactcatgaaggtgaggggaaaccttatgaaggtaagcagactaatcgaataaaagtggtcgagggcggtcctctgccattcgctttcgatattctggccactcactttatgtatgggtctaaggtctttattaaataccccgctgatttgccagactactttaaacagtccttccctgaaggattcacatgggagcgggtgatggtgttcgaggatggaggcgttcttactgcaactcaggatacttccttgcaagacggggaactgatctacaacgttaaggtccgcggcgtcaatttcccagccaatggtccagtgatgcagaagaaaaccttggggtgggagccctcaacggagacaatgtaccctgcggacggcgggcttgagggtagatgtgataaggcattgaaactcgtcgggggcggccaccttcatgtgaatttcaagactacatataaaagtaaaaaaccagtcaagatgcctggagtgcactacgtggatcgtaggttggagaggataaaagaagccgacaacgaaacttatgtagagcaatatgagcacgccgtggctcgttattccaacttgggcggaggaatggatgaactgtacaag

SEQ ID NO: 350 - protein, 395 aa, synthetic construct
  REGION 1..395: note = synthetic
MKHHHHHHFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSRHELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 351 - DNA, 1185 bp, synthetic construct
  misc_feature 1..1185: note = synthetic
  source 1..1185: mol_type = other DNA
atgaaacatcaccatcaccatcatttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcagacatgagctaatcaaggaaaatatgagaagtaagctatacttagaggggtccgtcaacggtcaccagtttaaatgcactcatgaaggtgaggggaaaccttatgaaggtaagcagactaatcgaataaaagtggtcgagggcggtcctctgccattcgctttcgatattctggccactcactttatgtatgggtctaaggtctttattaaataccccgctgatttgccagactactttaaacagtccttccctgaaggattcacatgggagcgggtgatggtgttcgaggatggaggcgttcttactgcaactcaggatacttccttgcaagacggggaactgatctacaacgttaaggtccgcggcgtcaatttcccagccaatggtccagtgatgcagaagaaaaccttggggtgggagccctcaacggagacaatgtaccctgcggacggcgggcttgagggtagatgtgataaggcattgaaactcgtcgggggcggccaccttcatgtgaatttcaagactacatataaaagtaaaaaaccagtcaagatgcctggagtgcactacgtggatcgtaggttggagaggataaaagaagccgacaacgaaacttatgtagagcaatatgagcacgccgtggctcgttattccaacttgggcggaggaatggatgaactgtacaag

SEQ ID NO: 352 - protein, 390 aa, synthetic construct
  REGION 1..390: note = synthetic
MKHHHHHHFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGGSGGSSGELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 353 - DNA, 1170 bp, synthetic construct
  misc_feature 1..1170: note = synthetic
  source 1..1170: mol_type = other DNA
atgaaacatcaccatcaccatcatttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggaggctcaggtggatcctcaggtgagctaatcaaggaaaatatgagaagtaagctatacttagaggggtccgtcaacggtcaccagtttaaatgcactcatgaaggtgaggggaaaccttatgaaggtaagcagactaatcgaataaaagtggtcgagggcggtcctctgccattcgctttcgatattctggccactcactttatgtatgggtctaaggtctttattaaataccccgctgatttgccagactactttaaacagtccttccctgaaggattcacatgggagcgggtgatggtgttcgaggatggaggcgttcttactgcaactcaggatacttccttgcaagacggggaactgatctacaacgttaaggtccgcggcgtcaatttcccagccaatggtccagtgatgcagaagaaaaccttggggtgggagccctcaacggagacaatgtaccctgcggacggcgggcttgagggtagatgtgataaggcattgaaactcgtcgggggcggccaccttcatgtgaatttcaagactacatataaaagtaaaaaaccagtcaagatgcctggagtgcactacgtggatcgtaggttggagaggataaaagaagccgacaacgaaacttatgtagagcaatatgagcacgccgtggctcgttattccaacttgggcggaggaatggatgaactgtacaag

SEQ ID NO: 354 - protein, 384 aa, synthetic construct
  REGION 1..384: note = synthetic
MKHHHHHHFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDRHELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 355 - DNA, 1152 bp, synthetic construct
  misc_feature 1..1152: note = synthetic
  source 1..1152: mol_type = other DNA
atgaaacatcaccatcaccatcatttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacagacatgagctaatcaaggaaaatatgagaagtaagctatacttagaggggtccgtcaacggtcaccagtttaaatgcactcatgaaggtgaggggaaaccttatgaaggtaagcagactaatcgaataaaagtggtcgagggcggtcctctgccattcgctttcgatattctggccactcactttatgtatgggtctaaggtctttattaaataccccgctgatttgccagactactttaaacagtccttccctgaaggattcacatgggagcgggtgatggtgttcgaggatggaggcgttcttactgcaactcaggatacttccttgcaagacggggaactgatctacaacgttaaggtccgcggcgtcaatttcccagccaatggtccagtgatgcagaagaaaaccttggggtgggagccctcaacggagacaatgtaccctgcggacggcgggcttgagggtagatgtgataaggcattgaaactcgtcgggggcggccaccttcatgtgaatttcaagactacatataaaagtaaaaaaccagtcaagatgcctggagtgcactacgtggatcgtaggttggagaggataaaagaagccgacaacgaaacttatgtagagcaatatgagcacgccgtggctcgttattccaacttgggcggaggaatggatgaactgtacaag

SEQ ID NO: 356 - protein, 155 aa, synthetic construct
  REGION 1..155: note = synthetic
MKHHHHHHVFTLEDFVGDWEQTAAYNLDQVLEQGGVSSLLQNLAVSVTPILRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPD

SEQ ID NO: 357 - DNA, 465 bp, synthetic construct
  misc_feature 1..465: note = synthetic
  source 1..465: mol_type = other DNA
atgaaacatcaccatcaccatcatgtcttcacactcgaagatttcgttggggactgggaacagaccgccgcctacaacctggaccaagtccttgaacagggaggtgtgtccagtttgctgcagaatctcgccgtgtccgtaactccgatcctaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgac

SEQ ID NO: 358 - DNA, 396 bp, synthetic construct
  misc_feature 1..396: note = synthetic
  source 1..396: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggctaa

SEQ ID NO: 359 - protein, 131 aa, synthetic construct
  REGION 1..131: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDG

SEQ ID NO: 360 - DNA, 654 bp, synthetic construct
  misc_feature 1..654: note = synthetic
  source 1..654: mol_type = other DNA
gggagctccggtggtggcgggagcggaggtggaggctcgagcggtatgacgtataagttaatccttaatggtaaaacattgaaaggcgagacaactactgaagctgttgatgctgctactgcagaaaaagtcttcaaacaatacgctaacgacaacggtgttgacggtgaatggacttacgacgatgcgacgaaaacctttacggtcaccgaaaaaccagaagtgatcgatgcgtctgaattaacaccagccgtgacaacttacaaacttgttattaatggtaaaacattgaaaggcgaaacaactactgaggctgttgatgctgctactgcagagaaggtgttcaaacaatatgcgaatgacaacggtgttgacggtgagtggacttacgacgatgcgactaagacctttacagttactgaaaaaccagaagtgatcgatgcgtctgagttaacaccagccgtgacaacttacaaacttgttattaatggtaaaacattgaaaggcgaaacaactactaaagcagtagacgcagaaactgcggagaaggccttcaaacaatacgctaacgacaacggtgttgatggtgtttggacttatgatgatgccacaaaaacctttacggtaactgagcatcatcaccatcaccactaa

SEQ ID NO: 361 - protein, 217 aa, synthetic construct
  REGION 1..217: note = synthetic
GSSGGGGSGGGGSSGMTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEHHHHHH

SEQ ID NO: 362 - DNA, 363 bp, synthetic construct
  misc_feature 1..363: note = synthetic
  source 1..363: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggataa

SEQ ID NO: 363 - protein, 120 aa, synthetic construct
  REGION 1..120: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFG

SEQ ID NO: 364 - DNA, 396 bp, synthetic construct; identical to SEQ ID NO: 358
  misc_feature 1..396: note = synthetic
  source 1..396: mol_type = other DNA

SEQ ID NO: 365 - protein, 131 aa, synthetic construct; identical to SEQ ID NO: 359
  REGION 1..131: note = synthetic

SEQ ID NO: 366 - DNA, 423 bp, synthetic construct
  misc_feature 1..423: note = synthetic
  source 1..423: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtaa

SEQ ID NO: 367 - protein, 140 aa, synthetic construct
  REGION 1..140: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTL

SEQ ID NO: 368 - DNA, 432 bp, synthetic construct
  misc_feature 1..432: note = synthetic
  source 1..432: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggctaa

SEQ ID NO: 369 - protein, 143 aa, synthetic construct
  REGION 1..143: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNG

SEQ ID NO: 370 - DNA, 483 bp, synthetic construct
  misc_feature 1..483: note = synthetic
  source 1..483: mol_type = other DNA
atggtttccgtgagcggctggcggctgttcaagaagattagcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgactaa

SEQ ID NO: 371 - protein, 160 aa, synthetic construct
  REGION 1..160: note = synthetic
MVSVSGWRLFKKISFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPD

SEQ ID NO: 372 - DNA, 495 bp, synthetic construct
  misc_feature 1..495: note = synthetic
  source 1..495: mol_type = other DNA
atggtttccgtgagcggctggcggctgttcaagaagattagcggcagctccggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgactaa

SEQ ID NO: 373 - protein, 164 aa, synthetic construct
  REGION 1..164: note = synthetic
MVSVSGWRLFKKISGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPD

SEQ ID NO: 374 - DNA, 507 bp, synthetic construct
  misc_feature 1..507: note = synthetic
  source 1..507: mol_type = other DNA
atggtttccgtgagcggctggcggctgttcaagaagattagcggctcgagcggtggctcgagcggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgactaa

SEQ ID NO: 375 - protein, 168 aa, synthetic construct
  REGION 1..168: note = synthetic
MVSVSGWRLFKKISGSSGGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPD

SEQ ID NO: 376 - DNA, 519 bp, synthetic construct
  misc_feature 1..519: note = synthetic
  source 1..519: mol_type = other DNA
atggtttccgtgagcggctggcggctgttcaagaagattagcggctcgagcggtggctcgagcggtggctcgagcggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgactaa

SEQ ID NO: 377 - protein, 172 aa, synthetic construct
  REGION 1..172: note = synthetic
MVSVSGWRLFKKISGSSGGSSGGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPD

SEQ ID NO: 378 - DNA, 531 bp, synthetic construct
  misc_feature 1..531: note = synthetic
  source 1..531: mol_type = other DNA
atggtttccgtgagcggctggcggctgttcaagaagattagcggctcgagcggtggctcgagcggtggctcgagcggtggctcgagcggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgactaa

SEQ ID NO: 379 - protein, 176 aa, synthetic construct
  REGION 1..176: note = synthetic
MVSVSGWRLFKKISGSSGGSSGGSSGGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPD

SEQ ID NO: 380 - DNA, 543 bp, synthetic construct
  misc_feature 1..543: note = synthetic
  source 1..543: mol_type = other DNA
atggtttccgtgagcggctggcggctgttcaagaagattagcggctcgagcggtggctcgagcggtggctcgagcggtggctcgagcggtggctcgagcggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgactaa

SEQ ID NO: 381 - protein, 180 aa, synthetic construct
  REGION 1..180: note = synthetic
MVSVSGWRLFKKISGSSGGSSGGSSGGSSGGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPD

SEQ ID NO: 382 - DNA, 537 bp, synthetic construct
  misc_feature 1..537: note = synthetic
  source 1..537: mol_type = other DNA
atggtgagcggctggcggctgttcaagaagattagcggctcgagcggtggctcgagcggtggctcgagcggtggctcgagcggtggctcgagcggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgactaa

SEQ ID NO: 383 - protein, 178 aa, synthetic construct
  REGION 1..178: note = synthetic
MVSGWRLFKKISGSSGGSSGGSSGGSSGGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPD

SEQ ID NO: 384 - DNA, 486 bp, synthetic construct
  misc_feature 1..486: note = synthetic
  source 1..486: mol_type = other DNA
atggtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacgtttccgtgagcggctggcggctgttcaagaagattagctaa

SEQ ID NO: 385 - protein, 161 aa, synthetic construct
  REGION 1..161: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDVSVSGWRLFKKIS

SEQ ID NO: 386 - DNA, 498 bp, synthetic construct
  misc_feature 1..498: note = synthetic
  source 1..498: mol_type = other DNA
atggtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggctcgagcggtgtttccgtgagcggctggcggctgttcaagaagattagctaa

SEQ ID NO: 387 - protein, 165 aa, synthetic construct
  REGION 1..165: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSSGVSVSGWRLFKKIS

SEQ ID NO: 388 - DNA, 510 bp, synthetic construct
  misc_feature 1..510: note = synthetic
  source 1..510: mol_type = other DNA
atggtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggctcgagcggtggctcgagcggtgtttccgtgagcggctggcggctgttcaagaagattagctaa

SEQ ID NO: 389 - protein, 169 aa, synthetic construct
  REGION 1..169: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSSGGSSGVSVSGWRLFKKIS

SEQ ID NO: 390 - DNA, 504 bp, synthetic construct
  misc_feature 1..504: note = synthetic
  source 1..504: mol_type = other DNA
atggtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggctcgagcggtggctcgagcggtgtgagcggctggcggctgttcaagaagattagctaa

SEQ ID NO: 391 - protein, 167 aa, synthetic construct
  REGION 1..167: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSSGGSSGVSGWRLFKKIS

SEQ ID NO: 392 - DNA, 501 bp, synthetic construct
  misc_feature 1..501: note = synthetic
  source 1..501: mol_type = other DNA
atggtttccgtgagcggctggcggctgttcaagaagattagcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgaccatcaccatcaccatcattaa

SEQ ID NO: 393 - protein, 166 aa, synthetic construct
  REGION 1..166: note = synthetic
MVSVSGWRLFKKISFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDHHHHHH

SEQ ID NO: 394 - DNA, 513 bp, synthetic construct
  misc_feature 1..513: note = synthetic
  source 1..513: mol_type = other DNA
atggtttccgtgagcggctggcggctgttcaagaagattagcggcagctccggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgaccatcaccatcaccatcattaa

SEQ ID NO: 395 - protein, 170 aa, synthetic construct
  REGION 1..170: note = synthetic
MVSVSGWRLFKKISGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDHHHHHH

SEQ ID NO: 396 - DNA, 525 bp, synthetic construct
  misc_feature 1..525: note = synthetic
  source 1..525: mol_type = other DNA
atggtttccgtgagcggctggcggctgttcaagaagattagcggctcgagcggtggctcgagcggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgaccatcaccatcaccatcattaa

SEQ ID NO: 397 - protein, 174 aa, synthetic construct
  REGION 1..174: note = synthetic
MVSVSGWRLFKKISGSSGGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDHHHHHH

SEQ ID NO: 398 - DNA, 537 bp, synthetic construct
  misc_feature 1..537: note = synthetic
  source 1..537: mol_type = other DNA
atggtttccgtgagcggctggcggctgttcaagaagattagcggctcgagcggtggctcgagcggtggctcgagcggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgaccatcaccatcaccatcattaa

SEQ ID NO: 399 - protein, 178 aa, synthetic construct
  REGION 1..178: note = synthetic
MVSVSGWRLFKKISGSSGGSSGGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDHHHHHH

SEQ ID NO: 400 - DNA, 549 bp, synthetic construct
  misc_feature 1..549: note = synthetic
  source 1..549: mol_type = other DNA
atggtttccgtgagcggctggcggctgttcaagaagattagcggctcgagcggtggctcgagcggtggctcgagcggtggctcgagcggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgaccatcaccatcaccatcattaa

SEQ ID NO: 401 - protein, 182 aa, synthetic construct
  REGION 1..182: note = synthetic
MVSVSGWRLFKKISGSSGGSSGGSSGGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDHHHHHH

SEQ ID NO: 402 - DNA, 555 bp, synthetic construct
  misc_feature 1..555: note = synthetic
  source 1..555: mol_type = other DNA
atggtgagcggctggcggctgttcaagaagattagcggctcgagcggtggctcgagcggtggctcgagcggtggctcgagcggtggctcgagcggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgaccatcaccatcaccatcattaa

SEQ ID NO: 403 - protein, 184 aa, synthetic construct
  REGION 1..184: note = synthetic
MVSGWRLFKKISGSSGGSSGGSSGGSSGGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDHHHHHH

SEQ ID NO: 404 - DNA, 561 bp, synthetic construct
  misc_feature 1..561: note = synthetic
  source 1..561: mol_type = other DNA
atggtttccgtgagcggctggcggctgttcaagaagattagcggctcgagcggtggctcgagcggtggctcgagcggtggctcgagcggtggctcgagcggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgaccatcaccatcaccatcattaa

SEQ ID NO: 405 - protein, 186 aa, synthetic construct
  REGION 1..186: note = synthetic
MVSVSGWRLFKKISGSSGGSSGGSSGGSSGGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDHHHHHH

SEQ ID NO: 406 - DNA, 507 bp, synthetic construct
  misc_feature 1..507: note = synthetic
  source 1..507: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacgtttccgtgagcggctggcggctgttcaagaagattagctaa

SEQ ID NO: 407 - protein, 168 aa, synthetic construct
  REGION 1..168: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDVSVSGWRLFKKIS

SEQ ID NO: 408 - DNA, 519 bp, synthetic construct
  misc_feature 1..519: note = synthetic
  source 1..519: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggctcgagcggtgtttccgtgagcggctggcggctgttcaagaagattagctaa

SEQ ID NO: 409 - protein, 172 aa, synthetic construct
  REGION 1..172: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSSGVSVSGWRLFKKIS

SEQ ID NO: 410 - DNA, 525 bp, synthetic construct
  misc_feature 1..525: note = synthetic
  source 1..525: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggctcgagcggtggctcgagcggtgtgagcggctggcggctgttcaagaagattagctaa

SEQ ID NO: 411 - protein, 174 aa, synthetic construct
  REGION 1..174: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSSGGSSGVSGWRLFKKIS

SEQ ID NO: 412 - DNA, 531 bp, synthetic construct
  misc_feature 1..531: note = synthetic
  source 1..531: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgacggctcgagcggtggctcgagcggtgtttccgtgagcggctggcggctgttcaagaagattagctaa

SEQ ID NO: 413 - protein, 176 aa, synthetic construct
  REGION 1..176: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSSGGSSGVSVSGWRLFKKIS

SEQ ID NO: 414 - DNA, 498 bp, synthetic construct; identical to SEQ ID NO: 126
  misc_feature 1..498: note = synthetic
  source 1..498: mol_type = other DNA

SEQ ID NO: 415 - protein, 165 aa, synthetic construct; identical to SEQ ID NO: 125
  REGION 1..165: note = synthetic

SEQ ID NO: 416 - DNA, 501 bp, synthetic construct; identical to SEQ ID NO: 203
  misc_feature 1..501: note = synthetic
  source 1..501: mol_type = other DNA

SEQ ID NO: 417 - protein, 166 aa, synthetic construct; identical to SEQ ID NO: 204
  REGION 1..166: note = synthetic

SEQ ID NO: 418 - DNA, 510 bp, synthetic construct; identical to SEQ ID NO: 205
  misc_feature 1..510: note = synthetic
  source 1..510: mol_type = other DNA

SEQ ID NO: 419 - protein, 169 aa, synthetic construct; identical to SEQ ID NO: 206
  REGION 1..169: note = synthetic

SEQ ID NO: 420 - DNA, 507 bp, synthetic construct
  misc_feature 1..507: note = synthetic
  source 1..507: mol_type = other DNA
atggtgagcggctggcggctgttcaagaagattagcggcagctccggtttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcacccccgaccatcaccatcaccatcattaa

SEQ ID NO: 421 - protein, 168 aa, synthetic construct
  REGION 1..168: note = synthetic
MVSGWRLFKKISGSSGFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDHHHHHH

SEQ ID NO: 422 - DNA, 498 bp, synthetic construct
  misc_feature 1..498: note = synthetic
  source 1..498: mol_type = other DNA
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacgagaacaaaattatcgacgagcgcctgatcacccccgacggctccatgctgttccgagtaaccatcaacagccatcatcaccatcaccactaa

SEQ ID NO: 423 - protein, 165 aa, synthetic construct
  REGION 1..165: note = synthetic
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNENKIIDERLITPDGSMLFRVTINSHHHHHH

SEQ ID NO: 424 - DNA, 498 bp, synthetic construct
  misc_feature 1..498: note = synthetic
  source 1..498: mol_type = other DNA
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcgttaaaattatcgacgagcgcctgatcacccccgacggctccatgctgttccgagtaaccatcaacagccatcatcaccatcaccactaa

SEQ ID NO: 425 - protein, 165 aa, synthetic construct
  REGION 1..165: note = synthetic
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGVKIIDERLITPDGSMLFRVTINSHHHHHH

SEQ ID NO: 426 - DNA, 498 bp, synthetic construct
  misc_feature 1..498: note = synthetic
  source 1..498: mol_type = other DNA
atggtcttcacactcgaagatttcgttggggactgggaacagacagccgcctacaacctggaccaagtccttgaacagggaggtgtgtccagtttgctgcagaatctcgccgtgtccgtaactccgatccaaaggatggtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 427 - protein, 165 aa, synthetic construct
  REGION 1..165: note = synthetic
MVFTLEDFVGDWEQTAAYNLDQVLEQGGVSSLLQNLAVSVTPIQRMVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 428 - DNA, 498 bp, synthetic construct
  misc_feature 1..498: note = synthetic
  source 1..498: mol_type = other DNA
atggtcttcacactcgaagatttcgttggggactggaagcagacagccgcctacaacctggaccaagtccttgaacagggaggtgtgtccagtttgctgcagaatctcgccgtgtccgtaactccgatccaaaggatggtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagccatcatcaccatcaccactaa

SEQ ID NO: 429 - protein, 165 aa, synthetic construct
  REGION 1..165: note = synthetic
MVFTLEDFVGDWKQTAAYNLDQVLEQGGVSSLLQNLAVSVTPIQRMVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 430 - DNA, 498 bp, synthetic construct
  misc_feature 1..498: note = synthetic
  source 1..498: mol_type = other DNA
atggtcttcacactcgaagatttcgttggggactggaagcagacagccgcctacaacctggaccaagtccttgaacagggaggtgtgtccagtttgctgcagaatctcgccgtgtccgtaactccgatccaaaggatggtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacgacgtcaaaattatcgacgagcgcctgatcacccccgacggctccatgctgttccgagtaaccatcaacagccatcatcaccatcaccactaa

SEQ ID NO: 431 - protein, 165 aa, synthetic construct
  REGION 1..165: note = synthetic
MVFTLEDFVGDWKQTAAYNLDQVLEQGGVSSLLQNLAVSVTPIQRMVRSGENALKIDIHVIIPYEGLSADQMAQIEEVFKVVYPVDDHHFKVILPYGTLVIDGVTPNMLNYFGRPYEGIAVFDGKKITVTGTLWNDVKIIDERLITPDGSMLFRVTINSHHHHHH

SEQ ID NO: 432 - DNA, 498 bp, synthetic construct
  misc_feature 1..498: note = synthetic
  source 1..498: mol_type = other DNA
atggtcttcacactcgaagatttcgttggggactggaagcagacagccgcctacaacctggaccaagtccttgaacagggaggtgtgtccagtttgctgcagaatctcgccgtgtccgtaactccgatccaaaggatggtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacgacgtcaaaattatcgacgagcgcctgatcacccccgacggctccatgtccttccgagtaaccatcaacagccatcatcaccatcaccactaa

SEQ ID NO: 433 - protein, 165 aa, synthetic construct
  REGION 1..165: note = synthetic
MVFTLEDFVGDWKQTAAYNLDQVLEQGGVSSLLQNLAVSVTPIQRMVRSGENALKIDIHVIIPYEGLSADQMAQIEEVFKVVYPVDDHHFKVILPYGTLVIDGVTPNMLNYFGRPYEGIAVFDGKKITVTGTLWNDVKIIDERLITPDGSMSFRVTINSHHHHHH

SEQ ID NO: 434 - DNA, 498 bp, synthetic construct
  misc_feature 1..498: note = synthetic
  source 1..498: mol_type = other DNA
atggtcttcacactcgaagatttcgttggggactggaagcagacagccgcctacaacctggaccaagtccttgaacagggaggtgtgtccagtttgctgcagaatctcgccgtgtccgtaactccgatccaaaggatggtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgtccttccgagtaaccatcaacagccatcatcaccatcaccactaa

SEQ ID NO: 435 - protein, 165 aa, synthetic construct
  REGION 1..165: note = synthetic
MVFTLEDFVGDWKQTAAYNLDQVLEQGGVSSLLQNLAVSVTPIQRMVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMSFRVTINSHHHHHH

SEQ ID NO: 436 - DNA, 498 bp, synthetic construct
  misc_feature 1..498: note = synthetic
  source 1..498: mol_type = other DNA
atggtcttcacactcgaagatttcgttggggactggaagcagacagccgcctacaacctggaccaagtccttgaacagggaggtgtgtccagtttgctgcagaatctcgccgtgtccgtaactccgatccaaaggatggtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcgtcaaaattatcgacgagcgcctgatcacccccgacggctccatgtccttccgagtaaccatcaacagccatcatcaccatcaccactaa

SEQ ID NO: 437 - protein, 165 aa, synthetic construct
  REGION 1..165: note = synthetic
MVFTLEDFVGDWKQTAAYNLDQVLEQGGVSSLLQNLAVSVTPIQRMVRSGENALKIDIHVIIPYEGLSADQMAQIEEVFKVVYPVDDHHFKVILPYGTLVIDGVTPNMLNYFGRPYEGIAVFDGKKITVTGTLWNGVKIIDERLITPDGSMSFRVTINSHHHHHH

SEQ ID NO: 438 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacacccgtatgaaggcatcgccgtgttcgacggcaaaaagatcactaccacagggaccctgtggaacggcaacaaaattatcgacgagcgcctgatcacccccgactaa

SEQ ID NO: 439 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGHPYEGIAVFDGKKITTTGTLWNGNKIIDERLITPD

SEQ ID NO: 440 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcgagaagatcactaccacagggaccctgtggaacggcaacaaaattatcgacgagcgcctgatcacccccgactaa

SEQ ID NO: 441 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGEKITTTGTLWNGNKIIDERLITPD

SEQ ID NO: 442 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgcctaacggcaacaaaattatcgacgagcgcctgatcacccccgactaa

SEQ ID NO: 443 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLPNGNKIIDERLITPD

SEQ ID NO: 444 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcgatcccgactaa

SEQ ID NO: 445 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLIDPD

SEQ ID NO: 446 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacggccgtatgaaggcatcgccgtgttcgacggcaaaaagatcactaccacagggaccctgtggaacggcaacaaaattatcgacgagcgcctgatcaccgatgactaa

SEQ ID NO: 447 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITDD

SEQ ID NO: 448 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacacccgtatgaaggcatcgccgtgttcgacggcgagaagatcactaccacagggaccctgtggaacggcaacaaaattatcgacgagcgcctgatcacccccgactaa

SEQ ID NO: 449 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGHPYEGIAVFDGEKITTTGTLWNGNKIIDERLITPD

SEQ ID NO: 450 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacacccgtatgaaggcatcgccgtgttcgacggcgagaagatcactaccacagggaccctgtggaacggcaacaaaattatcgacgagcgcctgatcgatcccgactaa

SEQ ID NO: 451 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGHPYEGIAVFDGEKITTTGTLWNGNKIIDERLIDPD

SEQ ID NO: 452 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacaagctgaactatttcggacacccgtatgaaggcatcgccgtgttcgacggcgagaagatcactaccacagggaccctgtggaacggcaacaaaattatcgacgagcgcctgatcgatgatgactaa

SEQ ID NO: 453 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGHPYEGIAVFDGEKITTTGTLWNGNKIIDERLIDDD

SEQ ID NO: 454 - DNA, 468 bp, synthetic construct
  misc_feature 1..468: note = synthetic
  source 1..468: mol_type = other DNA
atgaaacatcaccatcaccatcatgatttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgcccatcggcacactggtaatcgacggggttacgccgaacaagctgaactatttcggacacccgtatgaaggcatcgccgtgttcgacggcgagaagatcactaccacagggaccctgtggaacggcaacaaaattatcgacgagcgcctgatcgatcccgactaa

SEQ ID NO: 455 - protein, 155 aa, synthetic construct
  REGION 1..155: note = synthetic
MKHHHHHHDFTLDDFVGDWEQTAAYNLDQVLEQGGVSSLLQNLAVSVTPIMRIVRSGENALKIDIHVIIPYEGLSADQMAQIEEVFKVVYPVDDHHFKVILPIGTLVIDGVTPNKLNYFGHPYEGIAVFDGEKITTTGTLWNGNKIIDERLIDPD

SEQ ID NO: 456 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgcccatcggcacactggtaatcgacggggagacgccgaacaagctgaactatttcggacacccgtatgaaggcatcgccgtgttcgacggcgagaagatcactaccacagggaccctgtggaacggcaacaaaattatcgacgagcgcctgatcgatcccgactaa

SEQ ID NO: 457 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPIGTLVIDGETPNKLNYFGHPYEGIAVFDGEKITTTGTLWNGNKIIDERLIDPD

SEQ ID NO: 458 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgcccatcggcacactggtaatcgacggggttacgccgaacaagctgaactatttcggacacccgtatgaaggcatcgccgatttcgacggcgagaagatcactaccacagggaccctgtggaacggcaacaaaattatcgacgagcgcctgatcgatcccgactaa

SEQ ID NO: 459 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPIGTLVIDGVTPNKLNYFGHPYEGIADFDGEKITTTGTLWNGNKIIDERLIDPD

SEQ ID NO: 460 - DNA, 468 bp, synthetic construct
  misc_feature 1..468: note = synthetic
  source 1..468: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgcccatcggcacactggtaatcgacggggagacgccgaacaagctgaactatttcggacacccgtatgaaggcatcgccgatttcgacggcgagaagatcactaccacagggaccctgtggaacggcaacaaaattatcgacgagcgcctgatcgatcccgactaa

SEQ ID NO: 461 - protein, 155 aa, synthetic construct
  REGION 1..155: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPIGTLVIDGETPNKLNYFGHPYEGIADFDGEKITTTGTLWNGNKIIDERLIDPD

SEQ ID NO: 462 - DNA, 327 bp, synthetic construct
  misc_feature 1..327: note = synthetic
  source 1..327: mol_type = other DNA
atgaaacatcaccatcaccatcatgtcttcacactcgacgatttcgttggggactgggaacagacagccgcctacaacctggaccaagtccttgaacagggaggtgtgtccagtttgctgcagaatctcgccgtgtccgtaactccgatcatgaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgac

SEQ ID NO: 463 - protein, 109 aa, synthetic construct
  REGION 1..109: note = synthetic
MKHHHHHHVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVID

SEQ ID NO: 464 - protein, 11 aa, synthetic construct
  REGION 1..11: note = synthetic
VSGWRLFKKIS

SEQ ID NO: 465 - protein, 10 aa, synthetic construct
  REGION 1..10: note = synthetic
VSGWRLFKKI

SEQ ID NO: 466 - protein, 15 aa, synthetic construct
  REGION 1..15: note = synthetic
WNGNKIIDERLITPD

SEQ ID NO: 467 - protein, 13 aa, synthetic construct
  REGION 1..13: note = synthetic
KKITTTGTLWNGR

SEQ ID NO: 468 - protein, 12 aa, synthetic construct
  REGION 1..12: note = synthetic
RPYEGIAVFDGK

SEQ ID NO: 469 - protein, 24 aa, synthetic construct
  REGION 1..24: note = synthetic
GKMLFRVTIWKVSVSGWRLFKKIS

SEQ ID NO: 470 - protein, 22 aa, synthetic construct
  REGION 1..22: note = synthetic
GKMLFRVTIWKVSGWRLFKKIS

SEQ ID NO: 471 - protein, 26 aa, synthetic construct
  REGION 1..26: note = synthetic
GSMKFRVTINSWKVSVSGWRLFKKIS

SEQ ID NO: 472 - protein, 24 aa, synthetic construct
  REGION 1..24: note = synthetic
GSMKFRVTINSWKVSGWRLFKKIS

SEQ ID NO: 473 - protein, 26 aa, synthetic construct
  REGION 1..26: note = synthetic
GSMKFRVTINSWKNVTGYRLFKKISN

SEQ ID NO: 474 - protein, 24 aa, synthetic construct
  REGION 1..24: note = synthetic
GSMKFRVTINSWKVTGYRLFEKIS

SEQ ID NO: 475 - protein, 24 aa, synthetic construct
  REGION 1..24: note = synthetic
GSMKFRVTIWKVSVSGWRLFKKIS

SEQ ID NO: 476 - protein, 22 aa, synthetic construct
  REGION 1..22: note = synthetic
GSMKFRVTIWKVSGWRLFKKIS

SEQ ID NO: 477 - protein, 26 aa, synthetic construct
  REGION 1..26: note = synthetic
GRMLFRVTINSWKVSVSGWRLFKKIS

SEQ ID NO: 478 - protein, 24 aa, synthetic construct
  REGION 1..24: note = synthetic
GRMLFRVTINSWKVSGWRLFKKIS

SEQ ID NO: 479 - protein, 24 aa, synthetic construct
  REGION 1..24: note = synthetic
GRMLFRVTIWKVSVSGWRLFKKIS

SEQ ID NO: 480 - protein, 22 aa, synthetic construct
  REGION 1..22: note = synthetic
GRMLFRVTIWKVSGWRLFKKIS

SEQ ID NO: 481 - protein, 24 aa, synthetic construct
  REGION 1..24: note = synthetic
GSMLFRVTINSVSVSGWRLFKKIS

SEQ ID NO: 482 - protein, 22 aa, synthetic construct
  REGION 1..22: note = synthetic
GSMLFKVTINSVSGWRLFKKIS

SEQ ID NO: 483 - protein, 22 aa, synthetic construct
  REGION 1..22: note = synthetic
GSMLFQVTINSVSGWRLFKKIS

SEQ ID NO: 484 - protein, 22 aa, synthetic construct
  REGION 1..22: note = synthetic
GSMLFEVTINSVSGWRLFKKIS

SEQ ID NO: 485 - protein, 22 aa, synthetic construct
  REGION 1..22: note = synthetic
GSMLFNVTINSVSGWRLFKKIS

SEQ ID NO: 486 - protein, 21 aa, synthetic construct
  REGION 1..21: note = synthetic
GRPYEGIAVFDGKKITTTGTL

SEQ ID NO: 487 - protein, 24 aa, synthetic construct
  REGION 1..24: note = synthetic
GSMKFRVTINSWKVTGYRLFEKES

SEQ ID NO: 488 - protein, 24 aa, synthetic construct
  REGION 1..24: note = synthetic
GSMKFRVTINSWKVEGYRLFEKIS

SEQ ID NO: 489 - protein, 24 aa, synthetic construct
  REGION 1..24: note = synthetic
KKITTTGTLWNGNKIIDERLITPD

SEQ ID NO: 490 - protein, 26 aa, synthetic construct
  REGION 1..26: note = synthetic
WNGNKIIDERLITPDGSMLFRVTINS

SEQ ID NO: 491 - protein, 13 aa, synthetic construct
  REGION 1..13: note = synthetic
GKMLFRVTIQKWK

SEQ ID NO: 492 - protein, 13 aa, synthetic construct
  REGION 1..13: note = synthetic
GKMLFRVTIGKWK

SEQ ID NO: 493 - protein, 13 aa, synthetic construct
  REGION 1..13: note = synthetic
GKMLFRVTIGRWK

SEQ ID NO: 494 - protein, 13 aa, synthetic construct
  REGION 1..13: note = synthetic
GKMLFRVTIGNWK

SEQ ID NO: 495 - protein, 13 aa, synthetic construct
  REGION 1..13: note = synthetic
GKMLFRVTIQNWK

SEQ ID NO: 496 - protein, 13 aa, synthetic construct
  REGION 1..13: note = synthetic
GKMLFRVTIDKWK

SEQ ID NO: 497 - protein, 13 aa, synthetic construct
  REGION 1..13: note = synthetic
GKMLFRVTIEKWK

SEQ ID NO: 498 - protein, 13 aa, synthetic construct
  REGION 1..13: note = synthetic
EKMLFRVTIESWK

SEQ ID NO: 499 - protein, 13 aa, synthetic construct
  REGION 1..13: note = synthetic
EKLLFRVTIESWK

SEQ ID NO: 500 - protein, 13 aa, synthetic construct
  REGION 1..13: note = synthetic
EKLLFRVTIESYK

SEQ ID NO: 501 - protein, 13 aa, synthetic construct
  REGION 1..13: note = synthetic
GKMLFRVTIERWK

SEQ ID NO: 502 - protein, 13 aa, synthetic construct
  REGION 1..13: note = synthetic
GKMLFRVTIDRWK

SEQ ID NO: 503 - protein, 13 aa, synthetic construct
  REGION 1..13: note = synthetic
DKMLFRVTIQKWK

SEQ ID NO: 504 - protein, 13 aa, synthetic construct
  REGION 1..13: note = synthetic
DKMLFRVTIGKWK

SEQ ID NO: 505 - protein, 13 aa, synthetic construct
  REGION 1..13: note = synthetic
DKMLFRVTIGRWK

SEQ ID NO: 506 - protein, 13 aa, synthetic construct
  REGION 1..13: note = synthetic
DKMLFRVTIGNWK

SEQ ID NO: 507 - protein, 13 aa, synthetic construct
  REGION 1..13: note = synthetic
DKMLFRVTIQNWK

SEQ ID NO: 508 - protein, 13 aa, synthetic construct
  REGION 1..13: note = synthetic
DKMLFRVTIDKWK

SEQ ID NO: 509 - protein, 13 aa, synthetic construct
  REGION 1..13: note = synthetic
DKMLFRVTIEKWK

SEQ ID NO: 510 - protein, 13 aa, synthetic construct
  REGION 1..13: note = synthetic
DKMLFRVTIERWK

SEQ ID NO: 511 - protein, 13 aa, synthetic construct
  REGION 1..13: note = synthetic
DKMLFRVTIDRWK

SEQ ID NO: 512 - protein, 35 aa, synthetic construct
  REGION 1..35: note = synthetic
RPYEGIAVFDGKKITVTGTLWNGNKIIDERLITPD

SEQ ID NO: 513 - protein, 13 aa, synthetic construct
  REGION 1..13: note = synthetic
EKMLFRVTIQKWK

SEQ ID NO: 514 - protein, 13 aa, synthetic construct
  REGION 1..13: note = synthetic
EKMLFRVTIGKWK

SEQ ID NO: 515 - protein, 13 aa, synthetic construct
  REGION 1..13: note = synthetic
EKMLFRVTIGRWK

SEQ ID NO: 516 - protein, 22 aa, synthetic construct
  REGION 1..22: note = synthetic
DKMLFTVTIQKVSGWRLFKKIS

SEQ ID NO: 517 - protein, 22 aa, synthetic construct
  REGION 1..22: note = synthetic
DKLLFTVTIEKVSGWRLFKKIS

SEQ ID NO: 518 - protein, 24 aa, synthetic construct
  REGION 1..24: note = synthetic
DKLLFTVTIEKWKVSGWRLFKKIS

SEQ ID NO: 519 - protein, 24 aa, synthetic construct
  REGION 1..24: note = synthetic
DKLLFTVTIEKYKVSGWRLFKKIS

SEQ ID NO: 520 - protein, 26 aa, synthetic construct
  REGION 1..26: note = synthetic
DKLLFTVTIEKYKVSVSGWRLFKKIS

SEQ ID NO: 521 - protein, 22 aa, synthetic construct
  REGION 1..22: note = synthetic
KKMLFRVTIQKVSGWRLFKKIS

SEQ ID NO: 522 - protein, 26 aa, synthetic construct
  REGION 1..26: note = synthetic
KKMLFRVTIQKWKVSVSGWRLFKKIS

SEQ ID NO: 523 - protein, 24 aa, synthetic construct
  REGION 1..24: note = synthetic
KKMLFRVTIQKWKVSGWRLFKKIS

SEQ ID NO: 524 - protein, 22 aa, synthetic construct
  REGION 1..22: note = synthetic
DKLLFTVTIGKVSGWRLFKKIS

SEQ ID NO: 525 - protein, 24 aa, synthetic construct
  REGION 1..24: note = synthetic
DKLLFTVTIGKYKVSGWRLFKKIS

SEQ ID NO: 526 - protein, 26 aa, synthetic construct
  REGION 1..26: note = synthetic
DKLLFTVTIGKYKVSVSGWRLFKKIS

SEQ ID NO: 527 - protein, 26 aa, synthetic construct
  REGION 1..26: note = synthetic
DKLLFTVTIGKWKVSVSGWRLFKKIS

SEQ ID NO: 528 - protein, 22 aa, synthetic construct
  REGION 1..22: note = synthetic
DKLLFTVTIQKVSGWRLFKKIS

SEQ ID NO: 529 - protein, 22 aa, synthetic construct
  REGION 1..22: note = synthetic
KKMLFTVTIQKVSGWRLFKKIS

SEQ ID NO: 530 - protein, 22 aa, synthetic construct
  REGION 1..22: note = synthetic
KKLLFRVTIQKVSGWRLFKKIS

SEQ ID NO: 531 - protein, 21 aa, synthetic construct
  REGION 1..21: note = synthetic
DKLLFTVTIEKVSGWRLFKKI

SEQ ID NO: 532 - protein, 25 aa, synthetic construct
  REGION 1..25: note = synthetic
DKLLFTVTIEKYKVSVSGWRLFKKI

SEQ ID NO: 533 - protein, 22 aa, synthetic construct
  REGION 1..22: note = synthetic
DRLLFTVTIERVSGWRLFKKIS

SEQ ID NO: 534 - protein, 22 aa, synthetic construct
  REGION 1..22: note = synthetic
EKLLFTVTIEKVSGWRLFKKIS

SEQ ID NO: 535 - protein, 22 aa, synthetic construct
  REGION 1..22: note = synthetic
KKLLFTVTIGKVSGWRLFKKIS

SEQ ID NO: 536 - protein, 24 aa, synthetic construct
  REGION 1..24: note = synthetic
GSMRFRVTINSWRVTGYRLFERES

SEQ ID NO: 537 - protein, 22 aa, synthetic construct
  REGION 1..22: note = synthetic
GSMKFRVTINSVTGYRLFEKES

SEQ ID NO: 538 - protein, 17 aa, synthetic construct
  REGION 1..17: note = synthetic
KKITTTGTLWNGNKIID

SEQ ID NO: 539 - protein, 29 aa, synthetic construct
  REGION 1..29: note = synthetic
ERLITPDGSMLFRVTINSVSGWRLFKKIS

SEQ ID NO: 540 - protein, 58 aa, synthetic construct
  REGION 1..58: note = synthetic
GRPYEGIAVDFGKKITTTGTLWNGNKIIDERLITPDGSMLFRVTINSVSGWRLFKKIS

SEQ ID NO: 541 - protein, 68 aa, synthetic construct
  REGION 1..68: note = synthetic
GVTPNKLNYFGRPYEGIAVDFGKKITTTGTLWNGNKIIDERLITPDGSMLFRVTINSVSGWRLFKKIS

SEQ ID NO: 542 - protein, 13 aa, synthetic construct
  REGION 1..13: note = synthetic
EKMLFRVTIGNWK

SEQ ID NO: 543 - protein, 13 aa, synthetic construct
  REGION 1..13: note = synthetic
EKMLFRVTIQNWK

SEQ ID NO: 544 - protein, 13 aa, synthetic construct
  REGION 1..13: note = synthetic
EKMLFRVTIDKWK

SEQ ID NO: 545 - protein, 13 aa, synthetic construct
  REGION 1..13: note = synthetic
EKMLFRVTIEKWK

SEQ ID NO: 546 - protein, 13 aa, synthetic construct
  REGION 1..13: note = synthetic
EKMLFRVTIERWK

SEQ ID NO: 547 - protein, 13 aa, synthetic construct
  REGION 1..13: note = synthetic
EKMLFRVTIDRWK

SEQ ID NO: 548 - protein, 13 aa, synthetic construct
  REGION 1..13: note = synthetic
KKMLFRVTIGKWK

SEQ ID NO: 549 - protein, 13 aa, synthetic construct
  REGION 1..13: note = synthetic
KKMLFRVTIGRWK

SEQ ID NO: 550 - protein, 13 aa, synthetic construct
  REGION 1..13: note = synthetic
KKMLFRVTIGNWK

SEQ ID NO: 551 - protein, 13 aa, synthetic construct
  REGION 1..13: note = synthetic
KKMLFRVTIQNWK

SEQ ID NO: 552 - protein, 13 aa, synthetic construct
  REGION 1..13: note = synthetic
KKMLFRVTIDKWK

SEQ ID NO: 553 - protein, 13 aa, synthetic construct
  REGION 1..13: note = synthetic
KKMLFRVTIEKWK

SEQ ID NO: 554 - protein, 13 aa, synthetic construct
  REGION 1..13: note = synthetic
KKMLFRVTIERWK

SEQ ID NO: 555 - protein, 13 aa, synthetic construct
  REGION 1..13: note = synthetic
KKMLFRVTIDRWK

SEQ ID NO: 556 - protein, 13 aa, synthetic construct
  REGION 1..13: note = synthetic
RKMLFRVTIQKWK

SEQ ID NO: 557 - protein, 13 aa, synthetic construct
  REGION 1..13: note = synthetic
RKMLFRVTIGKWK

SEQ ID NO: 558 - protein, 13 aa, synthetic construct
  REGION 1..13: note = synthetic
RKMLFRVTIGRWK

SEQ ID NO: 559 - protein, 13 aa, synthetic construct
  REGION 1..13: note = synthetic
RKMLFRVTIGNWK

SEQ ID NO: 560 - protein, 13 aa, synthetic construct
  REGION 1..13: note = synthetic
RKMLFRVTIQNWK

SEQ ID NO: 561 - protein, 13 aa, synthetic construct
  REGION 1..13: note = synthetic
RKMLFRVTIDKWK

SEQ ID NO: 562 - protein, 13 aa, synthetic construct
  REGION 1..13: note = synthetic
RKMLFRVTIEKWK

SEQ ID NO: 563 - protein, 13 aa, synthetic construct
  REGION 1..13: note = synthetic
RKMLFRVTIERWK

SEQ ID NO: 564 - protein, 13 aa, synthetic construct
  REGION 1..13: note = synthetic
RKMLFRVTIDRWK

SEQ ID NO: 565 - protein, 13 aa, synthetic construct
  REGION 1..13: note = synthetic
EQMLFRVTINSWK

SEQ ID NO: 566 - protein, 13 aa, synthetic construct
  REGION 1..13: note = synthetic
SRMLFRVTINSWK

SEQ ID NO: 567 - protein, 13 aa, synthetic construct
  REGION 1..13: note = synthetic
GEMLFRVTINSWK

SEQ ID NO: 568 - protein, 13 aa, synthetic construct
  REGION 1..13: note = synthetic
GKMKFRVTINSWK

SEQ ID NO: 569 - protein, 13 aa, synthetic construct
  REGION 1..13: note = synthetic
GKMLFRVKINSWK

SEQ ID NO: 570 - protein, 13 aa, synthetic construct
  REGION 1..13: note = synthetic
GKMLFRVRINSWK

SEQ ID NO: 571 - protein, 13 aa, synthetic construct
  REGION 1..13: note = synthetic
GKMLFRVDINSWK

SEQ ID NO: 572 - protein, 13 aa, synthetic construct
  REGION 1..13: note = synthetic
GKMLFRVTIDSWK

SEQ ID NO: 573 - protein, 13 aa, synthetic construct
  REGION 1..13: note = synthetic
EKMLFKVTIQKWK

SEQ ID NO: 574 - protein, 13 aa, synthetic construct
  REGION 1..13: note = synthetic
EKMLFTVTIQKWK

SEQ ID NO: 575 - protein, 13 aa, synthetic construct
  REGION 1..13: note = synthetic
EKMLFKVTIDKWK

SEQ ID NO: 576 - protein, 13 aa, synthetic construct
  REGION 1..13: note = synthetic
EKMLFTVTIDKWK

SEQ ID NO: 577 - protein, 13 aa, synthetic construct
  REGION 1..13: note = synthetic
EKMLFKVTIGRWK

SEQ ID NO: 578 - protein, 13 aa, synthetic construct
  REGION 1..13: note = synthetic
DKMLFKVTIQKWK

SEQ ID NO: 579 - protein, 13 aa, synthetic construct
  REGION 1..13: note = synthetic
DKMLFTVTIQKWK

SEQ ID NO: 580 - protein, 13 aa, synthetic construct
  REGION 1..13: note = synthetic
DKMLFKVTIDKWK

SEQ ID NO: 581 - protein, 13 aa, synthetic construct
  REGION 1..13: note = synthetic
DKMLFTVTIDKWK

SEQ ID NO: 582 - protein, 13 aa, synthetic construct
  REGION 1..13: note = synthetic
GKMLFKVTIEKWK

SEQ ID NO: 583 - protein, 13 aa, synthetic construct
  REGION 1..13: note = synthetic
GKMLFTVTIEKWK

SEQ ID NO: 584 - protein, 13 aa, synthetic construct
  REGION 1..13: note = synthetic
DKMLFKVTIGKWK

SEQ ID NO: 585 - protein, 13 aa, synthetic construct
  REGION 1..13: note = synthetic
DKMLFTVTIGKWK

SEQ ID NO: 586 - protein, 13 aa, synthetic construct
  REGION 1..13: note = synthetic
DKMLFKVTIGNWK

SEQ ID NO: 587 - protein, 13 aa, synthetic construct
  REGION 1..13: note = synthetic
DKMLFKVTIQNWK

SEQ ID NO: 588 - protein, 13 aa, synthetic construct
  REGION 1..13: note = synthetic
GKMLFKVTINKWK

SEQ ID NO: 589 - protein, 13 aa, synthetic construct
  REGION 1..13: note = synthetic
GKMLFTVTINKWK

SEQ ID NO: 590 - protein, 13 aa, synthetic construct
  REGION 1..13: note = synthetic
DKMLFKVTIEKWK

SEQ ID NO: 591 - protein, 13 aa, synthetic construct
  REGION 1..13: note = synthetic
DKMLFTVTIEKWK

SEQ ID NO: 592 - protein, 13 aa, synthetic construct
  REGION 1..13: note = synthetic
DKLLFKVTIGKWK

SEQ ID NO: 593 - protein, 13 aa, synthetic construct
  REGION 1..13: note = synthetic
DKMLFTVTINKWK

SEQ ID NO: 594 - protein, 13 aa, synthetic construct
  REGION 1..13: note = synthetic
DKLLFTVTIQKWK

SEQ ID NO: 595 - protein, 13 aa, synthetic construct
  REGION 1..13: note = synthetic
DKLLFTVTIQKYK

SEQ ID NO: 596 - protein, 13 aa, synthetic construct
  REGION 1..13: note = synthetic
DKLLFTVTIEKWK

SEQ ID NO: 597 - protein, 13 aa, synthetic construct
  REGION 1..13: note = synthetic
DKLLFTVTIGKWK

SEQ ID NO: 598 - protein, 13 aa, synthetic construct
  REGION 1..13: note = synthetic
DKLLFTVTIGKYK

SEQ ID NO: 599 - protein, 13 aa, synthetic construct
  REGION 1..13: note = synthetic
DKLLFTVTINKWK

SEQ ID NO: 600 - protein, 13 aa, synthetic construct
  REGION 1..13: note = synthetic
DKLLFTVTINKYK

SEQ ID NO: 601 - protein, 11 aa, synthetic construct
  REGION 1..11: note = synthetic
GKMLFRVTINS

SEQ ID NO: 602 - protein, 11 aa, synthetic construct
  REGION 1..11: note = synthetic
DKMLFTVTIQK

SEQ ID NO: 603 - protein, 11 aa, synthetic construct
  REGION 1..11: note = synthetic
DKMLFKVTIQK

SEQ ID NO: 604 - protein, 11 aa, synthetic construct
  REGION 1..11: note = synthetic
DKLLFTVTIGK

SEQ ID NO: 605 - protein, 11 aa, synthetic construct
  REGION 1..11: note = synthetic
DKMLFTVTIGK

SEQ ID NO: 606 - protein, 11 aa, synthetic construct
  REGION 1..11: note = synthetic
DKMLFTVTIEK

SEQ ID NO: 607 - protein, 11 aa, synthetic construct
  REGION 1..11: note = synthetic
DKLLFTVTIEK

SEQ ID NO: 608 - protein, 13 aa, synthetic construct
  REGION 1..13: note = synthetic
DKMLFRVTINSWK

SEQ ID NO: 609 - protein, 13 aa, synthetic construct
  REGION 1..13: note = synthetic
EKMLFRVTINSWK

SEQ ID NO: 610 - protein, 13 aa, synthetic construct
  REGION 1..13: note = synthetic
RKMLFRVTINSWK

SEQ ID NO: 611 - protein, 13 aa, synthetic construct
  REGION 1..13: note = synthetic
KKMLFRVTINSWK

SEQ ID NO: 612 - protein, 13 aa, synthetic construct
  REGION 1..13: note = synthetic
HKMLFRVTINSWK

SEQ ID NO: 613 - protein, 13 aa, synthetic construct
  REGION 1..13: note = synthetic
GLMLFRVTINSWK

SEQ ID NO: 614 - protein, 13 aa, synthetic construct
  REGION 1..13: note = synthetic
GQMLFRVTINSWK

SEQ ID NO: 615 - protein, 13 aa, synthetic construct
  REGION 1..13: note = synthetic
GTMLFRVTINSWK

SEQ ID NO: 616 - protein, 13 aa, synthetic construct
  REGION 1..13: note = synthetic
GKLLFRVTINSWK

SEQ ID NO: 617 - protein, 13 aa, synthetic construct
  REGION 1..13: note = synthetic
GKMLFKVTINSWK

SEQ ID NO: 618 - protein, 13 aa, synthetic construct
  REGION 1..13: note = synthetic
GKMLFRVTIQSWK

SEQ ID NO: 619 - protein, 13 aa, synthetic construct
  REGION 1..13: note = synthetic
GKMLFRVTIDSWK

SEQ ID NO: 620 - protein, 13 aa, synthetic construct
  REGION 1..13: note = synthetic
GKMLFRVTIGSWK

SEQ ID NO: 621 - protein, 13 aa, synthetic construct
  REGION 1..13: note = synthetic
GKMLFRVTINTWK

SEQ ID NO: 622 - protein, 13 aa, synthetic construct
  REGION 1..13: note = synthetic
GKMLFRVTINNWK

SEQ ID NO: 623 - protein, 13 aa, synthetic construct
  REGION 1..13: note = synthetic
GKMLFRVTINQWK

SEQ ID NO: 624 - protein, 13 aa, synthetic construct
  REGION 1..13: note = synthetic
GKMLFRVTINPWK

SEQ ID NO: 625 - protein, 13 aa, synthetic construct
  REGION 1..13: note = synthetic
GKMLFRVTINKWK

SEQ ID NO: 626 - protein, 13 aa, synthetic construct
  REGION 1..13: note = synthetic
GKMLFRVTINSWQ

SEQ ID NO: 627 - protein, 13 aa, synthetic construct
  REGION 1..13: note = synthetic
GKMLFRVTINSWN

SEQ ID NO: 628 - protein, 13 aa, synthetic construct
  REGION 1..13: note = synthetic
GKMLFRVTINSWT

SEQ ID NO: 629 - protein, 13 aa, synthetic construct
  REGION 1..13: note = synthetic
GKMLFRVTINSWH

SEQ ID NO: 630 - protein, 13 aa, synthetic construct
  REGION 1..13: note = synthetic
GKMLFRVTINSWP

SEQ ID NO: 631 - protein, 13 aa, synthetic construct
  REGION 1..13: note = synthetic
GKMLFRVTINSWR

SEQ ID NO: 632 - protein, 13 aa, synthetic construct
  REGION 1..13: note = synthetic
GKMKFRVTIDSWK

SEQ ID NO: 633 - protein, 13 aa, synthetic construct
  REGION 1..13: note = synthetic
GKMLFRVEINSWK

SEQ ID NO: 634 - protein, 13 aa, synthetic construct
  REGION 1..13: note = synthetic
GKMLFRVQINSWK

SEQ ID NO: 635 - protein, 13 aa, synthetic construct
  REGION 1..13: note = synthetic
GKMKFRVKINSWK

SEQ ID NO: 636 - protein, 13 aa, synthetic construct
  REGION 1..13: note = synthetic
GKMKFRVRINSWK

SEQ ID NO: 637 - protein, 13 aa, synthetic construct
  REGION 1..13: note = synthetic
GKMKFRVEINSWK

SEQ ID NO: 638 - protein, 13 aa, synthetic construct
  REGION 1..13: note = synthetic
GKMKFRVDINSWK

SEQ ID NO: 639 - protein, 13 aa, synthetic construct
  REGION 1..13: note = synthetic
GKMKFRVQINSWK

SEQ ID NO: 640 - protein, 13 aa, synthetic construct
  REGION 1..13: note = synthetic
GKMKFRVNINSWK

SEQ ID NO: 641 - protein, 13 aa, synthetic construct
  REGION 1..13: note = synthetic
GKMKFRVSINSWK

SEQ ID NO: 642 - protein, 13 aa, synthetic construct
  REGION 1..13: note = synthetic
GKMLFRVNINSWK

SEQ ID NO: 643 - protein, 13 aa, synthetic construct
  REGION 1..13: note = synthetic
GKMLFRVSINSWK

SEQ ID NO: 644 - protein, 13 aa, synthetic construct
  REGION 1..13: note = synthetic
GKMLFRVWINSWK

SEQ ID NO: 645 - protein, 13 aa, synthetic construct
  REGION 1..13: note = synthetic
GKMSFRVTINSWK

SEQ ID NO: 646 - protein, 13 aa, synthetic construct
  REGION 1..13: note = synthetic
GKMWFRVTINSWK

SEQ ID NO: 647 - protein, 13 aa, synthetic construct
  REGION 1..13: note = synthetic
GKMNFRVTINSWK

SEQ ID NO: 648 - protein, 13 aa, synthetic construct
  REGION 1..13: note = synthetic
GSMLFRVTINSYK

SEQ ID NO: 649 - protein, 13 aa, synthetic construct
  REGION 1..13: note = synthetic
GKMLFRVTINSYK

SEQ ID NO: 650 - protein, 13 aa, synthetic construct
  REGION 1..13: note = synthetic
GKMLFRVTIKSWK

SEQ ID NO: 651 - protein, 13 aa, synthetic construct
  REGION 1..13: note = synthetic
GKMLFRVTIESWK

SEQ ID NO: 652 - protein, 13 aa, synthetic construct
  REGION 1..13: note = synthetic
GKMKFRVTIQSWK

SEQ ID NO: 653 - protein, 13 aa, synthetic construct
  REGION 1..13: note = synthetic
GKMKFRVTIESWK

SEQ ID NO: 654 - protein, 13 aa, synthetic construct
  REGION 1..13: note = synthetic
GKMKFRVTIKSWK

SEQ ID NO: 655 - protein, 13 aa, synthetic construct
  REGION 1..13: note = synthetic
GKMKFRVTIRSWK

SEQ ID NO: 656 - protein, 13 aa, synthetic construct
  REGION 1..13: note = synthetic
RLMLFRVTINSWK

SEQ ID NO: 657 - protein, 13 aa, synthetic construct
  REGION 1..13: note = synthetic
RQMLFRVTINSWK

SEQ ID NO: 658 - protein, 13 aa, synthetic construct
  REGION 1..13: note = synthetic
KLMLFRVTINSWK

SEQ ID NO: 659 - protein, 13 aa, synthetic construct
  REGION 1..13: note = synthetic
KQMLFRVTINSWK

SEQ ID NO: 660 - protein, 13 aa, synthetic construct
  REGION 1..13: note = synthetic
ELMLFRVTINSWK

SEQ ID NO: 661 - protein, 13 aa, synthetic construct
  REGION 1..13: note = synthetic
DLMLFRVTINSWK

SEQ ID NO: 662 - protein, 13 aa, synthetic construct
  REGION 1..13: note = synthetic
DQMLFRVTINSWK

SEQ ID NO: 663 - protein, 13 aa, synthetic construct
  REGION 1..13: note = synthetic
DKMLFRVTINSWK

SEQ ID NO: 664 - protein, 13 aa, synthetic construct
  REGION 1..13: note = synthetic
EKMLFRVTINSWK

SEQ ID NO: 665 - protein, 13 aa, synthetic construct
  REGION 1..13: note = synthetic
RKMLFRVTINSWK

SEQ ID NO: 666 - protein, 13 aa, synthetic construct
  REGION 1..13: note = synthetic
KKMLFRVTINSWK

SEQ ID NO: 667 - protein, 13 aa, synthetic construct
  REGION 1..13: note = synthetic
GKMLFRVTIGSWK

SEQ ID NO: 668 - protein, 13 aa, synthetic construct
  REGION 1..13: note = synthetic
GKMLFRVTINKWK

SEQ ID NO: 669 - protein, 13 aa, synthetic construct
  REGION 1..13: note = synthetic
GKMLFRVTISKWK

SEQ ID NO: 670 - protein, 13 aa, synthetic construct
  REGION 1..13: note = synthetic
GKMLFRVTIQKWK

SEQ ID NO: 671 - protein, 13 aa, synthetic construct
  REGION 1..13: note = synthetic
GKMLFRVTITKWK

SEQ ID NO: 672 - protein, 13 aa, synthetic construct
  REGION 1..13: note = synthetic
GKMLFRVTIKKWK

SEQ ID NO: 673 - protein, 13 aa, synthetic construct
  REGION 1..13: note = synthetic
GKMLFKVTINSWK

SEQ ID NO: 674 - protein, 13 aa, synthetic construct
  REGION 1..13: note = synthetic
RLMLFRVTIGKWK

SEQ ID NO: 675 - protein, 13 aa, synthetic construct
  REGION 1..13: note = synthetic
GKMLFRVTINRWK

SEQ ID NO: 676 - protein, 13 aa, synthetic construct
  REGION 1..13: note = synthetic
EKMLFTVTIGKWK

SEQ ID NO: 677 - protein, 13 aa, synthetic construct
  REGION 1..13: note = synthetic
EKLLFTVTIGKWK

SEQ ID NO: 678 - protein, 13 aa, synthetic construct
  REGION 1..13: note = synthetic
EKMLFTVTIGRWK

SEQ ID NO: 679 - protein, 13 aa, synthetic construct
  REGION 1..13: note = synthetic
EKMLFTVTIEKWK

SEQ ID NO: 680 - protein, 13 aa, synthetic construct
  REGION 1..13: note = synthetic
DKMLFRVTIESWK

SEQ ID NO: 681 - protein, 13 aa, synthetic construct
  REGION 1..13: note = synthetic
EKLLFRVTIGKYK

SEQ ID NO: 682 - protein, 13 aa, synthetic construct
  REGION 1..13: note = synthetic
DKLLFKVTIQKWK

SEQ ID NO: 683 - protein, 13 aa, synthetic construct
  REGION 1..13: note = synthetic
DKLLFKVTIQKYK

SEQ ID NO: 684 - protein, 13 aa, synthetic construct
  REGION 1..13: note = synthetic
DKLLFKVTIGKYK

SEQ ID NO: 685 - protein, 13 aa, synthetic construct
  REGION 1..13: note = synthetic
DKLLFKVTIEKWK

SEQ ID NO: 686 - protein, 13 aa, synthetic construct
  REGION 1..13: note = synthetic
DKLLFKVTIEKYK

SEQ ID NO: 687 - protein, 13 aa, synthetic construct
  REGION 1..13: note = synthetic
KKLLFRVTIQKWK

SEQ ID NO: 688 - protein, 13 aa, synthetic construct
  REGION 1..13: note = synthetic
DRMLFRVTIQRWR

SEQ ID NO: 689 - protein, 13 aa, synthetic construct
  REGION 1..13: note = synthetic
ERMLFRVTIGRWR

SEQ ID NO: 690 - protein, 13 aa, synthetic construct
  REGION 1..13: note = synthetic
GRMLFRVTINRWR

SEQ ID NO: 691 - protein, 13 aa, synthetic construct
  REGION 1..13: note = synthetic
DRMLFRVTIERWR

SEQ ID NO: 692 - protein, 13 aa, synthetic construct
  REGION 1..13: note = synthetic
DKMLFKVTIQKYK

SEQ ID NO: 693 - protein, 13 aa, synthetic construct
  REGION 1..13: note = synthetic
DKMLFRVTINKWK

SEQ ID NO: 694 - protein, 13 aa, synthetic construct
  REGION 1..13: note = synthetic
DKMLFKVTIEKYK

SEQ ID NO: 695 - protein, 13 aa, synthetic construct
  REGION 1..13: note = synthetic
DKMLFKVTINKWK

SEQ ID NO: 696 - protein, 13 aa, synthetic construct
  REGION 1..13: note = synthetic
GRMLFRVTINSWR

SEQ ID NO: 697 - protein, 13 aa, synthetic construct
  REGION 1..13: note = synthetic
GRLLFVVVIERYR

SEQ ID NO: 698 - protein, 12 aa, synthetic construct
  REGION 1..12: note = synthetic
VSGWRLFRRISC

SEQ ID NO: 699 - protein, 14 aa, synthetic construct
  REGION 1..14: note = synthetic
GRMLFRVTINSWRC

SEQ ID NO: 700 - protein, 14 aa, synthetic construct
  REGION 1..14: note = synthetic
GRLLFTVTIERYRC

SEQ ID NO: 701 - protein, 13 aa, synthetic construct
  REGION 1..13: note = synthetic
GKLLFVVVIEKYK

SEQ ID NO: 702 - protein, 21 aa, synthetic construct
  REGION 1..21: note = synthetic
GKLLFVTIEKVSGWRLFKKIS

SEQ ID NO: 703 - protein, 170 aa, synthetic construct
  REGION 1..170: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSMLFRVTINSVSGWRLFKKIS

SEQ ID NO: 704 - protein, 170 aa, synthetic construct
  REGION 1..170: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTLWNGNKIIDERLITPDGSMLFRVTINSVTGYRLFEEIL

SEQ ID NO: 705 - protein, 102 aa, synthetic construct
  REGION 1..102: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVID

SEQ ID NO: 706 - protein, 124 aa, synthetic construct
  REGION 1..124: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDG

SEQ ID NO: 707 - protein, 133 aa, synthetic construct
  REGION 1..133: note = synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITTTGTL

SEQ ID NO: 708 - protein, 148 aa, synthetic construct; identical to SEQ ID NO: 294
  REGION 1..148: note = synthetic

SEQ ID NO: 709 - protein, 68 aa, synthetic construct
  REGION 1..68: note = synthetic
GVTPNKLNYFGRPYEGIAVFDGKKITTTGTLWNGNKIIDERLITPDGSMLFRVTINSVSGWRLFKKIS

SEQ ID NO: 710 - protein, 46 aa, synthetic construct
  REGION 1..46: note = synthetic
KKITTTGTLWNGNKIIDERLITPDGSMLFRVTINSVSGWRLFKKIS

SEQ ID NO: 711 - protein, 37 aa, synthetic construct
  REGION 1..37: note = synthetic
WNGNKIIDERLITPDGSMLFRVTINSVSGWRLFKKIS

SEQ ID NO: 712 - protein, 22 aa, synthetic construct
  REGION 1..22: note = synthetic
GSMLFRVTINSVSGWRLFKKIS

SEQ ID NO: 713 - protein, 68 aa, synthetic construct
  REGION 1..68: note = synthetic
GVTPNKLNYFGRPYEGIAVFDGKKITTTGTLWNGNKIIDERLITPDGSMLFRVTINSVTGYRLFEEIL

SEQ ID NO: 714 - protein, 46 aa, synthetic construct
  REGION 1..46: note = synthetic
KKITTTGTLWNGNKIIDERLITPDGSMLFRVTINSVTGYRLFEEIL

SEQ ID NO: 715 - protein, 37 aa, synthetic construct
  REGION 1..37: note = synthetic
WNGNKIIDERLITPDGSMLFRVTINSVTGYRLFEEIL

SEQ ID NO: 716 - protein, 22 aa, synthetic construct
  REGION 1..22: note = synthetic
GSMLFRVTINSVTGYRLFEEIL

SEQ ID NO: 717 - protein, 22 aa, synthetic construct
  REGION 1..22: note = synthetic
GVTPNKLNYFGRPYEGIAVFDG

SEQ ID NO: 718 - protein, 9 aa, synthetic construct
  REGION 1..9: note = synthetic
KKITTTGTL

SEQ ID NO: 719 - protein, 15 aa, synthetic construct
  REGION 1..15: note = synthetic
WNGNKIIDERLITPD

SEQ ID NO: 720 - protein, 297 aa, synthetic construct
  REGION 1..297: note = synthetic
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 721 - DNA, 582 bp, synthetic construct
  misc_feature 1..582: note = synthetic
  source 1..582: mol_type = other DNA
atgaaacatcaccatcaccatcatgtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcgagttcaggtggtggcgggagcggtggagggagcagcggtggagtttccgtgagcggctggcggctgttcaagaagattagctaa

SEQ ID NO: 722 - protein, 193 aa, synthetic construct
  REGION 1..193: note = synthetic
MKHHHHHHVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALSSSGGGGSGGGSSGGVSVSGWRLFKKIS

SEQ ID NO: 723 - DNA, 696 bp, synthetic construct
  misc_feature 1..696: note = synthetic
  source 1..696: mol_type = other DNA
atgggcaagatgctgttccgagtaaccatcaacagctggaaggggagctccggtggtggcgggagcggaggtggaggctcgagcggtatgacgtataagttaatccttaatggtaaaacattgaaaggcgagacaactactgaagctgttgatgctgctactgcagaaaaagtcttcaaacaatacgctaacgacaacggtgttgacggtgaatggacttacgacgatgcgacgaaaacctttacggtcaccgaaaaaccagaagtgatcgatgcgtctgaattaacaccagccgtgacaacttacaaacttgttattaatggtaaaacattgaaaggcgaaacaactactgaggctgttgatgctgctactgcagagaaggtgttcaaacaatatgcgaatgacaacggtgttgacggtgagtggacttacgacgatgcgactaagacctttacagttactgaaaaaccagaagtgatcgatgcgtctgagttaacaccagccgtgacaacttacaaacttgttattaatggtaaaacattgaaaggcgaaacaactactaaagcagtagacgcagaaactgcggagaaggccttcaaacaatacgctaacgacaacggtgttgatggtgtttggacttatgatgatgccacaaaaacctttacggtaactgagcatcatcaccatcaccactaa

SEQ ID NO: 724 - protein, 231 aa, synthetic construct
  REGION 1..231: note = synthetic
MGKMLFRVTINSWKGSSGGGGSGGGGSSGMTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEHHHHHH

SEQ ID NO: 725 - DNA, 693 bp, synthetic construct
  misc_feature 1..693: note = synthetic
  source 1..693: mol_type = other DNA
atggacaagatgctgttccgagtaaccatcaacaagtggaaggggagctccggtggtggcgggagcggaggtggaggctcgagcggtatgacgtataagttaatccttaatggtaaaacattgaaaggcgagacaactactgaagctgttgatgctgctactgcagaaaaagtcttcaaacaatacgctaacgacaacggtgttgacggtgaatggacttacgacgatgcgacgaaaacctttacggtcaccgaaaaaccagaagtgatcgatgcgtctgaattaacaccagccgtgacaacttacaaacttgttattaatggtaaaacattgaaaggcgaaacaactactgaggctgttgatgctgctactgcagagaaggtgttcaaacaatatgcgaatgacaacggtgttgacggtgagtggacttacgacgatgcgactaagacctttacagttactgaaaaaccagaagtgatcgatgcgtctgagttaacaccagccgtgacaacttacaaacttgttattaatggtaaaacattgaaaggcgaaacaactactaaagcagtagacgcagaaactgcggagaaggccttcaaacaatacgctaacgacaacggtgttgatggtgtttggacttatgatgatgccacaaaaacctttacggtaactgagcatcatcaccatcaccac

SEQ ID NO: 726 - protein, 231 aa, synthetic construct
  REGION 1..231: note = synthetic
MDKMLFRVTINKWKGSSGGGGSGGGGSSGMTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEHHHHHH

SEQ ID NO: 727 - DNA, 693 bp, synthetic construct
  misc_feature 1..693: note = synthetic
  source 1..693: mol_type = other DNA
atggacaagctcctgttcacggtaaccatcgagaagtataaggggagctccggtggtggcgggagcggaggtggaggctcgagcggtatgacgtataagttaatccttaatggtaaaacattgaaaggcgagacaactactgaagctgttgatgctgctactgcagaaaaagtcttcaaacaatacgctaacgacaacggtgttgacggtgaatggacttacgacgatgcgacgaaaacctttacggtcaccgaaaaaccagaagtgatcgatgcgtctgaattaacaccagccgtgacaacttacaaacttgttattaatggtaaaacattgaaaggcgaaacaactactgaggctgttgatgctgctactgcagagaaggtgttcaaacaatatgcgaatgacaacggtgttgacggtgagtggacttacgacgatgcgactaagacctttacagttactgaaaaaccagaagtgatcgatgcgtctgagttaacaccagccgtgacaacttacaaacttgttattaatggtaaaacattgaaaggcgaaacaactactaaagcagtagacgcagaaactgcggagaaggccttcaaacaatacgctaacgacaacggtgttgatggtgtttggacttatgatgatgccacaaaaacctttacggtaactgagcatcatcaccatcaccac

SEQ ID NO: 728 - protein, 231 aa, synthetic construct
  REGION 1..231: note = synthetic
MDKLLFTVTIEKYKGSSGGGGSGGGGSSGMTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEHHHHHH

SEQ ID NO: 729 - DNA, 693 bp, synthetic construct
  misc_feature 1..693: note = synthetic
  source 1..693: mol_type = other DNA
atgaagaagatgctgttccgagtaaccatccagaagtggaaggggagctccggtggtggcgggagcggaggtggaggctcgagcggtatgacgtataagttaatccttaatggtaaaacattgaaaggcgagacaactactgaagctgttgatgctgctactgcagaaaaagtcttcaaacaatacgctaacgacaacggtgttgacggtgaatggacttacgacgatgcgacgaaaacctttacggtcaccgaaaaaccagaagtgatcgatgcgtctgaattaacaccagccgtgacaacttacaaacttgttattaatggtaaaacattgaaaggcgaaacaactactgaggctgttgatgctgctactgcagagaaggtgttcaaacaatatgcgaatgacaacggtgttgacggtgagtggacttacgacgatgcgactaagacctttacagttactgaaaaaccagaagtgatcgatgcgtctgagttaacaccagccgtgacaacttacaaacttgttattaatggtaaaacattgaaaggcgaaacaactactaaagcagtagacgcagaaactgcggagaaggccttcaaacaatacgctaacgacaacggtgttgatggtgtttggacttatgatgatgccacaaaaacctttacggtaactgagcatcatcaccatcaccac

SEQ ID NO: 730 - protein, 231 aa, synthetic construct
  REGION 1..231: note = synthetic
MKKMLFRVTIQKWKGSSGGGGSGGGGSSGMTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEHHHHHH